(19) United States
(12) Patent Application Publication
(10) Pub. No.: US 2023/0174926 A1
(43) Pub. Date: Jun. 8, 2023

(54) BACTERIA ENGINEERED TO TREAT DISORDERS INVOLVING THE CATABOLISM OF LEUCINE

(71) Applicant: Synlogic Operating Company, Inc., Cambridge, MA (US)

(72) Inventor: Ning Li, Winchester, MA (US)

(21) Appl. No.: 17/912,111

(22) PCT Filed: Mar. 17, 2021
(86) PCT No.: PCT/US2021/022676

§ 371 Date: Sep. 16, 2022

Related U.S. Application Data
(60) Provisional application No. 62/990,797, filed on Mar. 17, 2020.

(51) Int. Cl. C12N 1/20; C12N 15/52; A61P 3/00

(52) U.S. Cl. CPC C12N 1/205; A61P 3/00; C12N 15/52

(57) ABSTRACT

The present disclosure provides recombinant bacterial cells that have been engineered with genetic circuitry which allow the recombinant bacterial cells to sense a patient’s internal environment and respond by turning an engineered metabolic pathway on or off. When turned on, the recombinant bacterial cells complete all of the steps in a metabolic pathway to achieve a therapeutic effect in a host subject. These recombinant bacterial cells are designed to drive therapeutic effects throughout the body of a host from a point of origin of the microbiome. Specifically, the present disclosure provides recombinant bacterial cells comprising a heterologous gene encoding an improved leucine catabolism enzyme with higher activity and/or specificity for leucine. The disclosure further provides pharmaceutical compositions comprising the recombinant bacteria, and methods for treating disorders involving the catabolism of leucine using the pharmaceutical compositions disclosed herein.

### RELATED APPLICATIONS

The instant application claims priority to U.S. Provisional Application No. 62/990,797 filed Mar. 17, 2020; the entire contents of which are expressly incorporated by reference herein in their entirety.

### SEQUENCE LISTING

The instant application contains a sequence listing, which has been submitted electronically in ASCII format, hereby incorporated by reference in its entirety. Said ASCII copy, created on Mar. 14, 2021, is named 126046-05520_SL.txt and is 424,938 bytes in size.

### BACKGROUND

The branched chain amino acids (BCAAs), e.g., leucine, play an important role in the metabolism of living organisms. Transamination of branched chain amino acids gives rise to their corresponding branched chain α-keto acids (BCKAs) (α-keto-(β-methylvalerate, α-ketoisocaproate, and α-ketoisovalerate), which undergo further oxidative decarboxylation to produce acyl-CoA derivatives that enter the TCA cycle. Branched chain amino acids provide a nonspecific carbon source of oxidation for production of energy and also act as a precursor for muscle protein synthesis (Monirujjaman and Ferdouse, Advances in Molec. Biol., 2014, Article ID 36976, 6 pages, 2014).

Enzyme deficiencies or mutations which lead to the toxic accumulation of branched chain amino acids and their corresponding alpha-keto acids in the blood, cerebrospinal fluid, and tissues result in the development of metabolic disorders associated with the abnormal catabolism of branched chain amino acids in subjects, such as isovaleric acidemia, propionic acidemia, methylmalonic acidemia, maple syrup urine disease (MSUD), and diabetes ketoacidosis. Clinical manifestations of the disease vary depending on the degree of enzyme deficiency and include neurological dysfunction, seizures and death (Homanics et al. 2009).

Branched chain amino acids, such as leucine, or their corresponding alpha-keto acids, have also been linked to mTor activation (see, for example, Harlan et al., Cell Metabolism, 17:599-606, 2013) which is, in turn, associated with diseases such as cancer, obesity, type 2 diabetes, neurodegeneration, autism, Alzheimer’s disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson’s disease, Huntington’s disease, psoriasis, rheumatoid arthritis, lupus, multiple sclerosis, Leigh’s syndrome, and Friedrich’s ataxia (see Laplante and Sabatini, Cell, 149(2):74-293, 2012).

Currently available treatments for disorders involving the catabolism of branched chain amino acids are inadequate for the long term management of the disorders and have severe limitations (Svkvorak, J. Inherit. Metab. Dis., 32(2):229-246, 2009). A low protein/BCAA-restricted diet, with micronutrient and vitamin supplementation, as necessary, is the widely accepted long-term disease management strategy for many such disorders (Homanics et al., BMC Med. Genet., 7:33, 2006). However, BCAA-intake restrictions can be particularly problematic since branched chain amino acids can only be acquired through diet and are necessary for metabolic activities including protein synthesis and branched-chain fatty acid synthesis (Skvorak, 2009). Thus, even with proper monitoring and patient compliance, branched chain amino acid dietary restrictions result in a high incidence of mental retardation and mortality (Skvorak, 2009; Homanics et al., 2009). A few cases of MSUD have been treated by liver transplantation (Popescu and Dima, Liver Transpl., 1:22-28, 2012). However, the limited availability of donor organs, the costs associated with the transplantation itself, and the undesirable effects associated with continued immunosuppressant therapy limit the practicality of liver transplantation for treatment of disorders involving the catabolism of a branched chain amino acid (Homanics et al., 2012; Popescu and Dima, 2012). Therefore, there is significant unmet need for effective, reliable, and/or long-term treatment for disorders involving the catabolism of branched chain amino acids, e.g., leucine.

### SUMMARY

The present disclosure relates to improved compositions and therapeutic methods for reducing one or more excess branched chain amino acids, e.g., leucine, and/or an accumulated metabolite(s) thereof, for example, by converting the one or more excess branched chain amino acid(s) or accumulated metabolite(s) into alternate by product(s). In certain aspects, the disclosure relates to genetically engineered microorganisms, e.g., bacteria, yeast or viruses, that have been optimized to reduce one or more excess branched chain amino acids, e.g., leucine, and/or an accumulated metabolite(s) thereof, particularly in low-oxygen conditions, such as in the mammalian gut. In certain aspects, the compositions and methods disclosed herein may be used to treat disorders associated with excess branched chain amino acids, e.g., leucine, and/or an accumulated metabolite(s) thereof, e.g., isovaleric acidemia.

Specifically, the present disclosure provides recombinant microorganisms that have been engineered with optimized genetic circuitry, which allows the recombinant microorganism to import and/or metabolize BCAA, e.g., leucine, and/or one or more metabolite(s) thereof at an improved rate. In some embodiments, the engineered microorganism is capable of sensing a patient’s internal environment, e.g., the gut, and responding by turning an engineered metabolic pathway on or off. When turned on, the engineered microorganism, e.g., bacterial, yeast or virus cell, expresses one or more enzymes in a metabolic pathway to achieve a therapeutic effect in a host subject.

In certain aspects, the present disclosure provides engineered bacterial cells, pharmaceutical compositions thereof, and methods of modulating BCAA(s) and/or metabolite(s) thereof and treating diseases associated with the catabolism of branched chain amino acids. Specifically, the engineered bacteria disclosed herein have been modified to comprise optimized gene sequence(s) encoding one or more enzymes involved in branched chain amino acid catabolism, as well as other circuitry, e.g., to regulate gene expression, including, for example, sequences for one or more inducible promoter(s), sequences for ribosome binding sites, sequences for importing one or more BCAA(s) and/or metabolite(s) thereof into the bacterial cell (e.g., transporter sequence(s)), sequences for the secretion or non-secretion of BCAA(s), metabolites or by-products (e.g., exporter(s) or exporter knockouts), and circuitry to guarantee the safety and non-colonization of the subject that is administered the recombinant bacteria, such as auxotrophies, kill switches, etc. These engineered bacteria are safe and well tolerated and augment the innate activities of the subject’s microbiome to achieve a therapeutic effect.

In one aspect, disclosed herein is a recombinant bacterium comprising at least one gene sequence encoding a leucine decarboxylase (LDC) enzyme operably linked to at least one directly or indirectly inducible promoter that is not associated with a gene encoding the leucine decarboxylase enzyme in nature. In one embodiment, the at least one gene sequence encoding a leucine decarboxylase enzyme comprises a sequence having at least 90% identity to SEQ ID NO:145. In one embodiment, the at least one gene sequence encoding a leucine decarboxylase enzyme comprises a sequence having at least 91%, 92%, 93%, 94%, 95%, 96%, 97%, 98%, or 99% identity to SEQ ID NO:145. In one embodiment, the at least one gene sequence encoding a leucine decarboxylase enzyme comprises a sequence comprising SEQ ID NO:145 or consisting of SEQ ID NO:145. In one embodiment, the LDC enzyme is E.C. 4.1.1.14. In one embodiment, the at least one gene sequence encoding a LDC enzyme is a heterologous gene sequence.

In one embodiment, the recombinant bacterium further comprises at least one gene sequence encoding at least one transporter capable of importing leucine into the bacterium operably linked to at least one directly or indirectly inducible promoter that is not associated with a gene encoding the transporter in nature. In one embodiment, the at least one gene sequence encoding at least one transporter comprises a brnQ sequence. In one embodiment, the at least one gene sequence encoding at least one transporter comprises a brnQ sequence and a livKHMGF sequence. In one embodiment, the at least one gene sequence encoding at least one transporter comprises a livKHMGF sequence. In one embodiment, the at least one gene sequence encoding the at least one transporter is a heterologous gene sequence.

In one embodiment, the brnQ sequence comprises a sequence having at least 90% identity to SEQ ID NO:64. In one embodiment, the brnQ sequence comprises a sequence having at least 91%, 92%, 93%, 94%, 95%, 96%, 97%, 98%, or 99% identity to SEQ ID NO:64. In one embodiment, the brnQ sequence comprises a sequence comprising SEQ ID NO:64 or consisting of SEQ ID NO:64.

In one embodiment, the livKHMGF sequence comprises a sequence having at least 90% identity to SEQ ID NO:91. In one embodiment, the livKHMGF sequence comprises a sequence having at least 91%, 92%, 93%, 94%, 95%, 96%, 97%, 98%, or 99% identity to SEQ ID NO:91. In one embodiment, the livKHMGF sequence comprises a sequence comprising SEQ ID NO:91 or consisting of SEQ ID NO:91.

In one aspect, disclosed herein is a recombinant bacterium comprising at least one gene sequence encoding a leucine decarboxylase (LDC) enzyme operably linked to at least one directly or indirectly inducible promoter that is not associated with a gene encoding the leucine decarboxylase enzyme in nature, and at least one gene sequence encoding at least one transporter comprising a brnQ sequence operably linked to at least one directly or indirectly inducible promoter that is not associated with a gene encoding the transporter in nature. In one embodiment, the at least one gene sequence encoding at least one transporter comprises a brnQ sequence and a livKHMGF sequence. In one embodiment, the at least one gene sequence encoding at least one transporter comprises a livKHMGF sequence.

In one embodiment, the bacterium comprises a genetic modification in leuE that reduces leucine export from the bacterium.

In one embodiment, the bacterium comprises a genetic modification in ilvC that reduces endogenous biosynthesis of leucine in the bacterium.

In some embodiments, the bacterium comprising at least one sequence encoding a leucine decarboxylase (LDC) enzyme is an auxotroph. In some embodiments, the bacterium comprising at least one sequence encoding a leucine decarboxylase (LDC) enzyme and at least one gene sequence encoding at least one transporter comprising a brnQ sequence is an auxotroph. In one embodiment, the bacteria is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1 auxotroph. In some embodiments, the engineered bacteria have more than one auxotrophy, for example, they may be a ΔthyΔ and ΔdapA auxotroph. In some embodiments, the bacteria comprising gene sequence encoding a leucine catabolism enzyme lacks functional ilvC gene sequence, e.g., is an ilvC auxotroph.

In one embodiment, the at least one gene sequence encoding the at least one leucine catabolism enzyme is integrated into the chromosome of the bacterium or is present on a plasmid in the bacterium. In one embodiment, the promoter is selected from the group consisting of an FNRS promoter, a Pₜₑₜ promoter, a P_{cmt} promoter, a P_{cl857} promoter, and a P_{BAD} promoter. In one embodiment, the promoter is selected from the group consisting of an FNRS promoter.

In one embodiment, the bacterium is a probiotic bacterium selected from the group consisting of Bacteroides, Bifidobacterium, Clostridium, Escherichia, Lactobacillus, and Lactococcus. In one embodiment, the bacterium is Escherichia coli strain Nissle.

In one aspect, disclosed herein is a pharmaceutically acceptable composition comprising a recombinant bacterium disclosed herein and a pharmaceutically acceptable carrier.

In another aspect, disclosed herein is a method of reducing the level of leucine in a subject, the method comprising a step of administering to the subject a pharmaceutically acceptable composition disclosed herein.

In another aspect, disclosed herein is a method of treating a disease associated with excess leucine and/or a metabolic disorder involving the abnormal catabolism of leucine in a subject, the method comprising a step of administering to the subject a pharmaceutically acceptable composition disclosed herein. In one embodiment, the subject has, or is suspected of having, isovaleric acidemia.

In one aspect, disclosed herein is a recombinant bacterium capable of consuming leucine at a rate of at least about 0.5 µmol/10⁹ CFU/h in vitro. In one embodiment, the bacterium is capable of consuming leucine at a rate of at least about 0.75 µmol/10⁹ CFU/h. In one embodiment, the bacterium is capable of consuming leucine at a rate of at least about 1 µmol/10⁹ CFU/h. In one embodiment, the bacterium is capable of consuming leucine at a rate of at least about 1.25 µmol/10⁹ CFU/h. In one embodiment, the bacterium is capable of consuming leucine at a rate of at least about 1.5 µmol/10⁹ CFU/h. In one embodiment, the bacterium is capable of consuming leucine at a rate of at least about 1.75 µmol/10⁹ CFU/h. In one embodiment, the bacterium is capable of consuming leucine at a rate of at least about 2 µmol/10⁹ CFU/h. In one embodiment, the bacterium is capable of consuming leucine at a rate of at least about 2.25 µmol/10⁹ CFU/h. In one embodiment, the bacterium is capable of consuming leucine at a rate of at least about 2.5 µmol/10⁹ CFU/h. In one embodiment, the bacterium is capable of consuming leucine at a rate of at least about 2.75 µmol/10⁹ CFU/h.

In one embodiment, the bacterium is capable of consuming leucine at a rate of about 0.5 to about 2.75 µmol/10⁹ CFU/h. In one embodiment, the bacterium is capable of consuming leucine at a rate of about 0.75 to about 2.75 µmol/10⁹ CFU/h. In one embodiment, the bacterium is capable of consuming leucine at a rate of about 1 to about 2.75 µmol/10⁹ CFU/h. In one embodiment, the bacterium is capable of consuming leucine at a rate of about 1.25 to about 2.75 µmol/10⁹ CFU/h. In one embodiment, the bacterium is capable of consuming leucine at a rate of about 1.75 to about 2.75 µmol/10⁹ CFU/h. In one embodiment, the bacterium is capable of consuming leucine at a rate of about 2 to about 2.75 µmol/10⁹ CFU/h. In one embodiment, the bacterium is capable of consuming leucine at a rate of about 2.25 to about 2.75 µmol/10⁹ CFU/h. In one embodiment, the bacterium is capable of consuming leucine at a rate of about 2.5 to about 2.75 µmol/10⁹ CFU/h. In one embodiment, the bacterium is capable of consuming leucine at a rate of about 0.5 to about 2 µmol/10⁹ CFU/h. In one embodiment, the bacterium is capable of consuming leucine at a rate of about 1 to about 2 µmol/10⁹ CFU/h. In one embodiment, the bacterium is capable of consuming leucine at a rate of about 0.5 to about 2.5 µmol/10⁹ CFU/h.

In one embodiment, the at least one gene sequence encoding the at least one leucine catabolism enzyme and the inducible promoter, are integrated into the chromosome of the bacterium. In one embodiment, the at least one gene sequence encoding the at least one leucine catabolism enzyme and the inducible promoter, are located in a plasmid in the bacterium. In another embodiment, the operon and the inducible promoter are integrated into the chromosome of the bacterium. In another embodiment, the operon and the inducible promoter are located in a plasmid in the bacterium.

In one embodiment, the promoter is selected from the group consisting of an FNRS promoter, a Pₜₑₜ promoter, a P_{cmt} promoter, an FNRS promoter, a P_{cl857} promoter, and a P_{BAD} promoter. In one embodiment, the promoter is an FNRS promoter.

In one embodiment, the bacterium further comprises a second brnQ gene which encodes a second BrnQ protein, wherein the second brnQ gene is operably linked to an inducible promoter that is not associated with a brnQ gene in nature, and wherein the second brnQ gene is inserted into the malE/K or malP/T locus on a chromosome of the bacterium. In one embodiment, the promoter is selected from the group consisting of an FNRS promoter, a Pₜₑₜ promoter, a P_{cmt} promoter, a P_{cl857} promoter, and a P_{BAD} promoter. In one embodiment, the promoter is an FNRS promoter.

In one embodiment, the bacterium comprises a genetic modification in leuE that reduces leucine export from the bacterium. In one embodiment, the bacterium comprises a genetic modification in ilvC that reduces endogenous biosynthesis of leucine in the bacterium. In one embodiment, the bacterium further comprises a livKHMGF gene sequence encoding at least one transporter capable of transporting leucine. In one embodiment, the livKHMGF gene sequence is operably linked to at least one promoter that is not associated with a livKHMGF gene in nature.

In one embodiment, the bacterium is capable of consuming leucine at a rate of at least about 0.5 µmol/10⁹ CFU/h in vitro. In one embodiment, the bacterium is capable of consuming leucine at a rate of at least about 1 µmol/10⁹ CFU/h in vitro. In one embodiment, the bacterium is capable of consuming leucine at a rate of at least about 1.5 µmol/10⁹ CFU/h in vitro. In one embodiment, the bacterium is capable of consuming leucine at a rate of at least about 2.0 µmol/10⁹ CFU/h in vitro. In one embodiment, the bacterium is capable of consuming leucine at a rate of at least about 2.5 µmol/10⁹ CFU/h in vitro. In one embodiment, the bacterium is capable of consuming leucine at a rate of at least about 2.75 µmol/10⁹ CFU/h in vitro.

In one embodiment, the bacterium is capable of producing isopentylamine, which can be measured as a biomarker in the serum and/or urine.

In one embodiment, the bacterium exhibits preferentially consumes leucine over valine and isoleucine. In one embodiment, the bacterium exhibits a leucine/valine activity ratio of at least about 1.1 to at least about 2.75. In one embodiment, the bacterium exhibits a leucine/isoleucine activity ratio of at least about 1.1 to at least about 5.

In one embodiment, the bacterium is a probiotic bacterium selected from the group consisting of Bacteroides, Bifidobacterium, Clostridium, Escherichia, Lactobacillus, and Lactococcus. In one embodiment, the bacterium is Escherichia coli strain Nissle.

In one aspect, disclosed herein is a pharmaceutically acceptable composition comprising the recombinant bacterium disclosed herein, and a pharmaceutically acceptable carrier.

In one aspect, disclosed herein is a method of reducing the level of leucine in a subject, the method comprising a step of administering to the subject a pharmaceutically acceptable composition.

In one aspect, disclosed herein is a method of treating a disease associated with excess leucine and/or a metabolic disorder involving the abnormal catabolism of leucine in a subject, the method comprising a step of administering to the subject a pharmaceutically acceptable composition. In some embodiments, the subject has, or is suspected of having, isovaleric acidemia.

In some embodiments, the methods may include administration of the pharmaceutically acceptable compositions disclosed herein to reduce leucine, concentrations to undetectable levels, or to less than about 1%, 2%, 5%, 10%, 20%, 25%, 30%, 40%, 50%, 60%, 70%, 75%, 80%, 85%, 90%, or 95% of the subject’s leucine levels prior to treatment.

### BRIEF DESCRIPTION OF THE DRAWINGS

FIGS. 1A and 1B depict leucine decarboxylation as an alternative degradation route for leucine metabolism. FIG. 1A depicts a schematic view of the leucine metabolism via leucine decarboxylase (LDC) with isopentylamine as the end product. Also shown are the importers LivKHMG and BrnQ; a knock-out of the exporter LeuE, and a knock-out of IlvC. FIG. 1B depicts an in vitro assay that measures isopentylamine (IPM) production of four engineered bacterial strains over time. No detectable production of IPM occurs in strains SYN001 and SYN469.

FIGS. 2A, 2B, and 2C depict BCAA-amine production via LDC using leucine as a substrate. The assays use 3 E9 cells/mL and 10 mM single BCAA for 4 hours at 37° C. in anaerobic chamber (>30 PPM O₂). FIG. 2A is a graph depicting isopentylamine production. FIG. 2B is a graph depicting 2-methylbutylamine production, and FIG. 2C is a graph depicting isobutylamine production. Only isopentylamine is detected, demonstrating that LDC is specific for leucine.

### DETAILED DESCRIPTION

The disclosure includes optimized, engineered, and programmed microorganisms, e.g., bacteria, yeast, and viruses, pharmaceutical compositions thereof, and methods of modulating and treating disorders involving the catabolism of leucine. In some embodiments, the microorganism, e.g., bacterium, yeast, or virus, has been engineered to comprise heterologous gene sequence(s) encoding one or more leucine catabolism enzyme(s), such as leucine decarboxylase (LDC).

In some embodiments, the engineered microorganism, e.g., engineered bacterium, comprises optimized heterologous gene sequence(s) encoding leucine catabolism enzyme(s) and is capable of reducing the level of leucine and/or other corresponding metabolite(s). For example, the engineered bacterium, may comprise a BCAA transporter, such as LivKHMGF and/or BrnQ. In some embodiments, the engineered bacterium comprises heterologous gene sequence(s) encoding leucine catabolism enzyme(s) and is capable of metabolizing leucine into isopentylamine. In some embodiments, the engineered bacterium comprises heterologous gene sequence(s) encoding leucine catabolism enzyme(s) and is capable of transporting leucine into the bacterium. In some embodiments, the engineered bacterium comprises heterologous gene sequence(s) encoding leucine catabolism enzyme(s) and is capable of reducing the level of leucine and/or other corresponding metabolite(s) in low-oxygen environments, e.g., the gut. In some embodiments, the engineered bacteria convert leucine to a non-toxic or low toxicity metabolite, e.g., isopentylamine. In some embodiments, the engineered bacterium comprises a genetic modification that reduces export leucine from the bacterial cell, for example, the bacterial cell may comprise a knockout or knock-down of a gene that encodes a BCAA exporter, such as leuE (which encodes a leucine exporter). In some embodiments, the engineered bacterium comprises gene sequence(s) or gene cassette(s) encoding one or more transporters of leucine, which imports leucine into the bacterial cell. In some embodiments, the bacterium has been engineered to comprise a genetic modification that reduces or inhibits endogenous production of leucine. For example, the bacterium may comprise a knockout or knock-down of a gene that encodes a molecule required for BCAA synthesis, such as ilvC (which encodes keto acid reductoisomerase). In some embodiments, the bacterium has been engineered to comprise an auxotroph, including, for example, a BCAA auxotrophy, or other auxotrophy, as provided herein and known in the art, e.g., thyA auxotrophy. In some embodiments, the bacterium has been engineered to comprise a kill-switch, such as any of the kill-switches provided herein and known in the art. In some embodiments, the bacterium has been engineered to comprise antibiotic resistance, such as any of the antibiotic resistance provided herein and known in the art. In any of these embodiments, the gene sequence(s) encoding one or more leucine catabolism enzyme(s), transporter(s), and other molecules can be integrated into the bacterial chromosome and/or can be present on a plasmid(s) (low copy and/or high copy). In any of these embodiments, the gene sequence(s) encoding leucine catabolism enzyme(s), transporter(s), and other molecules can be under the control of an inducible or constitutive promoter. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline.

Thus, the recombinant bacterial cells and pharmaceutical compositions comprising the bacterial cells disclosed herein may be used to catabolize leucine to modify, ameliorate, treat and/or prevent conditions associated with disorders involving the catabolism of leucine. In one embodiment, the disorder involving the catabolism of leucine is a metabolic disorder involving the abnormal catabolism of leucine, including but not limited to isovaleric acidemia, maple syrup urine disease (MSUD), propionic acidemia, methylmalonic acidemia, diabetes ketoacidosis, 3-MCC Deficiency, 3-Methylglutaconyl-CoA hydratase Deficiency, HMG-CoA Lyase Deficiency, Acetyl-CoA Carboxylase Deficiency, Malonyl-CoA Decarboxylase Deficiency, short-branched chain acylCoA dehydrogenase deficiency, 2-methyl-3-hydroxybutyric acidemia, beta-ketothiolase deficiency, isobutyryl-CoA dehydrogenase deficiency, HIBCH deficiency), and 3-Hydroxyisobutyric aciduria. In another embodiment, the disorder involving the catabolism of leucine is a disorder caused by the activation of mTor, for example, cancer, obesity, type 2 diabetes, neurodegeneration, autism, Alzheimer’s disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson’s disease, Huntington’s disease, psoriasis, rheumatoid arthritis, lupus, multiple sclerosis, Leigh’s syndrome, and Friedrich’s ataxia.

### Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “recombinant microorganism” refers to a microorganism, e.g., bacterial, yeast or viral cell, or bacteria, yeast or virus, that has been genetically modified from its native state. Thus, a “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

A “programmed” or “engineered” microorganism refers to a microorganism, e.g., bacterial, yeast, or viral cell, or bacteria, yeast, or virus, that has been genetically modified from its native state to perform a specific function. Thus, a “programmed or engineered bacterial cell” or “programmed or engineered bacteria” refers to a bacterial cell or bacteria that has been genetically modified from its native state to perform a specific function, e.g., to metabolize leucine. In certain embodiments, the programmed or engineered bacterial cell has been modified to express one or more proteins, for example, one or more proteins that have a therapeutic activity or serve a therapeutic purpose. The programmed or engineered bacterial cell may additionally have the ability to stop growing or to destroy itself once the protein(s) of interest have been expressed.

As used herein, the term “gene” refers to a nucleic acid fragment that encodes a protein or fragment thereof, optionally including regulatory sequences preceding (5′ non-coding sequences) and following (3′ non-coding sequences) the coding sequence. In one embodiment, a “gene” does not include regulatory sequences preceding and following the coding sequence. A “native gene” refers to a gene as found in nature, optionally with its own regulatory sequences preceding and following the coding sequence. A “chimeric gene” refers to any gene that is not a native gene, optionally comprising regulatory sequences preceding and following the coding sequence, wherein the coding sequences and/or the regulatory sequences, in whole or in part, are not found together in nature. Thus, a chimeric gene may comprise regulatory sequences and coding sequences that are derived from different sources, or regulatory and coding sequences that are derived from the same source, but arranged differently than is found in nature.

As used herein, the term “gene sequence” is meant to refer to a genetic sequence, e.g., a nucleic acid sequence. The gene sequence or genetic sequence is meant to include a complete gene sequence or a partial gene sequence. The gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

As used herein, a “heterologous” gene or “heterologous sequence” refers to a nucleotide sequence that is not normally found in a given cell in nature. As used herein, a heterologous sequence encompasses a nucleic acid sequence that is exogenously introduced into a given cell and can be a native sequence (naturally found or expressed in the cell) or non-native sequence (not naturally found or expressed in the cell) and can be a natural or wild-type sequence or a variant, non-natural, or synthetic sequence. “Heterologous gene” includes a native gene, or fragment thereof, that has been introduced into the host cell in a form that is different from the corresponding native gene. For example, a heterologous gene may include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell, genome.

As used herein, a “non-native” nucleic acid sequence refers to a nucleic acid sequence not normally present in a microorganism, e.g., an extra copy of an endogenous sequence, or a heterologous sequence such as a sequence from a different species, strain, or substrain of bacteria, yeast, or virus, or a sequence that is modified and/or mutated as compared to the unmodified sequence from bacteria, yeast, or virus of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence may be present on a plasmid or chromosome. In some embodiments, the genetically engineered microorganism of the disclosure comprises a gene that is operably linked to a promoter that is not associated with said gene in nature. For example, in some embodiments, the genetically engineered bacteria disclosed herein comprise a gene that is operably linked to a directly or indirectly inducible promoter that is not associated with said gene in nature, e.g., an FNR responsive promoter (or other promoter disclosed herein) operably linked to a gene encoding leucine catabolism enzyme. In some embodiments, the genetically engineered yeast or virus of the disclosure comprises a gene that is operably linked to a directly or indirectly inducible promoter that is not associated with said gene in nature, e.g., a promoter operably linked to a gene encoding leucine catabolism enzyme.

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, signal sequences, and stem-loop structures. In one embodiment, the regulatory sequence comprises a promoter, e.g., an FNR responsive promoter or another promoter disclosed herein.

“Operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence. More specifically, operably linked refers to a nucleic acid sequence, e.g., a gene encoding leucine catabolism enzyme, that is joined to a regulatory sequence in a manner which allows expression of the nucleic acid sequence, e.g., the gene encoding the leucine catabolism enzyme. In other words, the regulatory sequence acts in cis. In one embodiment, a gene may be “directly linked” to a regulatory sequence in a manner which allows expression of the gene. In another embodiment, a gene may be “indirectly linked” to a regulatory sequence in a manner which allows expression of the gene. In one embodiment, two or more genes may be directly or indirectly linked to a regulatory sequence in a manner which allows expression of the two or more genes. A regulatory region or sequence is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive. A “constitutive promoter” refers to a promoter that allows for continual transcription of the coding sequence or gene under its control.

“Constitutive promoter” refers to a promoter that is capable of facilitating continuous transcription of a coding sequence or gene under its control and/or to which it is operably linked. Constitutive promoters and variants are well known in the art and include, but are not limited to, Ptac promoter, BBa_J23100, a constitutive Escherichia coli σ^{s} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), P_{lepA} (BBa_K823002), P_{veg} (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), and a bacteriophage SP6 promoter (e.g., SP6 promoter (BBa_J64998)).

An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition. A “directly inducible promoter” refers to a regulatory region, wherein the regulatory region is operably linked to a gene encoding a protein or polypeptide, where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.” Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein. Examples of other inducible promoters are provided herein below.

As used herein, “stably maintained” or “stable” bacterium is used to refer to a bacterial host cell carrying non-native genetic material, e.g., a gene encoding a leucine catabolism enzyme, which is incorporated into the host genome or propagated on a self-replicating extrachromosomal plasmid, such that the non-native genetic material is retained, expressed, and propagated. The stable bacterium is capable of survival and/or growth in vitro, e.g., in medium, and/or in vivo, e.g., in the gut. For example, the stable bacterium may be a genetically engineered bacterium comprising a gene encoding a leucine catabolism enzyme, in which the plasmid or chromosome carrying the gene is stably maintained in the bacterium, such that the leucine catabolism enzyme can be expressed in the bacterium, and the bacterium is capable of survival and/or growth in vitro and/or in vivo. In some embodiments, copy number affects the stability of expression of the non-native genetic material. In some embodiments, copy number affects the level of expression of the non-native genetic material.

As used herein, the term “expression” refers to the transcription and stable accumulation of sense (mRNA) or anti-sense RNA derived from a nucleic acid, and/or to translation of an mRNA into a polypeptide.

As used herein, the term “plasmid” or “vector” refers to an extrachromosomal nucleic acid, e.g., DNA, construct that is not integrated into a bacterial cell’s genome. Plasmids are usually circular and capable of autonomous replication. Plasmids may be low-copy, medium-copy, or high-copy, as is well known in the art. Plasmids may optionally comprise a selectable marker, such as an antibiotic resistance gene, which helps select for bacterial cells containing the plasmid and which ensures that the plasmid is retained in the bacterial cell. A plasmid disclosed herein may comprise a nucleic acid sequence encoding a heterologous gene, e.g., a gene encoding a leucine catabolism enzyme.

As used herein, the term “transform” or “transformation” refers to the transfer of a nucleic acid fragment into a host bacterial cell, resulting in genetically-stable inheritance. Host bacterial cells comprising the transformed nucleic acid fragment are referred to as “recombinant” or “transgenic” or “transformed” organisms.

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, base substitution, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising a leucine catabolism enzyme operably linked to a promoter, into a bacterial cell. Genetic modification can also involve a targeted replacement in the chromosome, e.g., to replace a native gene promoter with an inducible promoter, regulated promoter, strong promoter, or constitutive promoter. Genetic modification can also involve gene amplification, e.g., introduction of at least one additional copy of a native gene into the chromosome of the cell. Alternatively, chromosomal genetic modification can involve a genetic mutation.

As used herein, the term “genetic mutation” refers to a change or changes in a nucleotide sequence of a gene or related regulatory region that alters the nucleotide sequence as compared to its native or wild-type sequence. Mutations include, for example, substitutions, additions, and deletions, in whole or in part, within the wild-type sequence. Such substitutions, additions, or deletions can be single nucleotide changes (e.g., one or more point mutations), or can be two or more nucleotide changes, which may result in substantial changes to the sequence. Mutations can occur within the coding region of the gene as well as within the non-coding and regulatory sequence of the gene. The term “genetic mutation” is intended to include silent and conservative mutations within a coding region as well as changes which alter the amino acid sequence of the polypeptide encoded by the gene. A genetic mutation in a gene coding sequence may, for example, increase, decrease, or otherwise alter the activity (e.g., enzymatic activity) of the gene’s polypeptide product. A genetic mutation in a regulatory sequence may increase, decrease, or otherwise alter the expression of sequences operably linked to the altered regulatory sequence.

Specifically, the term “genetic modification that reduces export of leucine from the bacterial cell” refers to a genetic modification that reduces the rate of export or quantity of export of leucine from the bacterial cell, as compared to the rate of export or quantity of export of the leucine from a bacterial cell not having said modification, e.g., a wild-type bacterial cell. In one embodiment, a recombinant bacterial cell having a genetic modification that reduces export of leucine from the bacterial cell comprises a genetic mutation in a native gene, e.g., a leuE gene. In another embodiment, a recombinant bacterial cell having a genetic modification that reduces export of leucine from the bacterial cell comprises a genetic mutation in a native promoter, e.g., a leuE promoter, which reduces or inhibits transcription of the leuE gene. In another embodiment, a recombinant bacterial cell having a genetic modification that reduces export of leucine from the bacterial cell comprises a genetic mutation leading to overexpression of a repressor of an exporter of leucine. In another embodiment, a recombinant bacterial cell having a genetic modification that reduces export of leucine from the bacterial cell comprises a genetic mutation which reduces or inhibits translation of the gene encoding the exporter, e.g., the leuE gene.

Moreover, the term “genetic modification that increases import of leucine into the bacterial cell” refers to a genetic modification that increases the uptake rate or increases the uptake quantity of leucine into the cytosol of the bacterial cell, as compared to the uptake rate or uptake quantity of the leucine into the cytosol of a bacterial cell not having said modification, e.g., a wild-type bacterial cell. In some embodiments, an engineered bacterial cell having a genetic modification that increases import of leucine into the bacterial cell refers to a bacterial cell comprising heterologous gene sequence (native or non-native) encoding one or more importer(s) (transporter(s)) of leucine. In some embodiments, the genetically engineered bacteria comprising genetic modification that increases import of leucine into the bacterial cell comprise gene sequence(s) encoding a BCAA transporter, e.g., a leucine transporter, or other amino acid transporter that transports one or more BCAA(s), including leucine, into the bacterial cell, for example a transporter that is capable of transporting leucine into a bacterial cell. The transporter can be any transporter that assists or allows import of leucine into the cell. In certain embodiments, the leucine transporter is a high-affinity leucine transporter, e.g., LivKHMGF. In certain embodiments, the engineered bacterial cell contains gene sequence of one or more of livK, livH, livM, livG, and livF genes. In certain embodiments, the leucine transporter is a low affinity BCAA transporter, e.g., BrnQ. In certain embodiments, the engineered bacterial cell contains gene sequence encoding brnQ gene. In some embodiments, the engineered bacteria comprise more than one copy of gene sequence encoding a BCAA transporter, e.g., a leucine transporter. In some embodiments, the engineered bacteria comprise gene sequence(s) encoding more than one BCAA transporter, e.g., two or more different BCAA transporters, e.g., two or more different leucine transporters.

As used herein, the term “transporter” is meant to refer to a mechanism, e.g., protein, proteins, or protein complex, for importing a molecule, e.g., amino acid, peptide (di-peptide, tripeptide, polypeptide, etc.), toxin, metabolite, substrate, as well as other biomolecules into the microorganism from the extracellular milieu.

As used herein, the term “polypeptide of interest” or “polypeptides of interest”, “protein of interest”, “proteins of interest”, “payload”, “payloads” includes, but is not limited to, any or a plurality of any of the leucine catabolism enzymes, and/or leucine transporters, and/or leucine binding proteins and/or leucine exporters described herein. As used herein, the term “gene of interest” or “gene sequence of interest” includes any or a plurality of any of the gene(s) an/or gene sequence(s) and or gene cassette(s) encoding one or more leucine catabolism enzymes, ranched chain amino acid transporters, leucine binding proteins, and leucine exporters described herein.

The term “branched chain amino acid” or “BCAA,” as used herein, refers to an amino acid which comprises a branched side chain. Leucine, isoleucine, and valine are naturally occurring amino acids comprising a branched side chain. However, non-naturally occurring, usual, and/or modified amino acids comprising a branched side chain are also encompassed by the term branched chain amino acid.

As used herein, the phrase “exogenous environmental condition” or “exogenous environment signal” refers to settings, circumstances, stimuli, or biological molecules under which a promoter described herein is directly or indirectly induced. The phrase “exogenous environmental conditions” is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are low-oxygen, microaerobic, or anaerobic conditions, such as the environment of the mammalian gut. In some embodiments, exogenous environmental conditions are molecules or metabolites that are specific to the mammalian gut, e.g., propionate. In some embodiments, the exogenous environmental condition is a tissue-specific or disease-specific metabolite or molecule(s). In some embodiments, the exogenous environmental condition is specific to a leucine catabolism enzyme disease, e.g., isovaleric acidemia. In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprise an oxygen level-dependent promoter. In some aspects, bacteria have evolved transcription factors that are capable of sensing oxygen levels. Different signaling pathways may be triggered by different oxygen levels and occur with different kinetics. An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR (fumarate and nitrate reductase), ANR, and DNR. Corresponding FNR-responsive promoters, ANR (anaerobic nitrate respiration)-responsive promoters, and DNR (dissimilatory nitrate respiration regulator)-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 1.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrs, FNR, P-FNRS promoter and other such related designations to indicate the promoter PfnrS.

TABLE 1

Examples of transcription factors and responsive genes and regulatory regions

| | |
|---|---|
| Transcription Factor | Examples of responsive genes, promoters, and/or regulatory regions: |
| FNR | nirB, ydfZ, pdhR, focA, ndH, hlyE, narK, narX, narG, yfiD, tdcD |
| ANR | arcDABC |
| DNR | norb, norC |

In some embodiments, the exogenous environmental conditions are the presence or absence of reactive oxygen species (ROS). In other embodiments, the exogenous environmental conditions are the presence or absence of reactive nitrogen species (RNS). In some embodiments, exogenous environmental conditions are biological molecules that are involved in the inflammatory response, for example, molecules present in an inflammatory disorder of the gut. In some embodiments, the exogenous environmental conditions or signals exist naturally or are naturally absent in the environment in which the recombinant bacterial cell resides. In some embodiments, the exogenous environmental conditions or signals are artificially created, for example, by the creation or removal of biological conditions and/or the administration or removal of biological molecules.

In some embodiments, the exogenous environmental condition(s) and/or signal(s) stimulates the activity of an inducible promoter. In some embodiments, the exogenous environmental condition(s) and/or signal(s) that serves to activate the inducible promoter is not naturally present within the gut of a mammal. In some embodiments, the inducible promoter is stimulated by a molecule or metabolite that is administered in combination with the pharmaceutical composition of the disclosure, for example, tetracycline, arabinose, or any biological molecule that serves to activate an inducible promoter. In some embodiments, the exogenous environmental condition(s) and/or signal(s) is added to culture media comprising a recombinant bacterial cell of the disclosure. In some embodiments, the exogenous environmental condition that serves to activate the inducible promoter is naturally present within the gut of a mammal (for example, low oxygen or anaerobic conditions, or biological molecules involved in an inflammatory response). In some embodiments, the loss of exposure to an exogenous environmental condition (for example, in vivo) inhibits the activity of an inducible promoter, as the exogenous environmental condition is not present to induce the promoter (for example, an aerobic environment outside the gut). “Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

As used herein, the term “low oxygen” is meant to refer to a level, amount, or concentration of oxygen (O₂) that is lower than the level, amount, or concentration of oxygen that is present in the atmosphere (e.g., <21% O₂; <160 torr O₂)). Thus, the term “low oxygen condition or conditions” or “low oxygen environment” refers to conditions or environments containing lower levels of oxygen than are present in the atmosphere. In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr O₂) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45,46, 47, 48, 49, 50, 51, 52, 53, 54, 55, 56, 57, 58, 59, and 60 mmHg O₂), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg O2, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂ (inclusive), e.g., 0-5 mmHg O₂, < 1.5 mmHg O₂, 6-10 mmHg, < 8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971- 1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorporated by reference herewith in their entireties. In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nanoaerobic, microoxic, hypoxic, anoxic, and/or anaerobic conditions. For example, Table 2 summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L = 1 ppm), or in micromoles (umole) (1 umole O₂ = 0.022391 mg/L O₂). Fondriest Environmental, Inc., “Dissolved Oxygen”, Fundamentals of Environmental Measurements, 19 Nov. 2013. In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated. In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05 - 0.1%, 0.1-0.2%, 0.1-0.5%, 0.5 - 2.0%, 0-10%, 5-10%, 10-15%, 15-20%, 20-25%, 25-30%, etc.). The exemplary fractions and ranges listed here are for illustrative purposes and not meant to be limiting in any way. In some embodiments, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of O₂ saturation levels between 0-9%, inclusive (e.g., 0-5%, 0.05 - 0.1%, 0.1-0.2%, 0.1-0.5%, 0.5 - 2.0%, 0-8%, 5-7%, 0.3-4.2% O₂, etc.). The exemplary fractions and ranges listed here are for illustrative purposes and not meant to be limiting in any way.

TABLE 2

| | |
|---|---|
| Compartment | Oxygen Tension |
| stomach | ~60 torr (e.g., 58 +/- 15 torr) |
| duodenum and first part of jejunum | ~30 torr (e.g., 32 +/- 8 torr); ~20% oxygen in ambient air |
| Ileum (mid- small intestine) | ~10 torr; ~6% oxygen in ambient air (e.g., 11 +/- 3 torr) |
| Distal sigmoid colon | ~ 3 torr (e.g., 3 +/- 1 torr) |
| colon | <2 torr |
| Lumen of cecum | <1 torr |
| tumor | <32 torr (most tumors are <15 torr) |

“Microorganism” refers to an organism or microbe of microscopic, submicroscopic, or ultramicroscopic size that typically consists of a single cell. Examples of microorganisms include bacteria, viruses, parasites, fungi, certain algae, yeast, and protozoa. In some aspects, the microorganism is engineered (“engineered microorganism”) to produce one or more therapeutic molecules, e.g., leucine catabolism enzyme(s). In certain embodiments, the microorganism is a bacterium. In certain embodiments, the microorganism is a yeast or virus.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria do not contain lipopolysaccharides (LPS). In some embodiments, non-pathogenic bacteria are commensal bacteria. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis and Saccharomyces boulardii (Sonnenborn et al., 2009; Dinleyici et al., 2014; U.S. Pat. No. 6,835,376; U.S. Pat. No. 6,203,797; U.S. Pat. No. 5,589,168; U.S. Pat. No. 7,731,976). Non-pathogenic bacteria also include commensal bacteria, which are present in the indigenous microbiota of the gut. In one embodiment, the disclosure further includes non-pathogenic Saccharomyces, such as Saccharomyces boulardii. Naturally pathogenic bacteria may be genetically engineered to reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. In some embodiments, the probiotic bacteria are Gram-negative bacteria. In some embodiments, the probiotic bacteria are Gram-positive bacteria. Some species, strains, and/or subtypes of non-pathogenic bacteria are currently recognized as probiotic bacteria. Examples of probiotic bacteria include, but are not limited to, certain strains belonging to the genus Bifidobacteria, Escherichia Coli, Lactobacillus, and Saccharomyces e.g., Bifidobacterium bifidum, Enterococcus faecium, Escherichia coli strain Nissle, Lactobacillus acidophilus, Lactobacillus bulgaricus, Lactobacillus paracasei, and Lactobacillus plantarum, and Saccharomyces boulardii (Dinleyici et al., 2014; U.S. Pat. No. 5,589,168; U.S. Pat. No. 6,203,797; U.S. Pat. 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, the term “auxotroph” or “auxotrophic” refers to an organism that requires a specific factor, e.g., an amino acid, a sugar, or other nutrient) to support its growth. An “auxotrophic modification” is a genetic modification that causes the organism to die in the absence of an exogenously added nutrient essential for survival or growth because it is unable to produce said nutrient. As used herein, the term “essential gene” refers to a gene which is necessary to for cell growth and/or survival. Essential genes are described in more detail infra and include, but are not limited to, DNA synthesis genes (such as thyA), cell wall synthesis genes (such as dapA), and amino acid genes (such as serA and metA).

As used herein, the terms “modulate” and “treat” and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease. Diseases associated with the catabolism of leucine, e.g., isovaleric acidemia, may be caused by inborn genetic mutations for which there are no known cures. Diseases can also be secondary to other conditions, e.g., liver diseases. Treating diseases involving the catabolism of leucine, such as isovaleric acidemia, may encompass reducing normal levels of branched chain amino acids, e.g., leucine, reducing excess levels of branched chain amino acids, e.g., leucine, or eliminating branched chain amino acids, e.g., leucine, and does not necessarily encompass the elimination of the underlying disease.

As used herein, the term “catabolism” refers to the breakdown of a molecule into a smaller unit. As used herein, the term “leucine catabolism” refers to the conversion of leucine into a corresponding metabolite, for example, isopentylamine. The “leucine catabolism” refers to both native and non-native conversion of leucine into a corresponding metabolite. Thus, the term additionally covers catabolism of leucine that may not occur in nature and is artificially induced as a result of genetic engineering. In one embodiment, “abnormal catabolism” refers to a decrease in the rate or the level of conversion of leucine to a corresponding metabolite, e.g., isopentylamine, leading to the accumulation of leucine that is toxic or that accumulates to a toxic level in a subject. In one embodiment, the leucine, or other metabolite thereof, accumulates to a toxic level in the blood or the brain of a subject, leading to the development of a disease or disorder associated with the abnormal catabolism of leucine in the subject. In one embodiment, “abnormal leucine catabolism” refers to a level of greater than 4 mg/dL of leucine in the plasma of a subject. In another embodiment, “normal leucine catabolism” refers to a level of less than 4 mg/dL of leucine in the plasma of a subject.

As used herein, the term “disorder involving the abnormal catabolism of leucine” or “disease involving the abnormal catabolism of leucine” or “leucine disease” or “disease associated with excess of leucine” refers to a disease or disorder wherein the catabolism of leucine or leucine alpha-keto acid is abnormal. Such diseases are genetic disorders that result from deficiency in at least one of the enzymes required to catabolize leucine. As a result, individuals suffering from leucine disease have accumulated branched chain amino acids, e.g., leucine, in their cells and tissues. Examples of leucine diseases include, but are not limited to, isovaleric acidemia, MSUD, 3-MCC deficiency, 3-methylglutaconyl-CoA hydrolase deficiency, HMG-CoA lysate deficiency, Acetyl CoA carboxylase deficiency, malonylCoA decarboxylase deficiency, short branched chain acyl-CoA dehydrogenase, 2-methyl-3-hydroxybutyric acidemia, beta-ketothiolase deficiency, isobutyl-CoA dehydrogenase deficiency, HIBCH deficiency, 3-hydroxyisobutyric aciduria, proprionic acidemis, methylmalonic acidemia, as well as those diseases resulting from mTor activation, including but not limited to cancer, obesity, type 2 diabetes, neurodegeneration, autism, Alzheimer’s disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson’s disease, Huntington’s disease, psoriasis, rheumatoid arthritis, lupus, multiple sclerosis, Leigh’s syndrome, and Friedrich’s ataxia. In one embodiment, a “disease or disorder involving the catabolism of leucine” is a metabolic disease or disorder involving the abnormal catabolism of leucine.

In one embodiment, “abnormal catabolism” refers to a decrease in the rate or the level of conversion of leucine, leading to the accumulation of leucine in a subject. In one embodiment, accumulation of leucine in the blood or the brain of a subject becomes toxic and leads to the development of a disease or disorder associated with the abnormal catabolism of leucine in the subject.

As used herein, the term “disease caused by the activation of mTor” or “disorder caused by the activation of mTor” refers to a disease or a disorder wherein the levels of leucine may be normal, and wherein leucine causes the activation of mTor at a level higher than the normal level of mTor activity. In another embodiment, the subject having a disorder caused by the activation of mTor may have higher levels of leucine than normal. Diseases caused by the activation of mTor are known in the art. See, for example, Laplante and Sabatini, Cell, 149(2):74-293, 2012. As used herein, the term “disease caused by the activation of mTor” includes cancer, obesity, type 2 diabetes, neurodegeneration, autism, Alzheimer’s disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson’s disease, Huntington’s disease, psoriasis, rheumatoid arthritis, lupus, multiple sclerosis, Leigh’s syndrome, and Friedrich’s ataxia. In one embodiment, the subject has normal levels of leucine before administration of the engineered bacteria of the present disclosure. In another embodiment, the subject has decreased levels of leucine after the administration of the engineered bacteria of the present disclosure, thereby decreasing the levels of mTor or the activity of mTor, thereby treating the disorder in the subject. In one embodiment, the pharmaceutical composition disclosed herein decreases the activity of mTor by at least about 2-fold, 3-fold, 4-fold, or 5-fold in the subject.

As used herein a “pharmaceutical composition” refers to a preparation of genetically engineered microorganism of the disclosure, e.g., genetically engineered bacteria yeast or virus, with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial or viral compound. An adjuvant is included under these phrases.

The term “excipient” refers to an inert substance added to a pharmaceutical composition to further facilitate administration of an active ingredient. Examples include, but are not limited to, calcium bicarbonate, sodium bicarbonate calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., isovaleric acidemia. A therapeutically effective amount may, for example, be sufficient to treat, prevent, reduce the severity, delay the onset, and/or reduce the risk of occurrence of one or more symptoms of a disorder associated with leucine catabolism. A therapeutically effective amount, as well as a therapeutically effective frequency of administration, can be determined by methods known in the art and discussed below.

As used herein, the term “bacteriostatic” or “cytostatic” refers to a molecule or protein which is capable of arresting, retarding, or inhibiting the growth, division, multiplication or replication of recombinant bacterial cell of the disclosure.

As used herein, the term “bactericidal” refers to a molecule or protein which is capable of killing the recombinant bacterial cell of the disclosure.

As used herein, the term “toxin” refers to a protein, enzyme, or polypeptide fragment thereof, or other molecule which is capable of arresting, retarding, or inhibiting the growth, division, multiplication or replication of the recombinant bacterial cell of the disclosure, or which is capable of killing the recombinant bacterial cell of the disclosure. The term “toxin” is intended to include bacteriostatic proteins and bactericidal proteins. The term “toxin” is intended to include, but not limited to, lytic proteins, bacteriocins (e.g., microcins and colicins), gyrase inhibitors, polymerase inhibitors, transcription inhibitors, translation inhibitors, DNases, and RNases. The term “anti-toxin” or “antitoxin,” as used herein, refers to a protein or enzyme which is capable of inhibiting the activity of a toxin. The term anti-toxin is intended to include, but not limited to, immunity modulators, and inhibitors of toxin expression. Examples of toxins and antitoxins are known in the art and described in more detail infra.

As used herein, the term “leucine catabolism enzyme” or “leucine catabolism enzyme” or “catabolism enzyme” or “leucine metabolic enzyme” refers to any enzyme that is capable of metabolizing leucine, and/or capable of reducing accumulated leucine, and/or that can lessen, ameliorate, or prevent one or more diseases associated with leucine catabolism or disease symptoms. Examples of leucine catabolism enzymes include, but are not limited to, leucine decarboxylase (LCD). In one embodiment, the LDC enzyme is E.C. 4.1.1.14. In one embodiment, the at least one gene sequence encoding a leucine decarboxylase enzyme comprises a sequence having at least 90% identity to SEQ ID NO:145. In one embodiment, the at least one gene sequence encoding a leucine decarboxylase enzyme comprises a sequence having at least 91%, 92%, 93%, 94%, 95%, 96%, 97%, 98%, or 99% identity to SEQ ID NO:145. In one embodiment, the at least one gene sequence encoding a leucine decarboxylase enzyme comprises a sequence comprising SEQ ID NO:145 or consisting of SEQ ID NO:145.

Leucine catabolism enzymes of the present disclosure include both wild-type or modified leucine catabolism enzymes and can be produced using recombinant and synthetic methods or purified from nature sources. Leucine catabolism enzymes include full-length polypeptides and functional fragments thereof, as well as homologs and variants thereof. Leucine catabolism enzymes include polypeptides that have been modified from the wild-type sequence, including, for example, polypeptides having one or more amino acid deletions, insertions, and/or substitutions and may include, for example, fusion polypeptides and polypeptides having additional sequence, e.g., regulatory peptide sequence, linker peptide sequence, and other peptide sequence. Leucine catabolism enzymes also include codon optimized leucine catabolism enzymes.

As used herein, “payload” refers to one or more molecules of interest to be produced by a genetically engineered microorganism, such as a bacterium, yeast, or a virus. In some embodiments, the payload is a therapeutic payload, e.g., a leucine catabolism enzyme or a leucine transporter polypeptide. In some embodiments, the payload is a regulatory molecule, e.g., a transcriptional regulator such as FNR. In some embodiments, the payload comprises a regulatory element, such as a promoter or a repressor. In some embodiments, the payload comprises an inducible promoter, such as from FNRS. In some embodiments, the payload comprises a repressor element, such as a kill switch. In some embodiments, the payload comprises an antibiotic resistance gene or genes. In some embodiments, the payload is encoded by a gene, multiple genes, gene cassette, or an operon. In alternate embodiments, the payload is produced by a biosynthetic or biochemical pathway, wherein the biosynthetic or biochemical pathway may optionally be endogenous to the microorganism. In alternate embodiments, the payload is produced by a biosynthetic or biochemical pathway, wherein the biosynthetic or biochemical pathway is not endogenous to the microorganism. In some embodiments, the genetically engineered microorganism comprises two or more payloads.

As used herein, the term “conventional branched chain amino acid disease treatment” or “conventional branched chain amino acid disease therapy” refers to treatment or therapy that is currently accepted, considered current standard of care, and/or used by most healthcare professionals for treating a disease or disorder associated with BCAAs, e.g., leucine. It is different from alternative or complementary therapies, which are not as widely used.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides”, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the genetically engineered bacteria, yeast, or virus of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: Ala, Pro, Gly, Gln, Asn, Ser, Thr, Cys, Ser, Tyr, Thr, Val, Ile, Leu, Met, Ala, Phe, Lys, Arg, His, Phe, Tyr, Trp, His, Asp, and Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wild-type peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety.

As used herein the term “codon-optimized” refers to the modification of codons in the gene or coding regions of a nucleic acid molecule to reflect the typical codon usage of the host organism without altering the polypeptide encoded by the nucleic acid molecule. Such optimization includes replacing at least one, or more than one, or a significant number, of codons with one or more codons that are more frequently used in the genes of the host organism. A “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the terms “secretion system” or “secretion protein” refers to a native or non-native secretion mechanism capable of secreting or exporting a biomolecule, e.g., polypeptide from the microbial, e.g., bacterial cytoplasm. The secretion system may comprise a single protein or may comprise two or more proteins assembled in a complex e.g. HlyBD. Non-limiting examples of secretion systems for gram negative bacteria include the modified type III flagellar, type I (e.g., hemolysin secretion system), type II, type IV, type V, type VI, and type VII secretion systems, resistance-nodulation-division (RND) multi-drug efflux pumps, various single membrane secretion systems. Non-liming examples of secretion systems for gram positive bacteria include Sec and TAT secretion systems. In some embodiments, the polypeptide to be secreted include a “secretion tag” of either RNA or peptide origin to direct the polypeptide to specific secretion systems. In some embodiments, the secretion system is able to remove this tag before secreting the polypeptide from the engineered bacteria. For example, in Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the lysosomal enzyme(s) into the extracellular milieu. In some embodiments, the secretion system involves the generation of a “leaky” or de-stabilized outer membrane, which may be accomplished by deleting or mutagenizing genes responsible for tethering the outer membrane to the rigid peptidoglycan skeleton, including for example, 1pp, ompC, ompA, ompF, tolA, tolB, pal, degS, degP, and nlpl. Lpp functions as the primary ‘staple’ of the bacterial cell wall to the peptidoglycan. TolA-PAL and OmpA complexes function similarly to Lpp and are other deletion targets to generate a leaky phenotype. Additionally, leaky phenotypes have been observed when periplasmic proteases, such as degS, degP or nlpI, are deactivated. Thus, in some embodiments, the engineered bacteria have one or more deleted or mutated membrane genes, e.g., selected from lpp, ompA, ompA, ompF, tolA, tolB, and pal genes. In some embodiments, the engineered bacteria have one or more deleted or mutated periplasmic protease genes, e.g., selected from degS, degP, and nlpl. In some embodiments, the engineered bacteria have one or more deleted or mutated gene(s), selected from lpp, ompA, ompA, ompF, tolA, tolB, pal, degS, degP, and nlpl genes.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### Recombinant Bacteria

The microorganisms, or isolated microorganisms, such as bacteria, of the disclosure are capable of producing one or more enzymes for metabolizing leucine and/or a metabolite thereof. In some aspects, the disclosure provides a bacterial cell that comprises one or more optimized heterologous gene sequence(s) encoding a leucine catabolism enzyme or other protein that results in a decrease in leucine levels.

In certain embodiments, the bacteria are obligate anaerobic bacteria. In certain embodiments, the bacteria are facultative anaerobic bacteria. In certain embodiments, the bacteria are aerobic bacteria. In some embodiments, the bacteria are Gram-positive bacteria. In some embodiments, the bacteria are Gram-positive bacteria and lack LPS. In some embodiments, the bacteria are Gram-negative bacteria. In some embodiments, the bacteria are Gram-positive and obligate anaerobic bacteria. In some embodiments, the bacteria are Gram-positive and facultative anaerobic bacteria. In some embodiments, the bacteria are non-pathogenic bacteria. In some embodiments, the bacteria are commensal bacteria. In some embodiments, the bacteria are probiotic bacteria. In some embodiments, the bacteria are naturally pathogenic bacteria that are modified or mutated to reduce or eliminate pathogenicity. Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera. In certain embodiments, the genetically engineered bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus, lactis, and Saccharomyces boulardii. In certain embodiments, the genetically engineered bacteria are selected from Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli, Escherichia coli Nissle, Lactobacillus acidophilus, Lactobacillus plantarum, Lactobacillus reuteri, and Lactococcus, lactis bacterial cell. In one embodiment, the bacterial cell is a Bacteroides fragilis bacterial cell. In one embodiment, the bacterial cell is a Bacteroides thetaiotaomicron bacterial cell. In one embodiment, the bacterial cell is a Bacteroides subtilis bacterial cell. In one embodiment, the bacterial cell is a Bifidobacterium bifidum bacterial cell. In one embodiment, the bacterial cell is a Bifidobacterium infantis bacterial cell. In one embodiment, the bacterial cell is a Bifidobacterium lactis bacterial cell. In one embodiment, the bacterial cell is a Clostridium butyricum bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment, the bacterial cell is a Lactobacillus reuteri bacterial cell. In one embodiment, the bacterial cell is a Lactococcus lactis bacterial cell.

In some embodiments, the bacteria are Escherichia coli strain Nissle 1917 (E. coli Nissle), a Gram-negative bacterium of the Enterobacteriaceae family that has evolved into one of the best characterized probiotics (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008). In addition, it has been shown that E. coli Nissle does not carry pathogenic adhesion factors, does not produce any enterotoxins or cytotoxins, is not invasive, and not uropathogenic (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn’s disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle’s therapeutic efficacy and safety have convincingly been proven (Ukena et al., 2007).

One of ordinary skill in the art would appreciate that the genetic modifications disclosed herein may be adapted for other species, strains, and subtypes of bacteria. Furthermore, genes from one or more different species can be introduced into one another, e.g., the kivD gene from Lactococcus lactiscan be expressed in Escherichia coli. In one embodiment, the bacterial cell does not colonize the subject having the disorder. Unmodified E. coli Nissle and the bacteria of the invention may be destroyed, e.g., by defense factors in the gut or blood serum (Sonnenborn et al., 2009). In some embodiments, the residence time is calculated for a human subject. In some embodiments, residence time in vivo is calculated for the bacteria.

In some embodiments, the bacterial cell is a genetically engineered bacterial cell. In another embodiment, the bacterial cell is a recombinant bacterial cell. In some embodiments, the disclosure comprises a colony of bacterial cells disclosed herein.

In another aspect, the disclosure provides a bacterial culture which comprises bacterial cells disclosed herein. In one aspect, the disclosure provides a bacterial culture which reduces levels of leucine, in the media of the culture. In one embodiment, the levels of leucine, are reduced by about 50%, about 75%, or about 100% in the media of the cell culture. In another embodiment, the levels leucine, are reduced by about two-fold, three-fold, four-fold, five-fold, six-fold, seven-fold, eight-fold, nine-fold, or ten-fold in the media of the cell culture. In one embodiment, the levels of leucine, are reduced below the limit of detection in the media of the cell culture.

In some embodiments of the above described bacteria, the gene encoding a leucine catabolism enzyme is present on a plasmid in the bacterium and operatively linked on the plasmid to a promoter that is induced under low-oxygen or anaerobic conditions, such as any of the promoters disclosed herein. In other embodiments, the gene encoding a leucine catabolism enzyme is present in the bacterial chromosome and is operatively linked in the chromosome to the promoter that is induced under low-oxygen or anaerobic conditions, such as any of the promoters disclosed herein. In some embodiments of the above described bacteria, the gene encoding a leucine catabolism enzyme is present on a plasmid in the bacterium and operatively linked on the plasmid to the promoter that is induced under inflammatory conditions, such as any of the promoters disclosed herein. In other embodiments, the gene encoding a leucine catabolism enzyme is present in the bacterial chromosome and is operatively linked in the chromosome to the promoter that is induced under inflammatory conditions, such as any of the promoters disclosed herein.

In some embodiments, the bacteria comprising gene sequence encoding a leucine catabolic enzyme is an auxotroph. In one embodiment, the bacteria is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1 auxotroph. In some embodiments, the engineered bacteria have more than one auxotrophy, for example, they may be a ΔthyΔ and ΔdapA auxotroph. In some embodiments, the bacteria comprising gene sequence encoding a leucine catabolism enzyme lacks functional ilvC gene sequence, e.g., is a ilvC auxotroph. IlvC encodes keto acid reductoisomerase, which enzyme is required for BCAA synthesis. Knock out of ilvC creates an auxotroph and requires the bacterial cell to import isoleucine and valine to survive.

In some embodiments, the bacteria comprising gene sequence encoding a leucine catabolism enzyme further comprise gene sequence(s) encoding a leucine transporter or other amino acid transporter that transports leucine into the bacterial cell. In certain embodiments, the leucine transporter is a high-affinity leucine transporter. In certain embodiments, the bacterial cell contains the LDC gene. In certain embodiments, the leucine transporter is a low affinity leucine transporter. In certain embodiments, the bacterial cell contains gene sequence encoding brnQ gene.

In some embodiments, the bacteria comprising gene sequence encoding a leucine catabolism enzyme further comprise gene sequence(s) encoding a secretion protein or protein complex for secreting a biomolecule, such as any of the secretion systems disclosed herein.

In some embodiments, the bacteria comprising gene sequence encoding a leucine catabolism enzyme further comprise gene sequence(s) encoding one or more antibiotic gene(s), such as any of the antibiotic genes disclosed herein.

In some embodiments, the bacteria comprising a leucine catabolism enzyme further comprise a kill-switch circuit, such as any of the kill-switch circuits provided herein. For example, in some embodiments, the bacteria further comprise one or more genes encoding one or more recombinase(s) under the control of an inducible promoter, and an inverted toxin sequence. In some embodiments, the bacteria further comprise one or more genes encoding an antitoxin. In some embodiments, the bacteria further comprise one or more genes encoding one or more recombinase(s) under the control of an inducible promoter and one or more inverted excision genes, wherein the excision gene(s) encode an enzyme that deletes an essential gene. In some embodiments, the bacteria further comprise one or more genes encoding an antitoxin. In some embodiments, the bacteria further comprise one or more genes encoding a toxin under the control of a promoter having a TetR repressor binding site and a gene encoding the TetR under the control of an inducible promoter that is induced by arabinose, such as P_{araBAD}. In some embodiments, the bacteria further comprise one or more genes encoding an antitoxin.

In some embodiments, the gene encoding a leucine catabolism enzyme is present on a plasmid in the bacterium. In some embodiments, the gene encoding a leucine catabolism enzyme is present in the bacterial chromosome. In some embodiments, the gene sequence(s) encoding a leucine transporter is present on a plasmid in the bacterium. In some embodiments, the gene sequence(s) encoding a leucine transporter is present in the bacterial chromosome. In some embodiments, the gene sequence encoding a secretion protein or protein complex for secreting a biomolecule, such as any of the secretion systems disclosed herein, is present on a plasmid in the bacterium. In some embodiments, the gene sequence encoding a secretion protein or protein complex for secreting a biomolecule, such as any of the secretion systems disclosed herein, is present in the bacterial chromosome. In some embodiments, the gene sequence(s) encoding an antibiotic resistance gene is present on a plasmid in the bacterium. In some embodiments, the gene sequence(s) encoding an antibiotic resistance gene is present in the bacterial chromosome.

### Leucine Catabolism Enzymes

As used herein, the term “leucine catabolism enzyme” or “leucine catabolic enzyme” refers to an enzyme involved in the catabolism of leucine to, for example, isopentylamine. Enzymes involved the catabolism are well known to those of ordinary skill in the art. For example, in mice, leucine decarboxylase (LDC or LeuDC), which is encoded by the Gm853 or Ldc gene, converts leucine to isopentylamine, which is then excreted into the urine (see for example, Lambertos et al., BBA - General Subjects 1862 (2018): 365-376, the entire contents of which are expressly incorporated herein by reference). The other branched chain amino acids, valine and isoleucine, were found to be poor substrates of LDC.

In one embodiment, the branched chain amino acid catabolism enzyme is leucine decarboxylase, e.g., LDC. In a non-limiting example, LDC is from mus musculus. Thus, in some embodiments, the bacteria comprise gene sequence(s) encoding one or more copies of a leucine decarboxylase. In some embodiments, the engineered bacteria comprise gene sequence(s) encoding one, two, three, four, five, six, or more copies of a leucine decarboxylase. The one or more copies of a leucine decarboxylase can be one or more copies of the same gene or can be different genes encoding leucine decarboxylase, e.g., gene(s) from a different species or otherwise having a different gene sequence. The one or more copies of a leucine decarboxylase can be present in the bacterial chromosome or can be present in one or more plasmids.

As used herein “leucine decarboxylase” (referred to herein also as LDC or LeuDC) refers to any polypeptide having enzymatic activity that catalyzes the conversion of leucine to isopentylamine. The bacterial cells disclosed herein may comprise a heterologous gene encoding a leucine decarboxylase enzyme and are capable of converting leucine into isopentylamine.

In one embodiment, the leucine decarboxylase gene has been codon-optimized for use in the bacterial cell. In one embodiment, the leucine decarboxylase gene has been codon-optimized for use in Escherichia coli.

In one embodiment, the leucine decarboxylase gene is a ldc gene. In another embodiment, the ldc gene is a mus musculus ldc gene or a ldc gene derived from mus musculus. When a leucine decarboxylase is expressed in the bacterial cells disclosed herein, the bacterial cells catabolize more leucine than unmodified bacteria of the same bacterial subtype under the same conditions (e.g., culture or environmental conditions). Thus, the bacteria comprising a heterologous gene encoding a leucine decarboxylase may be used to catabolize excess leucine to treat a disease associated with the catabolism of leucine, such as isovaleric acidemia.

The present disclosure further comprises genes encoding functional fragments of a leucine decarboxylase or functional variants of a leucine decarboxylase gene. As used herein, the term “functional fragment thereof” or “functional variant thereof” of a leucine decarboxylase gene relates to a sequence having qualitative biological activity in common with the wild-type leucine decarboxylase from which the fragment or variant was derived. For example, a functional fragment or a functional variant of a mutated leucine decarboxylase protein is one which retains essentially the same ability to catabolize leucine as a leucine decarboxylase protein from which the functional fragment or functional variant was derived. For example, a polypeptide having leucine decarboxylase activity may be truncated at the N-terminus or C-terminus and the retention of leucine decarboxylase activity assessed using assays known to those of skill in the art, including the exemplary assays provided herein. In one embodiment, the bacterial cell disclosed herein comprises a heterologous gene encoding a leucine decarboxylase functional variant. In another embodiment, the bacterial cell disclosed herein comprises a heterologous gene encoding a leucine decarboxylase functional fragment.

The present disclosure encompasses genes encoding a leucine catabolism enzyme, leucine transporter, leucine binding protein, and/or other sequence comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T. Similarly contemplated is replacing a basic amino acid with another basic amino acid (e.g., replacement among Lys, Arg, His), replacing an acidic amino acid with another acidic amino acid (e.g., replacement among Asp and Glu), replacing a neutral amino acid with another neutral amino acid (e.g., replacement among Ala, Gly, Ser, Met, Thr, Leu, Ile, Asn, Gln, Phe, Cys, Pro, Trp, Tyr, Val).

The present disclosure encompasses leucine catabolism enzymes, leucine transporters, leucine binding proteins, and/or other sequences which have a certain percent identity to a gene or protein sequence described herein. For example, the disclosure encompasses leucine catabolism enzymes, leucine transporters, leucine binding proteins, and/or other sequences having at least 60%, 65%, 70%, 75%, 80%, 85%, 90%, 91%, 92%, 93%, 94%, 95%, 96%, 97%, 98%, 99%, or 100% identity to a nucleic acid sequence or amino acid sequence disclosed herein. As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

Assays for testing the activity of a leucine catabolism enzyme, leucine transporter, leucine binding protein, and/or other sequence, or a functional variant, or a functional fragment thereof are well known to one of ordinary skill in the art. For example, leucine catabolism and/or leucine transport can be assessed by expressing the protein, functional variant, or fragment thereof, in a recombinant bacterial cell that lacks endogenous leucine catabolism enzyme activity. Leucine catabolism can be assessed using the coupled enzymatic assay method as described by Zhang et al. (see, for example, Zhang et al., Proc. Natl. Acad. Sci., 105(52):20653-58, 2008) and as described in more detail herein in the Examples.

In some embodiments, the gene encoding a leucine decarboxylase, e.g., ldc, is mutagenized; mutants exhibiting increased activity are selected; and the mutagenized gene encoding the leucine decarboxylase, e.g., ldc, is isolated and inserted into the bacterial cell. The gene comprising the modifications described herein may be present on a plasmid or chromosome.

Accordingly, in some embodiments, the ldc gene has at least about 80% identity with the entire sequence of SEQ ID NO:145. Accordingly, in one embodiment, the ldc gene has at least about 90% identity with the entire sequence of SEQ ID NO:145. Accordingly, in one embodiment, the ldh gene has at least about 95% identity with the entire sequence of SEQ ID NO:145. Accordingly, in one embodiment, the ldc gene has at least about 85%, 86%, 87%, 88%, 89%, 90%, 91%, 92%, 93%, 94%, 95%, 96%, 97%, 98%, or 99% identity with the entire sequence of SEQ ID NO:145. In another embodiment, the ldc gene comprises the sequence of SEQ ID NO:1. In yet another embodiment, the ldc gene consists of SEQ ID NO:145.

In one embodiment, the ldc gene encodes a LDC protein having at least 90% identity with SEQ ID NO:167. In one embodiment, the LDC protein has at least 95% identity with SEQ ID NO:167. In one embodiment, the LDC protein has at least about 85%, 86%, 87%, 88%, 89%, 90%, 91%, 92%, 93%, 94%, 95%, 96%, 97%, 98%, or 99% identity with the entire sequence of SEQ ID NO:167. In another embodiment, the LDC protein comprises the sequence of SEQ ID NO:167. In yet another embodiment, the LDC gene consists of SEQ ID NO:167.

In some embodiments, the at least one leucine decarboxylase enzyme is coexpressed with one or more leucine transporter(s), for example, a high affinity leucine transporter, e.g., LivKHMGF and/or low affinity BCAA transporter, e.g., BrnQ. In some embodiments, the bacteria comprise gene sequence(s) encoding at least one leucine decarboxylase enzyme and gene sequence(s) encoding one or more leucine transporter(s) (e.g., livKHMGF, brnQ).

In some embodiments, the engineered bacteria comprise gene sequence(s) encoding at least one leucine decarboxylase enzyme and genetic modification that reduces export of leucine, e.g., a genetic mutation in a leuE gene or promoter thereof. In one embodiment, the bacteria comprise gene sequence(s) encoding at least one leucine decarboxylase enzyme and a genetic modification that reduces or eliminates leucine synthesis, e.g., a genetic mutation in a ilvC gene or promoter thereof.

In some embodiments, the gene sequence(s) encoding the one or more leucine decarboxylase enzyme(s) is expressed under the control of a constitutive promoter. In some embodiments, the gene sequence(s) encoding the one or more leucine decarboxylase enzyme(s) is expressed under the control of an inducible promoter. In some embodiments, the gene sequence(s) encoding the one or more leucine decarboxylase enzyme(s) is expressed under the control of a promoter that is directly or indirectly induced by exogenous environmental conditions. In some embodiments, the gene sequence(s) encoding the one or more leucine decarboxylase enzyme(s) is expressed under the control of a promoter that is directly or indirectly induced by low-oxygen or anaerobic conditions, wherein expression of the gene encoding the leucine decarboxylase enzyme is activated under low-oxygen or anaerobic environments, such as the environment of the mammalian gut. In some embodiments, the gene sequence(s) encoding the one or more leucine decarboxylase enzyme(s) is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein.

In some embodiments, the leucine catabolism enzyme increases the rate of leucine catabolism. In some embodiments, the leucine catabolism enzyme decreases the level of leucine, in a cell, tissue, or organism.

### Transporter (Importer) of Leucine

In some embodiments, a bacterial cell disclosed herein comprising gene sequence(s) encoding at least one leucine catabolism enzyme (e.g., in some embodiments expressed on a high-copy plasmid) does not increase leucine catabolism or decrease leucine levels in the absence of a heterologous transporter (importer) of the leucine, and additional copies of a native importer of the leucine, e.g., livKHMGF. It has been surprisingly discovered that in some embodiments, the rate-limiting step of leucine catabolism, e.g., leucine decarboxylase, is not expression of a leucine catabolism enzyme, but rather availability of, leucine. Thus, in some embodiments, it may be advantageous to increase leucine transport, e.g., leucine transport, into the cell, thereby enhancing leucine catabolism. Surprisingly, in conjunction with overexpression of a transporter of a leucine, e.g., LivKHMGF and/or BrnQ, even low copy number plasmids comprising a gene encoding at least one leucine catabolism enzyme are capable of almost completely eliminating leucine, from a sample. Furthermore, in some embodiments that incorporate a transporter of a leucine into the recombinant bacterial cell, there may be additional advantages to using a low-copy plasmid comprising the gene encoding the leucine catabolism enzyme in conjunction in order to enhance the stability of expression of the leucine catabolism enzyme, while maintaining high leucine catabolism and to reduce negative selection pressure on the transformed bacterium. In alternate embodiments, the leucine transporter is used in conjunction with a high-copy plasmid. In alternate embodiments, the gene(s)at least one BCAA catabolism enzyme is integrated in the bacterial chromosome.

In some embodiments, in which the bacterial cell comprises gene sequence encoding a leucine transporter, the bacterial cell comprises gene sequence encoding one leucine catabolism enzyme. In other embodiments, in which the bacterial cell comprises gene sequence encoding a leucine transporter, the bacterial cell comprises gene sequence(s) encoding two leucine catabolism enzymes. In other embodiments, in which the bacterial cell comprises gene sequence encoding a leucine transporter, the bacterial cell comprises gene sequence(s) encoding three or more leucine catabolism enzymes. In other embodiments, in which the bacterial cell comprises gene sequence encoding a leucine transporter, the bacterial cell comprises gene sequence(s) encoding four, five, six or more leucine catabolism enzymes.

In one embodiment, the bacterial cell comprises gene sequence encoding one or more transporter(s) of leucine, gene sequence(s) encoding one or more leucine catabolism enzyme(s), and at least one genetic modification that reduces export of leucine, e.g., a genetic modification in a leuE gene or promoter thereof. In one embodiment, the bacterial cell comprises gene sequence encoding one or more transporter(s) of leucine, gene sequence(s) encoding one or more leucine catabolism enzyme(s), and at least one genetic modification that reduces or eliminates leucine synthesis, e.g., a genetic mutation in a ilvC gene or promoter thereof. In one embodiment, the bacterial cell comprises gene sequence encoding one or more transporter(s) of leucine, gene sequence(s) encoding one or more leucine catabolism enzyme(s), at least one genetic modification that reduces export of leucine, and at least one genetic modification that reduces or eliminates leucine synthesis.

The uptake of branched chain amino acids, e.g., leucine, into bacterial cells is mediated by proteins well known to those of skill in the art. For example, two well characterized BCAA transport systems have been characterized in several bacteria, including Escherichia coli. BCAAs are transported by two systems into bacterial cells (i.e., imported), the osmotic-shock-sensitive systems designated LIV-I and LS (leucine-specific), and by an osmotic-shock resistant system, BrnQ, formerly known as LIV-II (see Adams et al., J. Biol. Chem. 265:11436-43 (1990); Anderson and Oxender, J. Bacteriol. 130:384-92 (1977); Anderson and Oxender, J. Bacteriol. 136:168-74 (1978); Haney et al., J. Bacteriol. 174:108-15 (1992); Landick and Oxender, J. Biol. Chem. 260:8257-61 (1985); Nazos et al., J. Bacteriol. 166:565-73 (1986); Nazos et al., J. Bacteriol. 163:1196-202 (1985); Oxender et al., Proc. Natl. Acad. Sci. USA 77:1412-16 (1980); Quay et al., J. Bacteriol. 129:1257-65 (1977); Rahmanian et al., J. Bacteriol. 116:1258-66 (1973); Wood, J. Biol. Chem. 250:4477-85 (1975); Guardiola et al., J. Bacteriol. 117:393-405 (1974); Guardiola and Iaccarino, J. Bacteriol. 108:1034-44 (1971); Ohnishi et al., Jpn. J. Genet. 63:343-57 )(1988); Yamato and Anraku, J. Bacteriol. 144:36-44 (1980); and Yamato et al., J. Bacteriol. 138:24-32 (1979)). Transport by the BrnQ system is mediated by a single membrane protein. Transport mediated by the LIV-I system is dependent on the substrate binding protein LivJ (also known as LIV-BP), while transport mediated by LS system is mediated by the substrate binding protein LivK (also known as LS-BP). LivJ is encoded by the livJ gene, and binds isoleucine, leucine and valine with K_{d} values of ~10⁻⁶ and ~10⁻⁷ M, while LivK is encoded by the livK gene, and binds leucine with a K_{d} value of ~10⁻⁶ M (See Landick and Oxender, J. Biol. Chem. 260:8257-61 (1985)). Both LivJ and LivK interact with the inner membrane components LivHMGF to enable ATP-hydrolysis-coupled transport of their substrates into the cell, forming the LIV-I and LS transport systems, respectively. The LIV-I system transports leucine, isoleucine and valine, and to a lesser extent serine threonine and alanine, whereas the LS system only transports leucine. The six genes encoding the E. coli LIV-I and LS systems are organized into two transcriptional units, with livKHMGF transcribed as a single operon, and livJ transcribed separately. LivKHMGF is an ABC transporter comprised of five subunits, including LivK, which is a periplasmic amino acid binding protein, LivHM, which are membrane subunits, and LivGF, which are ATP-binding subunits. The Escherichia coli liv genes can be grouped according to protein function, with the livJ and livK genes encoding periplasmic binding proteins with the binding affinities described above, the livH and livM genes encoding inner membrane permeases, and the livG and livF genes encoding cytoplasmic ATPases.

BrnQ is a branched chain amino acid transporter highly similar to the Salmonella typhimurium BrnQ branched chain amino acid transporter (Ohnishi et al., Cloning and nucleotide sequence of the brnQ gene, the structural gene for a membrane-associated component of the LIV-II transport system for branched-chain amino acids in Salmonella typhimurium. Jpn J Genet. 1988 Aug;63(4):343-57) and corresponds to the Liv-II branched chain amino acid transport system in E. coli, which has been shown to transport leucine, valine, and isoleucine (Guardiola et al., Mutations affecting the different transport systems for isoleucine, leucine, and valine in Escherichia coli K-12. J Bacteriol. 1974 Feb;117(2):393-405), Guardiola and Oxender, Genetic separation of high- and low-affinity transport systems for branched-chain amino acids in Escherichia coli K-12. J Bacteriol. 1978 Oct;136(1):168-74., Anderson and Oxender, Genetic separation of high- and low-affinity transport systems for branched-chain amino acids in Escherichia coli K-12 J Bacteriol. 1978 Oct;136(1):168-74.78). BrnQ is a member of the LIVCS family of branched chain amino acid transporters and likely functions as a sodium/branched chain amino acid symporter.

Leucine importers may be expressed or modified in the bacteria disclosed herein in order to enhance leucine transport into the cell. For example, the gene sequence(s) for endogenous transporter(s) may be modified (e.g., codon-optimized and/or expressed by a strong promoter) to overexpress the transporter and/or additional copies of the transporter may be added. Alternatively, or additionally, gene sequence(s) for one or more non-endogenous or non-native transporters may be expressed in the bacterial cell. Specifically, when the transporter is expressed in the recombinant bacterial cells disclosed herein, the bacterial cells import more leucine into the cell when the transporter is expressed than unmodified bacteria of the same bacterial subtype under the same conditions (not expressing the transporter). Thus, in some embodiments, the engineered bacteria comprise gene sequence(s) encoding one or more transporter(s) of leucine, which may be used to import leucine into the bacteria so that any gene encoding a leucine catabolism enzyme expressed in the bacteria catabolize the leucine to treat diseases associated with the catabolism of leucine.

In one embodiment, the bacterial cell comprises gene sequence(s) encoding one or more transporter(s) of branched chain amino acids. In one embodiment, the bacterial cell comprises gene sequence(s) encoding one or more transporter(s) of leucine and a gene sequence(s) encoding one or more leucine catabolism enzyme(s). In one embodiment, the bacterial cell comprises gene sequence(s) encoding a one or more transporter(s) of leucine and a genetic modification that reduces export of leucine, e.g., a genetic mutation in a leuE gene or promoter thereof. In one embodiment, the bacterial cell comprises gene sequence(s) encoding one or more transporter(s) of leucine and a genetic modification that reduces or eliminates leucine synthesis, e.g., a genetic mutation in a ilvC gene or promoter thereof. In one embodiment, the bacterial cell comprises gene sequence encoding one or more transporter(s) of leucine, gene sequence(s) encoding one or more leucine catabolism enzyme(s), and at least one genetic modification that reduces export of leucine. In one embodiment, the bacterial cell comprises gene sequence encoding one or more transporter(s) of leucine, gene sequence(s) encoding one or more leucine catabolism enzyme(s), and at least one genetic modification that reduces or eliminates leucine synthesis. In one embodiment, the bacterial cell comprises gene sequence encoding one or more transporter(s) of leucine, gene sequence(s) encoding one or more leucine catabolism enzyme(s), at least one genetic modification that reduces export of leucine, and at least one genetic modification that reduces or eliminates leucine synthesis.

In any of these embodiments, the bacterial cell may further comprise a gene sequence encoding livJ, which brings BCAA into the bacterial cell. In any of these embodiments, the transporter may be a native transporter, e.g., the bacteria may comprise additional copies of the native transporter. In any of these embodiments, the transporter may be a non-native transporter. In any of these embodiments, the transporter may be LivKHMGF. In any of these embodiments, the transporter may be BrnQ. In any of these embodiments, the bacterial cell may comprise gene sequence(s) encoding LivKHMGF and BrnQ.

In some embodiments, the engineered bacteria comprise gene sequence(s) encoding one or more leucine catabolism enzyme(s) and gene sequence(s) encoding one or more leucine transporters, in which the gene sequence(s) encoding one or more leucine catabolism enzyme(s) and the gene sequence(s) encoding one or more transporter(s) are operably linked to different copies of the same promoter. In some embodiments, the engineered bacteria comprise gene sequence(s) encoding one or more leucine catabolism enzyme(s) and gene sequence(s) encoding one or more leucine transporters, in which the gene sequence(s) encoding one or more leucine catabolism enzyme(s) and the gene sequence(s) encoding one or more transporter(s) are operably linked to different promoters. Thus, in some embodiments, the disclosure provides a bacterial cell that comprises gene sequence(s) encoding one or more leucine catabolism enzyme(s) operably linked to a first promoter and gene sequence encoding one or more transporter(s) of leucine. In some embodiments, a bacterial cell comprises gene sequence(s) encoding one or more transporters of leucine operably linked to the first promoter. In other embodiments, a bacterial cell comprises gene sequence(s) encoding one or more leucine catabolism enzyme(s) operably linked to a first promoter and gene sequence(s) encoding one or more transporter(s) of leucine operably linked to a second promoter. In one embodiment, the first promoter and the second promoter are separate copies of the same promoter. In another embodiment, the first promoter and the second promoter are different promoters. In one embodiment, the first promoter and the second promoter are inducible promoters. In another embodiment, the first promoter is an inducible promoter and the second promoter is a constitutive promoter. In some embodiments, the gene sequence(s) encoding the one or more leucine catabolism enzymes and the gene sequence(s) encoding the one or more transporters is expressed under the control of a constitutive promoter. In some embodiments, the gene sequence(s) encoding the one or more leucine catabolism enzymes and the gene sequence(s) encoding the one or more leucine transporters is expressed under the control of an inducible promoter. In some embodiments, the gene sequence(s) encoding the one or more leucine transporters is expressed under the control of an inducible promoter that is directly or indirectly induced by exogenous environmental conditions. In some embodiments, the gene sequence(s) encoding the one or more leucine catabolism enzymes and the gene sequence(s) encoding the one or more leucine transporters is expressed under the control of an inducible promoter that is directly or indirectly induced by exogenous environmental conditions. In some embodiments, the gene sequence(s) encoding the one or more leucine transporters is expressed under the control of an inducible promoter that is directly or indirectly induced by low-oxygen or anaerobic conditions, wherein expression of the gene encoding the one or more transporters is activated under low-oxygen or anaerobic environments, such as the environment of the mammalian gut. In some embodiments, the gene sequence(s) encoding the one or more leucine catabolism enzymes and the gene sequence(s) encoding the one or more leucine transporters is expressed under the control of an inducible promoter that is directly or indirectly induced by low-oxygen or anaerobic conditions, wherein expression of the gene(s) encoding the one or more leucine catabolism enzymes and expression of the gene(s) encoding the one or more leucine transporters is activated under low-oxygen or anaerobic environments, such as the environment of the mammalian gut. In some embodiments, the gene sequence(s) encoding the one or more leucine transporters is expressed under the control of an inducible promoter that is directly or indirectly induced by inflammatory conditions. In some embodiments, the gene sequence(s) encoding the one or more leucine catabolism enzymes and the gene sequence(s) encoding the one or more leucine transporters is expressed under the control of an inducible promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein.

In one embodiment, the bacterial cell comprises at least one gene encoding a transporter of leucine from a different organism, e.g., a different species of bacteria. In one embodiment, the bacterial cell comprises at least one native gene encoding a transporter of leucine. In some embodiments, the at least one native gene encoding a transporter is not modified. In another embodiment, the bacterial cell comprises more than one copy of at least one native gene encoding a transporter. In yet another embodiment, the bacterial cell comprises a copy of at least one gene encoding a native transporter of leucine, as well as at least one copy of at least one heterologous gene encoding a transporter of leucine. The heterologous gene sequence may encode an additional copy or copies of the native transporter, may encode one or more copies of a non-native transporter, and/or may encode one or more copies of a homologous or different transporter from a different bacterial species. In one embodiment, the bacterial cell comprises at least one, two, three, four, five, or six copies of the at least one heterologous gene encoding a transporter of leucine. In one embodiment, the bacterial cell comprises multiple copies of the at least one heterologous gene encoding a transporter of leucine. In one embodiment, the bacterial cell comprises gene sequence(s) encoding two or more different transporters of leucine. In one embodiment, the gene sequence(s) encoding two or more different transporters is under the control of one or more inducible promoters. In one embodiment, the gene sequence(s) encoding two or more different transporters is under the control of one or more constitutive promoters. In one embodiment, the gene sequence(s) encoding two or more different transporters is under the control of at least one inducible promoter and at least one constitutive promoter. In any of these embodiments, the gene sequence(s) encoding the one or more transporter(s) may be present on one or more plasmids. In any of these embodiments, the gene sequence(s) encoding the one or more transporter(s) may be present in the bacterial chromosome.

In one embodiment, the transporter of a branched chain amino acid imports a branched chain amino acid, and/or more than one branched chain amino acid, into the bacterial cell. In one embodiment, the transporter of a branched chain amino acid imports leucine into the bacterial cell. In one embodiment, the transporter of a branched chain amino acid imports isoleucine into the bacterial cell. In one embodiment, the transporter of a branched chain amino acid imports valine into the bacterial cell. In one embodiment, the transporter of a branched chain amino acid imports one or more of leucine, isoleucine, and/or valine into the bacterial cell.

In some embodiments, the transporter of leucine is encoded by a transporter of leucine gene derived from a bacterial genus or species, including but not limited to, Bacillus, Campylobacter, Clostridium, Escherichia, Lactobacillus, Pseudomonas, Salmonella, Staphylococcus, Bacillus subtilis, Campylobacter jejuni, Clostridium perfringens, Escherichia coli, Lactobacillus delbrueckii, Pseudomonas aeruginosa, Salmonella typhimurium, or Staphylococcus aureus. In some embodiments, the bacterial species is Escherichia coli. In some embodiments, the bacterial species is Escherichia coli strain Nissle.

Multiple distinct transporters of leucine are known in the art. In one embodiment, the at least one gene encoding a transporter of leucine is the brnQ gene. In one embodiment, the at least one gene encoding a transporter of leucine is the livKHMGF operon. In another embodiment, the livKHMGF operon is an Escherichia coli livKHMGF operon. In any of these embodiments, the bacterial cell may comprise more than one copy of any of one or more of these gene sequences. In any of these embodiments, the bacterial cell may over-express any one or more of these gene sequences. In any of these embodiments, the bacterial cell may further comprise gene sequence(s) encoding one or more additional leucine transporters, e.g., a BrnQ transporter.

The present disclosure further provides genes encoding functional fragments of a transporter of leucine or functional variants of an importer of leucine. As used herein, the term “functional fragment thereof” or “functional variant thereof” of a transporter of leucine relates to an element having qualitative biological activity in common with the wild-type transporter of leucine from which the fragment or variant was derived. For example, a functional fragment or a functional variant of a mutated transporter of leucine protein is one which retains essentially the same ability to import leucine into the bacterial cell as does the importer protein from which the functional fragment or functional variant was derived. In one embodiment, the recombinant bacterial cell disclosed herein comprises at least one heterologous gene encoding a functional fragment of a transporter of leucine. In another embodiment, the recombinant bacterial cell disclosed herein comprises a heterologous gene encoding a functional variant of a transporter of leucine.

Assays for testing the activity of an importer of leucine, a functional variant of a transporter of leucine, or a functional fragment of a transporter of leucine are well known to one of ordinary skill in the art. For example, import of leucine may be determined using the methods as described in Haney et al., J. Bact., 174(1):108-15, 1992; Rahmanian et al., J. Bact., 116(3):1258-66, 1973; and Ribardo and Hendrixson, J. Bact., 173(22):6233-43, 2011, the entire contents of each of which are expressly incorporated by reference herein.

In one embodiment, the gene(s) encoding the transporter of leucine have been codon-optimized for use in the host organism. In one embodiment, the gene(s) encoding the importer of leucine have been codon-optimized for use in Escherichia coli.

The present disclosure also encompasses genes encoding a transporter of leucine comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding a transporter of leucine, e.g., livKHMGF, is mutagenized; mutants exhibiting increased leucine, transport are selected; and the mutagenized at least one gene encoding a transporter of leucine, e.g., livKHMGF, is isolated and inserted into the bacterial cell. In some embodiments, the at least one gene encoding a transporter of leucine, e.g., livKHMGF, is mutagenized; mutants exhibiting decreased leucine transport are selected; and the mutagenized at least one gene encoding a transporter of leucine, e.g., livKHMGF, is isolated and inserted into the bacterial cell. The importer modifications described herein may be present on a plasmid or chromosome.

In one embodiment, the livKHMGF operon has at least about 80% identity with SEQ ID NO:91. Accordingly, in one embodiment, the livKHMGF operon has at least about 90% identity with SEQ ID NO:91. Accordingly, in one embodiment, the livKHMGF operon has at least about 95% identity with SEQ ID NO:91. Accordingly, in one embodiment, the livKHMGF operon has at least about 85%, 86%, 87%, 88%, 89%, 90%, 91%, 92%, 93%, 94%, 95%, 96%, 97%, 98%, or 99% identity with SEQ ID NO:91. In another embodiment, the livKHMGF operon comprises SEQ ID NO:91. In yet another embodiment the livKHMGF operon consists of SEQ ID NO:91.

In some embodiments, the brnQ gene has at least about 80% identity with the entire sequence of SEQ ID NO:64. Accordingly, in one embodiment, the brnQ gene has at least about 90% identity with the entire sequence of SEQ ID NO:64. Accordingly, in one embodiment, the brnQ gene has at least about 95% identity with the entire sequence of SEQ ID NO:64. Accordingly, in one embodiment, the brnQ gene has at least about 85%, 86%, 87%, 88%, 89%, 90%, 91%, 92%, 93%, 94%, 95%, 96%, 97%, 98%, or 99% identity with the entire sequence of SEQ ID NO:64. In another embodiment, the brnQ gene comprises the sequence of SEQ ID NO:64. In yet another embodiment, the brnQ gene consists of SEQ ID NO:64.

In some embodiments, the bacterial cell comprises a heterologous gene encoding a leucine catabolism enzyme operably linked to a first promoter and at least one heterologous gene encoding a transporter of leucine. In some embodiments, the at least one heterologous gene encoding a transporter of leucine is operably linked to the first promoter. In other embodiments, the at least one heterologous gene encoding a transporter of leucine is operably linked to a second promoter. In one embodiment, the at least one gene encoding a transporter of leucine is directly operably linked to the second promoter. In another embodiment, the at least one gene encoding a transporter of leucine is indirectly operably linked to the second promoter.

In some embodiments, expression of at least one gene encoding a transporter of leucine, e.g., livKHMGF and/or brnQ, is controlled by a different promoter than the promoter that controls expression of the gene encoding the leucine catabolism enzyme. In some embodiments, expression of the at least one gene encoding a transporter of leucine, e.g., livKHMGF and/or brnQ, is controlled by the same promoter that controls expression of the leucine catabolism enzyme. In some embodiments, at least one gene encoding a transporter of leucine, e.g., livKHMGF and/or brnQ, and the leucine catabolism enzyme are divergently transcribed from a promoter region. In some embodiments, expression of each of genes encoding the at least one gene encoding a transporter of leucine, e.g., livKHMGF and/or brnQ, and the gene encoding the leucine catabolism enzyme is controlled by different promoters.

In one embodiment, the at least one gene encoding a transporter of leucine is not operably linked to its native promoter. In some embodiments, the at least one gene encoding the transporter of leucine, e.g., livKHMGF, is controlled by its native promoter. In some embodiments, the at least one gene encoding a transporter of leucine, e.g., livKHMGF and/or brnQ, is controlled by an inducible promoter. In some embodiments, the at least one gene encoding a transporter of leucine, e.g., livKHMGF and/or brnQ, is controlled by a promoter that is stronger than its native promoter. In some embodiments, the at least one gene encoding a transporter of leucine, e.g., livKHMGF, is controlled by a constitutive promoter.

In another embodiment, the promoter is an inducible promoter. Inducible promoters are described in more detail infra.

In one embodiment, the at least one gene encoding a transporter of leucine is located on a plasmid in the bacterial cell. In another embodiment, the at least one gene encoding a transporter of leucine is in the chromosome of the bacterial cell. In yet another embodiment, a native copy of the at least one gene encoding a transporter of leucine is located in the chromosome of the bacterial cell, and a copy of at least one gene encoding a transporter of leucine from a different species of bacteria is located on a plasmid in the bacterial cell. In yet another embodiment, a native copy of the at least one gene encoding a transporter of leucine is located on a plasmid in the bacterial cell, and a copy of at least one gene encoding a transporter of leucine from a different species of bacteria is located on a plasmid in the bacterial cell. In yet another embodiment, a native copy of the at least one gene encoding a transporter of leucine is located in the chromosome of the bacterial cell, and a copy of the at least one gene encoding an importer of leucine from a different species of bacteria is located in the chromosome of the bacterial cell.

In some embodiments, the at least one native gene encoding a transporter, e.g., livKHMGF and/or brnQ, in the bacterial cell is not modified, and one or more additional copies of the native transporter, e.g., livKHMGF and/or brnQ, are inserted into the genome. In some embodiments, the at least one native gene encoding a transporter, e.g., livKHMGF and/or brnQ, in the bacterial cell is not modified, and one or more additional copies of the native transporter, e.g., livKHMGF and/or brnQ, are present on a plasmid, e.g., a high copy or low copy plasmid. In one embodiment, the one or more additional copies of the native a transporter, e.g., livKHMGF and/or brnQ, that is inserted into the genome are under the control of the same inducible promoter that controls expression of the gene encoding the leucine catabolism enzyme, e.g., the FNR responsive promoter, or a different inducible promoter than the one that controls expression of the leucine catabolism enzyme, or a constitutive promoter. In alternate embodiments, the at least one native gene encoding the transporter, e.g., livKHMGF and/or brnQ, is not modified, and one or more additional copies of the transporter, e.g., livKHMGF, from a different bacterial species is inserted into the genome of the bacterial cell. In one embodiment, the one or more additional copies of the transporter inserted into the genome of the bacterial cell are under the control of the same inducible promoter that controls expression of the gene encoding the leucine catabolism enzyme, e.g., the FNR responsive promoter, or a different inducible promoter than the one that controls expression of the gene encoding the leucine catabolism enzyme, or a constitutive promoter.

In some embodiments, at least one native gene encoding the transporter, e.g., livKHMGF and/or brnQ, in the genetically modified bacteria is not modified, and one or more additional copies of at least one native gene encoding the transporter, e.g., livKHMGF and/or brnQ, are present in the bacterial cell on a plasmid. In one embodiment, the at least one native gene encoding the transporter e.g., livKHMGF and/or brnQ, present in the bacterial cell on a plasmid is under the control of the same inducible promoter that controls expression of the gene encoding the leucine catabolism enzyme, e.g., the FNR responsive promoter, or a different inducible promoter than the one that controls expression of the gene encoding the leucine catabolism enzyme, or a constitutive promoter. In alternate embodiments, the at least one native gene encoding the transporter, e.g., livKHMGF and/or brnQ, is not modified, and a copy of at least one gene encoding the transporter, e.g., livKHMGF and/or brnQ, from a different bacterial species is present in the bacteria on a plasmid. In one embodiment, the copy of at least one gene encoding the transporter, e.g., livKHMGF and/or brnQ, from a different bacterial species is under the control of the same inducible promoter that controls expression of the gene encoding the leucine catabolism enzyme, e.g., the FNR responsive promoter, or a different inducible promoter than the one that controls expression of the gene encoding the leucine catabolism enzyme, or a constitutive promoter.

In some embodiments, the bacterium is E. coli Nissle, and the at least one native gene encoding the transporter, e.g., livKHMGF and/or brnQ, in E. coli Nissle is not modified; one or more additional copies at least one native gene encoding the transporter, e.g., livKHMGF and/or brnQ, from E. coli Nissle is inserted into the E. coli Nissle genome under the control of the same inducible promoter that controls expression of the gene encoding the leucine catabolism enzyme, e.g., the FNR responsive promoter, or a different inducible promoter than the one that controls expression of the gene encoding the leucine catabolism enzyme, or a constitutive promoter. In an alternate embodiment, the at least one native gene encoding the a transporter, e.g., livKHMGF and/or brnQ in E. coli Nissle is not modified, and a copy of at least one gene encoding the transporter, e.g., livKHMGF and/or brnQ, from a different bacterial species is inserted into the E. coli Nissle genome under the control of the same inducible promoter that controls expression of the gene encoding the leucine catabolism enzyme, e.g., the FNR responsive promoter, or a different inducible promoter than the one that controls expression of the gene encoding the leucine catabolism enzyme, or a constitutive promoter.

In some embodiments, the bacterial cell is E. coli Nissle, and the at least one native gene encoding the transporter, e.g., livKHMGF and/or brnQ, in E. coli Nissle is not modified; one or more additional copies the at least one native gene encoding the transporter, e.g., livKHMGF and/or brnQ, E. coli Nissle is present in the bacterium on a plasmid and under the control of the same inducible promoter that controls expression of the gene encoding the leucine catabolism enzyme, e.g., the FNR responsive promoter, or a different inducible promoter than the one that controls expression of the gene encoding the leucine catabolism enzyme, or a constitutive promoter. In an alternate embodiment, the at least one native gene encoding the transporter, e.g., livKHMGF, in E. coli Nissle is not modified, and a copy of at least one native gene encoding the transporter, e.g., livKHMGF and/or brnQ, from a different bacterial species of are present in the bacterium on a plasmid and under the control of the same inducible promoter that controls expression of the gene encoding the leucine catabolism enzyme, e.g., the FNR responsive promoter, or a different inducible promoter than the one that controls expression of the gene encoding the leucine catabolism enzyme, or a constitutive promoter.

In one embodiment, when the transporter of leucine is expressed in the bacterial cells disclosed herein, the bacterial cells import 10% more leucine into the bacterial cell when the transporter is expressed as compared to unmodified bacteria of the same bacterial subtype under the same conditions. In another embodiment, when the transporter of leucine is expressed in the bacterial cells disclosed herein, the bacterial cells import 20%, 30%, 40%, 50%, 60%, 70%, 80%, 90% or 100% more leucine into the bacterial cell when the transporter is expressed as compared with unmodified bacteria of the same bacterial subtype under the same conditions. In yet another embodiment, when the transporter of leucine is expressed in the bacterial cells disclosed herein, the bacterial cells import two-fold more branched chain amino acids, e.g., leucine, into the cell when the transporter is expressed than unmodified bacteria of the same bacterial subtype under the same conditions. In yet another embodiment, when the transporter of leucine is expressed in the recombinant bacterial cells disclosed herein, the bacterial cells import three-fold, four-fold, five-fold, six-fold, seven-fold, eight-fold, nine-fold, or ten-fold more leucine into the cell when a transporter is expressed as compared with unmodified bacteria of the same bacterial subtype under the same conditions.

### Exporter of Leucine

The bacterial cells disclosed herein may comprise a genetic modification that inhibits or decreases the export of leucine or other metabolite from the bacterial cell. Knocking-out or reducing export of leucine from a bacterial cell allows the bacterial cell to more efficiently retain and catabolize exogenous leucine or other metabolite counterparts in order to treat the diseases and disorders described herein. Any of the bacterial cells disclosed herein comprising gene sequence encoding one or more leucine catabolism enzymes and/or one or more leucine transporters may further a genetic modification that inhibits or decreases the export of leucine from the bacterial cell.

The export of branched chain amino acids, e.g., leucine, from bacterial cells is mediated by proteins well known to those of skill in the art. For example, one leucine exporter, the leucine exporter LeuE has been characterized in Escherichia coli (Kutukova et al., FEBS Letters 579:4629-34 (2005); incorporated herein by reference). LeuE is encoded by the leuE gene in Escherichia coli (also known as yeaS). Additionally, a two-gene encoded exporter of the branched chain amino acids isoleucine, valine and leucine, denominated BrnFE was identified in the bacteria Corynebacterium glutamicum (Kennerknecht et al., J. Bacteriol. 184:3947-56 (2002); incorporated herein by reference). The BrnFE system is encoded by the Corynebacterium glutamicum genes brnF and brnE, and homologues of said genes have been identified in several organisms, including Agrobacterium tumefaciens, Achaeoglobus fulgidus, Bacillus subtilis, Deinococcus radiodurans, Escherichia coli, Haemophilus influenzae, Helicobacter pylori, Lactococcus lactis, Streptococcus pneumoniae, and Vibrio cholerae (see Kennerknecht et al., 2002).

The bacterial cells disclosed herein comprise a genetic modification that reduces export of a branched chain amino acid from the bacterial cell. Multiple distinct exporters of branched chain amino acids, e.g., leucine, are known in the art. In one embodiment, the recombinant bacterial cell disclosed herein comprises a genetic modification that reduces export of a branched chain amino acid from the bacterial cell, wherein the endogenous gene encoding an exporter of a branched chain amino acid is a leuE gene.

In one embodiment, the recombinant bacterial cell disclosed herein comprises a genetic modification that reduces export of a branched chain amino acid from the bacterial cell and a heterologous gene encoding a leucine catabolism enzyme. When the recombinant bacterial cells disclosed herein comprise a genetic modification that reduces export of leucine, the bacterial cells retain more leucine in the bacterial cell than unmodified bacteria of the same bacterial subtype under the same conditions. Thus, the recombinant bacteria comprising a genetic modification that reduces export of leucine may be used to retain more leucine in the bacterial cell so that any leucine catabolism enzyme expressed in the organism, e.g., co-expressed leucine decarboxylase, can catabolize the leucine to treat diseases associated with the catabolism of leucine. In one embodiment, the recombinant bacteria further comprise a heterologous gene encoding an importer of leucine, e.g., a livKHMGF and/or brnQ gene.

In one embodiment, the genetic modification reduces export of a branched chain amino acid, e.g., leucine, from the bacterial cell. In one embodiment, the bacterial cell is from a bacterial genus or species that includes but is not limited to, Bacillus, Bacteroides, Bifidobacterium, Brevibacteria, Clostridium, Enterococcus, Escherichia, Lactobacillus, Lactococcus, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus, lactis. In another embodiment, the bacterial cell is an Escherichia coli bacterial cell. In another embodiment, the bacterial cell is an Escherichia coli strain Nissle bacterial cell.

In one embodiment, the genetic modification is a mutation in an endogenous gene encoding an exporter of leucine. In one embodiment, the genetic mutation is a deletion of the endogenous gene encoding an exporter, e.g., leuE, of leucine. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter, e.g., LeuE, having no activity, i.e., results in an exporter, e.g., LeuE, which cannot export leucine from the bacterial cell.

It is routine for one of ordinary skill in the art to make mutations in a gene of interest. Mutations include substitutions, insertions, deletions, and/or truncations of one or more specific amino acid residues or of one or more specific nucleotides or codons in the polypeptide or polynucleotide of the exporter of a branched chain amino acid. Mutagenesis and directed evolution methods are well known in the art for creating variants. See, e.g., U.S. Pat. No. 7,783,428; U.S. Pat. No. 6,586,182; U.S. Pat. No. 6,117,679; and Ling, et al., 1999, “Approaches to DNA mutagenesis: an overview,” Anal. Biochem., 254(2):157-78; Smith, 1985, “In vitro mutagenesis,” Ann. Rev. Genet., 19:423-462; Carter, 1986, “Site-directed mutagenesis,” Biochem. J., 237:1-7; and Minshull, et al., 1999, “Protein evolution by molecular breeding,” Current Opinion in Chemical Biology, 3:284-290. For example, the lambda red system can be used to knock-out genes in E. coli (see, for example, Datta et al., Gene, 379:109-115 (2006)).

The term “inactivated” as applied to a gene refers to any genetic modification that decreases or eliminates the expression of the gene and/or the functional activity of the corresponding gene product (mRNA and/or protein). The term “inactivated” encompasses complete or partial inactivation, suppression, deletion, interruption, blockage, promoter alterations, antisense RNA, dsRNA, or down-regulation of a gene. This can be accomplished, for example, by gene “knockout,” inactivation, mutation (e.g., insertion, deletion, point, or frameshift mutations that disrupt the expression or activity of the gene product), or by use of inhibitory RNAs (e.g., sense, antisense, or RNAi technology). A deletion may encompass all or part of a gene’s coding sequence. The term “knockout” refers to the deletion of most (at least about 95%, at least about 96%, at least about 97%, at least about 98%, or at least about 99%) or all (100%) of the coding sequence of a gene. In some embodiments, any number of nucleotides can be deleted, from a single base to an entire piece of a chromosome.

Assays for testing the activity of an exporter of a branched chain amino acid, e.g., leucine, are well known to one of ordinary skill in the art. For example, export of a branched chain amino acid, such as leucine, may be determined using the methods described by Haney et al., J. Bact., 174(1):108-15, 1992; Rahmanian et al., J. Bact., 116(3):1258-66, 1973; and Ribardo and Hendrixson, J. Bact., 173(22):6233-43, 2011, the entire contents of which are expressly incorporated herein by reference.

In another embodiment, the genetic modification is a mutation in a promoter of an endogenous gene encoding an exporter of leucine. In one embodiment, the genetic mutation results in decreased expression of the leuE gene. In one embodiment, leuE gene expression is reduced by about 50%, 75%, or 100%. In another embodiment, leuE gene expression is reduced about two-fold, three-fold, four-fold, or five-fold. In another embodiment, the genetic mutation completely inhibits expression of the leuE gene.

Assays for testing the level of expression of a gene, such as an exporter of leucine, e.g., leuE, are well known to one of ordinary skill in the art. For example, reverse-transcriptase polymerase chain reaction may be used to detect the level of mRNA expression of a gene. Alternatively, Western blots using antibodies directed against a protein may be used to determine the level of expression of the protein.

In another embodiment, the genetic modification is an overexpression of a repressor of an exporter of leucine. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

### Reduction of Endogenous Bacterial Branched Chain Amino Acid Production

The bacterial cells disclosed herein may comprise a genetic modification that inhibits or decreases the biosynthesis of branched chain amino acid, e.g., leucine, or other metabolite in the bacterial cell. Knocking-out or reducing production of endogenous BCAA in a bacterial cell allows the bacterial cell to more efficiently take up and catabolize exogenous BCAA in order to treat the diseases and disorders described herein. Knock-out or knock down of a gene encoding an enzyme required for BCAA biosynthesis creates an auxotroph, which requires the cell to import BCAA to survive. Any of the bacterial cells disclosed herein comprising gene sequence encoding one or more leucine catabolism enzymes and/or one or more leucine transporters may further a genetic modification that inhibits or decreases the biosynthesis of BCAA, e.g., leucine, in the bacterial cell.

As used herein, the term “branched chain amino acid biosynthesis” enzyme refers to an enzyme involved in the biosynthesis of a branched chain amino acid and/or its corresponding alpha-keto acid or other metabolite. Multiple distinct genes involved in biosynthetic pathways of branched chain amino acids, e.g., isoleucine, leucine, and valine, are known in the art. For example, the ilvC gene encodes a keto-acid reductoisomerase enzyme that catalyzes the conversion of acetohydroxy acids into dihydroxy valerates, which leads to the synthesis of the essential branched side chain amino acids valine and isoleucine (EC 1.1.1.86) and has been characterized in Escherichia coli (Wek and Hatfield, J. Biol. Chem.261:2441-50 (1986), the entire contents of which are expressly incorporated herein by reference). Additionally, homologues of ilvC have been identified in several organisms, including Candida albicans, Oryza sativa, Saccharomyces cerevisiae, Pseudomonas aeruginosa, Corynebacterium glutamicum, and Spinacia oleracea.

In one embodiment, the genetic modification is a mutation in an endogenous gene encoding a protein that is involved in the biosynthesis of a branched chain amino acid, e.g., ilvC. ilvC is an acetohydroxy acid isomeroreductase that is required for branched chain amino acid synthesis. In one embodiment, the genetic mutation is a deletion of an endogenous gene encoding a protein that is involved in the biosynthesis of a branched chain amino acid or an alpha-keto acid, or other BCAA metabolite, e.g., ilvC. In another embodiment, the genetic mutation results in an enzyme having reduced activity as compared to a wild-type enzyme. In one embodiment, the activity of the enzyme is reduced at least 50%, at least 75%, or at least 100%. In another embodiment, the activity of the enzyme is reduced at least two-fold, three-fold, four-fold, or five-fold. In another embodiment, the genetic mutation results in an enzyme, e.g., IlvC, having no activity, i.e., results in an enzyme, e.g., IlvC, which cannot catalyze the conversion of acetohydroxy acids into dihydroxy valerates, thereby inhibiting the endogenous synthesis of the branched chain amino acids valine and isoleucine in the recombinant bacterial cell.

It is routine for one of ordinary skill in the art to make mutations in a gene of interest. Mutations include substitutions, insertions, deletions, and/or truncations of one or more specific amino acid residues or of one or more specific nucleotides or codons in the polypeptide or polynucleotide of the exporter of a branched chain amino acid. Mutagenesis and directed evolution methods are well known in the art for creating variants. See, e.g., U.S. Pat. No. 7,783,428; U.S. Pat. No. 6,586,182; U.S. Pat. No. 6,117,679; and Ling, et al., 1999, “Approaches to DNA mutagenesis: an overview,” Anal. Biochem., 254(2):157-78; Smith, 1985, “In vitro mutagenesis,” Ann. Rev. Genet., 19:423-462; Carter, 1986, “Site-directed mutagenesis,” Biochem. J., 237:1-7; and Minshull, et al., 1999, “Protein evolution by molecular breeding,” Current Opinion in Chemical Biology, 3:284-290. For example, the lambda red system can be used to knock-out genes in E. coli (see, for example, Datta et al., Gene, 379:109-115 (2006)).

The term “inactivated,” as applied to a gene, refers to any genetic modification that decreases or eliminates the expression of the gene and/or the functional activity of the corresponding gene product (mRNA and/or protein). The term “inactivated” encompasses complete or partial inactivation, suppression, deletion, interruption, blockage, promoter alterations, antisense RNA, dsRNA, or down-regulation of a gene. This can be accomplished, for example, by gene “knockout,” inactivation, mutation (e.g., insertion, deletion, point, or frameshift mutations that disrupt the expression or activity of the gene product), or by use of inhibitory RNAs (e.g., sense, antisense, or RNAi technology). A deletion may encompass all or part of a gene’s coding sequence. The term “knockout” refers to the deletion of most (at least about 95%, at least about 96%, at least about 97%, at least about 98%, or at least about 99%) or all (100%) of the coding sequence of a gene. In some embodiments, any number of nucleotides can be deleted, from a single base to an entire piece of a chromosome.

Assays for testing the activity of enzymes involved in the biosynthesis of branched chain amino acids and/or alpha-keto acids, and/or other BCAA metabolite e.g., ilvC, are well known to one of ordinary skill in the art. For example, the activity of a ketol-acid reductoisomerase enzyme may be determined using the methods described by Durner et al., Plant Physiol., 103:903-910, 1993, the entire contents of which are expressly incorporated herein by reference.

In another embodiment, the genetic modification is a mutation in a promoter of an endogenous gene encoding the branched chain amino acid biosynthesis enzyme. In one embodiment, the genetic mutation results in decreased expression of the branched chain amino acid biosynthesis enzyme gene. In one embodiment, gene expression is reduced by about 50%, 75%, or 100%. In another embodiment, gene expression is reduced about two-fold, three-fold, four-fold, or five-fold. In another embodiment, the genetic mutation completely inhibits expression of the gene.

Assays for testing the level of expression of a gene, such as ilvC, are well known to one of ordinary skill in the art. For example, reverse-transcriptase polymerase chain reaction may be used to detect the level of mRNA expression of a gene. Alternatively, Western blots using antibodies directed against a protein may be used to determine the level of expression of the protein.

In another embodiment, the genetic modification is an overexpression of a repressor of a branched chain amino acid biosynthesis gene. In one embodiment, the overexpression of the repressor of the gene is caused by a mutation which renders the promoter of the repressor constitutively active. In another embodiment, the overexpression of the repressor of the branched chain amino acid biosynthesis gene is caused by the insertion of an inducible promoter in front of the repressor so that the expression of the repressor can be induced. Inducible promoters are described in more detail herein.

### Inducible Promoters

In some embodiments, the bacterial cell comprises a stably maintained plasmid and/or chromosome carrying the one or more optimized genes, e.g., ldc, livKHMGF and bmQ, such that the optimized enzymes can be expressed in the host cell, and the host cell is capable of survival and/or growth in vitro, e.g., in medium, and/or in vivo, e.g., in the gut.

In some embodiments, the gene encoding the leucine catabolism enzyme is present on a plasmid and operably linked to a directly or indirectly inducible promoter. In some embodiments, the gene encoding the leucine catabolism enzyme is present on a plasmid and operably linked to a promoter that is induced under low-oxygen or anaerobic conditions. In some embodiments, the gene encoding the leucine catabolism enzyme is present on a chromosome and operably linked to a directly or indirectly inducible promoter. In some embodiments, the gene encoding the leucine catabolism enzyme is present in the chromosome and operably linked to a promoter that is induced under low-oxygen or anaerobic conditions. In some embodiments, the gene encoding the leucine catabolism enzyme is present on a plasmid and operably linked to a promoter that is induced by exposure to tetracycline or arabinose.

In some embodiments, the bacterial cell comprises a stably maintained plasmid or chromosome carrying the at least one gene encoding a transporter of leucine, e.g., livKHMGF and/or brnQ, such that the transporter, e.g., LivKHMGF and/or BrnQ, can be expressed in the host cell, and the host cell is capable of survival and/or growth in vitro, e.g., in medium, and/or in vivo, e.g., in the gut. In some embodiments, bacterial cell comprises two or more distinct copies of the at least one gene encoding a transporter, e.g., livKHMGF and/or bmQ. In some embodiments, the genetically engineered bacteria comprise multiple copies of the same at least one gene encoding a transporter, e.g., livKHMGF and/or brnQ. In some embodiments, the at least one gene encoding a transporter, e.g., livKHMGF and/or brnQ, is present on a plasmid and operably linked to a directly or indirectly inducible promoter. In some embodiments, the at least one gene encoding a transporter, e.g., livKHMGF and/or brnQ, is present on a plasmid and operably linked to a promoter that is induced under low-oxygen or anaerobic conditions. In some embodiments, the at least one gene encoding a transporter, e.g., livKHMGF and/or bmQ, is present on a chromosome and operably linked to a directly or indirectly inducible promoter. In some embodiments, the at least one gene encoding a transporter, e.g., livKHMGF and/or bmQ, is present in the chromosome and operably linked to a promoter that is induced under low-oxygen or anaerobic conditions. In some embodiments, the at least one gene encoding a transporter, e.g., livKHMGF and/or brnQ, is present on a plasmid and operably linked to a promoter that is induced by exposure to tetracycline.

In some embodiments, the promoter that is operably linked to the gene encoding the leucine catabolism enzyme and the promoter that is operably linked to the gene encoding the transporter, e.g., livKHMGF and/or bmQ, is directly induced by exogenous environmental conditions. In some embodiments, the promoter that is operably linked to the gene encoding the leucine catabolism enzyme and the promoter that is operably linked to the gene encoding the transporter, e.g., livKHMGF and/or brnQ, is indirectly induced by exogenous environmental conditions. In some embodiments, the promoter is directly or indirectly induced by exogenous environmental conditions specific to the gut of a mammal. In some embodiments, the promoter is directly or indirectly induced by exogenous environmental conditions specific to the small intestine of a mammal. In some embodiments, the promoter is directly or indirectly induced by low-oxygen or anaerobic conditions such as the environment of the mammalian gut. In some embodiments, the promoter is directly or indirectly induced by molecules or metabolites that are specific to the gut of a mammal, e.g., propionate. In some embodiments, the promoter is directly or indirectly induced by a molecule that is co-administered with the bacterial cell.

In certain embodiments, the bacterial cell comprises optimized genes, e.g., ldh, and brnQ, which are expressed under the control of the fumarate and nitrate reductase regulator (FNR) promoter. In certain embodiments, the bacterial cell comprises at least one gene encoding a transporter, e.g., livKHMGF, which is expressed under the control of the fumarate and nitrate reductase regulator (FNR) promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive.

FNR responsive promoters include, but are not limited to, the FNR responsive promoters listed in the chart, below. Underlined sequences are predicted ribosome binding sites, and bolded sequences are restriction sites used for cloning.

TABLE 3

FNR responsive promoters

| | |
|---|---|
| FNR Responsive Promoter | Sequence |
| SEQ ID NO: 146 | GTCAGCATAACACCCTGACCTCTCATTAATTGTTCATGCCGGGCGGCACTATCGTCGTCCGGCCTTTTCCTCTCTTACTCTGCTACGTACATCTATTTCTATAAATCCGTTCAATTTGTCTGTTTTTTGCACAAACATGAAATATCAGACAATTCCGTGACTTAAGAAAATTTATACAAATCAGCAATATACCCCTTAAGGAGTATATAAAGGTGAATTTGATTTACATCAATAAGCGGGGTTGCTGAATCGTTAAGGTAGGCGGTAATAGAAAAGAAATCGAGGCAAAA |
| SEQ ID NO: 147 | ATTTCCTCTCATCCCATCCGGGGTGAGAGTCTTTTCCCCCGACTTATGGCTCATGCATGCATCAAAAAAGATGTGAGCTTGATCAAAAACAAAAAATATTTCACTCGACAGGAGTATTTATATTGCGCCCGTTACGTGGGCTTCGACTGTAAATCAGAAAGGAGAAAACACCT |
| SEQ ID NO: 148 | GTCAGCATAACACCCTGACCTCTCATTAATTGTTCATGCCGGGCGGCACTATCGTCGTCCGGCCTTTTCCTCTCTTACTCTGCTACGTACATCTATTTCTATAAATCCGTTCAATTTGTCTGTTTTTTGCACAAACATGAAATATCAGACAATTCCGTGACTTAAGAAAATTTATACAAATCAGCAATATACCCCTTAAGGAGTATATAAAGGTGAATTTGATTTACATCAATAAGCGGGGTTGCTGAATCGTTAAGGATCCCTCTAGAAATAATTTTGTTTAACTTTAAGAAGGAGATATACAT |
| SEQ ID NO: 149 | CATTTCCTCTCATCCCATCCGGGGTGAGAGTCTTTTCCCCCGACTTATGGCTCATGCATGCATCAAAAAAGATGTGAGCTTGATCAAAAACAAAAAATATTTCACTCGACAGGAGTATTTATATTGCGCCCGGATCCCTCTAGAAATAATTTTGTTTAACTTTAAGAAGGAGATATACAT |
| SEQ ID NO: 150 | AGTTGTTCTTATTGGTGGTGTTGCTTTATGGTTGCATCGTAGTAAATGGTTGTAACAAAAGCAATTTTTCCGGCTGTCTGTATACAAAAACGCCGTAAAGTTTGAGCGAAGTCAATAAACTCTCTACCCATTCAGGGCAATATCTCTCTTGGATCCCTCTAGAAATAATTTTGTTTAACTTTAAGAAGGAGATATACAT |

Additional FNR responsive promoters are shown below.

TABLE 4

FNR Promoter Sequences

| | |
|---|---|
| SEQ ID NO | FNR-responsive regulatory region Sequence |
| SEQ ID NO: 151 | ATCCCCATCACTCTTGATGGAGATCAATTCCCCAAGCTGCTAGAGCGTTACCTTGCCCTTAAACATTAGCAATGTCGATTTATCAGAGGGCCGACAGGCTCCCACAGGAGAAAACCG |
| SEQ ID NO: 152 | CTCTTGATCGTTATCAATTCCCACGCTGTTTCAGAGCGTTACCTTGCCCTTAAACATTAGCAATGTCGATTTATCAGAGGGCCGACAGGCTCCCACAGGAGAAAACCG |
| nirB1 SEQ ID NO: 153 | GTCAGCATAACACCCTGACCTCTCATTAATTGTTCATGCCGGGCGGCACTATCGTCGTCCGGCCTTTTCCTCTCTTACTCTGCTACGTACATCTATTTCTATAAATCCGTTCAATTTGTCTGTTTTTTGCACAAACATGAAATATCAGACAATTCCGTGACTTAAGAAAATTTATACAAATCAGCAATATACCCCTTAAGGAGTATATAAAGGTGAATTTGATTTACATCAATAAGCGGGGTTGCTGAATCGTTAAGGTAGGCGGTAATAGAAAAGAAATCGAGGCAAAA |
| nirB2 SEQ ID NO: 154 | CGGCCCGATCGTTGAACATAGCGGTCCGCAGGCGGCACTGCTTACAGCAAACGGTCTGTACGCTGTCGTCTTTGTGATGTGCTTCCTGTTAGGTTTCGTCAGCCGTCACCGTCAGCATAACACCCTGACCTCTCATTAATTGCTCATGCCGGACGGCACTATCGTCGTCCGGCCTTTTCCTCTCTTCCCCCGCTACGTGCATCTATTTCTATAAACCCGCTCATTTTGTCTATTTTTTGCACAAACATGAAATATCAGACAATTCCGTGACTTAAGAAAATTTATACAAATCAGCAATATACCCATTAAGGAGTATATAAAGGTGAATTTGATTTACATCAATAAGCGGGGTTGCTGAATCGTTAAGGTAGGCGGTAATAGAAAAGAAATCGAGGCAAAAatgtttgtttaactttaagaaggagatatacat |
| nirB3 SEQ ID NO: 155 | GTCAGCATAACACCCTGACCTCTCATTAATTGCTCATGCCGGACGGCACTATCGTCGTCCGGCCTTTTCCTCTCTTCCCCCGCTACGTGCATCTATTTCTATAAACCCGCTCATTTTGTCTATTTTTTGCACAAACATGAAATATCAGACAATTCCGTGACTTAAGAAAATTTATACAAATCAGCAATATACCCATTAAGGAGTATATAAAGGTGAATTTGATTTACATCAATAAGCGGGGTTGCTGAATCGTTAAGGTAGGCGGTAATAGAAAAGAAATCGAGGCAAAA |
| ydfZ SEQ ID NO: 156 | ATTTCCTCTCATCCCATCCGGGGTGAGAGTCTTTTCCCCCGACTTATGGCTCATGCATGCATCAAAAAAGATGTGAGCTTGATCAAAAACAAAAAATATTTCACTCGACAGGAGTATTTATATTGCGCCCGTTACGTGGGCTTCGACTGTAAATCAGAAAGGAGAAAACACCT |
| nirB+RBS SEQ ID NO: 157 | GTCAGCATAACACCCTGACCTCTCATTAATTGTTCATGCCGGGCGGCACTATCGTCGTCCGGCCTTTTCCTCTCTTACTCTGCTACGTACATCTATTTCTATAAATCCGTTCAATTTGTCTGTTTTTTGCACAAACATGAAATATCAGACAATTCCGTGACTTAAGAAAATTTATACAAATCAGCAATATACCCCTTAAGGAGTATATAAAGGTGAATTTGATTTACATCAATAAGCGGGGTTGCTGAATCGTTAAGGATCCCTCTAGAAATAATTTTGTTTAACTTTAAGAAGGAGATATACAT |
| ydfZ+RBS SEQ ID NO: 158 | CATTTCCTCTCATCCCATCCGGGGTGAGAGTCTTTTCCCCCGACTTATGGCTCATGCATGCATCAAAAAAGATGTGAGCTTGATCAAAAACAAAAAATATTTCACTCGACAGGAGTATTTATATTGCGCCCGGATCCCTCTAGAAATAATTTTGTTTAACTTTAAGAAGGAGATATACT |
| fnrS1 SEQ ID NO: 159 | AGTTGTTCTTATTGGTGGTGTTGCTTTATGGTTGCATCGTAGTAAATGGTTGTAACAAAAGCAATTTTTCCGGCTGTCTGTATACAAAAACGCCGTAAAGTTTGAGCGAAGTCAATAAACTCTCTACCCATTCAGGGCAATATCTCTCTTGGATCCCTCTAGAAATAATTTTGTTTAACTTTAAGAAGGAGATATACAT |
| fnrS2 SEQ ID NO: 160 | AGTTGTTCTTATTGGTGGTGTTGCTTTATGGTTGCATCGTAGTAAATGGTTGTAACAAAAGCAATTTTTCCGGCTGTCTGTATACAAAAACGCCGCAAAGTTTGAGCGAAGTCAATAAACTCTCTACCCATTCAGGGCAATATCTCTCTTGGATCCAAAGTGAACTCTAGAAATAATTTTGTTTAACTTTAAGAAGGAGATATACAT |
| nirB+crp SEQ ID NO: 161 | TCGTCTTTGTGATGTGCTTCCTGTTAGGTTTCGTCAGCCGTCACCGTCAGCATAACACCCTGACCTCTCATTAATTGCTCATGCCGGACGGCACTATCGTCGTCCGGCCTTTTCCTCTCTTCCCCCGCTACGTGCATCTATTTCTATAAACCCGCTCATTTTGTCTATTTTTTGCACAAACATGAAATATCAGACAATTCCGTGACTTAAGAAAATTTATACAAATCAGCAATATACCCATTAAGGAGTATATAAAGGTGAATTTGATTTACATCAATAAGCGGGGTTGCTGAATCGTTAAGGTAGaaatgtgatctagttcacatttGCGGTAATAGAAAAGAAATCGAGGCAAAAatgtttg tttaactttaagaaggagatatacat |
| fnrS+crp SEQ ID NO: 162 | AGTTGTTCTTATTGGTGGTGTTGCTTTATGGTTGCATCGTAGTAAATGGTTGTAACAAAAGCAATTTTTCCGGCTGTCTGTATACAAAAACGCCGCAAAGTTTGAGCGAAGTCAATAAACTCTCTACCCATTCAGGGCAATATCTCTCaaatgtgatctagttcacattttttgtttaactttaagaaggagatatacat |

In some embodiments, multiple distinct FNR nucleic acid sequences are inserted in the bacteria. In alternate embodiments, the bacteria comprise a gene encoding a leucine enzyme, or other enzyme disclosed herein, is expressed under the control of an alternate oxygen level-dependent promoter, e.g., DNR (Trunk et al., 2010) or ANR (Ray et al., 1997). In alternate embodiments, the bacteria comprise at least one gene encoding a transporter, e.g., livKHMGF and/or brnQ, is expressed under the control of an alternate oxygen level-dependent promoter, e.g., DNR (Trunk et al., 2010) or ANR (Ray et al., 1997). In these embodiments, catabolism of leucine is particularly activated in a low-oxygen or anaerobic environment, such as in the gut. In some embodiments, gene expression is further optimized by methods known in the art, e.g., by optimizing ribosomal binding sites and/or increasing mRNA stability. In one embodiment, the mammalian gut is a human mammalian gut.

In some embodiments, the bacterial cell comprises an oxygen-level dependent transcriptional regulator, e.g., FNR, ANR, or DNR, and corresponding promoter from a different bacterial species. The heterologous oxygen-level dependent transcriptional regulator and promoter increase the transcription of genes operably linked to said promoter, e.g., the gene encoding the leucine catabolism enzyme, and/or the at least one gene encoding a transporter, e.g., livKHMGF and/or brnQ, in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene encoding the leucine catabolism enzyme, and/or the at least one gene encoding a transporter, e.g., livKHMGF and/or brnQ, in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene encoding the leucine catabolism enzyme, and/or the at least one gene encoding a transporter, e.g., livKHMGF and/or brnQ, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., 2006).

In some embodiments, the bacterial cells disclosed herein comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding the leucine catabolism enzyme are present on different plasmids. In some embodiments, the gene encoding the oxygen level-sensing transcriptional regulator and the gene encoding the leucine catabolism enzyme and/or the at least one gene encoding a transporter of leucine. In some embodiments, the gene encoding the oxygen level-sensing transcriptional regulator and the gene encoding the leucine catabolism enzyme and/or the at least one gene encoding a transporter are present on the same plasmid.

In some embodiments, the gene encoding the oxygen level-sensing transcriptional regulator is present on a chromosome. In some embodiments, the gene encoding the oxygen level-sensing transcriptional regulator and the gene encoding the gene encoding the leucine catabolism enzyme and/or the at least one gene encoding a transporter are present on different chromosomes. In some embodiments, the gene encoding the oxygen level-sensing transcriptional regulator and the gene encoding the leucine catabolism enzyme and/or the at least one gene encoding a transporter are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the leucine catabolism enzyme and/or leucine transporter and/or leucine binding protein. In some embodiments, expression of the transcriptional regulator is controlled by the same promoter that controls expression of the leucine catabolism enzyme and/or leucine transporter and/or leucine binding protein. In some embodiments, the transcriptional regulator and the leucine catabolism enzyme are divergently transcribed from a promoter region.

### RNS-Dependent Regulation

In some embodiments, the bacteria comprise a gene encoding a leucine catabolism enzyme that is expressed under the control of an inducible promoter. In some embodiments, the bacterium that expresses a leucine catabolism enzyme and/or leucine transporter and/or leucine binding protein is under the control of a promoter that is activated by inflammatory conditions. In one embodiment, the gene for producing the leucine catabolism enzyme and/or leucine transporter and/or leucine binding protein is expressed under the control of an inflammatory-dependent promoter that is activated in inflammatory environments, e.g., a reactive nitrogen species or RNS promoter.

As used herein, “reactive nitrogen species” and “RNS” are used interchangeably to refer to highly active molecules, ions, and/or radicals derived from molecular nitrogen. RNS can cause deleterious cellular effects such as nitrosative stress. RNS includes, but is not limited to, nitric oxide (NO•), peroxynitrite or peroxynitrite anion (ONOO—), nitrogen dioxide (•NO2), dinitrogen trioxide (N2O3), peroxynitrous acid (ONOOH), and nitroperoxycarbonate (ONOOCO2—) (unpaired electrons denoted by •). Bacteria have evolved transcription factors that are capable of sensing RNS levels. Different RNS signaling pathways are triggered by different RNS levels and occur with different kinetics.

As used herein, “RNS-inducible regulatory region” refers to a nucleic acid sequence to which one or more RNS-sensing transcription factors is capable of binding, wherein the binding and/or activation of the corresponding transcription factor activates downstream gene expression; in the presence of RNS, the transcription factor binds to and/or activates the regulatory region. In some embodiments, the RNS-inducible regulatory region comprises a promoter sequence. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; in the presence of RNS, the transcription factor undergoes a conformational change, thereby activating downstream gene expression. The RNS-inducible regulatory region may be operatively linked to a gene or genes, e.g., a leucine catabolism enzymegene sequence(s), e.g., any of the amino acid catabolism enzymes described herein. For example, in the presence of RNS, a transcription factor senses RNS and activates a corresponding RNS-inducible regulatory region, thereby driving expression of an operatively linked gene sequence. Thus, RNS induces expression of the gene or gene sequences.

As used herein, “RNS-derepressible regulatory region” refers to a nucleic acid sequence to which one or more RNS-sensing transcription factors is capable of binding, wherein the binding of the corresponding transcription factor represses downstream gene expression; in the presence of RNS, the transcription factor does not bind to and does not repress the regulatory region. In some embodiments, the RNS-derepressible regulatory region comprises a promoter sequence. The RNS-derepressible regulatory region may be operatively linked to a gene or genes, e.g., a branched chain amino acid catabolism enzymegene sequence(s), BCAA transporter sequence(s), BCAA binding protein(s). For example, in the presence of RNS, a transcription factor senses RNS and no longer binds to and/or represses the regulatory region, thereby derepressing an operatively linked gene sequence or gene cassette. Thus, RNS derepresses expression of the gene or genes.

As used herein, “RNS-repressible regulatory region” refers to a nucleic acid sequence to which one or more RNS-sensing transcription factors is capable of binding, wherein the binding of the corresponding transcription factor represses downstream gene expression; in the presence of RNS, the transcription factor binds to and represses the regulatory region. In some embodiments, the RNS-repressible regulatory region comprises a promoter sequence. In some embodiments, the transcription factor that senses RNS is capable of binding to a regulatory region that overlaps with part of the promoter sequence. In alternate embodiments, the transcription factor that senses RNS is capable of binding to a regulatory region that is upstream or downstream of the promoter sequence. The RNS-repressible regulatory region may be operatively linked to a gene sequence or gene cassette. For example, in the presence of RNS, a transcription factor senses RNS and binds to a corresponding RNS-repressible regulatory region, thereby blocking expression of an operatively linked gene sequence or gene sequences. Thus, RNS represses expression of the gene or gene sequences.

As used herein, a “RNS-responsive regulatory region” refers to a RNS-inducible regulatory region, a RNS-repressible regulatory region, and/or a RNS-derepressible regulatory region. In some embodiments, the RNS-responsive regulatory region comprises a promoter sequence. Each regulatory region is capable of binding at least one corresponding RNS-sensing transcription factor. Examples of transcription factors that sense RNS and their corresponding RNS-responsive genes, promoters, and/or regulatory regions include, but are not limited to, those shown in Table 5.

TABLE 5

Examples of RNS-sensing transcription factors and RNS-responsive genes

| | | |
|---|---|---|
| RNS-sensing transcription factor: | Primarily capable of sensing: | Examples of responsive genes, promoters, and/or regulatory regions: |
| NsrR | NO | norB, aniA, nsrR, hmpA, ytfE, ygbA, hcp, hcr, nrfA, aox |
| NorR | NO | norVW, norR |
| DNR | NO | norCB, nir, nor, nos |

In some embodiments, the bacteria comprise a tunable regulatory region that is directly or indirectly controlled by a transcription factor that is capable of sensing at least one reactive nitrogen species. The tunable regulatory region is operatively linked to a gene or genes capable of directly or indirectly driving the expression of a leucine catabolism enzyme, leucine transporter, and/or leucine binding protein, thus controlling expression of the leucine catabolism enzyme, leucine transporter, and/or leucine binding protein relative to RNS levels. For example, the tunable regulatory region is a RNS-inducible regulatory region, and the payload is a leucine catabolism enzyme, leucine transporter, and/or leucine binding protein, such as any of the leucine catabolism enzymes, leucine transporters, and leucine binding proteins provided herein; when RNS is present, e.g., in an inflamed tissue, a RNS-sensing transcription factor binds to and/or activates the regulatory region and drives expression of the leucine catabolism enzyme, leucine transporter, and/or leucine binding protein gene or genes. Subsequently, when inflammation is ameliorated, RNS levels are reduced, and production of the leucine catabolism enzyme, leucine transporter, and/or leucine binding protein is decreased or eliminated.

In some embodiments, the tunable regulatory region is a RNS-inducible regulatory region; in the presence of RNS, a transcription factor senses RNS and activates the RNS-inducible regulatory region, thereby driving expression of an operatively linked gene or genes. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; when the transcription factor senses RNS, it undergoes a conformational change, thereby inducing downstream gene expression.

In some embodiments, the tunable regulatory region is a RNS-inducible regulatory region, and the transcription factor that senses RNS is NorR. NorR “is an NO-responsive transcriptional activator that regulates expression of the norVW genes encoding flavorubredoxin and an associated flavoprotein, which reduce NO to nitrous oxide” (Spiro 2006). The bacteria may comprise any suitable RNS-responsive regulatory region from a gene that is activated by NorR. Genes that are capable of being activated by NorR are known in the art (see, e.g., Spiro 2006; Vine et al., 2011; Karlinsey et al., 2012). In certain embodiments, the bacteria comprise a RNS-inducible regulatory region from norVW that is operatively linked to a gene or genes, e.g., one or more branched chain amino acid catabolism enzyme, BCAA transporter, and/or BCAA binding protein gene sequence(s). In the presence of RNS, a NorR transcription factor senses RNS and activates to the norVW regulatory region, thereby driving expression of the operatively linked gene(s) and producing the leucine catabolism enzyme, leucine transporter, and/or leucine binding protein.

In some embodiments, the tunable regulatory region is a RNS-inducible regulatory region, and the transcription factor that senses RNS is DNR. DNR (dissimilatory nitrate respiration regulator) “promotes the expression of the nir, the nor and the nos genes” in the presence of nitric oxide (Castiglione et al., 2009). The bacteria of the invention may comprise any suitable RNS-responsive regulatory region from a gene that is activated by DNR. Genes that are capable of being activated by DNR are known in the art (see, e.g., Castiglione et al., 2009; Giardina et al., 2008). In certain embodiments, the bacteria of the invention comprise a RNS-inducible regulatory region from norCB that is operatively linked to a gene or gene cassette. In the presence of RNS, a DNR transcription factor senses RNS and activates to the norCB regulatory region, thereby driving expression of the operatively linked gene or genes and producing one or more leucine catabolism enzymes. In some embodiments, the DNR is Pseudomonas aeruginosa DNR.

In some embodiments, the tunable regulatory region is a RNS-derepressible regulatory region, and binding of a corresponding transcription factor represses downstream gene expression; in the presence of RNS, the transcription factor no longer binds to the regulatory region, thereby derepressing the operatively linked gene or gene cassette.

In some embodiments, the tunable regulatory region is a RNS-derepressible regulatory region, and the transcription factor that senses RNS is NsrR. NsrR is “an Rrf2-type transcriptional repressor [that] can sense NO and control the expression of genes responsible for NO metabolism” (Isabella et al., 2009). The bacteria may comprise any suitable RNS-responsive regulatory region from a gene that is repressed by NsrR. In some embodiments, the NsrR is Neisseria gonorrhoeae NsrR. Genes that are capable of being repressed by NsrR are known in the art (see, e.g., Isabella et al., 2009; Dunn et al., 2010). In certain embodiments, the bacteria comprise a RNS-derepressible regulatory region from norB that is operatively linked to a gene or genes, e.g., a leucine catabolism enzyme gene or genes. In the presence of RNS, an NsrR transcription factor senses RNS and no longer binds to the norB regulatory region, thereby derepressing the operatively linked leucine catabolism enzyme, leucine transporter, and/or leucine binding protein gene or genes and producing the leucine catabolism enzyme(s).

In some embodiments, it is advantageous for the bacteria to express a RNS-sensing transcription factor that does not regulate the expression of a significant number of native genes in the bacteria. In some embodiments, the bacterium expresses a RNS-sensing transcription factor from a different species, strain, or substrain of bacteria, wherein the transcription factor does not bind to regulatory sequences in the bacterium of the invention. In some embodiments, the bacterium is Escherichia coli, and the RNS-sensing transcription factor is NsrR, e.g., from is Neisseria gonorrhoeae, wherein the Escherichia coli does not comprise binding sites for said NsrR. In some embodiments, the heterologous transcription factor minimizes or eliminates off-target effects on endogenous regulatory regions and genes in the bacteria.

In some embodiments, the tunable regulatory region is a RNS-repressible regulatory region, and binding of a corresponding transcription factor represses downstream gene expression; in the presence of RNS, the transcription factor senses RNS and binds to the RNS-repressible regulatory region, thereby repressing expression of the operatively linked gene or gene cassette. In some embodiments, the RNS-sensing transcription factor is capable of binding to a regulatory region that overlaps with part of the promoter sequence. In alternate embodiments, the RNS-sensing transcription factor is capable of binding to a regulatory region that is upstream or downstream of the promoter sequence.

In these embodiments, the bacteria may comprise a two repressor activation regulatory circuit, which is used to express a leucine catabolism enzyme. The two repressor activation regulatory circuit comprises a first RNS-sensing repressor and a second repressor, which is operatively linked to a gene or gene cassette, e.g., encoding a leucine catabolism enzyme. In one aspect of these embodiments, the RNS-sensing repressor inhibits transcription of the second repressor, which inhibits the transcription of the gene or gene cassette. Examples of second repressors useful in these embodiments include, but are not limited to, TetR, C1, and LexA. In the absence of binding by the first repressor (which occurs in the absence of RNS), the second repressor is transcribed, which represses expression of the gene or genes. In the presence of binding by the first repressor (which occurs in the presence of RNS), expression of the second repressor is repressed, and the gene or genes, e.g., a leucine catabolism enzyme, leucine transporter, and/or leucine binding protein gene or genes is expressed.

A RNS-responsive transcription factor may induce, derepress, or repress gene expression depending upon the regulatory region sequence used in the bacteria. One or more types of RNS-sensing transcription factors and corresponding regulatory region sequences may be present in the bacteria. In some embodiments, the bacteria comprise one type of RNS-sensing transcription factor, e.g., NsrR, and one corresponding regulatory region sequence, e.g., from norB. In some embodiments, the bacteria comprise one type of RNS-sensing transcription factor, e.g., NsrR, and two or more different corresponding regulatory region sequences, e.g., from norB and aniA. In some embodiments, the bacteria comprise two or more types of RNS-sensing transcription factors, e.g., NsrR and NorR, and two or more corresponding regulatory region sequences, e.g., from norB and norR, respectively. One RNS-responsive regulatory region may be capable of binding more than one transcription factor. In some embodiments, the bacteria comprise two or more types of RNS-sensing transcription factors and one corresponding regulatory region sequence. Nucleic acid sequences of several RNS-regulated regulatory regions are known in the art (see, e.g., Spiro 2006; Isabella et al., 2009; Dunn et al., 2010; Vine et al., 2011; Karlinsey et al., 2012).

In some embodiments, the bacteria comprise a gene encoding a RNS-sensing transcription factor, e.g., the nsrR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the RNS-sensing transcription factor under the control of an inducible promoter in order to enhance expression stability. In some embodiments, expression of the RNS-sensing transcription factor is controlled by a different promoter than the promoter that controls expression of the at least one leucine catabolism enzyme. In some embodiments, expression of the RNS-sensing transcription factor is controlled by the same promoter that controls expression of the at least one leucine catabolism enzyme. In some embodiments, the RNS-sensing transcription factor and therapeutic molecule are divergently transcribed from a promoter region.

In some embodiments, the bacteria comprise a gene for a RNS-sensing transcription factor from a different species, strain, or substrain of bacteria. In some embodiments, the bacteria comprise a RNS-responsive regulatory region from a different species, strain, or substrain of bacteria. In some embodiments, the bacteria comprise a RNS-sensing transcription factor and corresponding RNS-responsive regulatory region from a different species, strain, or substrain of bacteria. The heterologous RNS-sensing transcription factor and regulatory region may increase the transcription of genes operatively linked to said regulatory region in the presence of RNS, as compared to the native transcription factor and regulatory region from bacteria of the same subtype under the same conditions.

In some embodiments, the bacteria comprise a RNS-sensing transcription factor, NsrR, and corresponding regulatory region, nsrR, from Neisseria gonorrhoeae. In some embodiments, the native RNS-sensing transcription factor, e.g., NsrR, is left intact and retains wild-type activity. In alternate embodiments, the native RNS-sensing transcription factor, e.g., NsrR, is deleted or mutated to reduce or eliminate wild-type activity.

In some embodiments, the bacteria comprise multiple copies of the endogenous gene encoding the RNS-sensing transcription factor, e.g., the nsrR gene. In some embodiments, the gene encoding the RNS-sensing transcription factor is present on a plasmid. In some embodiments, the gene encoding the RNS-sensing transcription factor and the gene or gene cassette for producing the leucine catabolism enzyme are present on different plasmids. In some embodiments, the gene encoding the RNS-sensing transcription factor and the gene or gene cassette for producing the leucine catabolism enzyme are present on the same plasmid. In some embodiments, the gene encoding the RNS-sensing transcription factor is present on a chromosome. In some embodiments, the gene encoding the RNS-sensing transcription factor and the gene or gene cassette for producing the leucine catabolism enzyme are present on different chromosomes. In some embodiments, the gene encoding the RNS-sensing transcription factor and the gene or gene cassette for producing the therapeutic molecule are present on the same chromosome.

In some embodiments, the bacteria comprise a wild-type gene encoding a RNS-sensing transcription factor, e.g., the NsrR gene, and a corresponding regulatory region, e.g., a norB regulatory region, that is mutated relative to the wild-type regulatory region from bacteria of the same subtype. The mutated regulatory region increases the expression of the leucine catabolism enzyme in the presence of RNS, as compared to the wild-type regulatory region under the same conditions. In some embodiments, the bacteria comprise a wild-type RNS-responsive regulatory region, e.g., the norB regulatory region, and a corresponding transcription factor, e.g., NsrR, that is mutated relative to the wild-type transcription factor from bacteria of the same subtype. The mutant transcription factor increases the expression of the leucine catabolism enzymein the presence of RNS, as compared to the wild-type transcription factor under the same conditions. In some embodiments, both the RNS-sensing transcription factor and corresponding regulatory region are mutated relative to the wild-type sequences from bacteria of the same subtype in order to increase expression of theleucine catabolism enzyme, leucine transporter, and/or leucine binding protein in the presence of RNS.

In some embodiments, the gene or gene cassette for producing the leucine catabolism enzyme is present on a plasmid and operably linked to a promoter that is induced by RNS. In some embodiments, expression is further optimized by methods known in the art, e.g., by optimizing ribosomal binding sites, manipulating transcriptional regulators, and/or increasing mRNA stability.

In some embodiments, any of the gene(s) of the present disclosure may be integrated into the bacterial chromosome at one or more integration sites. For example, one or more copies of a leucine catabolism enzyme, leucine transporter, and/or leucine binding protein gene(s) may be integrated into the bacterial chromosome. Having multiple copies of the gene or gen(s) integrated into the chromosome allows for greater production of the leucine catabolism enzyme(s) and also permits fine-tuning of the level of expression. Alternatively, different circuits described herein, such as any of the secretion or exporter circuits, in addition to the therapeutic gene(s) or gene cassette(s) could be integrated into the bacterial chromosome at one or more different integration sites to perform multiple different functions.

### ROS-Dependent Regulation

In some embodiments, the bacteria comprise a gene for producing a leucine catabolism enzyme, leucine transporter, and/or leucine binding protein that is expressed under the control of an inducible promoter. In some embodiments, the bacterium that expresses a leucine catabolism enzyme, leucine transporter, and/or leucine binding protein under the control of a promoter that is activated by conditions of cellular damage. In one embodiment, the gene for producing the leucine catabolism enzymeis expressed under the control of a cellular damaged-dependent promoter that is activated in environments in which there is cellular or tissue damage, e.g., a reactive oxygen species or ROS promoter.

As used herein, “reactive oxygen species” and “ROS” are used interchangeably to refer to highly active molecules, ions, and/or radicals derived from molecular oxygen. ROS can be produced as byproducts of aerobic respiration or metal-catalyzed oxidation and may cause deleterious cellular effects such as oxidative damage. ROS includes, but is not limited to, hydrogen peroxide (H2O2), organic peroxide (ROOH), hydroxyl ion (OH—), hydroxyl radical (•OH), superoxide or superoxide anion (•O2—), singlet oxygen (102), ozone (O3), carbonate radical, peroxide or peroxyl radical (O2—2), hypochlorous acid (HOCl), hypochlorite ion (OCl—), sodium hypochlorite (NaOCl), nitric oxide (NO•), and peroxynitrite or peroxynitrite anion (ONOO—) (unpaired electrons denoted by •). Bacteria have evolved transcription factors that are capable of sensing ROS levels. Different ROS signaling pathways are triggered by different ROS levels and occur with different kinetics (Marinho et al., 2014).

As used herein, “ROS-inducible regulatory region” refers to a nucleic acid sequence to which one or more ROS-sensing transcription factors is capable of binding, wherein the binding and/or activation of the corresponding transcription factor activates downstream gene expression; in the presence of ROS, the transcription factor binds to and/or activates the regulatory region. In some embodiments, the ROS-inducible regulatory region comprises a promoter sequence. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; in the presence of ROS, the transcription factor undergoes a conformational change, thereby activating downstream gene expression. The ROS-inducible regulatory region may be operatively linked to a gene sequence or gene sequence, e.g., a sequence or sequences encoding one or more amino acid catabolism enzyme(s). For example, in the presence of ROS, a transcription factor, e.g., OxyR, senses ROS and activates a corresponding ROS-inducible regulatory region, thereby driving expression of an operatively linked gene sequence or gene sequences. Thus, ROS induces expression of the gene or genes.

As used herein, “ROS-derepressible regulatory region” refers to a nucleic acid sequence to which one or more ROS-sensing transcription factors is capable of binding, wherein the binding of the corresponding transcription factor represses downstream gene expression; in the presence of ROS, the transcription factor does not bind to and does not repress the regulatory region. In some embodiments, the ROS-derepressible regulatory region comprises a promoter sequence. The ROS-derepressible regulatory region may be operatively linked to a gene or genes, e.g., one or more genes encoding one or more leucine catabolism enzyme(s). For example, in the presence of ROS, a transcription factor, e.g., OhrR, senses ROS and no longer binds to and/or represses the regulatory region, thereby derepressing an operatively linked gene sequence or gene cassette. Thus, ROS derepresses expression of the gene or gene cassette.

As used herein, “ROS-repressible regulatory region” refers to a nucleic acid sequence to which one or more ROS-sensing transcription factors is capable of binding, wherein the binding of the corresponding transcription factor represses downstream gene expression; in the presence of ROS, the transcription factor binds to and represses the regulatory region. In some embodiments, the ROS-repressible regulatory region comprises a promoter sequence. In some embodiments, the transcription factor that senses ROS is capable of binding to a regulatory region that overlaps with part of the promoter sequence. In alternate embodiments, the transcription factor that senses ROS is capable of binding to a regulatory region that is upstream or downstream of the promoter sequence. The ROS-repressible regulatory region may be operatively linked to a gene sequence or gene sequences. For example, in the presence of ROS, a transcription factor, e.g., PerR, senses ROS and binds to a corresponding ROS-repressible regulatory region, thereby blocking expression of an operatively linked gene sequence or gene sequences. Thus, ROS represses expression of the gene or genes.

As used herein, a “ROS-responsive regulatory region” refers to a ROS-inducible regulatory region, a ROS-repressible regulatory region, and/or a ROS-derepressible regulatory region. In some embodiments, the ROS-responsive regulatory region comprises a promoter sequence. Each regulatory region is capable of binding at least one corresponding ROS-sensing transcription factor. Examples of transcription factors that sense ROS and their corresponding ROS-responsive genes, promoters, and/or regulatory regions include, but are not limited to, those shown in Table 6.

TABLE 6

Examples of ROS-sensing transcription factors and ROS-responsive genes

| | | |
|---|---|---|
| ROS-sensing transcription factor: | Primarily capable of sensing: | Examples of responsive genes, promoters, and/or regulatory regions: |
| OxyR | H₂O₂ | ahpC; ahpF; dps; dsbG; fhuF; flu; fur; gor; grxA; hemH; katG; oxyS; sufA; sufB; sufC; sufD; sufE; sufS; trxC; uxuA; yaaA; yaeH; yaiA; ybjM; ydcH; ydeN; ygaQ; yljA; ytfK |
| PerR | H₂O₂ | katA; ahpCF; mrgA; zoaA; fur; hemAXCDBL; srfA |
| OhrR | Organic peroxides NaOCl | ohrA |
| SoxR | •O₂⁻ NO• (also capable of sensing H₂O₂) | soxS |
| RosR | H₂O₂ | rbtT; tnp16a; rluC1; tnp5a; mscL; tnp2d; phoD; tnp15b; pstA; tnp5b; xylC; gabDl; rluC2; cgtS9; azlC; narKGHJI; rosR |

In some embodiments, the bacteria comprise a tunable regulatory region that is directly or indirectly controlled by a transcription factor that is capable of sensing at least one reactive oxygen species. The tunable regulatory region is operatively linked to a gene or gene cassette capable of directly or indirectly driving the expression of a leucine catabolism enzyme, thus controlling expression of the leucine catabolism enzyme relative to ROS levels. For example, the tunable regulatory region is a ROS-inducible regulatory region, and the molecule is a leucine catabolism enzyme; when ROS is present, e.g., in an inflamed tissue, a ROS-sensing transcription factor binds to and/or activates the regulatory region and drives expression of the gene sequence for the leucine catabolism enzyme, leucine transporter, and/or leucine binding protein thereby producing the leucine catabolism enzyme, leucine transporter, and/or leucine binding protein. Subsequently, when inflammation is ameliorated, ROS levels are reduced, and production of the leucine catabolism enzyme, leucine transporter, and/or leucine binding protein is decreased or eliminated.

In some embodiments, the tunable regulatory region is a ROS-inducible regulatory region; in the presence of ROS, a transcription factor senses ROS and activates the ROS-inducible regulatory region, thereby driving expression of an operatively linked gene or gene cassette. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; when the transcription factor senses ROS, it undergoes a conformational change, thereby inducing downstream gene expression.

In some embodiments, the tunable regulatory region is a ROS-inducible regulatory region, and the transcription factor that senses ROS is OxyR. OxyR “functions primarily as a global regulator of the peroxide stress response” and is capable of regulating dozens of genes, e.g., “genes involved in H₂O₂ detoxification (katE, ahpCF), heme biosynthesis (hemH), reductant supply (grxA, gor, trxC), thiol-disulfide isomerization (dsbG), Fe-S center repair (sufA-E, sufS), iron binding (yaaA), repression of iron import systems (fur)” and “OxyS, a small regulatory RNA” (Dubbs et al., 2012). The genetically engineered bacteria may comprise any suitable ROS-responsive regulatory region from a gene that is activated by OxyR. Genes that are capable of being activated by OxyR are known in the art (see, e.g., Zheng et al., 2001; Dubbs et al., 2012). In certain embodiments, the bacteria comprise a ROS-inducible regulatory region from oxyS that is operatively linked to a gene, e.g., a leucine catabolism enzyme, leucine transporter, and/or leucine binding protein gene. In the presence of ROS, e.g., H₂O₂, an OxyR transcription factor senses ROS and activates to the oxyS regulatory region, thereby driving expression of the operatively linked leucine catabolism enzyme, leucine transporter, and/or leucine binding protein gene and producing the leucine catabolism enzyme, leucine transporter, and/or leucine binding protein. In some embodiments, OxyR is encoded by an E. coli oxyR gene. In some embodiments, the oxyS regulatory region is an E. coli oxyS regulatory region. In some embodiments, the ROS-inducible regulatory region is selected from the regulatory region of katG, dps, and ahpC.

In alternate embodiments, the tunable regulatory region is a ROS-inducible regulatory region, and the corresponding transcription factor that senses ROS is SoxR. When SoxR is “activated by oxidation of its [2Fe-2S] cluster, it increases the synthesis of SoxS, which then activates its target gene expression” (Koo et al., 2003). “SoxR is known to respond primarily to superoxide and nitric oxide” (Koo et al., 2003), and is also capable of responding to H₂O₂. The bacteria may comprise any suitable ROS-responsive regulatory region from a gene that is activated by SoxR. Genes that are capable of being activated by SoxR are known in the art (see, e.g., Koo et al., 2003; Table 1). In certain embodiments, the bacteria comprise a ROS-inducible regulatory region from soxS that is operatively linked to a gene, e.g., a leucine catabolism enzyme. In the presence of ROS, the SoxR transcription factor senses ROS and activates the soxS regulatory region, thereby driving expression of the operatively linked leucine catabolism enzyme, leucine transporter, and/or leucine binding protein gene and producing a leucine catabolism enzyme, leucine transporter, and/or leucine binding protein.

In some embodiments, the tunable regulatory region is a ROS-derepressible regulatory region, and binding of a corresponding transcription factor represses downstream gene expression; in the presence of ROS, the transcription factor no longer binds to the regulatory region, thereby derepressing the operatively linked gene or gene cassette.

In some embodiments, the tunable regulatory region is a ROS-derepressible regulatory region, and the transcription factor that senses ROS is OhrR. OhrR “binds to a pair of inverted repeat DNA sequences overlapping the ohrA promoter site and thereby represses the transcription event,” but oxidized OhrR is “unable to bind its DNA target” (Duarte et al., 2010). OhrR is a “transcriptional repressor [that]... senses both organic peroxides and NaOCl” (Dubbs et al., 2012) and is “weakly activated by H2O2 but it shows much higher reactivity for organic hydroperoxides” (Duarte et al., 2010). The bacteria may comprise any suitable ROS-responsive regulatory region from a gene that is repressed by OhrR. Genes that are capable of being repressed by OhrR are known in the art (see, e.g., Dubbs et al., 2012; Table 1). In certain embodiments, the bacteria comprise a ROS-derepressible regulatory region from ohrA that is operatively linked to a gene or gene cassette, e.g., a leucine catabolism enzyme, leucine transporter, and/or leucine binding protein gene. In the presence of ROS, e.g., NaOCl, an OhrR transcription factor senses ROS and no longer binds to the ohrA regulatory region, thereby derepressing the operatively linked leucine catabolism enzyme, leucine transporter, and/or leucine binding protein gene and producing a leucine catabolism enzyme, leucine transporter, and/or leucine binding protein.

OhrR is a member of the MarR family of ROS-responsive regulators. “Most members of the MarR family are transcriptional repressors and often bind to the -10 or -35 region in the promoter causing a steric inhibition of RNA polymerase binding” (Bussmann et al., 2010). Other members of this family are known in the art and include, but are not limited to, OspR, MgrA, RosR, and SarZ. In some embodiments, the transcription factor that senses ROS is OspR, MgRA, RosR, and/or SarZ, and the bacteria comprises one or more corresponding regulatory region sequences from a gene that is repressed by OspR, MgRA, RosR, and/or SarZ. Genes that are capable of being repressed by OspR, MgRA, RosR, and/or SarZ are known in the art (see, e.g., Dubbs et al., 2012).

In some embodiments, the tunable regulatory region is a ROS-derepressible regulatory region, and the corresponding transcription factor that senses ROS is RosR. RosR is “a MarR-type transcriptional regulator” that binds to an “18-bp inverted repeat with the consensus sequence TTGTTGAYRYRTCAACWA (SEQ ID NO: 168)” and is “reversibly inhibited by the oxidant H₂O₂” (Bussmann et al., 2010). RosR is capable of repressing numerous genes and putative genes, including but not limited to “a putative polyisoprenoid-binding protein (cg1322, gene upstream of and divergent from rosR), a sensory histidine kinase (cgtS9), a putative transcriptional regulator of the Crp/FNR family (cg3291), a protein of the glutathione S-transferase family (cg1426), two putative FMN reductases (cg1150 and cg1850), and four putative monooxygenases (cg0823, cg1848, cg2329, and cg3084)” (Bussmann et al., 2010). The bacteria may comprise any suitable ROS-responsive regulatory region from a gene that is repressed by RosR. Genes that are capable of being repressed by RosR are known in the art (see, e.g., Bussmann et al., 2010). In certain embodiments, the bacteria comprise a ROS-derepressible regulatory region from cgtS9 that is operatively linked to a gene or gene cassette, e.g., a leucine catabolism enzyme, leucine transporter, and/or leucine binding protein. In the presence of ROS, e.g., H₂O₂, a RosR transcription factor senses ROS and no longer binds to the cgtS9 regulatory region, thereby derepressing the operatively linked leucine catabolism enzyme, leucine transporter, and/or leucine binding protein gene and producing the leucine catabolism enzyme, leucine transporter, and/or leucine binding protein.

In some embodiments, it is advantageous for the bacteria to express a ROS-sensing transcription factor that does not regulate the expression of a significant number of native genes in the bacteria. In some embodiments, the bacterium expresses a ROS-sensing transcription factor from a different species, strain, or substrain of bacteria, wherein the transcription factor does not bind to regulatory sequences in the bacterium of the invention. In some embodiments, the bacterium is Escherichia coli, and the ROS-sensing transcription factor is RosR, e.g., from Corynebacterium glutamicum, wherein the Escherichia coli does not comprise binding sites for said RosR. In some embodiments, the heterologous transcription factor minimizes or eliminates off-target effects on endogenous regulatory regions and genes in the bacteria.

In some embodiments, the tunable regulatory region is a ROS-repressible regulatory region, and binding of a corresponding transcription factor represses downstream gene expression; in the presence of ROS, the transcription factor senses ROS and binds to the ROS-repressible regulatory region, thereby repressing expression of the operatively linked gene or gene cassette. In some embodiments, the ROS-sensing transcription factor is capable of binding to a regulatory region that overlaps with part of the promoter sequence. In alternate embodiments, the ROS-sensing transcription factor is capable of binding to a regulatory region that is upstream or downstream of the promoter sequence.

In some embodiments, the tunable regulatory region is a ROS-repressible regulatory region, and the transcription factor that senses ROS is PerR. In Bacillus subtilis, PerR “when bound to DNA, represses the genes coding for proteins involved in the oxidative stress response (katA, ahpC, and mrgA), metal homeostasis (hemAXCDBL, fur, and zoaA) and its own synthesis (perR)” (Marinho et al., 2014). PerR is a “global regulator that responds primarily to H2O2” (Dubbs et al., 2012) and “interacts with DNA at the per box, a specific palindromic consensus sequence (TTATAATNATTATAA (SEQ ID NO: 169)) residing within and near the promoter sequences of PerR-controlled genes” (Marinho et al., 2014). PerR is capable of binding a regulatory region that “overlaps part of the promoter or is immediately downstream from it” (Dubbs et al., 2012). The bacteria may comprise any suitable ROS-responsive regulatory region from a gene that is repressed by PerR. Genes that are capable of being repressed by PerR are known in the art (see, e.g., Dubbs et al., 2012).

In these embodiments, the bacteria may comprise a two repressor activation regulatory circuit, which is used to express a leucine catabolism enzyme. The two repressor activation regulatory circuit comprises a first ROS-sensing repressor, e.g., PerR, and a second repressor, e.g., TetR, which is operatively linked to a gene or gene cassette, e.g., a leucine catabolism enzyme. In one aspect of these embodiments, the ROS-sensing repressor inhibits transcription of the second repressor, which inhibits the transcription of the gene or gene cassette. Examples of second repressors useful in these embodiments include, but are not limited to, TetR, C1, and LexA. In some embodiments, the ROS-sensing repressor is PerR. In some embodiments, the second repressor is TetR. In this embodiment, a PerR-repressible regulatory region drives expression of TetR, and a TetR-repressible regulatory region drives expression of the gene or gene cassette, e.g., an amino acid catabolism enzyme. In the absence of PerR binding (which occurs in the absence of ROS), tetR is transcribed, and TetR represses expression of the gene or gene cassette, e.g., leucine catabolism enzyme. In the presence of PerR binding (which occurs in the presence of ROS), tetR expression is repressed, and the gene or gene cassette, e.g., leucine catabolism enzyme, leucine transporter, and/or leucine binding protein is expressed.

A ROS-responsive transcription factor may induce, derepress, or repress gene expression depending upon the regulatory region sequence used in the bacteria. For example, although “OxyR is primarily thought of as a transcriptional activator under oxidizing conditions... OxyR can function as either a repressor or activator under both oxidizing and reducing conditions” (Dubbs et al., 2012), and OxyR “has been shown to be a repressor of its own expression as well as that of fhuF (encoding a ferric ion reductase) and flu (encoding the antigen 43 outer membrane protein)” (Zheng et al., 2001). The bacteria may comprise any suitable ROS-responsive regulatory region from a gene that is repressed by OxyR. In some embodiments, OxyR is used in a two repressor activation regulatory circuit, as described above. Genes that are capable of being repressed by OxyR are known in the art (see, e.g., Zheng et al., 2001; Table 1). Or, for example, although RosR is capable of repressing a number of genes, it is also capable of activating certain genes, e.g., the narKGHJI operon. In some embodiments, the bacteria comprise any suitable ROS-responsive regulatory region from a gene that is activated by RosR. In addition, “PerR-mediated positive regulation has also been observed...and appears to involve PerR binding to distant upstream sites” (Dubbs et al., 2012). In some embodiments, the bacteria comprise any suitable ROS-responsive regulatory region from a gene that is activated by PerR.

One or more types of ROS-sensing transcription factors and corresponding regulatory region sequences may be present in bacteria. For example, “OhrR is found in both Gram-positive and Gram-negative bacteria and can co-reside with either OxyR or PerR or both” (Dubbs et al., 2012). In some embodiments, the bacteria comprise one type of ROS-sensing transcription factor, e.g., OxyR, and one corresponding regulatory region sequence, e.g., from oxyS. In some embodiments, the bacteria comprise one type of ROS-sensing transcription factor, e.g., OxyR, and two or more different corresponding regulatory region sequences, e.g., from oxyS and katG. In some embodiments, the genetically engineered bacteria comprise two or more types of ROS-sensing transcription factors, e.g., OxyR and PerR, and two or more corresponding regulatory region sequences, e.g., from oxyS and katA, respectively. One ROS-responsive regulatory region may be capable of binding more than one transcription factor. In some embodiments, the bacteria comprise two or more types of ROS-sensing transcription factors and one corresponding regulatory region sequence.

Nucleic acid sequences of several exemplary OxyR-regulated regulatory regions are shown in Table 7. OxyR binding sites are underlined and bolded. In some embodiments, genetically engineered bacteria comprise a nucleic acid sequence that is at least about 80%, at least about 85%, at least about 90%, at least about 95%, or at least about 99% homologous to the DNA sequence, or a functional fragment thereof.

TABLE 7

Nucleotide sequences of exemplary OxyR-regulated regulatory regions

| | |
|---|---|
| Regulatory sequence | 01234567890123456789012345678901234567890123456789 |
| katG (SEQ ID NO: 163) | TGTGGCTTTTATGAAAATCACACAGTGATCACAAATTTTAAACAGAGC ACAAAATGCTGCCTCGAAATGAGGGCGGGAAAATAAGGTTATCAGCC TTGTTTTCTCCCTCATTACTTGAAGGATATGAAGCTAAAACCCTTTTTT ATAAAGCATTTGTCCGAATTCGGACATAATCAAAAAAGCTTAATTAAG ATCAATTTGATCTACATCTCTTTAACCAACAATATGTAAGATCTCAAC TATCGCATCCGTGGATTAATTCAATTATAACTTCTCTCTAACGCTGTG TATCGTAACGGTAACACTGTAGAGGGGAGCACATTGATGCGAATTCAT TAAAGAGGAGAAAGGTACC |
| dps (SEQ ID NO: 164) | TTCCGAAAATTCCTGGCGAGCAGATAAATAAGAATTGTTCTTATCAAT ATATCTAACTCATTGAATCTTTATTAGTTTTGTTTTTCACGCTTGTTAC CACTATTAGTGTGATAGGAACAGCCAGAATAGCGGAACACATAGCC GGTGCTATACTTAATCTCGTTAATTACTGGGACATAACATCAAGAGGA TATGAAATTCGAATTCATTAAAGAGGAGAAAGGTACC |
| ahpC (SEQ ID NO: 165) | GCTTAGATCAGGTGATTGCCCTTTGTTTATGAGGGTGTTGTAATCCATG TCGTTGTTGCATTTGTAAGGGCAACACCTCAGCCTGCAGGCAGGCACT GAAGATACCAAAGGGTAGTTCAGATTACACGGTCACCTGGAAAGGGG GCCATTTTACTTTTTATCGCCGCTGGCGGTGCAAAGTTCACAAAGTTGT CTTACGAAGGTTGTAAGGTAAAACTTATCGATTTGATAATGGAAAC GCATTAGCCGAATCGGCAAAAATTGGTTACCTTACATCTCATCGAAAA CACGGAGGAAGTATAGATGCGAATTCATTAAAGAGGAGAAAGGTACC |
| oxyS (SEQ ID NO: 166) | CTCGAGTTCATTATCCATCCTCCATCGCCACGATAGTTCATGGCGATA GGTAGAATAGCAATGAACGATTATCCCTATCAAGCATTCTGACTGAT AATTGCTCACACGAATTCATTAAAGAGGAGAAAGGTACC |

In some embodiments, the bacteria comprise a gene encoding a ROS-sensing transcription factor, e.g., the oxyR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the ROS-sensing transcription factor under the control of an inducible promoter in order to enhance expression stability. In some embodiments, expression of the ROS-sensing transcription factor is controlled by a different promoter than the promoter that controls expression of the therapeutic molecule. In some embodiments, expression of the ROS-sensing transcription factor is controlled by the same promoter that controls expression of the therapeutic molecule. In some embodiments, the ROS-sensing transcription factor and therapeutic molecule are divergently transcribed from a promoter region.

In some embodiments, the bacteria comprise a gene for a ROS-sensing transcription factor from a different species, strain, or substrain of bacteria. In some embodiments, the genetically engineered bacteria comprise a ROS-responsive regulatory region from a different species, strain, or substrain of bacteria. In some embodiments, the genetically engineered bacteria comprise a ROS-sensing transcription factor and corresponding ROS-responsive regulatory region from a different species, strain, or substrain of bacteria. The heterologous ROS-sensing transcription factor and regulatory region may increase the transcription of genes operatively linked to said regulatory region in the presence of ROS, as compared to the native transcription factor and regulatory region from bacteria of the same subtype under the same conditions.

In some embodiments, the genetically engineered bacteria comprise a ROS-sensing transcription factor, OxyR, and corresponding regulatory region, oxyS, from Escherichia coli. In some embodiments, the native ROS-sensing transcription factor, e.g., OxyR, is left intact and retains wild-type activity. In alternate embodiments, the native ROS-sensing transcription factor, e.g., OxyR, is deleted or mutated to reduce or eliminate wild-type activity.

In some embodiments, the bacteria comprise multiple copies of the endogenous gene encoding the ROS-sensing transcription factor, e.g., the oxyR gene. In some embodiments, the gene encoding the ROS-sensing transcription factor is present on a plasmid. In some embodiments, the gene encoding the ROS-sensing transcription factor and the gene or gene cassette for producing the therapeutic molecule are present on different plasmids. In some embodiments, the gene encoding the ROS-sensing transcription factor and the gene or gene cassette for producing the therapeutic molecule are present on the same. In some embodiments, the gene encoding the ROS-sensing transcription factor is present on a chromosome. In some embodiments, the gene encoding the ROS-sensing transcription factor and the gene or gene cassette for producing the therapeutic molecule are present on different chromosomes. In some embodiments, the gene encoding the ROS-sensing transcription factor and the gene or gene cassette for producing the therapeutic molecule are present on the same chromosome.

In some embodiments, the genetically engineered bacteria comprise a wild-type gene encoding a ROS-sensing transcription factor, e.g., the soxR gene, and a corresponding regulatory region, e.g., a soxS regulatory region, that is mutated relative to the wild-type regulatory region from bacteria of the same subtype. The mutated regulatory region increases the expression of the leucine catabolism enzyme, leucine transporter, and/or leucine binding protein in the presence of ROS, as compared to the wild-type regulatory region under the same conditions. In some embodiments, the genetically engineered bacteria comprise a wild-type ROS-responsive regulatory region, e.g., the oxyS regulatory region, and a corresponding transcription factor, e.g., OxyR, that is mutated relative to the wild-type transcription factor from bacteria of the same subtype. The mutant transcription factor increases the expression of the leucine catabolism enzyme, leucine transporter, and/or leucine binding protein in the presence of ROS, as compared to the wild-type transcription factor under the same conditions. In some embodiments, both the ROS-sensing transcription factor and corresponding regulatory region are mutated relative to the wild-type sequences from bacteria of the same subtype in order to increase expression of the leucine catabolism enzymein the presence of ROS.

In some embodiments, the gene or gene cassette for producing the leucine catabolism enzyme is present on a plasmid and operably linked to a promoter that is induced by ROS. In some embodiments, the gene or gene cassette for producing the leucine catabolism enzymeis present in the chromosome and operably linked to a promoter that is induced by ROS. In some embodiments, the gene or gene cassette for producing the leucine catabolism enzymeis present on a chromosome and operably linked to a promoter that is induced by exposure to tetracycline. In some embodiments, the gene or gene cassette for producing the leucine enzyme, leucine transporter, and/or leucine binding protein is present on a plasmid and operably linked to a promoter that is induced by exposure to tetracycline. In some embodiments, expression is further optimized by methods known in the art, e.g., by optimizing ribosomal binding sites, manipulating transcriptional regulators, and/or increasing mRNA stability.

In some embodiments, the genetically engineered bacteria may comprise multiple copies of the gene(s) capable of producing leucine catabolism enzyme(s), leucine transporter(s), and/or leucine binding protein(s). In some embodiments, the gene(s) capable of producing a leucine catabolism enzyme(s), leucine transporter(s), and/or leucine binding protein(s) is present on a plasmid and operatively linked to a ROS-responsive regulatory region. In some embodiments, the gene(s) capable of producing a leucine catabolism enzyme, leucine transporter, and/or leucine binding protein is present in a chromosome and operatively linked to a ROS-responsive regulatory region.

Thus, in some embodiments, the genetically engineered bacteria or genetically engineered yeast or virus produce one or more amino acid catabolism enzymes under the control of an oxygen level-dependent promoter, a reactive oxygen species (ROS)-dependent promoter, or a reactive nitrogen species (RNS)-dependent promoter, and a corresponding transcription factor.

In some embodiments, the genetically engineered bacteria comprise a stably maintained plasmid or chromosome carrying a gene for producing a leucine catabolism enzyme, transporter, and/or binding protein such that the leucine catabolism enzyme, leucine transporter, and/or leucine binding protein can be expressed in the host cell, and the host cell is capable of survival and/or growth in vitro, e.g., in medium, and/or in vivo. In some embodiments, a bacterium may comprise multiple copies of the gene encoding the leucine catabolism enzyme, leucine transporter, and/or leucine binding protein. In some embodiments, the gene encoding the leucine catabolism enzyme, leucine transporter, and/or leucine binding protein is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, the gene encoding the leucine catabolism enzyme, leucine transporter, and/or leucine binding protein is expressed on a high-copy plasmid. In some embodiments, the high-copy plasmid may be useful for increasing expression of the leucine catabolism enzyme, leucine transporter, and/or leucine binding protein. In some embodiments, the gene encoding the leucine catabolism enzyme, leucine transporter, and/or leucine binding protein is expressed on a chromosome.

In some embodiments, the bacteria are genetically engineered to include multiple mechanisms of action (MOAs), e.g., circuits producing multiple copies of the same product (e.g., to enhance copy number) or circuits performing multiple different functions. For example, the genetically engineered bacteria may include four copies of the gene encoding a particular leucine catabolism enzyme, leucine transporter, and/or leucine binding protein inserted at four different insertion sites. Alternatively, the bacteria may include three copies of the gene encoding a particular leucine catabolism enzyme, leucine transporter, and/or leucine binding protein inserted at three different insertion sites and three copies of the gene encoding a different leucine catabolism enzyme, leucine transporter, and/or leucine binding protein inserted at three different insertion sites.

In some embodiments, under conditions where the leucine catabolism enzyme, leucine transporter, and/or leucine binding protein is expressed, the bacteria of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the leucine catabolism enzyme, leucine transporter, and/or leucine binding protein and/or transcript of the gene(s) in the operon as compared to unmodified bacteria of the same subtype under the same conditions.

In some embodiments, quantitative PCR (qPCR) is used to amplify, detect, and/or quantify mRNA expression levels of the leucine catabolism enzyme, leucine transporter, and/or leucine binding protein gene(s). Primers specific of rleucine catabolism enzyme, leucine transporter, and/or leucine binding protein gene(s) may be designed and used to detect mRNA in a sample according to methods known in the art. In some embodiments, a fluorophore is added to a sample reaction mixture that may contain branched chain amino acid catabolism enzymemRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the leucine catabolism enzyme, leucine transporter, and/or leucine binding protein gene(s).

In some embodiments, quantitative PCR (qPCR) is used to amplify, detect, and/or quantify mRNA expression levels of the leucine catabolism enzyme, leucine transporter, and/or leucine binding protein gene(s). Primers specific for leucine catabolism enzyme, leucine transporter, and/or leucine binding protein gene(s) may be designed and used to detect mRNA in a sample according to methods known in the art. In some embodiments, a fluorophore is added to a sample reaction mixture that may contain leucine catabolism enzyme, leucine transporter, and/or leucine binding protein mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the leucine catabolism enzyme, leucine transporter, and/or leucine binding protein gene(s).

In other embodiments, the inducible promoter is a propionate responsive promoter. For example, the prpR promoter is a propionate responsive promoter.

### Inducible Promoters (Nutritional and/or Chemical Inducer(s) and/or Metabolite(s))

In some embodiments, one or more gene sequence(s), e.g., ldh, is present on a plasmid and operably linked to promoter a directly or indirectly inducible by one or more nutritional and/or chemical inducer(s) and/or metabolite(s). In some embodiments, the bacterial cell comprises a stably maintained plasmid or chromosome carrying the gene encoding the leucine catabolism enzyme, which is induced by one or more nutritional and/or chemical inducer(s) and/or metabolite(s), such that the leucine catabolism enzyme can be expressed in the host cell, and the host cell is capable of survival and/or growth in vitro, e.g., under culture conditions, and/or in vivo, e.g., in the gut. In some embodiments, bacterial cell comprises two or more distinct branched chain amino acid catabolism cassette(s), one or more of which are induced by one or more nutritional and/or chemical inducer(s) and/or metabolite(s). In some embodiments, the genetically engineered bacteria comprise multiple copies of the same branched chain amino acid catabolism enzyme gene(s) and/or gene cassette(s) which are induced by one or more nutritional and/or chemical inducer(s) and/or metabolite(s). In some embodiments, the genetically engineered bacteria comprise multiple copies of different branched chain amino acid catabolism enzyme genes or gene cassette(s), one or more of which are induced by one or more nutritional and/or chemical inducer(s) and/or metabolite(s).

In some embodiments, the gene encoding the branched chain amino acid catabolism enzyme is present on a plasmid and operably linked to a promoter that is induced by one or more nutritional and/or chemical inducer(s) and/or metabolite(s). In some embodiments, the gene encoding the branched chain amino acid catabolism enzyme is present in the chromosome and operably linked to a promoter that is induced by one or more nutritional and/or chemical inducer(s) and/or metabolite(s).

In some embodiments, the bacterial cell comprises a stably maintained plasmid or chromosome carrying the one or more gene sequences(s), inducible by one or more nutritional and/or chemical inducer(s) and/or metabolite(s), encoding a transporter of branched chain amino acid(s) and/or one or more metabolites thereof, e.g., livKHMGF and/or bmQ, such that the transporter can be expressed in the host cell, and the host cell is capable of survival and/or growth in vitro, e.g., in medium, and/or in vivo, e.g., in the gut. In some embodiments, bacterial cell comprises two or more distinct copies of the one or more gene sequences(s) encoding a branched chain amino acid transporter, which is controlled by a promoter inducible one or more nutritional and/or chemical inducer(s) and/or metabolite(s). In some embodiments, the genetically engineered bacteria comprise multiple copies of the same one or more gene sequences(s) encoding a branched chain amino acid transporter, which is controlled by a promoter inducible one or more nutritional and/or chemical inducer(s) and/or metabolite(s). In some embodiments, the one or more gene sequences(s) encoding a transporter of branched chain amino acid(s), is present on a plasmid and operably linked to a directly or indirectly inducible promoter inducible by one or more nutritional and/or chemical inducer(s) and/or metabolite(s). In some embodiments, the one or more gene sequences(s) encoding a branched chain amino acid transporter, is present on a chromosome and operably linked to a directly or indirectly inducible by one or more nutritional and/or chemical inducer(s) and/or metabolite(s).

In some embodiments, the gene encoding branched chain amino acid binding protein is present on a plasmid and operably linked to a promoter that is induced by one or more nutritional and/or chemical inducer(s) and/or metabolite(s). In some embodiments, the gene encoding branched chain amino acid binding protein is present in the chromosome and operably linked to a promoter that is induced by one or more nutritional and/or chemical inducer(s) and/or metabolite(s).

In some embodiments, the promoter that is operably linked to the gene encoding the branched chain amino acid catabolism enzyme and the promoter that is operably linked to the gene encoding the branched chain amino acid transporter and/or BCAA binding protein and/or BCAA exporter, is directly or indirectly induced by one or more nutritional and/or chemical inducer(s) and/or metabolite(s).

In some embodiments, one or more inducible promoter(s) are useful for or induced during in vivo expression of the one or more protein(s) of interest. In some embodiments, the promoters are induced during in vivo expression of one or more branched chain amino acid catabolism enzymes and/or branched chain amino acid transporter(s) and/or BCAA binding protein(s) and/or BCAA exporter(s). In some embodiments, expression of one or more branched chain amino acid catabolism enzyme(s) and/or branched chain amino acid transporter(s) and/or BCAA binding protein(s) and/or BCAA exporter(s) is driven directly or indirectly by one or more arabinose inducible promoter(s) in vivo. In some embodiments, the promoter is directly or indirectly induced by a chemical and/or nutritional inducer and/or metabolite which is co-administered with the bacteria.

In some embodiments, expression of one or more branched chain amino acid catabolism enzyme gene(s) and/or branched chain amino acid transporter(s) and/or BCAA binding protein(s) and/or BCAA exporter(s), is driven directly or indirectly by one or more promoter(s) induced by a chemical and/or nutritional inducer and/or metabolite during in vitro growth, preparation, or manufacturing of the strain prior to in vivo administration. In some embodiments, the promoter(s) induced by a chemical and/or nutritional inducer and/or metabolite are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with branched chain amino acid catabolism enzyme(s) and/or branched chain amino acid transporter(s) and/or BCAA binding protein(s) and/or BCAA exporter(s) prior to administration. In some embodiments, the cultures, which are induced by a chemical and/or nutritional inducer and/or metabolite, are grown aerobically. In some embodiments, the cultures, which are induced by a chemical and/or nutritional inducer and/or metabolite, are grown anaerobically.

In some embodiments, the genetically engineered bacteria encode one or more gene sequence(s) which are inducible through an arabinose inducible system.

The genes of arabinose metabolism are organized in one operon, AraBAD, which is controlled by the PAraBAD promoter. The PAraBAD (or Para) promoter suitably fulfills the criteria of inducible expression systems. PAraBAD displays tighter control of payload gene expression than many other systems, likely due to the dual regulatory role of AraC, which functions both as an inducer and as a repressor. Additionally, the level of ParaBAD-based expression can be modulated over a wide range of L-arabinose concentrations to fine-tune levels of expression of the payload. However, the cell population exposed to sub-saturating L-arabinose concentrations is divided into two subpopulations of induced and uninduced cells, which is determined by the differences between individual cells in the availability of L-arabinose transporter (Zhang et al., Development and Application of an Arabinose-Inducible Expression System by Facilitating Inducer Uptake in Corynebacterium glutamicum; Appl. Environ. Microbiol. August 2012 vol. 78 no. 16 5831-5838). Alternatively, inducible expression from the ParaBad can be controlled or fine-tuned through the optimization of the ribosome binding site (RBS), as described herein.

In one embodiment, expression of one or more enzyme(s), e.g., ldc, e.g., as described herein, is driven directly or indirectly by one or more arabinose inducible promoter(s). In one embodiment, the genetically engineered bacteria encode one or more branched chain amino acid transporter(s) e.g., livKHMGF and/or brnQ, described herein, whose expression is driven directly or indirectly by one or more arabinose inducible promoter(s). In one embodiment, expression of one or more branched chain amino acid exporter(s), e.g., as described herein, is driven directly or indirectly by one or more arabinose inducible promoter(s). In some embodiments, the arabinose inducible promoter is useful for or induced during in vivo expression of the one or more protein(s) of interest. In some embodiments, expression of one or more branched chain amino acid catabolism enzyme(s) and/or branched chain amino acid transporter(s) and/or BCAA binding protein(s) and/or BCAA exporter(s) is driven directly or indirectly by one or more arabinose inducible promoter(s) in vivo. In some embodiments, the promoter is directly or indirectly induced by a molecule (e.g., arabinose) that is co-administered with the bacteria.

In some embodiments, expression of one or more branched chain amino acid catabolism enzyme(s) and/or branched chain amino acid transporter(s) and/or BCAA binding protein(s) and/or BCAA exporter(s), is driven directly or indirectly by one or more arabinose inducible promoter(s) during in vitro growth, preparation, or manufacturing of the strain prior to in vivo administration. In some embodiments, the arabinose inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., arabinose, that is added to in the bacterial culture to induce expression and pre-load the bacterium with branched chain amino acid catabolism enzyme(s) and /or branched chain amino acid transporter(s) and/or BCAA binding protein(s) and/or BCAA exporter(s) prior to administration. In some embodiments, the cultures, which are induced by arabinose, are grown aerobically. In some embodiments, the cultures, which are induced by arabinose, are grown anaerobically.

In some embodiments, bacterial cell comprises two or more distinct branched chain amino acid catabolism cassette(s) or transporter(s) or binding protein(s) or exporter(s), one or more of which are induced by arabinose. In some embodiments, the genetically engineered bacteria comprise multiple copies of the same branched chain amino acid catabolism enzyme gene sequence(s) and/or transporter gene sequence(s), e.g., as described herein, which are induced by one or more nutritional and/or chemical inducer(s) and/or metabolite(s). In some embodiments, the genetically engineered bacteria comprise multiple copies of different branched chain amino acid catabolism enzyme genes sequence(s) and/or transporter gene sequence(s) and/or BCAA binding protein gene sequence(s) and/or BCAA exporter gene sequence(s), e.g., as described herein, one or more of which are induced by one or more nutritional and/or chemical inducer(s) and/or metabolite(s).

In a first example, the arabinose inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the arabinose inducible promoter drives expression under a first set of exogenous conditions, and the second promoter drives the expression under a second set of exogenous conditions. In second example, the arabinose promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., arabinose and IPTG). In another non-limiting example, the first inducing conditions are culture conditions, e.g., the presence of arabinose, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more arabinose promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by arabinose. In some embodiments, the gene sequence(s) encoding the branched chain amino acid catabolism enzyme(s) or branched chain amino acid transporter(s)is present in the chromosome and operably linked to a promoter that is induced by arabinose.

In some embodiments, the arabinose inducible construct further comprises a gene encoding AraC, which is divergently transcribed from the same promoter as the one or more one or more branched chain amino acid catabolism enzyme(s).

In some embodiments, the genetically engineered bacteria comprise one or more gene sequence(s) which are inducible through a rhamnose inducible system. The genes rhaBAD are organized in one operon which is controlled by the rhaP BAD promoter. The rhaP BAD promoter is regulated by two activators, RhaS and RhaR, and the corresponding genes belong to one transcription unit which divergently transcribed in the opposite direction of rhaBAD. In the presence of L-rhamnose, RhaR binds to the rhaP RS promoter and activates the production of RhaR and RhaS. RhaS together with L-rhamnose then bind to the rhaP BAD and the rhaP T promoter and activate the transcription of the structural genes. In contrast to the arabinose system, in which AraC is provided and divergently transcribed in the gene sequence(s), it is not necessary to express the regulatory proteins in larger quantities in the rhamnose expression system because the amounts expressed from the chromosome are sufficient to activate transcription even on multi-copy plasmids. Therefore, only the rhaP BAD promoter is cloned upstream of the gene that is to be expressed. Full induction of rhaBAD transcription also requires binding of the CRP-cAMP complex, which is a key regulator of catabolite repression. Alternatively, inducible expression from the rhaBAD can be controlled or fine-tuned through the optimization of the ribosome binding site (RBS), as described herein.

In one embodiment, expression of one or more branched chain amino acid catabolism enzyme(s), e.g., ldc, e.g., as described herein, is driven directly or indirectly by one or more rhamnose inducible promoter(s). In one embodiment, the genetically engineered bacteria encode one or more branched chain amino acid transporter(s), e.g., livKHMGF and/or brnQ, described herein, whose expression is driven directly or indirectly by one or more rhamnose inducible promoter(s). In one embodiment, the genetically engineered bacteria encode one or more branched chain amino acid exporter(s), described herein, whose expression is driven directly or indirectly by one or more rhamnose inducible promoter(s).

In some embodiments, the rhamnose inducible promoter is useful for or induced during in vivo expression of the one or more protein(s) of interest. In some embodiments, expression of one or more branched chain amino acid catabolism enzyme(s) and/or branched chain amino acid transporter(s) and/or BCAA binding protein(s) and/or BCAA exporter(s) is driven directly or indirectly by one or more rhamnose inducible promoter(s) in vivo. In some embodiments, the promoter is directly or indirectly induced by a molecule (e.g., rhamnose) that is co-administered with the bacteria.

In some embodiments, expression of one or more branched chain amino acid catabolism enzyme(s) and/or branched chain amino acid transporter(s) and/or BCAA binding protein(s) and/or BCAA exporter(s), is driven directly or indirectly by one or more rhamnose inducible promoter(s) during in vitro growth, preparation, or manufacturing of the strain prior to in vivo administration. In some embodiments, the rhamnose inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., rhamnose, that is added to in the bacterial culture to induce expression and pre-load the bacterium with branched chain amino acid catabolism enzyme(s) and/or BCAA transporter(s) and/or BCAA binding protein(s) and/or BCAA exporter(s) prior to administration. In some embodiments, the cultures, which are induced by rhamnose, are grown aerobically. In some embodiments, the cultures, which are induced by rhamnose, are grown anaerobically.

In some embodiments, bacterial cell comprises two or more distinct branched chain amino acid catabolism cassette(s) or other polypeptide(s) of interest, one or more of which are induced by rhamnose. In some embodiments, the genetically engineered bacteria comprise multiple copies of the same branched chain amino acid catabolism enzyme gene sequence(s) and/or transporter gene sequence(s) and/or BCAA binding protein gene sequence(s) and/or BCAA exporter gene sequence(s), e.g., as described herein, which are induced by rhamnose. In some embodiments, the genetically engineered bacteria comprise multiple copies of different branched chain amino acid catabolism enzyme genes sequence(s) and/or transporter gene sequence(s) and/or BCAA binding protein gene sequence(s) and/or BCAA exporter gene sequence(s), e.g., as described herein, one or more of which are induced by rhamnose.

In a first example, the rhamnose inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the rhamnose inducible promoter drives expression under a first set of exogenous conditions, and the second promoter drives the expression under a second set of exogenous conditions. In second example, the rhamnose promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., rhamnose and IPTG). In another non-limiting example, the first inducing conditions are culture conditions, e.g., the presence of rhamnose, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more rhamnose promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by rhamnose. In some embodiments, the gene sequence(s) encoding the branched chain amino acid catabolism enzyme(s) or branched chain amino acid transporter(s) and/or BCAA binding protein(s) and/or BCAA exporter(s) is present in the chromosome and operably linked to a promoter that is induced by rhamnose.

In some embodiments, the genetically engineered bacteria comprise one or more gene sequence(s) which are inducible through an Isopropyl β-D-1-thiogalactopyranoside (IPTG) inducible system or other compound which induced transcription from the Lac Promoter. IPTG is a molecular mimic of allolactose, a lactose metabolite that activates transcription of the lac operon. In contrast to allolactose, the sulfur atom in IPTG creates a non-hydrolyzable chemical blond, which prevents the degradation of IPTG, allowing the concentration to remain constant. IPTG binds to the lac repressor and releases the tetrameric repressor (lacI) from the lac operator in an allosteric manner, thereby allowing the transcription of genes in the lac operon. Since IPTG is not metabolized by E. coli, its concentration stays constant and the rate of expression of Lac promoter-controlled is tightly controlled, both in vivo and in vitro. IPTG intake is independent on the action of lactose permease, since other transport pathways are also involved. Inducible expression from the PLac can be controlled or fine-tuned through the optimization of the ribosome binding site (RBS), as described herein. Other compounds which inactivate LacI, can be used instead of IPTG in a similar manner.

In one embodiment, expression of the enzyme(s), e.g., ldc, e.g., as described herein, is driven directly or indirectly by one or more IPTG inducible promoter(s). In one embodiment, the genetically engineered bacteria encode one or more branched chain amino acid transporter(s), e.g., livKHMGF and/or brnQ, described herein, whose expression is driven directly or indirectly by one or more IPTG inducible promoter(s). In one embodiment, the genetically engineered bacteria encode one or more branched chain amino acid exporter(s)described herein, whose expression is driven directly or indirectly by one or more IPTG inducible promoter(s).

In some embodiments, the IPTG inducible promoter is useful for or induced during in vivo expression of the one or more protein(s) of interest. In some embodiments, expression of one or more branched chain amino acid catabolism enzyme(s) and/or branched chain amino acid transporter(s) is driven directly or indirectly by one or more IPTG inducible promoter(s) in vivo. In some embodiments, the promoter is directly or indirectly induced by a molecule (e.g., IPTG) that is co-administered with the bacteria.

In some embodiments, expression of one or more branched chain amino acid catabolism enzyme(s) and/or branched chain amino acid transporter(s) and/or BCAA binding protein(s) and/or BCAA exporter(s), is driven directly or indirectly by one or more IPTG inducible promoter(s) during in vitro growth, preparation, or manufacturing of the strain prior to in vivo administration. In some embodiments, the IPTG inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., IPTG, that is added to in the bacterial culture to induce expression and pre-load the bacterium with branched chain amino acid catabolism enzyme(s) prior to administration. In some embodiments, the cultures, which are induced by IPTG, are grown aerobically. In some embodiments, the cultures, which are induced by IPTG, are grown anaerobically.

In some embodiments, bacterial cell comprises two or more distinct branched chain amino acid catabolism cassette(s) or other polypeptide(s) of interest, one or more of which are induced by IPTG. In some embodiments, the genetically engineered bacteria comprise multiple copies of the same branched chain amino acid catabolism enzyme gene sequence(s) and/or transporter gene sequence(s), e.g., as described herein, which are induced IPTG. In some embodiments, the genetically engineered bacteria comprise multiple copies of different branched chain amino acid catabolism enzyme genes sequence(s) and/or transporter gene sequence(s) and/or other gene sequence(s) of interest, as described herein, one or more of which are induced by IPTG.

In a first example, the IPTG inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the IPTG inducible promoter drives expression under a first set of exogenous conditions, and the second promoter drives the expression under a second set of exogenous conditions. In second example, the IPTG promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., IPTG and IPTG). In another non-limiting example, the first inducing conditions are culture conditions, e.g., the presence of IPTG, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more IPTG promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by IPTG. In some embodiments, the gene sequence(s) encoding the branched chain amino acid catabolism enzyme(s) or branched chain amino acid transporter(s)is present in the chromosome and operably linked to a promoter that is induced by IPTG.

In some embodiments, the IPTG inducible construct further comprises a gene encoding lacI, which is divergently transcribed from the same promoter as the one or more one or more branched chain amino acid catabolism enzyme(s) and/or transporters described herein.

In some embodiments, the genetically engineered bacteria comprise one or more gene sequence(s) which are inducible through a tetracycline inducible system. The initial system Gossen and Bujard (Gossen M & Bujard H.PNAS, 1992 Jun 15;89(12):5547-51) developed is known as tetracycline off: in the presence of tetracycline, expression from a tet-inducible promoter is reduced. Tetracycline-controlled transactivator (tTA) was created by fusing tetR with the C-terminal domain of VP16 (virion protein 16) from herpes simplex virus. In the absence of tetracycline, the tetR portion of tTA will bind tetO sequences in the tet promoter, and the activation domain promotes expression. In the presence of tetracycline, tetracycline binds to tetR, precluding tTA from binding to the tetO sequences. Next, a reverse Tet repressor (rTetR), was developed which created a reliance on the presence of tetracycline for induction, rather than repression. The new transactivator rtTA (reverse tetracycline-controlled transactivator) was created by fusing rTetR with VP16. The tetracycline on system is also known as the rtTA-dependent system.

In one embodiment, expression of the enzyme(s), e.g., ldc, e.g., as described herein, is driven directly or indirectly by one or more tetracycline inducible promoter(s). In one embodiment, the genetically engineered bacteria encode one or more branched chain amino acid transporter(s), e.g., livKHMGF and/or brnQ, described herein, whose expression is driven directly or indirectly by one or more tetracycline inducible promoter(s).

In one embodiment, the genetically engineered bacteria encode one or more branched chain amino acid exporter(s).

In some embodiments, the tetracycline inducible promoter is useful for or induced during in vivo expression of the one or more protein(s) of interest. In some embodiments, expression of one or more branched chain amino acid catabolism enzyme(s) and/or branched chain amino acid transporter(s) and or other polypeptide(s) of interest is driven directly or indirectly by one or more tetracycline inducible promoter(s) in vivo. In some embodiments, the promoter is directly or indirectly induced by a molecule (e.g., tetracycline) that is co-administered with the bacteria.

In some embodiments, expression of one or more branched chain amino acid catabolism enzyme(s) and/or branched chain amino acid transporter(s) and or other polypeptide(s) of interest, is driven directly or indirectly by one or more tetracycline inducible promoter(s) during in vitro growth, preparation, or manufacturing of the strain prior to in vivo administration. In some embodiments, the tetracycline inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., tetracycline, that is added to in the bacterial culture to induce expression and pre-load the bacterium with branched chain amino acid catabolism enzyme(s) prior to administration. In some embodiments, the cultures, which are induced by tetracycline, are grown aerobically. In some embodiments, the cultures, which are induced by tetracycline, are grown anaerobically.

. In some embodiments, bacterial cell comprises two or more distinct branched chain amino acid catabolism cassette(s) and/or other polypeptide(s) of interest, one or more of which are induced by tetracycline. In some embodiments, the genetically engineered bacteria comprise multiple copies of the same branched chain amino acid catabolism enzyme gene sequence(s) and/or transporter gene sequence(s) and/or gene sequence(s) for the expression of other polypeptide(s) of interest, e.g., as described herein, which are induced by tetracycline. In some embodiments, the genetically engineered bacteria comprise multiple copies of different branched chain amino acid catabolism enzyme genes sequence(s) and/or transporter gene sequence(s) and gene sequence(s) for the expression of other polypeptide(s) of interest, e.g., as described herein, one or more of which are induced by tetracycline.

In a first example, the tetracycline inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the tetracycline inducible promoter drives expression under a first set of exogenous conditions, and the second promoter drives the expression under a second set of exogenous conditions. In second example, the tetracycline promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., tetracycline and IPTG). In another non-limiting example, the first inducing conditions are culture conditions, e.g., the presence of tetracycline, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more tetracycline promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by tetracycline. In some embodiments, the gene sequence(s) encoding the branched chain amino acid catabolism enzyme(s) or branched chain amino acid transporter(s)is present in the chromosome and operably linked to a promoter that is induced by tetracycline.

In some embodiments, the tetracycline inducible construct further comprises a gene encoding AraC, which is divergently transcribed from the same promoter as the one or more one or more branched chain amino acid catabolism enzyme(s) and/or transporters described herein.

In some embodiments, the genetically engineered bacteria comprise one or more gene sequence(s) whose expression is controlled by a temperature sensitive mechanism. Thermoregulators are advantageous because of strong transcriptional control without the use of external chemicals or specialized media (see, e.g., Nemani et al., Magnetic nanoparticle hyperthermia induced cytosine deaminase expression in microencapsulated E. coli for enzyme-prodrug therapy; J Biotechnol. 2015 Jun 10; 203: 32-40, and references therein). Thermoregulated protein expression using the mutant cI857 repressor and the pL and/or pR phage λ promoters have been used to engineer recombinant bacterial strains. The gene of interest cloned downstream of the λ promoters can then be efficiently regulated by the mutant thermolabile cI857 repressor of bacteriophage λ. At temperatures below 37° C., cI857 binds to the oL or regions of the pR promoter and blocks transcription by RNA polymerase. At higher temperatures, the functional cI857 dimer is destabilized, binding to the oL or oR DNA sequences is abrogated, and mRNA transcription is initiated. Inducible expression from the thermoregulated promoter can be controlled or further fine-tuned through the optimization of the ribosome binding site (RBS), as described herein.

In one embodiment, expression of one or more protein(s) of interest is driven directly or indirectly by one or more thermoregulated promoter(s). In one embodiment, expression of the enzyme(s), e.g., ldc, e.g., as described herein, is driven directly or indirectly by one or more thermoregulated inducible promoter(s). In one embodiment, the genetically engineered bacteria encode one or more branched chain amino acid transporter(s), e.g., livKHMGF and/or brnQ, described herein, whose expression is driven directly or indirectly by one or more thermoregulated inducible promoter(s). In one embodiment, the genetically engineered bacteria encode one or more branched chain amino acid exporter(s) described herein, whose expression is driven directly or indirectly by one or more thermoregulated inducible promoter(s).

In some embodiments, the thermoregulated promoter is useful for or induced during in vivo expression of the one or more protein(s) of interest. In some embodiments, expression of one or more branched chain amino acid catabolism enzyme(s) and/or branched chain amino acid transporter(s) and/or other protein(s) of interest is driven directly or indirectly by one or more thermoregulated promoter(s) in vivo.

In some embodiments, expression of one or more protein(s) of interest is driven directly or indirectly by one or more thermoregulated promoter(s) during in vitro growth, preparation, or manufacturing of the strain prior to in vivo administration. In some embodiments, it may be advantageous to shut off production of the one or more branched chain amino acid catabolism enzyme(s) and/or branched chain amino acid transporter(s). This can be done in a thermoregulated system by growing the strain at lower temperatures, e.g., 30° C. Expression can then be induced by elevating the temperature to 37° C. and/or 42° C. In some embodiments, the thermoregulated promoter(s) are induced in culture, e.g., grown in a flask, fermenter or other appropriate culture vessel, e.g., used during cell growth, cell expansion, fermentation, recovery, purification, formulation, and/or manufacture. In some embodiments, the cultures, which are induced by temperatures between 37° C. and 42° C., are grown aerobically. In some embodiments, the cultures, which are induced by induced by temperatures between 37° C. and 42° C., are grown anaerobically.

In some embodiments, bacterial cell comprises two or more distinct branched chain amino acid catabolism cassette(s) or branched chain amino acid transporter(s) and/or cassette(s) for the expression of other protein(s) of interest, one or more of which are induced by temperature. In some embodiments, the genetically engineered bacteria comprise multiple copies of the same branched chain amino acid catabolism enzyme gene sequence(s) and/or transporter gene sequence(s) and/or gene sequence(s) for the expression of other proteins of interest, e.g., as described herein, which are induced by temperature. In some embodiments, the genetically engineered bacteria comprise multiple copies of different branched chain amino acid catabolism enzyme genes sequence(s) and/or transporter gene sequence(s) or other gene sequence(s) of interest, e.g., as described herein, one or more of which are induced by temperature.

In a first example, the temperature inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the temperature inducible promoter drives expression under a first set of exogenous conditions, and the second promoter drives the expression under a second set of exogenous conditions. In second example, the temperature promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., temperature regulation and IPTG). In another non-limiting example, the first inducing conditions are culture conditions, e.g., the permissive temperature, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more temperature regulated promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by temperature. In some embodiments, the gene sequence(s) encoding the branched chain amino acid catabolism enzyme(s) or branched chain amino acid transporter(s)is present in the chromosome and operably linked to a promoter that is induced by temperature.

In some embodiments, the thermoregulated construct further comprises a gene encoding mutant cI857 repressor, which is divergently transcribed from the same promoter as the one or more one or more branched chain amino acid catabolism enzyme(s) and/or transporters described herein. In some embodiments, the genetically engineered bacteria comprise one or more gene sequence(s) which are indirectly inducible through a system driven by the PssB promoter. The Pssb promoter is active under aerobic conditions, and shuts off under anaerobic conditions.

This promoter can be used to express a gene of interest under aerobic conditions. This promoter can also be used to tightly control the expression of a gene product such that it is only expressed under anaerobic conditions. In this case, the oxygen induced PssB promoter induces the expression of a repressor, which represses the expression of a gene of interest. As a result, the gene of interest is only expressed in the absence of the repressor, i.e., under anaerobic conditions. This strategy has the advantage of an additional level of control for improved fine-tuning and tighter control.

In one embodiment, expression of the enzyme(s), e.g., ldc, e.g., as described herein, is driven directly or indirectly by one or more PssB promoter(s). In one embodiment, the genetically engineered bacteria encode one or more branched chain amino acid transporter(s), e.g., livKHMGF and/or bmQ, described herein, whose expression is driven directly or indirectly by one or more PssB promoter(s). In one embodiment, the genetically engineered bacteria encode one or more branched chain amino acid exporter(s), described herein, whose expression is driven directly or indirectly by one or more PssB promoter(s).

In some embodiments, the PssB promoter is useful for or induced during in vivo expression of the one or more protein(s) of interest. In some embodiments, expression of one or more branched chain amino acid catabolism enzyme(s) and/or branched chain amino acid transporter(s) and/or other protein(s) of interest is driven directly or indirectly by one or more PssB promoter(s) in vivo. In some embodiments, the promoter is directly or indirectly induced by a molecule (e.g., arabinose) that is co-administered with the bacteria.

In some embodiments, expression of one or more branched chain amino acid catabolism enzyme(s) and/or branched chain amino acid transporter(s) and/or other protein(s) of interest, is driven directly or indirectly by one or more PssB promoter(s) during in vitro growth, preparation, or manufacturing of the strain prior to in vivo administration. In some embodiments, the PssB promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., arabinose, that is added to in the bacterial culture to induce expression and pre-load the bacterium with branched chain amino acid catabolism enzyme(s) prior to administration. In some embodiments, the cultures, which are induced by arabinose, are grown aerobically. In some embodiments, the cultures, which are induced by arabinose, are grown anaerobically.

. In some embodiments, bacterial cell comprises two or more distinct branched chain amino acid catabolism cassette(s) or other polypeptide(s) of interest, one or more of which are induced by arabinose. In some embodiments, the genetically engineered bacteria comprise multiple copies of the same branched chain amino acid catabolism enzyme gene sequence(s) and/or transporter gene sequence(s) and/or other gene sequence(s) of interest, e.g., as described herein, which are induced by one or more nutritional and/or chemical inducer(s) and/or metabolite(s). In some embodiments, the genetically engineered bacteria comprise multiple copies of different branched chain amino acid catabolism enzyme genes sequence(s) and/or transporter gene sequence(s), e.g., as described herein, one or more of which are induced by one or more nutritional and/or chemical inducer(s) and/or metabolite(s).

In a first example, the PssB promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the PssB promoter drives expression under a first set of exogenous conditions, and the second promoter drives the expression under a second set of exogenous conditions. In second example, the PssB promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., PssB and IPTG). In another non-limiting example, the first inducing conditions are culture conditions, e.g., the presence of arabinose, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more PssB promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by arabinose. In some embodiments, the gene sequence(s) encoding the branched chain amino acid catabolism enzyme(s) or branched chain amino acid transporter(s)is present in the chromosome and operably linked to a promoter that is induced by arabinose.

In another non-limiting example, this strategy can be used to control expression of thyA and/or dapA, e.g., to make a conditional auxotroph. The chromosomal copy of dapA or ThyA is knocked out. Under anaerobic conditions, dapA or thyA -as the case may be- are expressed, and the strain can grow in the absence of dap or thymidine. Under aerobic conditions, dapA or thyA expression is shut off, and the strain cannot grow in the absence of dap or thymidine. Such a strategy can, for example be employed to allow survival of bacteria under anaerobic conditions, e.g., the gut, but prevent survival under aerobic conditions (biosafety switch).

### Essential Genes and Auxotrophs

As used herein, the term “essential gene” refers to a gene which is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37:D455-D458 and Gerdes et al., Essential genes on metabolic maps, Curr. Opin. Biotechnol., 17(5):448-456, the entire contents of each of which are expressly incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the genetically engineered bacteria described herein also comprise a deletion or mutation in one or more gene(s) required for cell survival and/or growth.

In some embodiments, the bacterial cell comprises a genetic mutation in one or more endogenous gene(s) encoding a branched chain amino acid biosynthesis gene, wherein the genetic mutation reduces biosynthesis of one or more branched chain amino acids in the bacterial cell. In some embodiments, the endogenous gene encoding a branched chain amino acid biosynthesis gene is a keto acid reductoisomerase gene. Keto acid reductoisomerase gene is required for branched chain amino acid synthesis. Knock-out of this gene creates an auxotroph and requires the cell to import leucine to survive. In some embodiments, the bacterial cell comprises a genetic mutation in ilvC gene.

In one embodiment, the essential gene is an oligonucleotide synthesis gene, for example, thyA. In another embodiment, the essential gene is a cell wall synthesis gene, for example, dapA. In yet another embodiment, the essential gene is an amino acid gene, for example, serA or MetA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1, as long as the corresponding wild-type gene product is not produced in the bacteria.

Table 8 lists depicts exemplary bacterial genes which may be disrupted or deleted to produce an auxotrophic strain. These include, but are not limited to, genes required for oligonucleotide synthesis, amino acid synthesis, and cell wall synthesis.

TABLE 8

Non-limiting Examples of Bacterial Genes Useful for Generation of an Auxotroph

| | | |
|---|---|---|
| Amino Acid | Oligonucleotide | Cell Wall |
| cysE | thyA | dapA |
| glnA | uraA | dapB |
| ilvD | | dapD |
| leuB | | dapE |
| lysA | | dapF |
| serA | | |
| metA | | |
| glyA | | |
| hisB | | |
| ilvA | | |
| pheA | | |
| proA | | |
| thrC | | |
| trpC | | |
| tyrA | | |

Table 9 shows the survival of various amino acid auxotrophs in the mouse gut, as detected 24 hrs and 48 hrs post-gavage. These auxotrophs were generated using BW25113, a non-Nissle strain of E. coli.

TABLE 9

Survival of amino acid auxotrophs in the mouse gut

| | | | | |
|---|---|---|---|---|
| Gene | AA Auxotroph | Pre-Gavage | 24 hours | 48 hours |
| argA | Arginine | Present | Present | Absent |
| cysE | Cysteine | Present | Present | Absent |
| glnA | Glutamine | Present | Present | Absent |
| glyA | Glycine | Present | Present | Absent |
| hisB | Histidine | Present | Present | Present |
| ilvA | Isoleucine | Present | Present | Absent |
| leuB | Leucine | Present | Present | Absent |
| lysA | Lysine | Present | Present | Absent |
| metA | Methionine | Present | Present | Present |
| pheA | Phenylalanine | Present | Present | Present |
| proA | Proline | Present | Present | Absent |
| serA | Serine | Present | Present | Present |
| thrC | Threonine | Present | Present | Present |
| trpC | Tryptophan | Present | Present | Present |
| tyrA | Tyrosine | Present | Present | Present |
| ilvD | Valine/Isoleucine/ Leucine | Present | Present | Absent |
| thyA | Thiamine | Present | Absent | Absent |
| uraA | Uracil | Present | Absent | Absent |
| flhD | FlhD | Present | Present | Present |

For example, thymine is a nucleic acid that is required for bacterial cell growth; in its absence, bacteria undergo cell death. The thyA gene encodes thimidylate synthetase, an enzyme that catalyzes the first step in thymine synthesis by converting dUMP to dTMP (Sat et al., 2003). In some embodiments, the bacterial cell of the disclosure is a thyA auxotroph in which the thyA gene is deleted and/or replaced with an unrelated gene. A thyA auxotroph can grow only when sufficient amounts of thymine are present, e.g., by adding thymine to growth media in vitro, or in the presence of high thymine levels found naturally in the human gut in vivo. In some embodiments, the bacterial cell of the disclosure is auxotrophic in a gene that is complemented when the bacterium is present in the mammalian gut. Without sufficient amounts of thymine, the thyA auxotroph dies. In some embodiments, the auxotrophic modification is used to ensure that the bacterial cell does not survive in the absence of the auxotrophic gene product (e.g., outside of the gut).

Diaminopimelic acid (DAP) is an amino acid synthetized within the lysine biosynthetic pathway and is required for bacterial cell wall growth (Meadow et al., 1959; Clarkson et al., 1971). In some embodiments, any of the genetically engineered bacteria described herein is a dapD auxotroph in which dapD is deleted and/or replaced with an unrelated gene. A dapD auxotroph can grow only when sufficient amounts of DAP are present, e.g., by adding DAP to growth media in vitro. Without sufficient amounts of DAP, the dapD auxotroph dies. In some embodiments, the auxotrophic modification is used to ensure that the bacterial cell does not survive in the absence of the auxotrophic gene product (e.g., outside of the gut).

In other embodiments, the genetically engineered bacterium of the present disclosure is a uraA auxotroph in which uraA is deleted and/or replaced with an unrelated gene. The uraA gene codes for UraA, a membrane-bound transporter that facilitates the uptake and subsequent metabolism of the pyrimidine uracil (Andersen et al., 1995). A uraA auxotroph can grow only when sufficient amounts of uracil are present, e.g., by adding uracil to growth media in vitro. Without sufficient amounts of uracil, the uraA auxotroph dies. In some embodiments, auxotrophic modifications are used to ensure that the bacteria do not survive in the absence of the auxotrophic gene product (e.g., outside of the gut).

In complex communities, it is possible for bacteria to share DNA. In very rare circumstances, an auxotrophic bacterial strain may receive DNA from a non-auxotrophic strain, which repairs the genomic deletion and permanently rescues the auxotroph. Therefore, engineering a bacterial strain with more than one auxotroph may greatly decrease the probability that DNA transfer will occur enough times to rescue the auxotrophy. In some embodiments, the genetically engineered bacteria comprise a deletion or mutation in two or more genes required for cell survival and/or growth.

Other examples of essential genes include, but are not limited to yhbV, yagG, hemB, secD, secF, ribD, ribE, thiL, dxs, ispA, dnaX, adk, hemH, lpxH, cysS, fold, rplT, infC, thrS, nadE, gapA, yeaZ, aspS, argS, pgsA, yefM, metG, folE, yejM, gyrA, nrdA, nrdB, folC, accD, fabB, gltX, ligA, zipA, dapE, dapA, der, hisS, ispG, suhB, tadA, acpS, era, rnc, ftsB, eno, pyrG, chpR, lgt, fbaA, pgk, yqgD, metK, yqgF, plsC, ygiT, pare, ribB, cca, ygjD, tdcF, yraL, yihA, ftsN, murI, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, dnaC, ribF, lspA, ispH, dapB, folA, imp, yabQ, ftsL, ftsI, murE, murF, mraY, murD, ftsW, murG, murC, ftsQ, ftsA, ftsZ, lpxC, secM, secA, can, folK, hemL, yadR, dapD, map, rpsB, infB ,nusA, ftsH, obgE, rpmA, rplU, ispB, murA, yrbB, yrbK, yhbN, rpsI, rplM, degS, mreD, mreC, mreB, accB, accC, yrdC, def, fmt, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsJ, fusA, rpsG, rpsL, trpS, yrfF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsN, rplE, rplX, rplN, rpsQ, rpmC, rplP, rpsC, rplV, rpsS, rplB, cdsA, yaeL, yaeT, lpxD, fabZ, lpxA, lpxB, dnaE, accA, tilS, proS, yafF, tsf, pyrH, olA, rlpB, leuS, lnt, glnS, fldA, cydA, infA, cydC, ftsK, lolA, serS, rpsA, msbA, lpxK, kdsB, mukF, mukE, mukB, asnS, fabA, mviN, rne, yceQ, fabD, fabG, acpP, tmk, holB, lolC, lolD, lolE, purB, ymfK, minE, mind, pth, rsA, ispE, lolB, hemA, prfA, prmC, kdsA, topA, ribA, fabI, racR, dicA, ydfB, tyrS, ribC, ydiL, pheT, pheS, yhhQ, bcsB, glyQ, yibJ, and gpsA. Other essential genes are known to those of ordinary skill in the art.

In some embodiments, the genetically engineered bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain, “ACS Synthetic Biology (2015) DOI: 10.1021/acssynbio.5b00085, the entire contents of which are expressly incorporated herein by reference).

In some embodiments, the SLiDE bacterial cell comprises a mutation in an essential gene. In some embodiments, the essential gene is selected from the group consisting of pheS, dnaN, tyrS, metG and adk. In some embodiments, the essential gene is dnaN comprising one or more of the following mutations: H191N, R240C, I317S, F319V, L340T, V347I, and S345C. In some embodiments, the essential gene is dnaN comprising the mutations H191N, R240C, I317S, F319V, L340T, V347I, and S345C. In some embodiments, the essential gene is pheS comprising one or more of the following mutations: F125G, P183T, P184A, R186A, and I188L. In some embodiments, the essential gene is pheS comprising the mutations F125G, P183T, P184A, R186A, and I188L. In some embodiments, the essential gene is tyrS comprising one or more of the following mutations: L36V, C38A and F40G. In some embodiments, the essential gene is tyrS comprising the mutations L36V, C38A and F40G. In some embodiments, the essential gene is metG comprising one or more of the following mutations: E45Q, N47R, I49G, and A51C. In some embodiments, the essential gene is metG comprising the mutations E45Q, N47R, I49G, and A51C. In some embodiments, the essential gene is adk comprising one or more of the following mutations: I4L, L5I and L6G. In some embodiments, the essential gene is adk comprising the mutations I4L, L5I and L6G.

In some embodiments, the genetically engineered bacterium is complemented by a ligand. In some embodiments, the ligand is selected from the group consisting of benzothiazole, indole, 2-aminobenzothiazole, indole-3-butyric acid, indole-3-acetic acid, and L-histidine methyl ester. For example, bacterial cells comprising mutations in metG (E45Q, N47R, I49G, and A51C) are complemented by benzothiazole, indole, 2-aminobenzothiazole, indole-3-butyric acid, indole-3-acetic acid or L-histidine methyl ester. Bacterial cells comprising mutations in dnaN (H191N, R240C, I317S, F319V, L340T, V347I, and S345C) are complemented by benzothiazole, indole or 2-aminobenzothiazole. Bacterial cells comprising mutations in pheS (F125G, P183T, P184A, R186A, and I188L) are complemented by benzothiazole or 2-aminobenzothiazole. Bacterial cells comprising mutations in tyrS (L36V, C38A, and F40G) are complemented by benzothiazole or 2-aminobenzothiazole. Bacterial cells comprising mutations in adk (I4L, L5I and L6G) are complemented by benzothiazole or indole.

In some embodiments, the genetically engineered bacterium comprises more than one mutant essential gene that renders it auxotrophic to a ligand. In some embodiments, the bacterial cell comprises mutations in two essential genes. For example, in some embodiments, the bacterial cell comprises mutations in tyrS (L36V, C38A, and F40G) and metG (E45Q, N47R, I49G, and A51C). In other embodiments, the bacterial cell comprises mutations in three essential genes. For example, in some embodiments, the bacterial cell comprises mutations in tyrS (L36V, C38A, and F40G), metG (E45Q, N47R, I49G, and A51C), and pheS (F125G, P183T, P184A, R186A, and I188L).

In some embodiments, the genetically engineered bacterium is a conditional auxotroph whose essential gene(s) is replaced using the arabinose system described herein.

### Genetic Regulatory Circuits

In some embodiments, the genetically engineered bacteria comprise multi-layered genetic regulatory circuits for expressing the constructs described herein (see, e.g., U.S. Provisional Application No. 62/184,811, incorporated herein by reference in its entirety). The genetic regulatory circuits are useful to screen for mutant bacteria that produce a branched chain amino acid catabolism enzyme, BCAA transporter, and/or BCAA binding protein or rescue an auxotroph. In certain embodiments, the invention provides methods for selecting genetically engineered bacteria that produce one or more genes of interest.

In some embodiments, the invention provides genetically engineered bacteria comprising a gene or gene cassette for producing a payload and a T7 polymerase-regulated genetic regulatory circuit. For example, the genetically engineered bacteria comprise a first gene encoding a T7 polymerase, wherein the first gene is operably linked to a fumarate and nitrate reductase regulator (FNR)-responsive promoter; a second gene or gene cassette for producing a payload, wherein the second gene or gene cassette is operably linked to a T7 promoter that is induced by the T7 polymerase; and a third gene encoding an inhibitory factor, lysY, that is capable of inhibiting the T7 polymerase. In the presence of oxygen, FNR does not bind the FNR-responsive promoter, and the payload is not expressed. LysY is expressed constitutively (P-lac constitutive) and further inhibits T7 polymerase. In the absence of oxygen, FNR dimerizes and binds to the FNR-responsive promoter, T7 polymerase is expressed at a level sufficient to overcome lysY inhibition, and the payload is expressed. In some embodiments, the lysY gene is operably linked to an additional FNR binding site. In the absence of oxygen, FNR dimerizes to activate T7 polymerase expression as described above, and also inhibits lysY expression.

In some embodiments, the invention provides genetically engineered bacteria comprising a gene or gene cassette for producing a payload and a protease-regulated genetic regulatory circuit. For example, the genetically engineered bacteria comprise a first gene encoding an mf-lon protease, wherein the first gene is operably linked to a FNR-responsive promoter; a second gene or gene cassette for producing a payload operably linked to a tet regulatory region (tetO); and a third gene encoding an mf-lon degradation signal linked to a tet repressor (tetR), wherein the tetR is capable of binding to the tet regulatory region and repressing expression of the second gene or gene cassette. The mf-lon protease is capable of recognizing the mf-lon degradation signal and degrading the tetR. In the presence of oxygen, FNR does not bind the FNR-responsive promoter, the repressor is not degraded, and the payload is not expressed. In the absence of oxygen, FNR dimerizes and binds the FNR-responsive promoter, thereby inducing expression of mf-lon protease. The mf-lon protease recognizes the mf-lon degradation signal and degrades the tetR, and the payload is expressed.

In some embodiments, the invention provides genetically engineered bacteria comprising a gene or gene cassette for producing a payload and a repressor-regulated genetic regulatory circuit. For example, the genetically engineered bacteria comprise a first gene encoding a first repressor, wherein the first gene is operably linked to a FNR-responsive promoter; a second gene or gene cassette for producing a payload operably linked to a first regulatory region comprising a constitutive promoter; and a third gene encoding a second repressor, wherein the second repressor is capable of binding to the first regulatory region and repressing expression of the second gene or gene cassette. The third gene is operably linked to a second regulatory region comprising a constitutive promoter, wherein the first repressor is capable of binding to the second regulatory region and inhibiting expression of the second repressor. In the presence of oxygen, FNR does not bind the FNR-responsive promoter, the first repressor is not expressed, the second repressor is expressed, and the payload is not expressed. In the absence of oxygen, FNR dimerizes and binds the FNR-responsive promoter, the first repressor is expressed, the second repressor is not expressed, and the payload is expressed.

Examples of repressors useful in these embodiments include, but are not limited to, ArgR, TetR, ArsR, AscG, LacI, CscR, DeoR, DgoR, FruR, GalR, GatR, CI, LexA, RafR, QacR, and PtxS (US20030166191).

In some embodiments, the invention provides genetically engineered bacteria comprising a gene or gene cassette for producing a payload and a regulatory RNA-regulated genetic regulatory circuit. For example, the genetically engineered bacteria comprise a first gene encoding a regulatory RNA, wherein the first gene is operably linked to a FNR-responsive promoter, and a second gene or gene cassette for producing a payload. The second gene or gene cassette is operably linked to a constitutive promoter and further linked to a nucleotide sequence capable of producing an mRNA hairpin that inhibits translation of the payload. The regulatory RNA is capable of eliminating the mRNA hairpin and inducing payload translation via the ribosomal binding site. In the presence of oxygen, FNR does not bind the FNR-responsive promoter, the regulatory RNA is not expressed, and the mRNA hairpin prevents the payload from being translated. In the absence of oxygen, FNR dimerizes and binds the FNR-responsive promoter, the regulatory RNA is expressed, the mRNA hairpin is eliminated, and the payload is expressed.

In some embodiments, the invention provides genetically engineered bacteria comprising a gene or gene cassette for producing a payload and a CRISPR-regulated genetic regulatory circuit. For example, the genetically engineered bacteria comprise a Cas9 protein; a first gene encoding a CRISPR guide RNA, wherein the first gene is operably linked to a FNR-responsive promoter; a second gene or gene cassette for producing a payload, wherein the second gene or gene cassette is operably linked to a regulatory region comprising a constitutive promoter; and a third gene encoding a repressor operably linked to a constitutive promoter, wherein the repressor is capable of binding to the regulatory region and repressing expression of the second gene or gene cassette. The third gene is further linked to a CRISPR target sequence that is capable of binding to the CRISPR guide RNA, wherein said binding to the CRISPR guide RNA induces cleavage by the Cas9 protein and inhibits expression of the repressor. In the presence of oxygen, FNR does not bind the FNR-responsive promoter, the guide RNA is not expressed, the repressor is expressed, and the payload is not expressed. In the absence of oxygen, FNR dimerizes and binds the FNR-responsive promoter, the guide RNA is expressed, the repressor is not expressed, and the payload is expressed.

In some embodiments, the invention provides genetically engineered bacteria comprising a gene or gene cassette for producing a payload and a recombinase-regulated genetic regulatory circuit. For example, the genetically engineered bacteria comprise a first gene encoding a recombinase, wherein the first gene is operably linked to a FNR-responsive promoter, and a second gene or gene cassette for producing a payload operably linked to a constitutive promoter. The second gene or gene cassette is inverted in orientation (3′ to 5′) and flanked by recombinase binding sites, and the recombinase is capable of binding to the recombinase binding sites to induce expression of the second gene or gene cassette by reverting its orientation (5′ to 3′). In the presence of oxygen, FNR does not bind the FNR-responsive promoter, the recombinase is not expressed, the payload remains in the 3′ to 5′ orientation, and no functional payload is produced. In the absence of oxygen, FNR dimerizes and binds the FNR-responsive promoter, the recombinase is expressed, the payload is reverted to the 5′ to 3′ orientation, and functional payload is produced.

In some embodiments, the invention provides genetically engineered bacteria comprising a gene or gene cassette for producing a payload and a polymerase- and recombinase-regulated genetic regulatory circuit. For example, the genetically engineered bacteria comprise a first gene encoding a recombinase, wherein the first gene is operably linked to a FNR-responsive promoter; a second gene or gene cassette for producing a payload operably linked to a T7 promoter; a third gene encoding a T7 polymerase, wherein the T7 polymerase is capable of binding to the T7 promoter and inducing expression of the payload. The third gene encoding the T7 polymerase is inverted in orientation (3′ to 5′) and flanked by recombinase binding sites, and the recombinase is capable of binding to the recombinase binding sites to induce expression of the T7 polymerase gene by reverting its orientation (5′ to 3′). In the presence of oxygen, FNR does not bind the FNR-responsive promoter, the recombinase is not expressed, the T7 polymerase gene remains in the 3′ to 5′ orientation, and the payload is not expressed. In the absence of oxygen, FNR dimerizes and binds the FNR-responsive promoter, the recombinase is expressed, the T7 polymerase gene is reverted to the 5′ to 3′ orientation, and the payload is expressed.

### Isolated Plasmids

In other embodiments, the disclosure provides an isolated plasmid comprising a first nucleic acid encoding a first payload operably linked to a first inducible promoter, and a second nucleic acid encoding a second payload operably linked to a second inducible promoter. In other embodiments, the disclosure provides an isolated plasmid further comprising a third nucleic acid encoding a third payload operably linked to a third inducible promoter. In other embodiments, the disclosure provides a plasmid comprising four, five, six, or more nucleic acids encoding four, five, six, or more payloads operably linked to inducible promoters. In any of the embodiments described here, the first, second, third, fourth, fifth, sixth, etc “payload(s)” can be a branched chain amino acid catabolism enzyme, a transporter of branched chain amino acids, a binding protein of branched chain amino acids, or other sequence described herein. In one embodiment, the nucleic acid encoding the first payload and the nucleic acid encoding the second payload are operably linked to the first inducible promoter. In one embodiment, the nucleic acid encoding the first payload is operably linked to a first inducible promoter and the nucleic acid encoding the second payload is operably linked to a second inducible promoter. In one embodiment, the first inducible promoter and the second inducible promoter are separate copies of the same inducible promoter. In another embodiment, the first inducible promoter and the second inducible promoter are different inducible promoters. In other embodiments comprising a third nucleic acid, the nucleic acid encoding the third payload and the nucleic acid encoding the first and second payloads are all operably linked to the same inducible promoter. In other embodiments, the nucleic acid encoding the first payload is operably linked to a first inducible promoter, the nucleic acid encoding the second payload is operably linked to a second inducible promoter, and the nucleic acid encoding the third payload is operably linked to a third inducible promoter. In some embodiments, the first, second, and third inducible promoters are separate copies of the same inducible promoter. In other embodiments, the first inducible promoter, the second inducible promoter, and the third inducible promoter are different inducible promoters. In some embodiments, the first promoter, the second promoter, and the optional third promoter, or the first promoter and the second promoter and the optional third promoter, are each directly or indirectly induced by low-oxygen or anaerobic conditions. In other embodiments, the first promoter, the second promoter, and the optional third promoter, or the first promoter and the second promoter and the optional third promoter, are each a fumarate and nitrate reduction regulator (FNR) responsive promoter. In other embodiments, the first promoter, the second promoter, and the optional third promoter, or the first promoter and the second promoter and the optional third promoter are each a ROS-inducible regulatory region. In other embodiments, the first promoter, the second promoter, and the optional third promoter, or the first promoter and the second promoter and the optional third promoter are each a RNS-inducible regulatory region.

In some embodiments, the heterologous gene encoding a branched chain amino acid catabolism enzyme is operably linked to a constitutive promoter. In one embodiment, the constitutive promoter is a lac promoter. In another embodiment, the constitutive promoter is a tet promoter. In another embodiment, the constitutive promoter is a constitutive Escherichia coli σ³² promoter. In another embodiment, the constitutive promoter is a constitutive Escherichia coli σ⁷⁰ promoter. In another embodiment, the constitutive promoter is a constitutive Bacillus subtilis σ^{A} promoter. In another embodiment, the constitutive promoter is a constitutive Bacillus subtilis σ^{B} promoter. In another embodiment, the constitutive promoter is a Salmonella promoter. In other embodiments, the constitutive promoter is a bacteriophage T7 promoter. In other embodiments, the constitutive promoter is and a bacteriophage SP6 promoter. In any of the above-described embodiments, the plasmid further comprises a heterologous gene encoding a transporter of a branched chain amino acid, a BCAA binding protein, and/or a kill switch construct, which may be operably linked to a constitutive promoter or an inducible promoter.

In some embodiments, the isolated plasmid comprises at least one heterologous branched chain amino acid catabolism enzyme gene operably linked to a first inducible promoter; a heterologous gene encoding a TetR protein operably linked to a P_{araBAD} promoter, a heterologous gene encoding AraC operably linked to a P_{araC} promoter, a heterologous gene encoding an antitoxin operably linked to a constitutive promoter, and a heterologous gene encoding a toxin operably linked to a P_{TetR} promoter. In another embodiment, the isolated plasmid comprises at least one heterologous gene encoding a branched chain amino acid catabolism enzyme operably linked to a first inducible promoter; a heterologous gene encoding a TetR protein and an anti-toxin operably linked to a P_{araBAD} promoter, a heterologous gene encoding AraC operably linked to a P_{araC} promoter, and a heterologous gene encoding a toxin operably linked to a P_{TetR} promoter.

In one embodiment, the plasmid is a high-copy plasmid. In another embodiment, the plasmid is a low-copy plasmid.

In another aspect, the disclosure provides a recombinant bacterial cell comprising an isolated plasmid described herein. In another embodiment, the disclosure provides a pharmaceutical composition comprising the recombinant bacterial cell.

### Integration

In some embodiments, any of the gene(s) or gene cassette(s) of the present disclosure may be integrated into the bacterial chromosome at one or more integration sites. One or more copies of the gene (for example, an amino acid catabolism gene, BCAA transporter gene, and/or BCAA binding protein gene) or gene cassette (for example, a gene cassette comprising an amino acid catabolism gene, an amino acid transporter gene, a BCAA binding protein gene) may be integrated into the bacterial chromosome. Having multiple copies of the gene or gene cassette integrated into the chromosome allows for greater production of the payload, e.g., amino acid catabolism enzyme, BCAA transporter gene, and/or BCAA binding protein gene and other enzymes of a gene cassette, and also permits fine-tuning of the level of expression. Alternatively, different circuits described herein, such as any of the kill-switch circuits, in addition to the therapeutic gene(s) or gene cassette(s) could be integrated into the bacterial chromosome at one or more different integration sites to perform multiple different functions.

### Pharmaceutical Compositions and Formulations

Pharmaceutical compositions comprising the genetically engineered microorganisms of the invention may be used to treat, manage, ameliorate, and/or prevent a disorder associated with branched chain amino acid catabolism or symptom(s) associated with diseases or disorders associated with branched chain amino acid catabolism. Pharmaceutical compositions of the invention comprising one or more genetically engineered bacteria, and/or one or more genetically engineered yeast or virus, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise one or more of the genetic modifications described herein, e.g., selected from expression of at least one branched chain amino acid catabolism enzyme, BCAA transporter, BCAA binding protein, auxotrophy, kill-switch, exporter knock-out, etc. In alternate embodiments, the pharmaceutical composition comprises two or more species, strains, and/or subtypes of bacteria that are each engineered to comprise the genetic modifications described herein.

The pharmaceutical compositions of the invention described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington’s Pharmaceutical Sciences,” Mack Publishing Co., Easton, PA). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, immediate-release, pulsatile-release, delayed-release, or sustained release). Suitable dosage amounts for the genetically engineered bacteria may range from about 104 to 1012 bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal

The genetically engineered bacteria or genetically engineered yeast or virus may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the bacteria may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered microorganisms may be administered intravenously, e.g., by infusion or injection.

The genetically engineered microorganisms of the disclosure may be administered intrathecally. In some embodiments, the genetically engineered microorganisms of the invention may be administered orally. The genetically engineered microorganisms disclosed herein may be administered topically and formulated in the form of an ointment, cream, transdermal patch, lotion, gel, shampoo, spray, aerosol, solution, emulsion, or other form well known to one of skill in the art. See, e.g., “Remington’s Pharmaceutical Sciences,” Mack Publishing Co., Easton, PA. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the recombinant bacteria of the invention may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The genetically engineered microorganisms disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane / polydimethylsiloxane (PEG/PD5/PDMS), poly N,N- dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered microorganisms are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

In one embodiment, the genetically engineered microorganisms of the disclosure may be formulated in a composition suitable for administration to pediatric subjects. As is well known in the art, children differ from adults in many aspects, including different rates of gastric emptying, pH, gastrointestinal permeability, etc. (Ivanovska et al., Pediatrics, 134(2):361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered microorganisms may be orally administered, for example, with an inert diluent or an assimilable edible carrier. The compound may also be enclosed in a hard or soft shell gelatin capsule, compressed into tablets, or incorporated directly into the subject’s diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria of the invention may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art. For example, see U.S. 2015/0359894 and US 2015/0238545, the entire contents of each of which are expressly incorporated herein by reference. In yet another embodiment, the pharmaceutical composition of the invention is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered microorganisms described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered microorganisms may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N- vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD50, ED50, EC50, and IC50 may be determined, and the dose ratio between toxic and therapeutic effects (LD50/ED50) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the genetically engineered viruses are prepared for delivery, taking into consideration the need for efficient delivery and for overcoming the host antiviral immune response. Approaches to evade antiviral response include the administration of different viral serotypes as part of the treatment regimen (serotype switching), formulation, such as polymer coating to mask the virus from antibody recognition and the use of cells as delivery vehicles.

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N- vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### In Vivo Methods

The recombinant bacteria disclosed herein may be evaluated in vivo, e.g., in an animal model. Any suitable animal model of a disease or condition associated with catabolism of a branched chain amino acid, e.g., leucine, may be used (see, e.g., Skvorak, J. Inherit. Metab. Dis., 2009, 32:229-246 and Homanics et al., BMC Med. Genet., 2006, 7(33):1-13), including the Dbt-/-model (E2 subunit of BCKDH, which has a 3-fold increase in blood and urine BCAA levels and results in neonatal lethalthy) (serves as classic MSUD model). This model is partially rescued by two transgenes (LAP-tTA and TRE-E2), allowing 5-6% of normal BCKDH activity and an increase in mice survival to three or four weeks (serves as an intermediate MSUD model) (as described in Homanics et al., 2006, the contents of which is herein incorporated by reference in its entirety). In addition, intermediate MSUD mice can be used to show development of neuropathology with striking similarity to human MSUD. In this model, branched-chain amino acid accumulation was associated with neurotransmitter deficiency, behavioral changes and limited survival, and providing intermediate MSUD mice with a choice between normal and branched-chain amino acid free diet prevented brain injury and dramatically improved survival (Zinnanti et al., Dual mechanism of brain injury and novel treatment strategy in maple syrup urine disease; Brain 2009: 132; 903-918, the contents of which is herein incorporated by reference in its entirety). In some embodiments, the animal model is a mouse model of Maple Syrup Urine Disease. In one embodiment, the mouse model of MSUD is a branched-chain amino transferase knockout mouse (Wu et al., J. Clin. Invest, 113:434-440, 2004 or She et al., Cell Metabol., 6:181-194, 2007). In another embodiment, the mouse model of MSUD is a dihydrolipoamine dehydrogenase (E3) subunit knock-out mouse (Johnson et al., Proc. Natl. Acad. Sci. U.S.A., 94:14512-14517, 1997). In another embodiment, the mouse model of classic MSUD is a deletion of exon 5 and part of exon 4 of the E2 subunit of the branched-chain alpha-keto acid dehydrogenase (Homanics et al., BMC Med. Genet., 7:33, 2006) or the mouse model of intermediate MSUD (Homanics et al., BMC Med. Genet., 7:33, 2006). In another embodiment, the model is a Polled Shorthorn calf model of disease or a Polled Hereford calf model of disease (Harper et al., Aus. Vet. J., 66(2):46-49, 1988). Other relevant animal models include those described in She et al., Cell Metab. 2007 September ; 6(3): 181-194; Wu et al., J. Clin. Invest. 113:434-440 (2004); Bridi, et al., J Neurosci Methods. 2006 Sep 15;155(2):224-30.

The recombinant bacterial cells disclosed herein may administered to the animal, e.g., by oral gavage, and treatment efficacy is determined, e.g., by measuring blood leucine levels before and after treatment. The animal may be sacrificed, and tissue samples may be collected and analyzed.

### Methods of Screening

### Generation of Bacterial Strains with Enhanced Ability to Transport Amino Acids

Due to their ease of culture, short generation times, very high population densities and small genomes, microbes can be evolved to unique phenotypes in abbreviated timescales. Adaptive laboratory evolution (ALE) is the process of passaging microbes under selective pressure to evolve a strain with a preferred phenotype. Most commonly, this is applied to increase utilization of carbon/energy sources or adapting a strain to environmental stresses (e.g., temperature, pH), whereby mutant strains more capable of growth on the carbon substrate or under stress will outcompete the less adapted strains in the population and will eventually come to dominate the population.

This same process can be extended to any essential metabolite by creating an auxotroph. An auxotroph is a strain incapable of synthesizing an essential metabolite and must therefore have the metabolite provided in the media to grow. In this scenario, by making an auxotroph and passaging it on decreasing amounts of the metabolite, the resulting dominant strains should be more capable of obtaining and incorporating this essential metabolite.

For example, if the biosynthetic pathway for producing an amino acid is disrupted a strain capable of high-affinity capture of said amino acid can be evolved via ALE. First, the strain is grown in varying concentrations of the auxotrophic amino acid, until a minimum concentration to support growth is established. The strain is then passaged at that concentration, and diluted into lowering concentrations of the amino acid at regular intervals. Over time, cells that are most competitive for the amino acid — at growth-limiting concentrations — will come to dominate the population. These strains will likely have mutations in their amino acid-transporters resulting in increased ability to import the essential and limiting amino acid.

Similarly, by using an auxotroph that cannot use an upstream metabolite to form an amino acid, a strain can be evolved that not only can more efficiently import the upstream metabolite, but also convert the metabolite into the essential downstream metabolite. These strains will also evolve mutations to increase import of the upstream metabolite, but may also contain mutations which increase expression or reaction kinetics of downstream enzymes, or that reduce competitive substrate utilization pathways.

A metabolite innate to the microbe can be made essential via mutational auxotrophy and selection applied with growth-limiting supplementation of the endogenous metabolite. However, phenotypes capable of consuming non-native compounds can be evolved by tying their consumption to the production of an essential compound. For example, if a gene from a different organism is isolated which can produce an essential compound or a precursor to an essential compound this gene can be recombinantly introduced and expressed in the heterologous host. This new host strain will now have the ability to synthesize an essential nutrient from a previously non-metabolizable substrate.

Hereby, a similar ALE process can be applied by creating an auxotroph incapable of converting an immediately downstream metabolite and selecting in growth-limiting amounts of the non-native compound with concurrent expression of the recombinant enzyme. This will result in mutations in the transport of the non-native substrate, expression and activity of the heterologous enzyme and expression and activity of downstream native enzymes. It should be emphasized that the key requirement in this process is the ability to tether the consumption of the non-native metabolite to the production of a metabolite essential to growth.

Once the basis of the selection mechanism is established and minimum levels of supplementation have been established, the actual ALE experimentation can proceed. Throughout this process several parameters must be vigilantly monitored. It is important that the cultures are maintained in an exponential growth phase and not allowed to reach saturation/stationary phase. This means that growth rates must be check during each passaging and subsequent dilutions adjusted accordingly. If growth rate improves to such a degree that dilutions become large, then the concentration of auxotrophic supplementation should be decreased such that growth rate is slowed, selection pressure is increased and dilutions are not so severe as to heavily bias subpopulations during passaging. In addition, at regular intervals cells should be diluted, grown on solid media and individual clones tested to confirm growth rate phenotypes observed in the ALE cultures.

Predicting when to halt the stop the ALE experiment also requires vigilance. As the success of directing evolution is tied directly to the number of mutations “screened” throughout the experiment and mutations are generally a function of errors during DNA replication, the cumulative cell divisions (CCD) acts as a proxy for total mutants which have been screened. Previous studies have shown that beneficial phenotypes for growth on different carbon sources can be isolated in about 10^{11.2} CCD¹. This rate can be accelerated by the addition of chemical mutagens to the cultures — such as N-methyl-N-nitro-N-nitrosoguanidine (NTG) — which causes increased DNA replication errors. However, when continued passaging leads to marginal or no improvement in growth rate the population has converged to some fitness maximum and the ALE experiment can be halted.

At the conclusion of the ALE experiment, the cells should be diluted, isolated on solid media and assayed for growth phenotypes matching that of the culture flask. Best performers from those selected are then prepped for genomic DNA and sent for whole genome sequencing. Sequencing with reveal mutations occurring around the genome capable of providing improved phenotypes, but will also contain silent mutations (those which provide no benefit but do not detract from desired phenotype). In cultures evolved in the presence of NTG or other chemical mutagen, there will be significantly more silent, background mutations. If satisfied with the best performing strain in its current state, the user can proceed to application with that strain. Otherwise the contributing mutations can be deconvoluted from the evolved strain by reintroducing the mutations to the parent strain by genome engineering techniques. See Lee, D.-H., Feist, A. M., Barrett, C. L. & Palsson, B. Ø. Cumulative Number of Cell Divisions as a Meaningful Timescale for Adaptive Laboratory Evolution of Escherichia coli. PLoS ONE 6, e26172 (2011).

Similar methods can be used to generate E. coli Nissle mutants that consume or import branched chain amino acids, e.g., leucine, valine, and/or isoleucine.

### Specific Screen to Identify Strains with Improved BCAA Degradation Enzyme Activity

Screens using genetic selection are conducted to improve BCAA consumption in the genetically engineered bacteria. Toxic BCAA analogs exert their mechanism of action (MOA) by being incorporated into cellular protein, causing cell death. These compounds, e.g., fluoro-leucine and/or aza-leucine, have utility in an untargeted approach to select BCAA enzymes with increased activity. Assuming that these toxic compounds can be metabolized by BCAA enzymes into a nontoxic metabolite, rather than being incorporated into cellular protein, genetically engineered bacteria which have improved BCAA degradation activity can tolerate higher levels of these compounds, and can be screened for and selected on this basis.

### Use of Valine and Leucine Sensitivity to Identify Strains with Improved BCAA Degradation Enzyme Activity

Valine and Leucine sensitivity can be used as a genetic screening tool using the E.coli K12 strain. There are three AHAS (acetohydroxybutanoate synthase) isozymes in E. coli (AHAS I: ilvBN, AHAS II: ilvGM, and AHAS III: ilvIH). Valine and leucine exert feedback inhibition on AHAS I and AHAS III; AHAS II is resistant to Val and Leu inhibition. E. coli K12 has a frameshift mutation in ilvG (AHAS II) and is unable to produce BCAA endogenously in the presence of valine and leucine. In contrast, E. coli Nissle has a functional ilvG and is insensitive to valine and leucine and therefore cannot be used for this screen. A genetically engineered strain derived from E. coli K12, which more efficiently degrades leucine, has a greater reduction in sensitivity to leucine (through relieving the feedback inhibition on AHAS I and III). As a result, this pathway can be used as a tool to select and identify a strain with improved resistance to leucine.

### Use of Leucine Auxotrophy and D-Leucine as a Method to Identify Strains with Improved BCAA Uptake Ability

Bacterial mutants with increased leucine transport into the bacterial cell may be identified using a leucine auxotroph and providing D-leucine instead of L-leucine in the media, as D-leucine can be imported through the same transporters. The bacteria can grow in the presence of D-leucine, because the bacterial stain has a racemase, which can convert D-leucine to L-leucine. However, the uptake of D-leucine through LivKHMGF is less efficient than the uptake of L-leucine. The leucine auxotroph can still grow if high concentrations of D-Leucine are provided, even though the D-leucine uptake is less efficient than L-leucine uptake. When concentrations of D-leucine in the media are lowered, the cells can no longer grow, unless transport efficiency is increased, ergo, mutants with increased D-leucine uptake can be selected.

### Methods of Treatment

Further disclosed herein are methods of treating a disease or disorder associated with the catabolism of leucine. In some embodiments, disclosed herein are methods for reducing, ameliorating, or eliminating one or more symptom(s) associated with these diseases or disorders. In one embodiment, the disorder involving the catabolism of leucine is a metabolic disorder involving the abnormal catabolism of leucine. Metabolic diseases associated with abnormal catabolism of leucine include isovaleric acidemia, maple syrup urine disease (MSUD), propionic acidemia, methylmalonic acidemia, diabetes ketoacidosis, MCC Deficiency, 3-Methylglutaconyl-CoA hydratase Deficiency, HMG-CoA Lyase Deficiency, Acetyl-CoA Carboxylase Deficiency, Malonyl-CoA Decarboxylase Deficiency, short-branched chain acylCoA dehydrogenase deficiency, 2-methyl-3-hydroxybutyric acidemia, beta-ketothiolase deficiency, isobutyryl-CoA dehydrogenase deficiency, HIBCH deficiency), and 3-Hydroxyisobutyric aciduria.

In one embodiment, the disease associated with abnormal catabolism of leucine is isovaleric acidemia. In one embodiment, the disease associated with abnormal catabolism of leucine is propionic acidemia. In one embodiment, the disease associated with abnormal catabolism of leucine is methylmalonic acidemia. In another embodiment, the disease associated with abnormal catabolism of leucine is diabetes. In one embodiment, the disease is maple syrup urine disease (MSUD). The present disclosure surprisingly demonstrates that pharmaceutical compositions comprising the recombinant bacterial cells disclosed herein may be used to treat metabolic diseases involving the abnormal catabolism of leucine, such as isovaleric acidemia. In one embodiment, the metabolic disease is selected from the group consisting of classic MSUD, intermediate MSUD, intermittent MSUD, E3-Deficient MSUD, and thiamine-responsive MSUD. In one embodiment, the disease is classic MSUD. In another embodiment, the disease is intermediate MSUD. In another embodiment, the disease is intermittent MSUD. In another embodiment, the disease is E3-deficient MSUD. In another embodiment, the disease is thiamine-responsive MSUD.

In one embodiment, the subject having MSUD has a mutation in an E1α gene. In another embodiment, the subject having MSUD has a mutation in the E1β gene. In another embodiment, the subject having MSUD has a mutation in the E2 gene. In another embodiment, the subject having MSUD has a mutation in the E3 gene.

The leucine consumption kinetics and dosing are set forth herein. Food intake is based on adult recommended daily allowance of 40 mg/kg/day. MSUD patients are primarily children with restricted protein intake.

In another embodiment, the disorder involving the catabolism of leucine is a disorder caused by the activation of mTor (mammalian target of rapamycin). mTor is a serine-threonine kinase and has been implicated in a wide range of biological processes including transcription, translation, autophagy, actin organization and ribosome biogenesis, cell growth, cell proliferation, cell motility, and survival. mTOR exists in two complexes, mTORC1 and mTORC2. mTORC1 contains the raptor subunit and mTORC2 contains rictor. These complexes are differentially regulated, and have distinct substrate specificities and rapamycin sensitivity. For example, mTORC1 phosphorylates S6 kinase (S6K) and 4EBP1, promoting increased translation and ribosome biogenesis to facilitate cell growth and cell cycle progression. S6K also acts in a feedback pathway to attenuate PI3K/Akt activation. mTORC2 is generally insensitive to rapamycin and is thought to modulate growth factor signaling by phosphorylating the C-terminal hydrophobic motif of some AGC kinases, such as Akt.

It is known in the art that mTor activation is caused by branched chain amino acids or alpha keto acids in the subject (see, for example, Harlan et al., Cell Metabolism, 17:599-606, 2013). Specifically, activation of mTorC1 (mTor complex 1) is caused by leucine (see Han et al., Cell, 149:410-424, 2012 and Lynch, J. Nutr., 131(3):861S-865S, 2001). Thus, in one embodiment, the disclosure provides methods of treating disorders involving the catabolism of leucine, caused by the activation of mTor by leucine in the subject. In one embodiment, the leucine levels in the subject are normal, and lowering leucine levels in the subject leads to the decreased activity of mTor and, thus, treatment of the disease. In another embodiment, the leucine levels in the subject are increased, and lowering leucine levels in the subject leads to the decreased activity of mTor and, thus, treatment of the disease. In one embodiment, the activation of mTor is increased as compared to the normal level of activation of mTor in a healthy subject, and lowering leucine levels in the subject leads to the decreased activation of mTor and, thus, treatment of the disease. In one embodiment, the level of activity of mTor is increased as compared to the normal level of activity of mTor in a healthy subject, and lowering leucine levels in the subject leads to the decreased activity of mTor and, thus, treatment of the disease. In another embodiment, the expression of mTor is increased as compared to the normal level of expression of mTor in a healthy subject, and lowering leucine levels in the subject leads to the decreased activity of mTor and, thus, treatment of the disease. In one embodiment, the activation of mTor is an abnormal activation of mTor.

Diseases caused by the activation of mTor are known in the art. See, for example, Laplante and Sabatini, Cell, 149(2):74-293, 2012. As used herein, the term “disease caused by the activation of mTor” includes cancer, obesity, type 2 diabetes, neurodegeneration, autism, Alzheimer’s disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson’s disease, Huntington’s disease, psoriasis, rheumatoid arthritis, lupus, multiple sclerosis, Leigh’s syndrome, and Friedrich’s ataxia.

In another aspect, the disclosure provides methods for decreasing the plasma level of leucine in a subject by administering a pharmaceutical composition comprising a bacterial cell disclosed herein to the subject, thereby decreasing the plasma level of leucine in the subject.

In some embodiments, the disclosure provides methods for reducing, ameliorating, or eliminating one or more symptom(s) associated with these diseases, including but not limited to neurological deficits, mental retardation, brain damage, brain oedema, blindness, branched chain α-keto acid acidosis, myelinization failure, hyperammonaemia, coma, developmental delay, neurological impairment, failure to thrive, ketoacidosis, seizure, ataxia, neurodegeneration, hypotonia, lactic acidosis, recurrent myoglobinuria, and/or liver failure. In some embodiments, the disease is secondary to other conditions, e.g., liver disease.

In certain embodiments, the bacterial cells disclosed herein are capable of catabolizing leucine, in the diet of the subject in order to treat a disease associated with catabolism of leucine, e.g., isovaleric acidemia. In these embodiments, the bacterial cells are delivered simultaneously with dietary protein. In another embodiment, the bacterial cells are delivered simultaneously with phenylbutyrate. In another embodiment, the bacterial cells are delivered simultaneously with a thiamine supplement. In some embodiments, the bacterial cells and dietary protein are delivered after a period of fasting or leucine-restricted dieting. In these embodiments, a patient suffering from a disorder involving the catabolism of leucine, e.g., isovaleric acidemia, may be able to resume a substantially normal diet, or a diet that is less restrictive than a leucine-free diet. In some embodiments, the bacterial cells may be capable of catabolizing leucine from additional sources, e.g., the blood, in order to treat a disease associated with the catabolism of leucine, e.g., isovaleric acidemia. In these embodiments, the bacterial cells need not be delivered simultaneously with dietary protein, and a leucine gradient is generated, e.g., from blood to gut, and the recombinant bacteria catabolize, leucine, and reduce plasma levels of leucine.

The method may comprise preparing a pharmaceutical composition with at least one genetically engineered species, strain, or subtype of bacteria described herein, and administering the pharmaceutical composition to a subject in a therapeutically effective amount. In some embodiments, the bacterial cells disclosed herein are administered orally, e.g., in a liquid suspension. In some embodiments, the bacterial cells disclosed herein are lyophilized in a gel cap and administered orally. In some embodiments, the bacterial cells disclosed herein are administered via a feeding tube or gastric shunt. In some embodiments, the bacterial cells disclosed herein are administered rectally, e.g., by enema. In some embodiments, the genetically engineered bacteria are administered topically, intraintestinally, intrajejunally, intraduodenally, intraileally, and/or intracolically.

In certain embodiments, the administering the pharmaceutical composition described herein reduces leucine levels in a subject. In some embodiments, the methods of the present disclosure reduce leucine levels in a subject by at least about 10%, 20%, 25%, 30%, 40%, 50%, 60%, 70%, 75%, 80%, 85%, 90%, 95%, or more. In another embodiment, the methods of the present disclosure reduce leucine levels in a subject by at least two-fold, three-fold, four-fold, five-fold, six-fold, seven-fold, eight-fold, nine-fold, or ten-fold. In some embodiments, reduction is measured by comparing leucine level in a subject before and after administration of the pharmaceutical composition. In one embodiment, leucine level is reduced in the gut of the subject. In another embodiment, leucine level is reduced in the blood of the subject. In another embodiment, leucine level is reduced in the plasma of the subject. In another embodiment, leucine level is reduced in the brain of the subject.

In one embodiment, the pharmaceutical composition described herein is administered to reduce leucine levels in a subject to normal levels. In another embodiment, the pharmaceutical composition described herein is administered to reduce leucine levels in a subject below normal levels to, for example, decrease the activation of mTor.

In some embodiments, the method of treating the disorder involving the catabolism of leucine, e.g., isovaleric acidemia, allows one or more symptoms of the condition or disorder to improve by at least about 10%, 20%, 30%, 40%, 50%, 60%, 70%, 80%, 90%, 95%, or more. In some embodiments, the method of treating the disorder involving the catabolism of leucine, e.g., isovaleric acidemia, allows one or more symptoms of the condition or disorder to improve by at least about two-fold, three-fold, four-fold, five-fold, six-fold, seven-fold, eight-fold, nine-fold, or ten-fold.

Before, during, and after the administration of the pharmaceutical composition, leucine levels in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions disclosed herein to reduce levels of leucine. In some embodiments, the methods may include administration of the compositions disclosed herein to reduce leucine to undetectable levels in a subject. In some embodiments, the methods may include administration of the compositions disclosed herein to reduce leucine, concentrations to undetectable levels, or to less than about 1%, 2%, 5%, 10%, 20%, 25%, 30%, 40%, 50%, 60%, 70%, 75%, 80%, 85%, 90%, or 95% of the subject’s leucine levels prior to treatment.

In some embodiments, the recombinant bacterial cells disclosed herein produce a leucine catabolism enzyme, e.g., leucine decarboxylase (LDC), LivKHMGF and BrnQ, etc., under exogenous environmental conditions, such as the low-oxygen environment of the mammalian gut, to reduce levels of leucine in the blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

In one embodiment, the bacteria disclosed herein reduce plasma levels of leucine to less than 4 mg/dL. In one embodiment, the bacteria disclosed herein reduce plasma levels of leucine, to less than 3.9 mg/dL. In one embodiment, the bacteria disclosed herein reduce plasma levels of leucine to less than 3.8 mg/dL, 3.7 mg/dL, 3.6 mg/dL, 3.5 mg/dL, 3.4 mg/dL, 3.3 mg/dL, 3.2 mg/dL, 3.1 mg/dL, 3.0 mg/dL, 2.9 mg/dL, 2.8 mg/dL, 2.7 mg/dL, 2.6 mg/dL, 2.5 mg/dL, 2.0 mg/dL, 1.75 mg/dL, 1.5 mg/dL, 1.0 mg/dL, or 0.5 mg/dL.

In one embodiment, the subject has plasma levels of at least 4 mg/dL prior to administration of the pharmaceutical composition disclosed herein. In another embodiment, the subject has plasma levels of at least 4.1 mg/dL, 4.2 mg/dL, 4.3 mg/dL, 4.4 mg/dL, 4.5 mg/dL, 4.75 mg/dL, 5.0 mg/dL, 5.5 mg/dL, 6 mg/dL, 7 mg/dL, 8 mg/dL, 9 mg/dL, or 10 mg/dL prior to administration of the pharmaceutical composition disclosed herein.

Certain unmodified bacteria will not have appreciable levels of leucine, processing. In embodiments using genetically modified forms of these bacteria, processing of leucine, will be appreciable under exogenous environmental conditions.

Leucine levels, may be measured by methods known in the art, e.g., blood sampling and mass spectrometry. In some embodiments, leucine decarboxylase expression is measured by methods known in the art. In another embodiment, leucine decarboxylase activity is measured by methods known in the art to assess leucine decarboxylase activity. In one embodiment, leucine decarboxylase activity is measured by determining the amount of isopentylamine excreted in the media or urine.

In certain embodiments, the recombinant bacteria are E. coli Nissle. The recombinant bacteria may be destroyed, e.g., by defense factors in the gut or blood serum (Sonnenborn et al., 2009) or by activation of a kill switch, several hours or days after administration. Thus, the pharmaceutical composition comprising the recombinant bacteria may be re-administered at a therapeutically effective dose and frequency. In alternate embodiments, the recombinant bacteria are not destroyed within hours or days after administration and may propagate and colonize the gut.

In one embodiments, the bacterial cells disclosed herein are administered to a subject once daily. In another embodiment, the bacterial cells disclosed herein are administered to a subject twice daily. In another embodiment, the bacterial cells disclosed herein are administered to a subject in combination with a meal. In another embodiment, the bacterial cells disclosed herein are administered to a subject prior to a meal. In another embodiment, the bacterial cells disclosed herein are administered to a subject after a meal. The dosage of the pharmaceutical composition and the frequency of administration may be selected based on the severity of the symptoms and the progression of the disease. The appropriate therapeutically effective dose and/or frequency of administration can be selected by a treating clinician.

The methods disclosed herein may comprise administration of a composition disclosed herein alone or in combination with one or more additional therapies, e.g., the phenylbutyrate, thiamine supplementation, and/or a low-branched chain amino acid, e.g., a low-leucine, diet. An important consideration in the selection of the one or more additional therapeutic agents is that the agent(s) should be compatible with the bacteria disclosed herein, e.g., the agent(s) must not interfere with or kill the bacteria. In some embodiments, the genetically engineered bacteria are administered in combination with a low protein diet. In some embodiments, administration of the genetically engineered bacteria provides increased tolerance, so that the patient can consume more protein.

The methods disclosed herein may further comprise isolating a plasma sample from the subject prior to administration of a composition disclosed herein and determining the level of leucine in the sample. In some embodiments, the methods disclosed herein may further comprise isolating a plasma sample from the subject after to administration of a composition disclosed herein and determining the level of leucine in the sample.

In one embodiment, the methods disclosed herein further comprise comparing the level of leucine or isopentylamine in the plasma sample from the subject after administration of a composition disclosed herein to the subject to the plasma sample from the subject before administration of a composition disclosed herein to the subject. In one embodiment, a reduced level of leucine or isopentylamine in the plasma sample from the subject after administration of a composition disclosed herein indicates that the plasma levels of leucine or isopentylamine are decreased, thereby treating the disorder involving the catabolism of leucine in the subject. In one embodiment, the plasma level of leucine or isopentylamine is decreased at least 10%, 20%, 30%, 40 \$, 50%, 60%, 70%, 80%, 90%, or 100% in the sample after administration of the pharmaceutical composition as compared to the plasma level in the sample before administration of the pharmaceutical composition. In another embodiment, the plasma level of leucine or isopentylamine is decreased at least two-fold, three-fold, four-fold, or five-fold in the sample after administration of the pharmaceutical composition as compared to the plasma level in the sample before administration of the pharmaceutical composition.

In one embodiment, the methods disclosed herein further comprise comparing the level of leucine or isopentylamine in the plasma sample from the subject after administration of a composition disclosed herein to a control level of leucine or isopentylamine. In one embodiment, the control level of leucine is 4 mg/dL. In one embodiment, the subject is considered treated if the level of leucine in the plasma sample from the subject after administration of the pharmaceutical composition disclosed herein, is less than 4 mg/dL. In one embodiment, the subject is considered treated if the level of leucine in the plasma sample from the subject after administration of the pharmaceutical composition disclosed herein is less than 3.9, 3.8, 3.7, 3.6, 3.5, 3.4, 3.3, 3.2, 3.1, 3.0, 2.5, 2.0, 1.5, 1.0 or 0.5 mg/dL.

### EXEMPLIFICATION

The present disclosure is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are also expressly incorporated herein by reference.

### Example 1: Generation and Testing of Strain Expressing Leucine Decarboxylase (LDC)

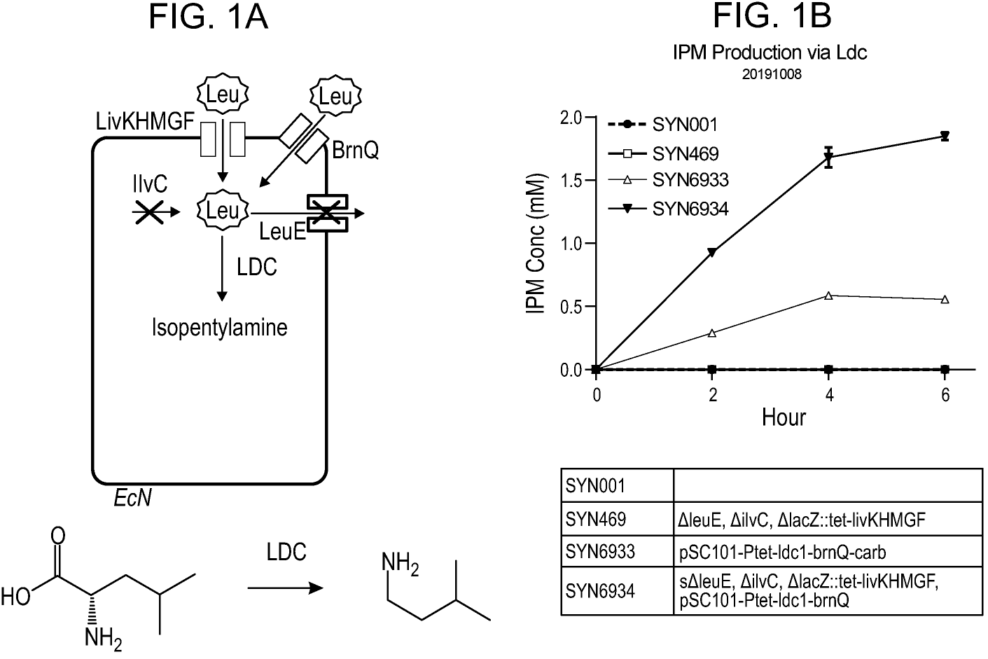

Two strains containing leucine decarboxylase (LDC) were constructed. Wild type E. coli Nissle (EcN) was transformed with a pSC101-Ptet-ldc-brnQ plasmid, resulting in SYN6933. The same plasmid was also transformed into a strain with leucine biosynthesis and leucine export knocked out (FIGS. 1A and 1B; SYN6934). The isopentylamine (IPM) production was then determined in an in vitro assay compared to their respective parent strains. SYN6934 demonstrates that active import of leucine was advantageous (FIG. 1B).

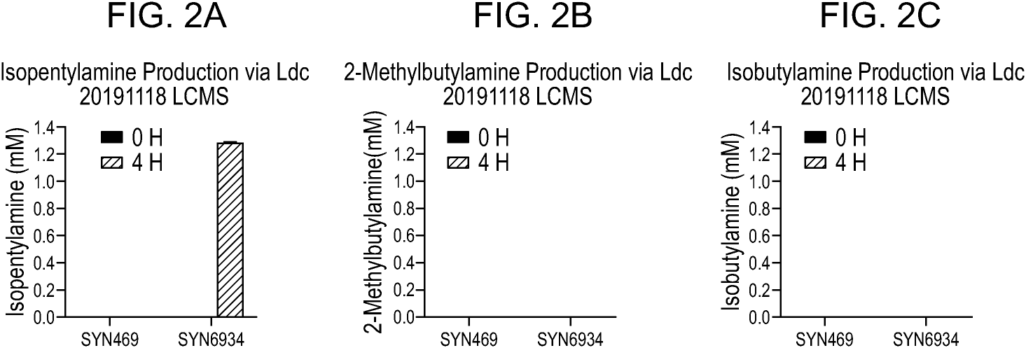

FIGS. 2A-2C depict BCAA-amine production via LDC using leucine as a substrate. The assays use 3 E9 cells/mL and 10 mM single BCAA for 4 hours at 37° C. in anaerobic chamber (>30 PPM O₂). FIG. 2A is a graph depicting isopentylamine production. FIG. 2B is a graph depicting 2-methylbutylamine production, and FIG. 2C is a graph depicting isobutylamine production. Only isopentylamine is detected, demonstrating that LDC is specific for leucine.

Table of Sequences

| SEQ ID NO: | Name |
|---|---|
| 145 | LDC nucleic acid (mouse) |
| 167 | LDC Protein (mouse) |

1. A recombinant bacterium comprising
at least one gene sequence encoding a leucine decarboxylase (LDC) enzyme operably linked to at least one directly or indirectly inducible promoter that is not associated with a gene encoding the leucine decarboxylase enzyme in nature; and
at least one gene sequence encoding at least one transporter capable of importing leucine into the bacterium operably linked to at least one directly or indirectly inducible promoter that is not associated with a gene encoding the transporter in nature.

2. The recombinant bacterium of claim 1, wherein the at least one gene sequence encoding a leucine decarboxylase enzyme comprises a sequence having at least 90% identity to SEQ ID NO:145.

3. The recombinant bacterium of claim 1 or claim 2, wherein the at least one gene sequence encoding at least one transporter comprises a brnQ sequence and/or a livKHMGF sequence.

4. The recombinant bacterium of claim 3, wherein the brnQ sequence comprises a sequence having at least 90% identity to SEQ ID NO:64.

5. The recombinant bacterium of claim 3 or claim 4, wherein the livKHMGF sequence comprises a sequence having at least 90% identity to SEQ ID NO:91.

6. The recombinant bacterium of any one of the previous claims, wherein the bacterium comprises a genetic modification in leuE that reduces leucine export from the bacterium.

7. The recombinant bacterium of any one of the previous claims, wherein the bacterium comprises a genetic modification in ilvC that reduces endogenous biosynthesis of leucine in the bacterium.

8. The recombinant bacterium of any one of the previous claims, wherein the at least one gene sequence encoding the at least one leucine catabolism enzyme is integrated into the chromosome of the bacterium or is present on a plasmid in the bacterium.

9. The recombinant bacterium of any one of the previous claims, wherein the promoter is selected from the group consisting of an FNRS promoter, a Pₜₑₜ promoter, a P_{cmt} promoter, a P_{c1857} promoter, and a P_{BAD} promoter.

10. The recombinant bacterium of any one of the previous claims, wherein the bacterium is a probiotic bacterium selected from the group consisting of Bacteroides, Bifidobacterium, Clostridium, Escherichia, Lactobacillus, and Lactococcus,.

11. The recombinant bacterium of claim 10, wherein the bacterium is Escherichia coli strain Nissle.

12. A pharmaceutically acceptable composition comprising the recombinant bacterium of any one of the previous claims and a pharmaceutically acceptable carrier.

13. A method of reducing the level of leucine in a subject, the method comprising a step of administering to the subject the pharmaceutically acceptable composition of claim 12.

14. A method of treating a disease associated with excess leucine and/or a metabolic disorder involving the abnormal catabolism of leucine in a subject, the method comprising a step of administering to the subject the pharmaceutically acceptable composition of claim 12.

15. The method of claim 13 or claim 14, wherein the subject has, or is suspected of having, isovaleric acidemia.


SEQUENCE LISTING (169 sequences)

SEQ ID NO: 1 - DNA, 1647 bp, Lactococcus lactis
  kivD gene from Lactococcus lactis IFPL730
atgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataa

SEQ ID NO: 2 - DNA, 2433 bp, Artificial Sequence
  Synthetic: Tet-kivD construct
gaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataatacgcatggcatggatgaattgtataaataa

SEQ ID NO: 3 - DNA, 5739 bp, Artificial Sequence
  Synthetic: Tet-bkd construct sequence
gtaaaacgacggccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgtccgactacgagccactccgcttgcacgtgccggagccgacaggtcgtcccggctgcaaaacggatttctcttacctgcacttatctcccgcaggtgaagtccgcaaaccgcctgtcgacgtggagcctgcagaaaccagcgatttggcatattcgctggtgcgtgtgctcgatgatgatggacatgcagtgggtccgtggaatccgcagctctcaaacgaacagctgctgcgtggaatgcgcgcgatgctgaagacgcgtctgttcgatgctcgcatgttgactgcgcagcgccaaaaaaaattgagtttttatatgcagtgcttaggagaagaggcaatcgcgactgcccatacactggccctgcgcgatggtgatatgtgttttccgacgtaccgtcagcaggggattcttattacacgtgagtatccgcttgtggatatgatctgccagctgctgtcgaatgaagcggaccccctgaaaggccgtcaactgccgatcatgtacagcagtaaggaggctggcttctttagcatctcgggcaatcttgcgactcagtttattcaggcggtggggtgggggatggcaagcgcaatcaaaggggatacccgcattgcatccgcatggattggcgatggcgctaccgcggaaagcgattttcatacggcgctgacctttgctcacgtttatcgcgcaccggtgatcctcaatgtggtcaacaaccagtgggcgatttcgacgtttcaggccatcgcgggcggcgagggcaccacgttcgcgaaccgtggcgtgggttgcggcattgcgagcctccgtgtggacgggaacgattttttggccgtgtatgcggcgagcgaatgggcggcagaacgcgcacgccgtaacttgggaccgtccctgatcgaatgggtaacttatcgcgcgggcccacacagcacgagcgacgatccgtcaaagtatcgccctgcggatgattggaccaattttccgctgggtgacccgattgcgcgtctgaaacgtcacatgatcggtttgggtatttggagcgaagaacagcacgaagctacgcacaaagcgctggaagcggaagtcctggcggcgcagaagcaggccgaaagccatggcactctgattgacggccgtgtgccgtctgcagcctctatgttcgaagatgtttatgccgagttacccgagcacttacgtcgccagcgccaggagctcggggtatgaacgccatgaacccgcagcatgaaaacgcgcaaaccgtgacctccatgacgatgattcaggccctgcgctcggcgatggatattatgttagaacgtgacgatgacgtcgtggtgtttggtcaggacgtagggtattttgggggagtgtttcgttgtaccgaggggttgcaaaagaagtatggtacgagtcgcgtcttcgatgcaccgatcagcgaatcaggcattatcggcgctgccgtgggcatgggtgcatatggcttgcgccctgtggttgaaattcagtttgcagattatgtatatcccgcgtctgaccaactgattagtgaggcggcacgcctccgctaccgtagcgcgggcgatttcattgtcccgatgaccgtccgcatgccttgtggagggggcatttacggtggccaaacgcattctcagagtccagaagccatgttcacacaagtgtgcggtcttcgcaccgtgatgccatctaatccttatgacgccaaaggattactgattgcgtgcatcgaaaacgacgatccggttatctttttagaacccaaacgtctgtacaacggtcctttcgacggtcatcacgaccgtcctgtcacgccgtggagcaaacatccggcatcgcaagtcccggatgggtattataaagtgcctctggacaaagcagcgattgtccgccctggtgcagcccttacagtcctgacgtatggtaccatggtgtacgtggcgcaggccgcggcagatgaaaccggcctcgatgcggagattatcgacctccgcagtctgtggccgctggacttggaaactatcgtcgcgagtgtgaaaaagaccggtcgttgtgttattgcccatgaagcgactcgtacctgcggctttggcgccgaactgatgtccctggtgcaggaacactgttttcaccatcttgaggctccgattgaacgcgtcactggctgggacacaccgtaccctcatgcgcaggaatgggcctatttcccgggcccagcgcgcgtgggagccgcctttaaacgcgtgatggaggtctgaatgggtacccacgttattaaaatgcctgatattggtgaaggcatcgcggaggtagagctggttgaatggcacgttcaagtgggtgatagcgtgaatgaagatcaggtactcgcggaagtaatgacggacaaagcaacggttgaaatcccgtcccctgttgctggccgcatcttggcactgggtggccagccgggacaagttatggcggtgggaggagaattaattcgcctggaagtggagggtgccggaaacctggcggagtctccggccgcagctacgcccgccgctccggtggcagcaactccggaaaaacctaaagaagcaccggttgcagcgccaaaagcagctgccgaagcaccccgtgcgcttcgtgattctgaagcgccgcgccaacgccgccagccgggggaacgcccattagcatcaccggccgtccgtcagcgtgcccgcgacctgggaatcgagctgcagtttgttcagggctctggcccagccggccgcgtgcttcatgaggacctggatgcgtatcttacgcaggatggaagtgttgctcgttcaggcggcgctgcgcagggttacgcggaacgccatgatgaacaggcagtcccggtgatcggtctgcgccgcaaaattgcccagaagatgcaggatgctaaacgccgcattcctcacttcagttacgtcgaagagattgacgtaaccgatctggaagccctgcgcgctcacttgaatcagaaatggggcgggcaacgtggtaaactgacgctgctgcctttcctcgtccgcgcaatggtcgtcgcattacgcgatttcccgcaactgaatgctcgctatgatgatgaagcggaagtagtgacgcgttacggggccgttcatgttggtatcgcgacccagtcagataatgggctcatggttccggtgttgcgccatgcagaaagccgtgacctgtggggtaatgcgtcggaagttgcgcgtctggccgaagcggcgcgttccggtaaagcgcaacgtcaggaactgagcggctccacgattaccctgtcaagccttggtgtgttgggagggattgtatccacgccagtcattaatcacccggaagttgcaatcgttggtgttaaccgtattgtggagcgccctatggttgttggtggtaatattgtagtacgtaaaatgatgaatctgagctcttcgtttgatcatcgcgtggtggacggcatggatgctgcggcttttattcaagccgtgcgcggtttgttagaacatcctgccaccctgttcctggagtaagcgatgagtcagattttaaaaacctcgctcctgatcgttggcggcgggccaggcggctatgtggcggcgatccgcgccggccagctggggattccaacggtgttggttgagggcgccgctttgggcggtacttgcctgaatgtggggtgcattccgagcaaagcgttgatccatgctgccgaagagtaccttaaagcgcgccactatgcatcacgttccgcgctgggcatccaggtgcaagcaccttcaattgacatcgcccgcaccgtggaatggaaagacgccattgtggaccgtttgacttcgggtgtggcggctctgctgaaaaagcatggtgtggatgtagtacaaggatgggcacgcatcctcgacggcaagagcgtggcggttgaactggcgggcggggggtcgcagcgcatcgagtgtgaacatctgcttctggcggcgggctcacaaagcgttgaattacccatcctgcctctggggggcaaagtaatcagcagcaccgaagcattagctccggggtcgttgccaaaacgtctggtggttgtgggtggcggttatattggtctggagctgggcactgcatatcgcaagctgggtgttgaagttgctgtggtggaggcacaaccccgcatcctgccgggctacgatgaggaactgactaagccggtggcccaagcgctgcgccgtctgggtgtagaactgtacctgggtcattcattgctgggaccgagtgaaaacggcgttcgcgtgcgtgatggggctggcgaagaacgtgagatcgccgcggaccaggtccttgtcgcagttggccgcaaaccgcgttcagagggttggaacctggagtctctcggtttagacatgaatgggcgtgccgtaaaagtggacgatcagtgccgtacaagcatgcgtaacgtatgggccattggcgacctggcgggcgaaccgatgctggcgcaccgcgctatggcgcaaggagaaatggtcgccgaattgattgcgggcaaacgccgtcagtttgcgccggttgcaattcctgcagtctgttttacggatccggaagtggtggtggcgggtctgagtccggaacaggccaaagatgcgggtctggattgcctggtcgcgtcattcccgttcgcagccaacggccgcgccatgacgttggaagctaacgaaggctttgtccgcgtggtggcacgtcgtgacaaccatctggtggttggttggcaggcggtcggtaaagctgtgtctgaattaagcaccgcgttcgcacaatctctggaaatgggcgctcgcctcgaagacattgcaggcacaatccacgcgcaccccaccctgggtgaagctgttcaggaagcggcactccgtgccttaggtcacgccctgcacatttga

SEQ ID NO: 4 - DNA, 6781 bp, Artificial Sequence
  Synthetic: Tet-leuDH-bkd construct
gtaaaacgacggccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgttcgatatgatggatgcagcccgcctggaaggcctgcacctcgcccaggatccagcgacgggcctgaaagcgatcatcgcgatccattccactcgcctcggcccggccttaggcggctgtcgttacctcccatatccgaatgatgaagcggccatcggcgatgccattcgcctggcgcagggcatgtcctacaaagctgcacttgcgggtctggaacaaggtggtggcaaggcggtgatcattcgcccaccccacttggataatcgcggtgccttgtttgaagcgtttggacgctttattgaaagcctgggtggccgttatatcaccgccgttgactcaggaacaagtagtgccgatatggattgcatcgcccaacagacgcgccatgtgacttcaacgacacaagccggcgacccatctccacatacggctctgggcgtctttgccggcatccgcgcctccgcgcaggctcgcctggggtccgatgacctggaaggcctgcgtgtcgcggttcagggccttggccacgtaggttatgcgttagcggagcagctggcggcggtcggcgcagaactgctggtgtgcgacctggaccccggccgcgtccagttagcggtggagcaactgggggcgcacccactggcccctgaagcattgctctctactccgtgcgacattttagcgccttgtggcctgggcggcgtgctcaccagccagtcggtgtcacagttgcgctgcgcggccgttgcaggcgcagcgaacaatcaactggagcgcccggaagttgcagacgaactggaggcgcgcgggattttatatgcgcccgattacgtgattaactcgggaggactgatttatgtggcgctgaagcatcgcggtgctgatccgcatagcattaccgcccacctcgctcgcatccctgcacgcctgacggaaatctatgcgcatgcgcaggcggatcatcagtcgcctgcgcgcatcgccgatcgtctggcggagcgcattctgtacggcccgcaataatgaaggagatatacatatgtccgactacgagccactccgcttgcacgtgccggagccgacaggtcgtcccggctgcaaaacggatttctcttacctgcacttatctcccgcaggtgaagtccgcaaaccgcctgtcgacgtggagcctgcagaaaccagcgatttggcatattcgctggtgcgtgtgctcgatgatgatggacatgcagtgggtccgtggaatccgcagctctcaaacgaacagctgctgcgtggaatgcgcgcgatgctgaagacgcgtctgttcgatgctcgcatgttgactgcgcagcgccaaaaaaaattgagtttttatatgcagtgcttaggagaagaggcaatcgcgactgcccatacactggccctgcgcgatggtgatatgtgttttccgacgtaccgtcagcaggggattcttattacacgtgagtatccgcttgtggatatgatctgccagctgctgtcgaatgaagcggaccccctgaaaggccgtcaactgccgatcatgtacagcagtaaggaggctggcttctttagcatctcgggcaatcttgcgactcagtttattcaggcggtggggtgggggatggcaagcgcaatcaaaggggatacccgcattgcatccgcatggattggcgatggcgctaccgcggaaagcgattttcatacggcgctgacctttgctcacgtttatcgcgcaccggtgatcctcaatgtggtcaacaaccagtgggcgatttcgacgtttcaggccatcgcgggcggcgagggcaccacgttcgcgaaccgtggcgtgggttgcggcattgcgagcctccgtgtggacgggaacgattttttggccgtgtatgcggcgagcgaatgggcggcagaacgcgcacgccgtaacttgggaccgtccctgatcgaatgggtaacttatcgcgcgggcccacacagcacgagcgacgatccgtcaaagtatcgccctgcggatgattggaccaattttccgctgggtgacccgattgcgcgtctgaaacgtcacatgatcggtttgggtatttggagcgaagaacagcacgaagctacgcacaaagcgctggaagcggaagtcctggcggcgcagaagcaggccgaaagccatggcactctgattgacggccgtgtgccgtctgcagcctctatgttcgaagatgtttatgccgagttacccgagcacttacgtcgccagcgccaggagctcggggtatgaacgccatgaacccgcagcatgaaaacgcgcaaaccgtgacctccatgacgatgattcaggccctgcgctcggcgatggatattatgttagaacgtgacgatgacgtcgtggtgtttggtcaggacgtagggtattttgggggagtgtttcgttgtaccgaggggttgcaaaagaagtatggtacgagtcgcgtcttcgatgcaccgatcagcgaatcaggcattatcggcgctgccgtgggcatgggtgcatatggcttgcgccctgtggttgaaattcagtttgcagattatgtatatcccgcgtctgaccaactgattagtgaggcggcacgcctccgctaccgtagcgcgggcgatttcattgtcccgatgaccgtccgcatgccttgtggagggggcatttacggtggccaaacgcattctcagagtccagaagccatgttcacacaagtgtgcggtcttcgcaccgtgatgccatctaatccttatgacgccaaaggattactgattgcgtgcatcgaaaacgacgatccggttatctttttagaacccaaacgtctgtacaacggtcctttcgacggtcatcacgaccgtcctgtcacgccgtggagcaaacatccggcatcgcaagtcccggatgggtattataaagtgcctctggacaaagcagcgattgtccgccctggtgcagcccttacagtcctgacgtatggtaccatggtgtacgtggcgcaggccgcggcagatgaaaccggcctcgatgcggagattatcgacctccgcagtctgtggccgctggacttggaaactatcgtcgcgagtgtgaaaaagaccggtcgttgtgttattgcccatgaagcgactcgtacctgcggctttggcgccgaactgatgtccctggtgcaggaacactgttttcaccatcttgaggctccgattgaacgcgtcactggctgggacacaccgtaccctcatgcgcaggaatgggcctatttcccgggcccagcgcgcgtgggagccgcctttaaacgcgtgatggaggtctgaatgggtacccacgttattaaaatgcctgatattggtgaaggcatcgcggaggtagagctggttgaatggcacgttcaagtgggtgatagcgtgaatgaagatcaggtactcgcggaagtaatgacggacaaagcaacggttgaaatcccgtcccctgttgctggccgcatcttggcactgggtggccagccgggacaagttatggcggtgggaggagaattaattcgcctggaagtggagggtgccggaaacctggcggagtctccggccgcagctacgcccgccgctccggtggcagcaactccggaaaaacctaaagaagcaccggttgcagcgccaaaagcagctgccgaagcaccccgtgcgcttcgtgattctgaagcgccgcgccaacgccgccagccgggggaacgcccattagcatcaccggccgtccgtcagcgtgcccgcgacctgggaatcgagctgcagtttgttcagggctctggcccagccggccgcgtgcttcatgaggacctggatgcgtatcttacgcaggatggaagtgttgctcgttcaggcggcgctgcgcagggttacgcggaacgccatgatgaacaggcagtcccggtgatcggtctgcgccgcaaaattgcccagaagatgcaggatgctaaacgccgcattcctcacttcagttacgtcgaagagattgacgtaaccgatctggaagccctgcgcgctcacttgaatcagaaatggggcgggcaacgtggtaaactgacgctgctgcctttcctcgtccgcgcaatggtcgtcgcattacgcgatttcccgcaactgaatgctcgctatgatgatgaagcggaagtagtgacgcgttacggggccgttcatgttggtatcgcgacccagtcagataatgggctcatggttccggtgttgcgccatgcagaaagccgtgacctgtggggtaatgcgtcggaagttgcgcgtctggccgaagcggcgcgttccggtaaagcgcaacgtcaggaactgagcggctccacgattaccctgtcaagccttggtgtgttgggagggattgtatccacgccagtcattaatcacccggaagttgcaatcgttggtgttaaccgtattgtggagcgccctatggttgttggtggtaatattgtagtacgtaaaatgatgaatctgagctcttcgtttgatcatcgcgtggtggacggcatggatgctgcggcttttattcaagccgtgcgcggtttgttagaacatcctgccaccctgttcctggagtaagcgatgagtcagattttaaaaacctcgctcctgatcgttggcggcgggccaggcggctatgtggcggcgatccgcgccggccagctggggattccaacggtgttggttgagggcgccgctttgggcggtacttgcctgaatgtggggtgcattccgagcaaagcgttgatccatgctgccgaagagtaccttaaagcgcgccactatgcatcacgttccgcgctgggcatccaggtgcaagcaccttcaattgacatcgcccgcaccgtggaatggaaagacgccattgtggaccgtttgacttcgggtgtggcggctctgctgaaaaagcatggtgtggatgtagtacaaggatgggcacgcatcctcgacggcaagagcgtggcggttgaactggcgggcggggggtcgcagcgcatcgagtgtgaacatctgcttctggcggcgggctcacaaagcgttgaattacccatcctgcctctggggggcaaagtaatcagcagcaccgaagcattagctccggggtcgttgccaaaacgtctggtggttgtgggtggcggttatattggtctggagctgggcactgcatatcgcaagctgggtgttgaagttgctgtggtggaggcacaaccccgcatcctgccgggctacgatgaggaactgactaagccggtggcccaagcgctgcgccgtctgggtgtagaactgtacctgggtcattcattgctgggaccgagtgaaaacggcgttcgcgtgcgtgatggggctggcgaagaacgtgagatcgccgcggaccaggtccttgtcgcagttggccgcaaaccgcgttcagagggttggaacctggagtctctcggtttagacatgaatgggcgtgccgtaaaagtggacgatcagtgccgtacaagcatgcgtaacgtatgggccattggcgacctggcgggcgaaccgatgctggcgcaccgcgctatggcgcaaggagaaatggtcgccgaattgattgcgggcaaacgccgtcagtttgcgccggttgcaattcctgcagtctgttttacggatccggaagtggtggtggcgggtctgagtccggaacaggccaaagatgcgggtctggattgcctggtcgcgtcattcccgttcgcagccaacggccgcgccatgacgttggaagctaacgaaggctttgtccgcgtggtggcacgtcgtgacaaccatctggtggttggttggcaggcggtcggtaaagctgtgtctgaattaagcaccgcgttcgcacaatctctggaaatgggcgctcgcctcgaagacattgcaggcacaatccacgcgcaccccaccctgggtgaagctgttcaggaagcggcactccgtgccttaggtcacgccctgcacatttga

SEQ ID NO: 5 - DNA, 5597 bp, Artificial Sequence
  Synthetic: Tet-livKHMGF construct
ccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgaaacggaatgcgaaaactatcatcgcagggatgattgcactggcaatttcacacaccgctatggctgacgatattaaagtcgccgttgtcggcgcgatgtccggcccgattgcccagtggggcgatatggaatttaacggcgcgcgtcaggcaattaaagacattaatgccaaagggggaattaagggcgataaactggttggcgtggaatatgacgacgcatgcgacccgaaacaagccgttgcggtcgccaacaaaatcgttaatgacggcattaaatacgttattggtcatctgtgttcttcttctacccagcctgcgtcagatatctatgaagacgaaggtattctgatgatctcgccgggagcgaccaacccggagctgacccaacgcggttatcaacacattatgcgtactgccgggctggactcttcccaggggccaacggcggcaaaatacattcttgagacggtgaagccccagcgcatcgccatcattcacgacaaacaacagtatggcgaagggctggcgcgttcggtgcaggacgggctgaaagcggctaacgccaacgtcgtcttcttcgacggtattaccgccggggagaaagatttctccgcgctgatcgcccgcctgaaaaaagaaaacatcgacttcgtttactacggcggttactacccggaaatggggcagatgctgcgccaggcccgttccgttggcctgaaaacccagtttatggggccggaaggtgtgggtaatgcgtcgttgtcgaacattgccggtgatgccgccgaaggcatgttggtcactatgccaaaacgctatgaccaggatccggcaaaccagggcatcgttgatgcgctgaaagcagacaagaaagatccgtccgggccttatgtctggatcacctacgcggcggtgcaatctctggcgactgcccttgagcgtaccggcagcgatgagccgctggcgctggtgaaagatttaaaagctaacggtgcaaacaccgtgattgggccgctgaactgggatgaaaaaggcgatcttaagggatttgattttggtgtcttccagtggcacgccgacggttcatccacggcagccaagtgatcatcccaccgcccgtaaaatgcgggcgggtttagaaaggttaccttatgtctgagcagtttttgtatttcttgcagcagatgtttaacggcgtcacgctgggcagtacctacgcgctgatagccatcggctacaccatggtttacggcattatcggcatgatcaacttcgcccacggcgaggtttatatgattggcagctacgtctcatttatgatcatcgccgcgctgatgatgatgggcattgataccggctggctgctggtagctgcgggattcgtcggcgcaatcgtcattgccagcgcctacggctggagtatcgaacgggtggcttaccgcccggtgcgtaactctaagcgcctgattgcactcatctctgcaatcggtatgtccatcttcctgcaaaactacgtcagcctgaccgaaggttcgcgcgacgtggcgctgccgagcctgtttaacggtcagtgggtggtggggcatagcgaaaacttctctgcctctattaccaccatgcaggcggtgatctggattgttaccttcctcgccatgctggcgctgacgattttcattcgctattcccgcatgggtcgcgcgtgtcgtgcctgcgcggaagatctgaaaatggcgagtctgcttggcattaacaccgaccgggtgattgcgctgacctttgtgattggcgcggcgatggcggcggtggcgggtgtgctgctcggtcagttctacggcgtcattaacccctacatcggctttatggccgggatgaaagcctttaccgcggcggtgctcggtgggattggcagcattccgggagcgatgattggcggcctgattctggggattgcggaggcgctctcttctgcctatctgagtacggaatataaagatgtggtgtcattcgccctgctgattctggtgctgctggtgatgccgaccggtattctgggtcgcccggaggtagagaaagtatgaaaccgatgcatattgcaatggcgctgctctctgccgcgatgttctttgtgctggcgggcgtctttatgggcgtgcaactggagctggatggcaccaaactggtggtcgacacggcttcggatgtccgttggcagtgggtgtttatcggcacggcggtggtctttttcttccagcttttgcgaccggctttccagaaagggttgaaaagcgtttccggaccgaagtttattctgcccgccattgatggctccacggtgaagcagaaactgttcctcgtggcgctgttggtgcttgcggtggcgtggccgtttatggtttcacgcgggacggtggatattgccaccctgaccatgatctacattatcctcggtctggggctgaacgtggttgttggtctttctggtctgctggtgctggggtacggcggtttttacgccatcggcgcttacacttttgcgctgctcaatcactattacggcttgggcttctggacctgcctgccgattgctggattaatggcagcggcggcgggcttcctgctcggttttccggtgctgcgtttgcgcggtgactatctggcgatcgttaccctcggtttcggcgaaattgtgcgcatattgctgctcaataacaccgaaattaccggcggcccgaacggaatcagtcagatcccgaaaccgacactcttcggactcgagttcagccgtaccgctcgtgaaggcggctgggacacgttcagtaatttctttggcctgaaatacgatccctccgatcgtgtcatcttcctctacctggtggcgttgctgctggtggtgctaagcctgtttgtcattaaccgcctgctgcggatgccgctggggcgtgcgtgggaagcgttgcgtgaagatgaaatcgcctgccgttcgctgggcttaagcccgcgtcgtatcaagctgactgcctttaccataagtgccgcgtttgccggttttgccggaacgctgtttgcggcgcgtcagggctttgtcagcccggaatccttcacctttgccgaatcggcgtttgtgctggcgatagtggtgctcggcggtatgggctcgcaatttgcggtgattctggcggcaattttgctggtggtgtcgcgcgagttgatgcgtgatttcaacgaatacagcatgttaatgctcggtggtttgatggtgctgatgatgatctggcgtccgcagggcttgctgcccatgacgcgcccgcaactgaagctgaaaaacggcgcagcgaaaggagagcaggcatgagtcagccattattatctgttaacggcctgatgatgcgcttcggcggcctgctggcggtgaacaacgtcaatcttgaactgtacccgcaggagatcgtctcgttaatcggccctaacggtgccggaaaaaccacggtttttaactgtctgaccggattctacaaacccaccggcggcaccattttactgcgcgatcagcacctggaaggtttaccggggcagcaaattgcccgcatgggcgtggtgcgcaccttccagcatgtgcgtctgttccgtgaaatgacggtaattgaaaacctgctggtggcgcagcatcagcaactgaaaaccgggctgttctctggcctgttgaaaacgccatccttccgtcgcgcccagagcgaagcgctcgaccgcgccgcgacctggcttgagcgcattggtttgctggaacacgccaaccgtcaggcgagtaacctggcctatggtgaccagcgccgtcttgagattgcccgctgcatggtgacgcagccggagattttaatgctcgacgaacctgcggcaggtcttaacccgaaagagacgaaagagctggatgagctgattgccgaactgcgcaatcatcacaacaccactatcttgttgattgaacacgatatgaagctggtgatgggaatttcggaccgaatttacgtggtcaatcaggggacgccgctggcaaacggtacgccggagcagatccgtaataacccggacgtgatccgtgcctatttaggtgaggcataagatggaaaaagtcatgttgtcctttgacaaagtcagcgcccactacggcaaaatccaggcgctgcatgaggtgagcctgcatatcaatcagggcgagattgtcacgctgattggcgcgaacggggcggggaaaaccaccttgctcggcacgttatgcggcgatccgcgtgccaccagcgggcgaattgtgtttgatgataaagacattaccgactggcagacagcgaaaatcatgcgcgaagcggtggcgattgtcccggaagggcgtcgcgtcttctcgcggatgacggtggaagagaacctggcgatgggcggtttttttgctgaacgcgaccagttccaggagcgcataaagtgggtgtatgagctgtttccacgtctgcatgagcgccgtattcagcgggcgggcaccatgtccggcggtgaacagcagatgctggcgattggtcgtgcgctgatgagcaacccgcgtttgctactgcttgatgagccatcgctcggtcttgcgccgattatcatccagcaaattttcgacaccatcgagcagctgcgcgagcaggggatgactatctttctcgtcgagcagaacgccaaccaggcgctaaagctggcggatcgcggctacgtgctggaaaacggccatgtagtgctttccgatactggtgatgcgctgctggcgaatgaagcggtgagaagtgcgtatttaggcgggtaa

SEQ ID NO: 6 - DNA, 4657 bp, Artificial Sequence
  Synthetic: pKIKO-lacZ
agattgcagcattacacgtcttgagcgattgtgtaggctggagctgcttcgaagttcctatactttctagagaataggaacttcggaataggaacttcatttaaatggcgcgccttacgccccgccctgccactcatcgcagtactgttgtattcattaagcatctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgtaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtaggcgcgccgaagttcctatactttctagagaataggaacttcggaataggaactaaggaggatattcatatggaccatggctaattccttgccgttttcatcatatttaatcagcgactgatccacccagtcccagacgaagccgccctgtaaacgggggtactgacgaaacgcctgccagtatttagcgaagccgccaagactgttacccatcgcgtgggcatattcgcaaaggatcagcgggcgcatttctccaggcagcgaaagccattttttgatggaccatttcggcaccgccgggaagggctggtcttcatccacgcgcgcgtacatcgggcaaataatatcggtggccgtggtgtcggctccgccgccttcatactgtaccgggcgggaaggatcgacagatttgatccagcgatacagcgcgtcgtgattagcgccgtggcctgattcattccccagcgaccagatgatcacactcgggtgattacgatcgcgctgcaccatccgcgttacgcgttcgctcatcgcgggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgccgcgaatcagcaacggcttgccgttcagcagcagcagaccattttcaatccgcacctcgcggaaaccgacgtcgcaggcttctgcttcaatcagcgtgccgtcggcggtgtgcagttcaaccactgcacgatagagattcgggatttcggcgctccacagttccggattttcaacgttcaggcgtagtgtgacgcgatcggcataaccgccacgctcatcgataatttcacccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgcggatcttgcggccgcaaaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgactgacgggctccaggagtcgtcgccaccaatccccatatggaaaccgtcgatattcagccatgtgccttcttccgcgtgcagcagatggcgatggctggtttccatcagttgttgttggctgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggctgcagtaaggcggtcgggatagttttcttgcggccccaggccgagccagtttacccgctctgagacctgcgccagctggcaggtcaggccaatccgcgccggatgcggtgtatcgcttgccaccgcaacatccacattgatgaccatctcaccgtgcccatcaatccggtaggttttccggctgataaataaggttttcccctgatgctgccacgcgtgggcggttgtaatcagcaccgcgtcggcaagtgtatctgccgtgcactgcaacaacgccgcttcggcctggtaatggcccgccgccttccagcgttcgacccaggcgttagggtcaatgcgggtcgcttcacttacgccaatgtcgttatccagcggcgcacgggtgaactgatcgcgcagcggggtcagcagttgtttttcatcgccaatccacatctgtgaaagaaagcctgactggcggttaaattgccaacgcttattacccagctcgatgcaaaaatccgttccgctggtggtcagttgagggatggcgtgggacgcggaggggagtgtcacgctgaggttttccgccagacgccattgctgccaggcgctgatgtgtccggcttctgaccatgcggtcgcgtttggttgcactacgcgtaccgttagccagagtcacatttccccgaaaagtgccacctgcatcgatggccccccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgc

SEQ ID NO: 7 - DNA, 10254 bp, Artificial Sequence
  Synthetic: pTet-livKHMGF sequence
agattgcagcattacacgtcttgagcgattgtgtaggctggagctgcttcgaagttcctatactttctagagaataggaacttcggaataggaacttcatttaaatggcgcgccttacgccccgccctgccactcatcgcagtactgttgtattcattaagcatctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgtaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtaggcgcgccgaagttcctatactttctagagaataggaacttcggaataggaactaaggaggatattcatatggaccatggctaattccttgccgttttcatcatatttaatcagcgactgatccacccagtcccagacgaagccgccctgtaaacgggggtactgacgaaacgcctgccagtatttagcgaagccgccaagactgttacccatcgcgtgggcatattcgcaaaggatcagcgggcgcatttctccaggcagcgaaagccattttttgatggaccatttcggcaccgccgggaagggctggtcttcatccacgcgcgcgtacatcgggcaaataatatcggtggccgtggtgtcggctccgccgccttcatactgtaccgggcgggaaggatcgacagatttgatccagcgatacagcgcgtcgtgattagcgccgtggcctgattcattccccagcgaccagatgatcacactcgggtgattacgatcgcgctgcaccatccgcgttacgcgttcgctcatcgcgggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgccgcgaatcagcaacggcttgccgttcagcagcagcagaccattttcaatccgcacctcgcggaaaccgacgtcgcaggcttctgcttcaatcagcgtgccgtcggcggtgtgcagttcaaccactgcacgatagagattcgggatttcggcgctccacagttccggattttcaacgttcaggcgtagtgtgacgcgatcggcataaccgccacgctcatcgataatttcacccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgcggatcttgcggccgcaaaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgactgacgggctccaggagtcgtcgccaccaatccccatatggaaaccgtcgatattcagccatgtgccttcttccgcgtgcagcagatggcgatggctggtttccatcagttgttgttggctgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggctgcagtaaggcggtcgggatagttttcttgcggccccaggccgagccagtttacccgctctgagacctgcgccagctggcaggtcaggccaatccgcgccggatgcggtgtatcgcttgccaccgcaacatccacattgatgaccatctcaccgtgcccatcaatccggtaggttttccggctgataaataaggttttcccctgatgctgccacgcgtgggcggttgtaatcagcaccgcgtcggcaagtgtatctgccgtgcactgcaacaacgccgcttcggcctggtaatggcccgccgccttccagcgttcgacccaggcgttagggtcaatgcgggtcgcttcacttacgccaatgtcgttatccagcggcgcacgggtgaactgatcgcgcagcggggtcagcagttgtttttcatcgccaatccacatctgtgaaagaaagcctgactggcggttaaattgccaacgcttattacccagctcgatgcaaaaatccgttccgctggtggtcagttgagggatggcgtgggacgcggaggggagtgtcacgctgaggttttccgccagacgccattgctgccaggcgctgatgtgtccggcttctgaccatgcggtcgcgtttggttgcactacgcgtaccgttagccagagtcacatttccccgaaaagtgccacctgcatcgatggccccccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgaaacggaatgcgaaaactatcatcgcagggatgattgcactggcaatttcacacaccgctatggctgacgatattaaagtcgccgttgtcggcgcgatgtccggcccgattgcccagtggggcgatatggaatttaacggcgcgcgtcaggcaattaaagacattaatgccaaagggggaattaagggcgataaactggttggcgtggaatatgacgacgcatgcgacccgaaacaagccgttgcggtcgccaacaaaatcgttaatgacggcattaaatacgttattggtcatctgtgttcttcttctacccagcctgcgtcagatatctatgaagacgaaggtattctgatgatctcgccgggagcgaccaacccggagctgacccaacgcggttatcaacacattatgcgtactgccgggctggactcttcccaggggccaacggcggcaaaatacattcttgagacggtgaagccccagcgcatcgccatcattcacgacaaacaacagtatggcgaagggctggcgcgttcggtgcaggacgggctgaaagcggctaacgccaacgtcgtcttcttcgacggtattaccgccggggagaaagatttctccgcgctgatcgcccgcctgaaaaaagaaaacatcgacttcgtttactacggcggttactacccggaaatggggcagatgctgcgccaggcccgttccgttggcctgaaaacccagtttatggggccggaaggtgtgggtaatgcgtcgttgtcgaacattgccggtgatgccgccgaaggcatgttggtcactatgccaaaacgctatgaccaggatccggcaaaccagggcatcgttgatgcgctgaaagcagacaagaaagatccgtccgggccttatgtctggatcacctacgcggcggtgcaatctctggcgactgcccttgagcgtaccggcagcgatgagccgctggcgctggtgaaagatttaaaagctaacggtgcaaacaccgtgattgggccgctgaactgggatgaaaaaggcgatcttaagggatttgattttggtgtcttccagtggcacgccgacggttcatccacggcagccaagtgatcatcccaccgcccgtaaaatgcgggcgggtttagaaaggttaccttatgtctgagcagtttttgtatttcttgcagcagatgtttaacggcgtcacgctgggcagtacctacgcgctgatagccatcggctacaccatggtttacggcattatcggcatgatcaacttcgcccacggcgaggtttatatgattggcagctacgtctcatttatgatcatcgccgcgctgatgatgatgggcattgataccggctggctgctggtagctgcgggattcgtcggcgcaatcgtcattgccagcgcctacggctggagtatcgaacgggtggcttaccgcccggtgcgtaactctaagcgcctgattgcactcatctctgcaatcggtatgtccatcttcctgcaaaactacgtcagcctgaccgaaggttcgcgcgacgtggcgctgccgagcctgtttaacggtcagtgggtggtggggcatagcgaaaacttctctgcctctattaccaccatgcaggcggtgatctggattgttaccttcctcgccatgctggcgctgacgattttcattcgctattcccgcatgggtcgcgcgtgtcgtgcctgcgcggaagatctgaaaatggcgagtctgcttggcattaacaccgaccgggtgattgcgctgacctttgtgattggcgcggcgatggcggcggtggcgggtgtgctgctcggtcagttctacggcgtcattaacccctacatcggctttatggccgggatgaaagcctttaccgcggcggtgctcggtgggattggcagcattccgggagcgatgattggcggcctgattctggggattgcggaggcgctctcttctgcctatctgagtacggaatataaagatgtggtgtcattcgccctgctgattctggtgctgctggtgatgccgaccggtattctgggtcgcccggaggtagagaaagtatgaaaccgatgcatattgcaatggcgctgctctctgccgcgatgttctttgtgctggcgggcgtctttatgggcgtgcaactggagctggatggcaccaaactggtggtcgacacggcttcggatgtccgttggcagtgggtgtttatcggcacggcggtggtctttttcttccagcttttgcgaccggctttccagaaagggttgaaaagcgtttccggaccgaagtttattctgcccgccattgatggctccacggtgaagcagaaactgttcctcgtggcgctgttggtgcttgcggtggcgtggccgtttatggtttcacgcgggacggtggatattgccaccctgaccatgatctacattatcctcggtctggggctgaacgtggttgttggtctttctggtctgctggtgctggggtacggcggtttttacgccatcggcgcttacacttttgcgctgctcaatcactattacggcttgggcttctggacctgcctgccgattgctggattaatggcagcggcggcgggcttcctgctcggttttccggtgctgcgtttgcgcggtgactatctggcgatcgttaccctcggtttcggcgaaattgtgcgcatattgctgctcaataacaccgaaattaccggcggcccgaacggaatcagtcagatcccgaaaccgacactcttcggactcgagttcagccgtaccgctcgtgaaggcggctgggacacgttcagtaatttctttggcctgaaatacgatccctccgatcgtgtcatcttcctctacctggtggcgttgctgctggtggtgctaagcctgtttgtcattaaccgcctgctgcggatgccgctggggcgtgcgtgggaagcgttgcgtgaagatgaaatcgcctgccgttcgctgggcttaagcccgcgtcgtatcaagctgactgcctttaccataagtgccgcgtttgccggttttgccggaacgctgtttgcggcgcgtcagggctttgtcagcccggaatccttcacctttgccgaatcggcgtttgtgctggcgatagtggtgctcggcggtatgggctcgcaatttgcggtgattctggcggcaattttgctggtggtgtcgcgcgagttgatgcgtgatttcaacgaatacagcatgttaatgctcggtggtttgatggtgctgatgatgatctggcgtccgcagggcttgctgcccatgacgcgcccgcaactgaagctgaaaaacggcgcagcgaaaggagagcaggcatgagtcagccattattatctgttaacggcctgatgatgcgcttcggcggcctgctggcggtgaacaacgtcaatcttgaactgtacccgcaggagatcgtctcgttaatcggccctaacggtgccggaaaaaccacggtttttaactgtctgaccggattctacaaacccaccggcggcaccattttactgcgcgatcagcacctggaaggtttaccggggcagcaaattgcccgcatgggcgtggtgcgcaccttccagcatgtgcgtctgttccgtgaaatgacggtaattgaaaacctgctggtggcgcagcatcagcaactgaaaaccgggctgttctctggcctgttgaaaacgccatccttccgtcgcgcccagagcgaagcgctcgaccgcgccgcgacctggcttgagcgcattggtttgctggaacacgccaaccgtcaggcgagtaacctggcctatggtgaccagcgccgtcttgagattgcccgctgcatggtgacgcagccggagattttaatgctcgacgaacctgcggcaggtcttaacccgaaagagacgaaagagctggatgagctgattgccgaactgcgcaatcatcacaacaccactatcttgttgattgaacacgatatgaagctggtgatgggaatttcggaccgaatttacgtggtcaatcaggggacgccgctggcaaacggtacgccggagcagatccgtaataacccggacgtgatccgtgcctatttaggtgaggcataagatggaaaaagtcatgttgtcctttgacaaagtcagcgcccactacggcaaaatccaggcgctgcatgaggtgagcctgcatatcaatcagggcgagattgtcacgctgattggcgcgaacggggcggggaaaaccaccttgctcggcacgttatgcggcgatccgcgtgccaccagcgggcgaattgtgtttgatgataaagacattaccgactggcagacagcgaaaatcatgcgcgaagcggtggcgattgtcccggaagggcgtcgcgtcttctcgcggatgacggtggaagagaacctggcgatgggcggtttttttgctgaacgcgaccagttccaggagcgcataaagtgggtgtatgagctgtttccacgtctgcatgagcgccgtattcagcgggcgggcaccatgtccggcggtgaacagcagatgctggcgattggtcgtgcgctgatgagcaacccgcgtttgctactgcttgatgagccatcgctcggtcttgcgccgattatcatccagcaaattttcgacaccatcgagcagctgcgcgagcaggggatgactatctttctcgtcgagcagaacgccaaccaggcgctaaagctggcggatcgcggctacgtgctggaaaacggccatgtagtgctttccgatactggtgatgcgctgctggcgaatgaagcggtgagaagtgcgtatttaggcgggtaaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgc

SEQ ID NO: 8 - DNA, 639 bp, Escherichia coli
  E. coli Nissle 1917 leucine exporter gene leuE
gtgttcgctgaatacggggttctgaattactggacctatctggttggggccatttttattgtgttggtgccagggccaaataccctgtttgtactcaaaaatagcgtcagtagcggtatgaaaggcggttatcttgcggcctgtggtgtatttattggcgatgcggtattgatgtttctggcatgggctggagtggcgacattaattaagaccaccccgatattattcaacatcgtacgttatcttggtgcgttttatttgctctatctggggagtaaaattctctacgcgaccctgaaaggtaaaaatagcgagaccaaatccgatgagccccaatacggtgccatttttaaacgcgcgttaattttgagcctgactaatccgaaagccattttgttctatgtgtcgtttttcgtacagtttatcgatgttaatgccccacatacgggaatttcattctttattctggcgacgacgctggaactggtgagtttctgctatttgagcttcctgattatttctggggcttttgtcacgcagtacatacgtaccaaaaagaaactggctaaagtgggcaactcactgattggtttgatgttcgtgggtttcgccgcccgactggcgacgctgcaatcctga

SEQ ID NO: 9 - DNA, 1707 bp, Artificial Sequence
  Synthetic: leuE deletion construct
cattttaaataccatttattggttactttttagcaccatatcagcgaagaatcagggaggattatagatgggaagcccatgcagattgcagcattacacgtcttgagcgattgtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatggaccatggctaattcccaattaacctctttaattatctttcgatcatgcgcgattaaaggtgaatatgctaaccaatctgtagcggcttagaaaggagaaaatcaggttttaacctga

SEQ ID NO: 10 - DNA, 8864 bp, Artificial Sequence
  Synthetic: Tet-livKHMGF fragment
aataggggttccgcgactgacgggctccaggagtcgtcgccaccaatccccatatggaaaccgtcgatattcagccatgtgccttcttccgcgtgcagcagatggcgatggctggtttccatcagttgttgttggctgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggctgcagtaaggcggtcgggatagttttcttgcggccccaggccgagccagtttacccgctctgagacctgcgccagctggcaggtcaggccaatccgcgccggatgcggtgtatcgcttgccaccgcaacatccacattgatgaccatctcaccgtgcccatcaatccggtaggttttccggctgataaataaggttttcccctgatgctgccacgcgtgggcggttgtaatcagcaccgcgtcggcaagtgtatctgccgtgcactgcaacaacgccgcttcggcctggtaatggcccgccgccttccagcgttcgacccaggcgttagggtcaatgcgggtcgcttcacttacgccaatgtcgttatccagcggcgcacgggtgaactgatcgcgcagcggggtcagcagttgtttttcatcgccaatccacatctgtgaaagaaagcctgactggcggttaaattgccaacgcttattacccagctcgatgcaaaaatccgttccgctggtggtcagttgagggatggcgtgggacgcggaggggagtgtcacgctgaggttttccgccagacgccattgctgccaggcgctgatgtgtccggcttctgaccatgcggtcgcgtttggttgcactacgcgtaccgttagccagagtcacatttccccgaaaagtgccacctgcatcgatggccccccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgaaacggaatgcgaaaactatcatcgcagggatgattgcactggcaatttcacacaccgctatggctgacgatattaaagtcgccgttgtcggcgcgatgtccggcccgattgcccagtggggcgatatggaatttaacggcgcgcgtcaggcaattaaagacattaatgccaaagggggaattaagggcgataaactggttggcgtggaatatgacgacgcatgcgacccgaaacaagccgttgcggtcgccaacaaaatcgttaatgacggcattaaatacgttattggtcatctgtgttcttcttctacccagcctgcgtcagatatctatgaagacgaaggtattctgatgatctcgccgggagcgaccaacccggagctgacccaacgcggttatcaacacattatgcgtactgccgggctggactcttcccaggggccaacggcggcaaaatacattcttgagacggtgaagccccagcgcatcgccatcattcacgacaaacaacagtatggcgaagggctggcgcgttcggtgcaggacgggctgaaagcggctaacgccaacgtcgtcttcttcgacggtattaccgccggggagaaagatttctccgcgctgatcgcccgcctgaaaaaagaaaacatcgacttcgtttactacggcggttactacccggaaatggggcagatgctgcgccaggcccgttccgttggcctgaaaacccagtttatggggccggaaggtgtgggtaatgcgtcgttgtcgaacattgccggtgatgccgccgaaggcatgttggtcactatgccaaaacgctatgaccaggatccggcaaaccagggcatcgttgatgcgctgaaagcagacaagaaagatccgtccgggccttatgtctggatcacctacgcggcggtgcaatctctggcgactgcccttgagcgtaccggcagcgatgagccgctggcgctggtgaaagatttaaaagctaacggtgcaaacaccgtgattgggccgctgaactgggatgaaaaaggcgatcttaagggatttgattttggtgtcttccagtggcacgccgacggttcatccacggcagccaagtgatcatcccaccgcccgtaaaatgcgggcgggtttagaaaggttaccttatgtctgagcagtttttgtatttcttgcagcagatgtttaacggcgtcacgctgggcagtacctacgcgctgatagccatcggctacaccatggtttacggcattatcggcatgatcaacttcgcccacggcgaggtttatatgattggcagctacgtctcatttatgatcatcgccgcgctgatgatgatgggcattgataccggctggctgctggtagctgcgggattcgtcggcgcaatcgtcattgccagcgcctacggctggagtatcgaacgggtggcttaccgcccggtgcgtaactctaagcgcctgattgcactcatctctgcaatcggtatgtccatcttcctgcaaaactacgtcagcctgaccgaaggttcgcgcgacgtggcgctgccgagcctgtttaacggtcagtgggtggtggggcatagcgaaaacttctctgcctctattaccaccatgcaggcggtgatctggattgttaccttcctcgccatgctggcgctgacgattttcattcgctattcccgcatgggtcgcgcgtgtcgtgcctgcgcggaagatctgaaaatggcgagtctgcttggcattaacaccgaccgggtgattgcgctgacctttgtgattggcgcggcgatggcggcggtggcgggtgtgctgctcggtcagttctacggcgtcattaacccctacatcggctttatggccgggatgaaagcctttaccgcggcggtgctcggtgggattggcagcattccgggagcgatgattggcggcctgattctggggattgcggaggcgctctcttctgcctatctgagtacggaatataaagatgtggtgtcattcgccctgctgattctggtgctgctggtgatgccgaccggtattctgggtcgcccggaggtagagaaagtatgaaaccgatgcatattgcaatggcgctgctctctgccgcgatgttctttgtgctggcgggcgtctttatgggcgtgcaactggagctggatggcaccaaactggtggtcgacacggcttcggatgtccgttggcagtgggtgtttatcggcacggcggtggtctttttcttccagcttttgcgaccggctttccagaaagggttgaaaagcgtttccggaccgaagtttattctgcccgccattgatggctccacggtgaagcagaaactgttcctcgtggcgctgttggtgcttgcggtggcgtggccgtttatggtttcacgcgggacggtggatattgccaccctgaccatgatctacattatcctcggtctggggctgaacgtggttgttggtctttctggtctgctggtgctggggtacggcggtttttacgccatcggcgcttacacttttgcgctgctcaatcactattacggcttgggcttctggacctgcctgccgattgctggattaatggcagcggcggcgggcttcctgctcggttttccggtgctgcgtttgcgcggtgactatctggcgatcgttaccctcggtttcggcgaaattgtgcgcatattgctgctcaataacaccgaaattaccggcggcccgaacggaatcagtcagatcccgaaaccgacactcttcggactcgagttcagccgtaccgctcgtgaaggcggctgggacacgttcagtaatttctttggcctgaaatacgatccctccgatcgtgtcatcttcctctacctggtggcgttgctgctggtggtgctaagcctgtttgtcattaaccgcctgctgcggatgccgctggggcgtgcgtgggaagcgttgcgtgaagatgaaatcgcctgccgttcgctgggcttaagcccgcgtcgtatcaagctgactgcctttaccataagtgccgcgtttgccggttttgccggaacgctgtttgcggcgcgtcagggctttgtcagcccggaatccttcacctttgccgaatcggcgtttgtgctggcgatagtggtgctcggcggtatgggctcgcaatttgcggtgattctggcggcaattttgctggtggtgtcgcgcgagttgatgcgtgatttcaacgaatacagcatgttaatgctcggtggtttgatggtgctgatgatgatctggcgtccgcagggcttgctgcccatgacgcgcccgcaactgaagctgaaaaacggcgcagcgaaaggagagcaggcatgagtcagccattattatctgttaacggcctgatgatgcgcttcggcggcctgctggcggtgaacaacgtcaatcttgaactgtacccgcaggagatcgtctcgttaatcggccctaacggtgccggaaaaaccacggtttttaactgtctgaccggattctacaaacccaccggcggcaccattttactgcgcgatcagcacctggaaggtttaccggggcagcaaattgcccgcatgggcgtggtgcgcaccttccagcatgtgcgtctgttccgtgaaatgacggtaattgaaaacctgctggtggcgcagcatcagcaactgaaaaccgggctgttctctggcctgttgaaaacgccatccttccgtcgcgcccagagcgaagcgctcgaccgcgccgcgacctggcttgagcgcattggtttgctggaacacgccaaccgtcaggcgagtaacctggcctatggtgaccagcgccgtcttgagattgcccgctgcatggtgacgcagccggagattttaatgctcgacgaacctgcggcaggtcttaacccgaaagagacgaaagagctggatgagctgattgccgaactgcgcaatcatcacaacaccactatcttgttgattgaacacgatatgaagctggtgatgggaatttcggaccgaatttacgtggtcaatcaggggacgccgctggcaaacggtacgccggagcagatccgtaataacccggacgtgatccgtgcctatttaggtgaggcataagatggaaaaagtcatgttgtcctttgacaaagtcagcgcccactacggcaaaatccaggcgctgcatgaggtgagcctgcatatcaatcagggcgagattgtcacgctgattggcgcgaacggggcggggaaaaccaccttgctcggcacgttatgcggcgatccgcgtgccaccagcgggcgaattgtgtttgatgataaagacattaccgactggcagacagcgaaaatcatgcgcgaagcggtggcgattgtcccggaagggcgtcgcgtcttctcgcggatgacggtggaagagaacctggcgatgggcggtttttttgctgaacgcgaccagttccaggagcgcataaagtgggtgtatgagctgtttccacgtctgcatgagcgccgtattcagcgggcgggcaccatgtccggcggtgaacagcagatgctggcgattggtcgtgcgctgatgagcaacccgcgtttgctactgcttgatgagccatcgctcggtcttgcgccgattatcatccagcaaattttcgacaccatcgagcagctgcgcgagcaggggatgactatctttctcgtcgagcagaacgccaaccaggcgctaaagctggcggatcgcggctacgtgctggaaaacggccatgtagtgctttccgatactggtgatgcgctgctggcgaatgaagcggtgagaagtgcgtatttaggcgggtaaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcagattgcagcattacacgtcttgagcgattgtgtaggctggagctgcttcgaagttcctatactttctagagaataggaacttcggaataggaacttcatttaaatggcgcgccttacgccccgccctgccactcatcgcagtactgttgtattcattaagcatctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgtaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtaggcgcgccgaagttcctatactttctagagaataggaacttcggaataggaactaaggaggatattcatatggaccatggctaattccttgccgttttcatcatatttaatcagcgactgatccacccagtcccagacgaagccgccctgtaaacgggggtactgacgaaacgcctgccagtatttagcgaagccgccaagactgttacccatcgcgtgggcatattcgcaaaggatcagcgggcgcatttctccaggcagcgaaagccattttttgatggaccatttcggcaccgccgggaagggctggtcttcatccacgcgcgcgtacatcgggcaaataatatcggtggccgtggtgtcggctccgccgccttcatactgtaccgggcgggaaggatcgacagatttgatccagcgatacagcgcgtcgtgattagcgccgtggcctgattcattccccagcgaccagatgatcacactcgggtgattacgatcgcgctgcaccatccgcgttacgcgttcgctcatcgcgggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgccgcgaatcagcaacggcttgccgttcagcagcagcagaccattttcaatccgcacctcgcggaaaccgacgtcgcaggcttctgcttcaatcagcgtgccgtcggcggtgtgcagttcaaccactgcacgatagagattcgggatttcggcgctccacagttccggattttcaacgttcaggcgtagtgtgacgcgatcggcataaccgccacgctcatcgataatttcacccatgtcagccgttaag

SEQ ID NO: 11 - DNA, 2344 bp, Artificial Sequence
  Synthetic: Ptac-livJ construct
agacaacaagtccacgttgcaggaactggctgaccgttacggtgtttccgctgagcgtgtgcgtcagctggaaaagaacgcgatgaaaaaattgcgcgctgccattgaagcgtaatttccgctattaagcagagaaccctggatgagagtccggggtttttgttttttgggcctctacaataatcaattccccctccggcaaaacgccaatccccacgcagattgttaataaactgtcaaaatagctataacacatttccccgaaaagtgccgatggccccccgatggtagtgtggcccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcagattgcagcattacacgtcttgagcgattgtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatggaccatggctaattcccatgttgacaattaatcatcggctcgtataatgttagcagagtatgctgctaaagcacgggtagctacgtataaaacgaaataaagtgctgcacaacaacatcacaacacacgtaataaccagaagagtggggattctcaggatgaacataaagggtaaagcgttactggcaggatgtatcgcgctggcattcagcaatatggctctggcagaagatattaaagtcgccgtcgtaggcgcaatgtccggtccggtggcgcag

SEQ ID NO: 12 - DNA, 1104 bp, Artificial Sequence
  Synthetic: livJ sequence
atgaacataaagggtaaagcgttactggcaggatgtatcgcgctggcattcagcaatatggctctggcagaagatattaaagtcgcggtcgtgggcgcaatgtccggtccggttgcgcagtacggtgaccaggagtttaccggcgcagagcaggcggttgcggatatcaacgctaaaggcggcattaaaggcaacaaactgcaaatcgtaaaatatgacgatgcctgtgacccgaaacaggcggttgcggtggcgaacaaagtcgttaacgacggcattaaatatgtgattggtcacctctgttcttcatcaacgcagcctgcgtctgacatctacgaagacgaaggcattttaatgatcaccccagcggcaaccgcgccggagctgaccgcccgtggctatcagctgatcctgcgcaccaccggcctggactccgaccaggggccgacggcggcgaaatatattcttgagaaagtgaaaccgcagcgtattgctatcgttcacgacaaacagcaatacggcgaaggtctggcgcgagcggtgcaggacggcctgaagaaaggcaatgcaaacgtggtgttctttgatggcatcaccgccggggaaaaagatttctcaacgctggtggcgcgtctgaaaaaagagaatatcgacttcgtttactacggcggttatcacccggaaatggggcaaatcctgcgtcaggcacgcgcggcagggctgaaaactcagtttatggggccggaaggtgtggctaacgtttcgctgtctaacattgcgggcgaatcagcggaagggctgctggtgaccaagccgaagaactacgatcaggttccggcgaacaaacccattgttgacgcgatcaaagcgaaaaaacaggacccaagtggcgcattcgtttggaccacctacgccgcgctgcaatctttgcaggcgggcctgaatcagtctgacgatccggctgaaatcgccaaatacctgaaagcgaactccgtggataccgtaatgggaccgctgacctgggatgagaaaggcgatctgaaaggctttgagttcggcgtatttgactggcacgccaacggcacggcgaccgatgcgaagtaa

SEQ ID NO: 13 - DNA, 1921 bp, Artificial Sequence
  Synthetic: Prp promoter
ttacccgtctggattttcagtacgcgcttttaaacgacgccacagcgtggtacggctgatccccaaataacgtgcggcggcgcgcttatcgccattaaagcgtgcgagcacctcctgcaatggaagcgcttctgctgacgagggcgtgatttctgctgtggtccccaccagttcaggtaataattgccgcataaattgtctgtccagtgttggtgcgggatcgacgcttaaaaaaagcgccaggcgttccatcatattccgcagttcgcgaatattaccgggccaatgatagttcagtagaagcggctgacactgcgtcagcccatgacgcaccgattcggtaaaagggatctccatcgcggccagcgattgttttaaaaagttttccgccagaggcagaatatcaggctgtcgctcgcgcaaggggggaagcggcagacgcagaatgctcaaacggtaaaacagatcggtacgaaaacgtccttgcgttatctcccgatccagatcgcaatgcgtggcgctgatcacccggacatctaccgggatcggctgatgcccgccaacgcgggtgacggctttttcctccagtacgcgtagaaggcgggtttgtaacggcagcggcatttcgccaatttcgtcaagaaacagcgtgccgccgtgggcgacctcaaacagccccgcacgtccacctcgtcttgagccggtaaacgctccctcctcatagccaaacagttcagcctccagcaacgactcggtaatcgcgccgcaattaacggcgacaaagggcggagaaggcttgttctgacggtggggctgacggttaaacaacgcctgatgaatcgcttgcgccgccagctctttcccggtccctgtttccccctgaatcagcactgccgcgcgggaacgggcatagagtgtaatcgtatggcgaacctgctccatttgtggtgaatcgccgaggatatcgctcagcgcataacgggtctgtaatcccttgctggaggtatgctggctatactgacgccgtgtcaggcgggtcatatccagcgcatcatggaaagcctgacgtacggtggccgctgaataaataaagatggcggtcattcctgcctcttccgccaggtcggtaattagtcctgccccaattacagcctcaatgccgttagctttgagctcgttaatttgcccgcgagcatcctcttcagtgatatagcttcgctgttcaagacggaggtgaaacgttttctgaaaggcgaccagagccggaatggtctcctgataggtcacgattcccattgaggaagtcagctttcccgcttttgccagagcctgtaatacatcgaatccgctgggtttgatgaggatgacaggtaccgacagtcggctttttaaataagcgccgttggaacctgccgcgataatcgcgtcgcagcgttcggttgccagttttttgcgaatgtaggctactgccttttcaaaaccgagctgaataggcgtgatcgtcgccagatgatcaaactccaggctgatatcccgaaatagttcgaacaggcgcgttaccgagaccgtccagatcaccggtttatcgctattatcgcgcgaagcgctatgcacagtaaccatcgtcgtagattcatgtttaaggaacgaattcttgttttatagatgtttcgttaatgttgcaatgaaacacaggcctccgtttcatgaaacgttagctgactcgtttttcttgtgactcgtctgtcagtattaaaaaagatttttcatttaactgattgtttttaaattgaattttatttaatggtttctcggtttttgggtctggcatatcccttgctttaatgagtgcatcttaattaacaattcaataacaagagggctgaatagtaatttcaacaaaataacgagcattcgaatg

SEQ ID NO: 14 - DNA, 290 bp, Artificial Sequence
  Synthetic: FNR Responsive Promoter
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaa

SEQ ID NO: 15 - DNA, 173 bp, Artificial Sequence
  Synthetic: FNR Responsive Promoter
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacct

SEQ ID NO: 16 - DNA, 305 bp, Artificial Sequence
  Synthetic: FNR Responsive Promoter
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacat

SEQ ID NO: 17 - DNA, 180 bp, Artificial Sequence
  Synthetic: FNR Responsive Promoter
catttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacat

SEQ ID NO: 18 - DNA, 199 bp, Artificial Sequence
  Synthetic: FNR Responsive Promoter
agttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacat

SEQ ID NO: 19 - protein, 341 aa, Pseudomonas aeruginosa
  LeuDH Amino acid sequence; Leucine dehydrogenase LeuDH
MFDMMDAARLEGLHLAQDPATGLKAIIAIHSTRLGPALGGCRYLPYPNDEAAIGDAIRLAQGMSYKAALAGLEQGGGKAVIIRPPHLDNRGALFEAFGRFIESLGGRYITAVDSGTSSADMDCIAQQTRHVTSTTQAGDPSPHTALGVFAGIRASAQARLGSDDLEGLRVAVQGLGHVGYALAEQLAAVGAELLVCDLDPGRVQLAVEQLGAHPLAPEALLSTPCDILAPCGLGGVLTSQSVSQLRCAAVAGAANNQLERPEVADELEARGILYAPDYVINSGGLIYVALKHRGADPHSITAHLARIPARLTEIYAHAQADHQSPARIADRLAERILYGPQ

SEQ ID NO: 20 - DNA, 1026 bp, Artificial Sequence
  Synthetic: leuDH codon-optimized nucleotide sequence
atgttcgacatgatggatgcagcccgcctggaaggcctgcacctcgcccaggatccagcgacgggcctgaaagcgatcatcgcgatccattccactcgcctcggcccggccttaggcggctgtcgttacctcccatatccgaatgatgaagcggccatcggcgatgccattcgcctggcgcagggcatgtcctacaaagctgcacttgcgggtctggaacaaggtggtggcaaggcggtgatcattcgcccaccccacttggataatcgcggtgccttgtttgaagcgtttggacgctttattgaaagcctgggtggccgttatatcaccgccgttgactcaggaacaagtagtgccgatatggattgcatcgcccaacagacgcgccatgtgacttcaacgacacaagccggcgacccatctccacatacggctctgggcgtctttgccggcatccgcgcctccgcgcaggctcgcctggggtccgatgacctggaaggcctgcgtgtcgcggttcagggccttggccacgtaggttatgcgttagcggagcagctggcggcggtcggcgcagaactgctggtgtgcgacctggaccccggccgcgtccagttagcggtggagcaactgggggcgcacccactggcccctgaagcattgctctctactccgtgcgacattttagcgccttgtggcctgggcggcgtgctcaccagccagtcggtgtcacagttgcgctgcgcggccgttgcaggcgcagcgaacaatcaactggagcgcccggaagttgcagacgaactggaggcgcgcgggattttatatgcgcccgattacgtgattaactcgggaggactgatttatgtggcgctgaagcatcgcggtgctgatccgcatagcattaccgcccacctcgctcgcatccctgcacgcctgacggaaatctatgcgcatgcgcaggcggatcatcagtcgcctgcgcgcatcgccgatcgtctggcggagcgcattctgtacggcccgcagtga

SEQ ID NO: 21 - protein, 360 aa, Escherichia coli
  IlvE Amino acid sequence; Branched-chain amino acid aminotransferase IlvE
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 22 - DNA, 930 bp, Artificial Sequence
  Synthetic: ilvE nucleotide sequence
atgaccacgaagaaagctgattacatttggttcaatggggagatggttcgctgggaagacgcgaaggtgcatgtgatgtcgcacgcgctgcactatggcacctcggtttttgaaggcatccgttgctacgactcgcacaaaggaccggttgtattccgccatcgtgagcatatgcagcgtctgcatgactccgccaaaatctatcgcttcccggtttcgcagagcattgatgagctgatggaagcttgtcgtgacgtgatccgcaaaaacaatctcaccagcgcctatatccgtccgctgatcttcgttggtgatgttggcatgggcgtaaacccgccagcgggatactcaaccgacgtgattatcgccgctttcccgtggggagcgtatctgggcgcagaagcgctggagcaggggatcgatgcgatggtttcctcctggaaccgcgcagcaccaaacaccatcccgacggcggcaaaagccggtggtaactacctctcttccctgctggtgggtagcgaagcgcgccgccacggttatcaggaaggtatcgcgttggatgtgaatggttacatctctgaaggcgcaggcgaaaacctgtttgaagtgaaagacggcgtgctgttcaccccaccgttcacctcatccgcgctgccgggtattacccgtgatgccatcatcaaactggcaaaagagctgggaattgaagtgcgtgagcaggtgctgtcgcgcgaatccctgtacctggcggatgaagtgtttatgtccggtacggcggcagaaatcacgccagtgcgcagcgtagacggtattcaggttggcgaaggccgttgtggcccggttaccaaacgcattcagcaagccttcttcggcctcttcactggcgaaaccgaagataaatggggctggttagatcaagttaatcaataa

SEQ ID NO: 23 - protein, 471 aa, Proteus vulgaris
  L-AAD Amino acid sequence
MAISRRKFIIGGTVVAVAAGAGILTPMLTREGRFVPGTPRHGFVEGTEGALPKQADVVVVGAGILGIMTAINLVERGLSVVIVEKGNIAGEQSSRFYGQAISYKMPDETFLLHHLGKHRWREMNAKVGIDTTYRTQGRVEVPLDEEDLVNVRKWIDERSKNVGSDIPFKTRIIEGAELNQRLRGATTDWKIAGFEEDSGSFDPEVATFVMAEYAKKMGVRIYTQCAARGLETQAGVISDVVTEKGAIKTSQVVVAGGVWSRLFMQNLNVDVPTLPAYQSQQLISGSPTAPGGNVALPGGIFFREQADGTYATSPRVIVAPVVKESFTYGYKYLPLLALPDFPVHISLNEQLINSFMQSTHWNLDEVSPFEQFRNMTALPDLPELNASLEKLKAEFPAFKESKLIDQWSGAMAIAPDENPIISEVKEYPGLVINTATGWGMTESPVSAELTADLLLGKKPVLDPKPFSLYRF

SEQ ID NO: 24 - DNA, 1416 bp, Artificial Sequence
  Synthetic: L-AAD Codon-optimized nucleotide sequence
atggccatcagtcgtcgcaaattcattatcggtggaacggtcgtcgccgttgccgccggtgcggggattttgaccccgatgctgacgcgcgaagggcgctttgtgccgggcactccacgccacggtttcgttgaagggaccgagggggctttacccaaacaagcggacgtggtggtcgtaggcgctggaattcttggtattatgacggccattaatttggttgagcgtgggctgtcagtggtaattgtggagaagggcaatatcgcgggagaacaaagctctcgcttctacggacaggcaattagctataagatgccagatgagacatttttgctgcaccatcttgggaagcaccgctggcgtgagatgaatgcgaaagtagggattgatacaacgtaccgtactcaaggacgcgtggaagtaccgcttgacgaggaagatttggtaaacgtccgcaaatggattgacgaacgttcaaaaaatgttggatctgacattccttttaagacccgcattatcgagggggcagaattaaatcagcgtctgcgcggcgccacaacagattggaagatcgctggcttcgaggaggacagcgggtcattcgatcccgaggtagcgacctttgtaatggcagagtacgcgaagaagatgggtgttcgtatctatacgcaatgcgcggcccgcggtctggaaacccaggccggtgtcatttcagatgttgtcacggaaaaaggtgcgattaagacctcccaagtggtagtggctggtggggtgtggagtcgtctgttcatgcagaatttaaacgtcgacgtcccaacccttcctgcgtatcagtcacagcagttgattagtggttcccctaccgcaccgggggggaacgtcgcattacctggtggaatcttcttccgcgaacaggcggacgggacatacgcgacttctcctcgtgtgattgttgccccagttgtgaaggagagcttcacttatggttacaagtacttaccattattagcattgcctgatttccctgttcacattagcctgaatgaacagttaattaattcgtttatgcaaagtacccactggaacttagacgaagtgtcgccgttcgaacaatttcgcaacatgacagcattacctgacttgcccgaacttaacgccagcttagaaaagttaaaggcagagttccctgctttcaaagaatccaagttgatcgaccagtggtctggagcaatggcaattgcgcccgacgaaaatccaatcatttccgaggtgaaggagtacccaggtctggtaattaacacggcgacaggttggggcatgactgaaagtccagtgtctgctgaacttaccgccgatcttctgctggggaagaagccggtgttagatcctaagccattctcactttatcgcttttga

SEQ ID NO: 25 - protein, 471 aa, Proteus mirabilis
  L-AAD Amino acid sequence
MAISRRKFILGGTVVAVAAGAGVLTPMLTREGRFVPGTPRHGFVEGTGGPLPKQDDVVVIGAGILGIMTAINLAERGLSVTIVEKGNIAGEQSSRFYGQAISYKMPDETFLLHHLGKHRWREMNAKVGIDTTYRTQGRVEVPLDEEDLENVRKWIDAKSKDVGSDIPFRTKMIEGAELKQRLRGATTDWKIAGFEEDSGSFDPEVATFVMAEYAKKMGIKIFTNCAARGLETQAGVISDVVTEKGPIKTSRVVVAGGVGSRLFMQNLNVDVPTLPAYQSQQLISAAPNAPGGNVALPGGIFFRDQADGTYATSPRVIVAPVVKESFTYGYKYLPLLALPDFPVHISLNEQLINSFMQSTHWDLNEESPFEKYRDMTALPDLPELNASLEKLKKEFPAFKESTLIDQWSGAMAIAPDENPIISDVKEYPGLVINTATGWGMTESPVSAEITADLLLGKKPVLDAKPFSLYRF

SEQ ID NO: 26 - DNA, 1416 bp, Artificial Sequence
  Synthetic: L-AAD Nucleotide sequence
atggcaataagtagaagaaaatttattcttggtggcacagtggttgctgttgctgcaggcgctggggttttaacacctatgttaacgcgagaagggcgttttgttcctggtacgccgagacatggttttgttgagggaactggcggtccattaccgaaacaagatgatgttgttgtaattggtgcgggtattttaggtatcatgaccgcgattaaccttgctgagcgtggcttatctgtcacaatcgttgaaaaaggaaatattgccggcgaacaatcatctcgattctatggtcaagctattagctataaaatgccagatgaaaccttcttattacatcacctcgggaagcaccgctggcgtgagatgaacgctaaagttggtattgataccacttatcgtacacaaggtcgtgtagaagttcctttagatgaagaagatttagaaaacgtaagaaaatggattgatgctaaaagcaaagatgttggctcagacattccatttagaacaaaaatgattgaaggcgctgagttaaaacagcgtttacgtggcgctaccactgattggaaaattgctggtttcgaagaagactcaggaagcttcgatcctgaagttgcgacttttgtgatggcagaatatgccaaaaaaatgggtatcaaaattttcacaaactgtgcagcccgtggtttagaaacgcaagctggtgttatttctgatgttgtaacagaaaaaggaccaattaaaacctctcgtgttgttgtcgccggtggtgttgggtcacgtttatttatgcagaacctaaatgttgatgtaccaacattacctgcttatcaatcacagcaattaattagcgcagcaccaaatgcgccaggtggaaacgttgctttacccggcggaattttctttcgtgatcaagcggatggaacgtatgcaacttctcctcgtgtcattgttgctccggtagtaaaagaatcatttacttacggctataaatatttacctctgctggctttacctgatttcccagtacatatttcgttaaatgagcagttgattaattcctttatgcaatcaacacattgggatcttaatgaagagtcgccatttgaaaaatatcgtgatatgaccgctctgcctgatctgccagaattaaatgcctcactggaaaaactgaaaaaagagttcccagcatttaaagaatcaacgttaattgatcagtggagtggtgcgatggcgattgcaccagatgaaaacccaattatctctgatgttaaagagtatccaggtctagttattaatactgcaacaggttggggaatgactgaaagccctgtatcagcagaaattacagcagatttattattaggcaaaaaaccagtattagatgccaaaccatttagtctgtatcgtttctaa

SEQ ID NO: 27 - protein, 548 aa, Lactococcus lactis
  KivD Amino acid sequence
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 28 - DNA, 1647 bp, Artificial Sequence; identical to SEQ ID NO: 1
  Synthetic: kivD Nucleotide sequence

SEQ ID NO: 29 - DNA, 1647 bp, Artificial Sequence
  Synthetic: kivD Codon-optimized sequence
atgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataa

SEQ ID NO: 30 - protein, 548 aa, Lactococcus lactis
  KdcA Amino acid sequence
MYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNKS

SEQ ID NO: 31 - DNA, 1647 bp, Artificial Sequence
  Synthetic: kdcA Nucleotide sequence
atgtatacagtaggagattacctattagaccgattacacgagttgggaattgaagaaatttttggagttcctggtgactataacttacaatttttagatcaaattatttcacgcgaagatatgaaatggattggaaatgctaatgaattaaatgcttcttatatggctgatggttatgctcgtactaaaaaagctgccgcatttctcaccacatttggagtcggcgaattgagtgcgatcaatggactggcaggaagttatgccgaaaatttaccagtagtagaaattgttggttcaccaacttcaaaagtacaaaatgacggaaaatttgtccatcatacactagcagatggtgattttaaacactttatgaagatgcatgaacctgttacagcagcgcggactttactgacagcagaaaatgccacatatgaaattgaccgagtactttctcaattactaaaagaaagaaaaccagtctatattaacttaccagtcgatgttgctgcagcaaaagcagagaagcctgcattatctttagaaaaagaaagctctacaacaaatacaactgaacaagtgattttgagtaagattgaagaaagtttgaaaaatgcccaaaaaccagtagtgattgcaggacacgaagtaattagttttggtttagaaaaaacggtaactcagtttgtttcagaaacaaaactaccgattacgacactaaattttggtaaaagtgctgttgatgaatctttgccctcatttttaggaatatataacgggaaactttcagaaatcagtcttaaaaattttgtggagtccgcagactttatcctaatgcttggagtgaagcttacggactcctcaacaggtgcattcacacatcatttagatgaaaataaaatgatttcactaaacatagatgaaggaataattttcaataaagtggtagaagattttgattttagagcagtggtttcttctttatcagaattaaaaggaatagaatatgaaggacaatatattgataagcaatatgaagaatttattccatcaagtgctcccttatcacaagaccgtctatggcaggcagttgaaagtttgactcaaagcaatgaaacaatcgttgctgaacaaggaacctcattttttggagcttcaacaattttcttaaaatcaaatagtcgttttattggacaacctttatggggttctattggatatacttttccagcggctttaggaagccaaattgcggataaagagagcagacaccttttatttattggtgatggttcacttcaacttaccgtacaagaattaggactatcaatcagagaaaaactcaatccaatttgttttatcataaataatgatggttatacagttgaaagagaaatccacggacctactcaaagttataacgacattccaatgtggaattactcgaaattaccagaaacatttggagcaacagaagatcgtgtagtatcaaaaattgttagaacagagaatgaatttgtgtctgtcatgaaagaagcccaagcagatgtcaatagaatgtattggatagaactagttttggaaaaagaagatgcgccaaaattactgaaaaaaatgggcaaactatttgctgaacaaaataaatcataa

SEQ ID NO: 32 - DNA, 1647 bp, Artificial Sequence
  Synthetic: kdcA Codon-optimized kdcA sequence
atgtatacagtaggagattaccttttagatcgtttgcacgaattgggcattgaggaaatttttggcgtccctggcgactacaatttacaattcttagatcagattatttcacgtgaggatatgaagtggattgggaatgccaatgagctgaacgcgagctatatggcggacggttacgctcgtacaaaaaaggcagcagcgtttcttactacttttggcgtaggcgaattgtcggccatcaacgggcttgcgggttcgtatgcggaaaacttaccggttgtcgagattgtcggttcccctacttcgaaggtgcagaatgatggcaaattcgttcatcacaccttggcagacggcgactttaaacatttcatgaaaatgcacgaacctgtgactgccgcccgcacacttctgacagctgaaaacgcgacatacgaaattgatcgcgtgctttcgcagttgttgaaagagcgtaaacccgtatatatcaatctgccggtggatgtagcggctgcaaaagccgaaaaaccggcgctgtcactggaaaaagaatcgtctacgactaatacaacggaacaagtaatcctgtcaaaaatcgaagagagcttgaaaaacgcccagaagcctgtcgtgattgccgggcacgaggtcattagttttgggttagaaaagactgttacccagttcgtgagtgagacgaagttgcccatcaccacccttaactttggcaagtctgcggtagacgagagcttaccgtcttttttaggtatctacaatgggaaactttcagaaatttcactgaaaaacttcgtggagtcggcagactttattttaatgttgggtgttaaattaactgatagcagcactggcgcgttcacgcatcacttggatgagaataaaatgatctcgcttaacatcgacgaaggtatcatttttaataaagttgtagaggacttcgactttcgtgctgttgtatcgagcctttccgaattaaagggtatcgagtacgaaggtcagtacattgacaagcaatacgaggaatttatcccctccagcgcgcctcttagccaagaccgcctttggcaggccgtagagagtcttacacaaagtaatgaaactattgttgcagaacagggtacaagcttctttggcgcctcgacgattttcttaaaatcgaacagtcgctttatcgggcaacctctttgggggtcgattgggtacacctttcctgcggccttaggctctcaaattgcggacaaagaatctcgccatttattattcatcggcgacggctcgttacagcttacagtgcaagagttgggattatcgattcgcgagaagctgaatccgatttgctttatcattaacaacgacgggtacacagtcgaacgcgaaatccatggcccgacacaatcatataatgacatccctatgtggaattattctaagcttccagagacattcggcgcaactgaagaccgcgtcgtgtcaaaaattgtccgcactgagaatgaattcgtgtcagtgatgaaggaagctcaggccgatgtcaaccgcatgtactggattgaattagttttggagaaagaggatgcccccaaattacttaagaagatggggaaactatttgctgaacaaaataaatcataa

SEQ ID NO: 33 - protein, 609 aa, Saccharomyces cerevisiae
  THI3/KID1 Amino acid sequence
MNSSYTQRYALPKCIAISDYLFHRLNQLNIHTIFGLSGEFSMPLLDKLYNIPNLRWAGNSNELNAAYAADGYSRLKGLGCLITTFGVGELSAINGVAGSYAEHVGILHIVGMPPTSAQTKQLLLHHTLGNGDFTVFHRIASDVACYTTLIIDSELCADEVDKCIKKAWIEQRPVYMGMPVNQVNLPIESARLNTPLDLQLHKNDPDVEKEVISRILSFIYKSQNPAIIVDACTSRQNLIEETKELCNRLKFPVFVTPMGKGTVNETDPQFGGVFTGSISAPEVREVVDFADFIIVIGCMLSEFSTSTFHFQYKTKNCALLYSTSVKLKNATYPDLSIKLLLQKILANLDESKLSYQPSEQPSMMVPRPYPAGNVLLRQEWVWNEISHWFQPGDIIITETGASAFGVNQTRFPVNTLGISQALWGSVGYTMGACLGAEFAVQEINKDKFPATKHRVILFMGDGAFQLTVQELSTIVKWGLTPYIFVMNNQGYSVDRFLHHRSDASYYDIQPWNYLGLLRVFGCTNYETKKIITVGEFRSMISDPNFATNDKIRMIEIMLPPRDVPQALLDRWVVEKEQSKQVQEENENSSAVNTPTPEFQPLLKKNQVGY

SEQ ID NO: 34 - DNA, 1830 bp, Artificial Sequence
  Synthetic: THI3/KID1 Nucleotide sequence
atgaattctagctatacacagagatatgcactgccgaagtgtatagcaatatcagattatcttttccatcggctcaaccagctgaacatacataccatatttggactctccggagaatttagcatgccgttgctggataaactatacaacattccgaacttacgatgggccggtaattctaatgagttaaatgctgcctacgcagcagatggatactcacgactaaaaggcttgggatgtctcataacaacctttggtgtaggcgaattatcggcaatcaatggcgtggccggatcttacgctgaacatgtaggaatacttcacatagtgggtatgccgccaacaagtgcacaaacgaaacaactactactgcatcatactctgggcaatggtgatttcacggtatttcatagaatagccagtgatgtagcatgctatacaacattgattattgactctgaattatgtgccgacgaagtcgataagtgcatcaaaaaggcttggatagaacagaggccagtatacatgggcatgcctgtcaaccaggtaaatctcccgattgaatcagcaaggcttaatacacctctggatttacaattgcataaaaacgacccagacgtagagaaagaagttatttctcgaatattgagttttatatacaaaagccagaatccggcaatcatcgtagatgcatgtactagtcgacagaatttaatcgaggagactaaagagctttgtaataggcttaaatttccagtttttgttacacctatgggtaagggtacagtaaacgaaacagacccgcaatttgggggcgtattcacgggctcgatatcagccccagaagtaagagaagtagttgattttgccgattttatcatcgtcattggttgcatgctctccgaattcagcacgtcaactttccacttccaatataaaactaagaattgtgcgctactatattctacatctgtgaaattgaaaaatgccacatatcctgacttgagcattaaattactactacagaaaatattagcaaatcttgatgaatctaaactgtcttaccaaccaagcgaacaacccagtatgatggttccaagaccttacccagcaggaaatgtcctcttgagacaagaatgggtctggaatgaaatatcccattggttccaaccaggtgacataatcataacagaaactggtgcttctgcatttggagttaaccagaccagatttccggtaaatacactaggtatttcgcaagctctttggggatctgtcggatatacaatgggggcgtgtcttggggcagaatttgctgttcaagagataaacaaggataaattccccgcaactaaacatagagttattctgtttatgggtgacggtgctttccaattgacagttcaagaattatccacaattgttaagtggggattgacaccttatatttttgtgatgaataaccaaggttactctgtggacaggtttttgcatcacaggtcagatgctagttattacgatatccaaccttggaactacttgggattattgcgagtatttggttgcacgaactacgaaacgaaaaaaattattactgttggagaattcagatccatgatcagtgacccaaactttgcgaccaatgacaaaattcggatgatagagattatgctaccaccaagggatgttccacaggctctgcttgacaggtgggtggtagaaaaagaacagagcaaacaagtgcaagaggagaacgaaaattctagcgcagtaaatacgccaactccagaattccaaccacttctaaaaaaaaatcaagttggatactga

SEQ ID NO: 35 - protein, 635 aa, Saccharomyces cerevisiae
  ARO10 Amino acid sequence
MAPVTIEKFVNQEERHLVSNRSATIPFGEYIFKRLLSIDTKSVFGVPGDFNLSLLEYLYSPSVESAGLRWVGTCNELNAAYAADGYSRYSNKIGCLITTYGVGELSALNGIAGSFAENVKVLHIVGVAKSIDSRSSNFSDRNLHHLVPQLHDSNFKGPNHKVYHDMVKDRVACSVAYLEDIETACDQVDNVIRDIYKYSKPGYIFVPADFADMSVTCDNLVNVPRISQQDCIVYPSENQLSDIINKITSWIYSSKTPAILGDVLTDRYGVSNFLNKLICKTGIWNFSTVMGKSVIDESNPTYMGQYNGKEGLKQVYEHFELCDLVLHFGVDINEINNGHYTFTYKPNAKIIQFHPNYIRLVDTRQGNEQMFKGINFAPILKELYKRIDVSKLSLQYDSNVTQYTNETMRLEDPTNGQSSIITQVHLQKTMPKFLNPGDVVVCETGSFQFSVRDFAFPSQLKYISQGFFLSIGMALPAALGVGIAMQDHSNAHINGGNVKEDYKPRLILFEGDGAAQMTIQELSTILKCNIPLEVIIWNNNGYTIERAIMGPTRSYNDVMSWKWTKLFEAFGDFDGKYTNSTLIQCPSKLALKLEELKNSNKRSGIELLEVKLGELDFPEQLKCMVEAAALKRNKK

SEQ ID NO: 36 - DNA, 1908 bp, Artificial Sequence
  Synthetic: ARO10 Nucleotide sequence
atggcacctgttacaattgaaaagttcgtaaatcaagaagaacgacaccttgtttccaaccgatcagcaacaattccgtttggtgaatacatatttaaaagattgttgtccatcgatacgaaatcagttttcggtgttcctggtgacttcaacttatctctattagaatatctctattcacctagtgttgaatcagctggcctaagatgggtcggcacgtgtaatgaactgaacgccgcttatgcggccgacggatattcccgttactctaataagattggctgtttaataaccacgtatggcgttggtgaattaagcgccttgaacggtatagccggttcgttcgctgaaaatgtcaaagttttgcacattgttggtgtggccaagtccatagattcgcgttcaagtaactttagtgatcggaacctacatcatttggtcccacagctacatgattcaaattttaaagggccaaatcataaagtatatcatgatatggtaaaagatagagtcgcttgctcggtagcctacttggaggatattgaaactgcatgtgaccaagtcgataatgttatccgcgatatttacaagtattctaaacctggttatatttttgttcctgcagattttgcggatatgtctgttacatgtgataatttggttaatgttccacgtatatctcaacaagattgtatagtatacccttctgaaaaccaattgtctgacataatcaacaagattactagttggatatattccagtaaaacacctgcgatccttggagacgtactgactgataggtatggtgtgagtaactttttgaacaagcttatctgcaaaactgggatttggaatttttccactgttatgggaaaatctgtaattgatgagtcaaacccaacttatatgggtcaatataatggtaaagaaggtttaaaacaagtctatgaacattttgaactgtgcgacttggtcttgcattttggagtcgacatcaatgaaattaataatgggcattatacttttacttataaaccaaatgctaaaatcattcaatttcatccgaattatattcgccttgtggacactaggcagggcaatgagcaaatgttcaaaggaatcaattttgcccctattttaaaagaactatacaagcgcattgacgtttctaaactttctttgcaatatgattcaaatgtaactcaatatacgaacgaaacaatgcggttagaagatcctaccaatggacaatcaagcattattacacaagttcacttacaaaagacgatgcctaaatttttgaaccctggtgatgttgtcgtttgtgaaacaggctcttttcaattctctgttcgtgatttcgcgtttccttcgcaattaaaatatatatcgcaaggatttttcctttccattggcatggcccttcctgccgccctaggtgttggaattgccatgcaagaccactcaaacgctcacatcaatggtggcaacgtaaaagaggactataagccaagattaattttgtttgaaggtgacggtgcagcacagatgacaatccaagaactgagcaccattctgaagtgcaatattccactagaagttatcatttggaacaataacggctacactattgaaagagccatcatgggccctaccaggtcgtataacgacgttatgtcttggaaatggaccaaactatttgaagcattcggagacttcgacggaaagtatactaatagcactctcattcaatgtccctctaaattagcactgaaattggaggagcttaagaattcaaacaaaagaagcgggatagaacttttagaagtcaaattaggcgaattggatttccccgaacagctaaagtgcatggttgaagcagcggcacttaaaagaaataaaaaatag

SEQ ID NO: 37 - protein, 348 aa, Saccharomyces cerevisiae
  Adh2 Amino acid sequence
MSIPETQKAIIFYESNGKLEHKDIPVPKPKPNELLINVKYSGVCHTDLHAWHGDWPLPTKLPLVGGHEGAGVVVGMGENVKGWKIGDYAGIKWLNGSCMACEYCELGNESNCPHADLSGYTHDGSFQEYATADAVQAAHIPQGTDLAEVAPILCAGITVYKALKSANLRAGHWAAISGAAGGLGSLAVQYAKAMGYRVLGIDGGPGKEELFTSLGGEVFIDFTKEKDIVSAVVKATNGGAHGIINVSVSEAAIEASTRYCRANGTVVLVGLPAGAKCSSDVFNHVVKSISIVGSYVGNRADTREALDFFARGLVKSPIKVVGLSSLPEIYEKMEKGQIAGRYVVDTSK

SEQ ID NO: 38 - DNA, 1047 bp, Artificial Sequence
  Synthetic: adh2 Nucleotide sequence
atgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataa

SEQ ID NO: 39 - DNA, 1047 bp, Artificial Sequence
  Synthetic: adh2 Codon-optimized sequence
atgtctattccagaaacgcagaaagccatcatattttatgaatcgaacggaaaacttgagcacaaggacatccccgtcccgaagccaaaacctaatgagttgcttatcaacgttaagtattcgggcgtatgccacacagacttgcacgcatggcacggggattggcccttaccgactaagttgccgttagtgggcggacatgagggggcgggagtcgtagtgggaatgggagagaacgtgaagggttggaagattggagattatgctgggattaagtggttgaatgggagctgcatggcctgcgaatattgtgaacttggaaatgagagcaattgcccacatgctgacttgtccggttacacacatgacggttcattccaggaatatgctacggctgatgcagtccaagcagcgcatatcccgcaagggacggacttagcagaagtagcgcccattctttgcgctgggatcaccgtatataaagcgttaaagagcgcaaatttacgggccggacattgggcggcgatcagcggggccgcaggggggctgggcagcttggccgtccagtacgctaaagctatgggttatcgggttttgggcattgacggaggaccgggaaaggaggaattattcacgtccttgggaggagaggtattcattgactttaccaaggaaaaagatatcgtctctgctgtagtaaaggctaccaatggcggtgcccacggaatcataaatgtttcagtttctgaagcggcgatcgaagcgtccactagatattgccgtgcaaatgggacagtcgtacttgtaggacttccggctggcgccaaatgcagctccgatgtatttaatcatgtcgtgaagtcaatctctatcgttggttcatatgtaggaaaccgcgccgatactcgtgaggctcttgacttttttgccagaggcctggttaagtcccccataaaagttgttggcttatccagcttacccgaaatatacgagaagatggagaagggccagatcgcggggagatacgttgttgacacttctaaataa

SEQ ID NO: 40 - protein, 360 aa, Saccharomyces cerevisiae; identical to SEQ ID NO: 21
  Adh6 Amino acid sequence

SEQ ID NO: 41 - DNA, 1083 bp, Artificial Sequence
  Synthetic: adh6 Codon-optimized sequence
atgtcataccctgaaaaattcgagggtatcgccattcagagtcacgaagattggaagaatcccaagaagaccaaatacgaccccaagccgttctatgaccatgatatcgacatcaaaatcgaggcatgtggtgtgtgtggcagtgatattcattgcgcagcgggccattgggggaacatgaagatgcctctggtagtaggacatgagatcgttggaaaggttgtgaaattgggtccgaaaagtaactccggtcttaaagtaggtcagcgtgttggggtcggggcgcaagttttcagttgcctggagtgtgatcgttgtaagaacgataacgagccgtactgcacaaagtttgtaacgacgtattcacagccatatgaggatgggtatgtttctcaagggggctatgcaaactacgtccgcgtacatgaacactttgtggtgcctattcctgagaacattccgtctcacttggccgctcctttgttgtgcggaggtcttaccgtctactcgccattggttcgcaatgggtgcggtccgggcaaaaaggtagggatcgttggccttggtggtatcggatctatgggaacgttaatcagtaaggcgatgggagctgagacctacgttatttcccgttcatcacgtaagcgtgaggatgcgatgaagatgggtgcagatcactacatcgcaacgttagaagagggagattggggcgaaaaatattttgacacttttgacttgattgtggtttgtgcatcgtcacttacagacattgactttaatattatgccaaaggcaatgaaggtaggtgggcgtattgtgtccatttctatcccggaacaacacgagatgctttctctgaaaccctacggacttaaagctgtgtccatttcgtacagtgcccttggatctatcaaggaactgaatcagctgctgaagcttgtttcggagaaagacattaagatttgggtggagacattgccagtgggggaggccggcgttcacgaggcgtttgaacgcatggagaagggagatgttcgctatcgcttcacgctggttggttatgataaagaattcagtgattag

SEQ ID NO: 42 - protein, 348 aa, Saccharomyces cerevisiae
  Adh1 Amino acid sequence
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 43 - DNA, 1047 bp, Artificial Sequence
  Synthetic: adh1 Nucleotide sequence
atgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaacaatacgctaccgctgacgctgttcaagccgctcacattcctcaaggtaccgacttggcccaagtcgcccccatcttgtgtgctggtatcaccgtctacaaggctttgaagtctgctaacttgatggccggtcactgggttgctatctccggtgctgctggtggtctaggttctttggctgttcaatacgccaaggctatgggttacagagtcttgggtattgacggtggtgaaggtaaggaagaattattcagatccatcggtggtgaagtcttcattgacttcactaaggaaaaggacattgtcggtgctgttctaaaggccactgacggtggtgctcacggtgtcatcaacgtttccgtttccgaagccgctattgaagcttctaccagatacgttagagctaacggtaccaccgttttggtcggtatgccagctggtgccaagtgttgttctgatgtcttcaaccaagtcgtcaagtccatctctattgttggttcttacgtcggtaacagagctgacaccagagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataa

SEQ ID NO: 44 - protein, 375 aa, Saccharomyces cerevisiae [sequence omitted]

SEQ ID NO: 45 - DNA, 1128 bp, Artificial Sequence
  Synthetic: adh3 Nucleotide sequence
atgttgagaacgtcaacattgttcaccaggcgtgtccaaccaagcctattttctagaaacattcttagattgcaatccacagctgcaatccctaagactcaaaaaggtgtcatcttttatgagaataaggggaagctgcattacaaagatatccctgtccccgagcctaagccaaatgaaattttaatcaacgttaaatattctggtgtatgtcacaccgatttacatgcttggcacggcgattggccattacctgttaaactaccattagtaggtggtcatgaaggtgctggtgtagttgtcaaactaggttccaatgtcaagggctggaaagtcggtgatttagcaggtatcaaatggctgaacggttcttgtatgacatgcgaattctgtgaatcaggtcatgaatcaaattgtccagatgctgatttatctggttacactcatgatggttctttccaacaatttgcgaccgctgatgctattcaagccgccaaaattcaacagggtaccgacttggccgaagtagccccaatattatgtgctggtgttactgtatataaagcactaaaagaggcagacttgaaagctggtgactgggttgccatctctggtgctgcaggtggcttgggttccttggccgttcaatatgcaactgcgatgggttacagagttctaggtattgatgcaggtgaggaaaaggaaaaacttttcaagaaattggggggtgaagtattcatcgactttactaaaacaaagaatatggtttctgacattcaagaagctaccaaaggtggccctcatggtgtcattaacgtttccgtttctgaagccgctatttctctatctacggaatatgttagaccatgtggtaccgtcgttttggttggtttgcccgctaacgcctacgttaaatcagaggtattctctcatgtggtgaagtccatcaatatcaagggttcttatgttggtaacagagctgatacgagagaagccttagacttctttagcagaggtttgatcaaatcaccaatcaaaattgttggattatctgaattaccaaaggtttatgacttgatggaaaagggcaagattttgggtagatacgtcgtcgatactagtaaataa

SEQ ID NO: 46 - protein, 382 aa, Saccharomyces cerevisiae
  Adh4 Amino acid sequence
MSSVTGFYIPPISFFGEGALEETADYIKNKDYKKALIVTDPGIAAIGLSGRVQKMLEERDLNVAIYDKTQPNPNIANVTAGLKVLKEQNSEIVVSIGGGSAHDNAKAIALLATNGGEIGDYEGVNQSKKAALPLFAINTTAGTASEMTRFTIISNEEKKIKMAIIDNNVTPAVAVNDPSTMFGLPPALTAATGLDALTHCIEAYVSTASNPITDACALKGIDLINESLVAAYKDGKDKKARTDMCYAEYLAGMAFNNASLGYVHALAHQLGGFYHLPHGVCNAVLLPHVQEANMQCPKAKKRLGEIALHFGASQEDPEETIKALHVLNRTMNIPRNLKELGVKTEDFEILAEHAMHDACHLTNPVQFTKEQVVAIIKKAYEY

SEQ ID NO: 47 - DNA, 1149 bp, Artificial Sequence
  Synthetic: adh4 Nucleotide sequence
atgtcttccgttactgggttttacattccaccaatctctttctttggtgaaggtgctttagaagaaaccgctgattacatcaaaaacaaggattacaaaaaggctttgatcgttactgatcctggtattgcagctattggtctctccggtagagtccaaaagatgttggaagaacgtgacttaaacgttgctatctatgacaaaactcaaccaaacccaaatattgccaatgtcacagctggtttgaaggttttgaaggaacaaaactctgaaattgttgtttccattggtggtggttctgctcacgacaatgctaaggccattgctttattggctactaacggtggggaaatcggagactatgaaggtgtcaatcaatctaagaaggctgctttaccactatttgccatcaacactactgctggtactgcttccgaaatgaccagattcactattatctctaatgaagaaaagaaaatcaagatggctatcattgacaacaacgtcactccagctgttgctgtcaacgatccatctaccatgtttggtttgccacctgctttgactgctgctactggtctagatgctttgactcactgtatcgaagcttatgtttccaccgcctctaacccaatcaccgatgcctgtgctttgaagggtattgatttgatcaatgaaagcttagtcgctgcatacaaagacggtaaagacaagaaggccagaactgacatgtgttacgctgaatacttggcaggtatggctttcaacaatgcttctctaggttatgttcatgcccttgctcatcaacttggtggtttctaccacttgcctcatggtgtttgtaacgctgtcttgttgcctcatgttcaagaggccaacatgcaatgtccaaaggccaagaagagattaggtgaaattgctttgcatttcggtgcttctcaagaagatccagaagaaaccatcaaggctttgcacgttttaaacagaaccatgaacattccaagaaacttgaaagaattaggtgttaaaaccgaagattttgaaattttggctgaacacgccatgcatgatgcctgccatttgactaacccagttcaattcaccaaagaacaagtggttgccattatcaagaaagcctatgaatattaa

SEQ ID NO: 48 - protein, 351 aa, Saccharomyces cerevisiae
  Adh5 Amino acid sequence
MPSQVIPEKQKAIVFYETDGKLEYKDVTVPEPKPNEILVHVKYSGVCHSDLHAWHGDWPFQLKFPLIGGHEGAGVVVKLGSNVKGWKVGDFAGIKWLNGTCMSCEYCEVGNESQCPYLDGTGFTHDGTFQEYATADAVQAAHIPPNVNLAEVAPILCAGITVYKALKRANVIPGQWVTISGACGGLGSLAIQYALAMGYRVIGIDGGNAKRKLFEQLGGEIFIDFTEEKDIVGAIIKATNGGSHGVINVSVSEAAIEASTRYCRPNGTVVLVGMPAHAYCNSDVFNQVVKSISIVGSCVGNRADTREALDFFARGLIKSPIHLAGLSDVPEIFAKMEKGEIVGRYVVETSK

SEQ ID NO: 49 - DNA, 1056 bp, Artificial Sequence
  Synthetic: adh5 Nucleotide sequence
atgccttcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaatga

SEQ ID NO: 50 - protein, 361 aa, Saccharomyces cerevisiae
  Adh7 Amino acid sequence
MLYPEKFQGIGISNAKDWKHPKLVSFDPKPFGDHDVDVEIEACGICGSDFHIAVGNWGPVPENQILGHEIIGRVVKVGSKCHTGVKIGDRVGVGAQALACFECERCKSDNEQYCTNDHVLTMWTPYKDGYISQGGFASHVRLHEHFAIQIPENIPSPLAAPLLCGGITVFSPLLRNGCGPGKRVGIVGIGGIGHMGILLAKAMGAEVYAFSRGHSKREDSMKLGADHYIAMLEDKGWTEQYSNALDLLVVCSSSLSKVNFDSIVKIMKIGGSIVSIAAPEVNEKLVLKPLGLMGVSISSSAIGSRKEIEQLLKLVSEKNVKIWVEKLPISEEGVSHAFTRMESGDVKYRFTLVDYDKKFHK

SEQ ID NO: 51 - DNA, 1086 bp, Artificial Sequence
  Synthetic: adh7 Nucleotide sequence
atgctttacccagaaaaatttcagggcatcggtatttccaacgcaaaggattggaagcatcctaaattagtgagttttgacccaaaaccctttggcgatcatgacgttgatgttgaaattgaagcctgtggtatctgcggatctgattttcatatagccgttggtaattggggtccagtcccagaaaatcaaatccttggacatgaaataattggccgcgtggtgaaggttggatccaagtgccacactggggtaaaaatcggtgaccgtgttggtgttggtgcccaagccttggcgtgttttgagtgtgaacgttgcaaaagtgacaacgagcaatactgtaccaatgaccacgttttgactatgtggactccttacaaggacggctacatttcacaaggaggctttgcctcccacgtgaggcttcatgaacactttgctattcaaataccagaaaatattccaagtccgctagccgctccattattgtgtggtggtattacagttttctctccactactaagaaatggctgtggtccaggtaagagggtaggtattgttggcatcggtggtattgggcatatggggattctgttggctaaagctatgggagccgaggtttatgcgttttcgcgaggccactccaagcgggaggattctatgaaactcggtgctgatcactatattgctatgttggaggataaaggctggacagaacaatactctaacgctttggaccttcttgtcgtttgctcatcatctttgtcgaaagttaattttgacagtatcgttaagattatgaagattggaggctccatcgtttcaattgctgctcctgaagttaatgaaaagcttgttttaaaaccgttgggcctaatgggagtatcaatctcaagcagtgctatcggatctaggaaggaaatcgaacaactattgaaattagtttccgaaaagaatgtcaaaatatgggtggaaaaacttccgatcagcgaagaaggcgtcagccatgcctttacaaggatggaaagcggagacgtcaaatacagatttactttggtcgattatgataagaaattccataaatag

SEQ ID NO: 52 - protein, 386 aa, Saccharomyces cerevisiae
  SFA1 Amino acid sequence
MSAATVGKPIKCIAAVAYDAKKPLSVEEITVDAPKAHEVRIKIEYTAVCHTDAYTLSGSDPEGLFPCVLGHEGAGIVESVGDDVITVKPGDHVIALYTAECGKCKFCTSGKTNLCGAVRATQGKGVMPDGTTRFHNAKGEDIYHFMGCSTFSEYTVVADVSVVAIDPKAPLDAACLLGCGVTTGFGAALKTANVQKGDTVAVFGCGTVGLSVIQGAKLRGASKIIAIDINNKKKQYCSQFGATDFVNPKEDLAKDQTIVEKLIEMTDGGLDFTFDCTGNTKIMRDALEACHKGWGQSIIIGVAAAGEEISTRPFQLVTGRVWKGSAFGGIKGRSEMGGLIKDYQKGALKVEEFITHRRPFKEINQAFEDLHNGDCLRTVLKSDEIK

SEQ ID NO: 53 - DNA, 1161 bp, Artificial Sequence
  Synthetic: sfa1 Nucleotide sequence
atgtccgccgctactgttggtaaacctattaagtgcattgctgctgttgcgtatgatgcgaagaaaccattaagtgttgaagaaatcacggtagacgccccaaaagcgcacgaagtacgtatcaaaattgaatatactgctgtatgccacactgatgcgtacactttatcaggctctgatccagaaggacttttcccttgcgttctgggccacgaaggagccggtatcgtagaatctgtaggcgatgatgtcataacagttaagcctggtgatcatgttattgctttgtacactgctgagtgtggcaaatgtaagttctgtacttccggtaaaaccaacttatgtggtgctgttagagctactcaagggaaaggtgtaatgcctgatgggaccacaagatttcataatgcgaaaggtgaagatatataccatttcatgggttgctctactttttccgaatatactgtggtggcagatgtctctgtggttgccatcgatccaaaagctcccttggatgctgcctgtttactgggttgtggtgttactactggttttggggcggctcttaagacagctaatgtgcaaaaaggcgataccgttgcagtatttggctgcgggactgtaggactctccgttatccaaggtgcaaagttaaggggcgcttccaagatcattgccattgacattaacaataagaaaaaacaatattgttctcaatttggtgccacggattttgttaatcccaaggaagatttggccaaagatcaaactatcgttgaaaagttaattgaaatgactgatgggggtctggattttacttttgactgtactggtaataccaaaattatgagagatgctttggaagcctgtcataaaggttggggtcaatctattatcattggtgtggctgccgctggtgaagaaatttctacaaggccgttccagctggtcactggtagagtgtggaaaggctctgcttttggtggcatcaaaggtagatctgaaatgggcggtttaattaaagactatcaaaaaggtgccttaaaagtcgaagaatttatcactcacaggagaccattcaaagaaatcaatcaagcctttgaagatttgcataacggtgattgcttaagaaccgtcttgaagtctgatgaaataaaatag

SEQ ID NO: 54 - protein, 491 aa, Escherichia coli
  IlvC amino acid sequence from E. coli Nissle
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVVNLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNAQLRDVNEAIRSHAIEQVGKKLRGYMTDMKRIAVAG

SEQ ID NO: 55 - DNA, 1476 bp, Escherichia coli
  ilvC gene from E. coli Nissle Nucleotide sequence
atggctaactacttcaatacactgaatctgcgccagcagttggcacagctgggcaaatgtcgctttatggggcgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgctgagaagcgcgcatcctggcgtaaagcaaccgaaaatggttttaaagtgggtacttacgaagaactgatcccgcaggcggatctggtggttaacctgacgccggacaagcagcactctgatgtagtgcgcaccgtacagccactgatgaaagacggcgcggcgctgggctactctcatggtttcaatatcgtagaagtgggtgagcagatccgtaaagacatcaccgtcgtaatggttgcgccgaaatgccctggcaccgaagtacgtgaagagtacaaacgtggattcggcgtaccgacgctgattgccgttcacccggaaaacgatccgaaaggcgaaggcatggcgatcgctaaagcatgggcggctgcaaccggcggtcaccgtgcgggcgttctggaatcctctttcgttgcggaagtgaaatctgacctgatgggcgagcaaaccatcctgtgcggtatgttgcaagctggttctctgctgtgcttcgacaagctggtggaagaaggcaccgatccggcatacgcagaaaaactgattcagttcggttgggaaaccatcaccgaagcgctgaaacagggcggcatcaccctgatgatggaccgtctttctaacccggcgaaactgcgtgcttacgcgctttctgagcaactgaaagagatcatggcgccgctgttccagaaacatatggacgacatcatctccggcgaattctcctccggcatgatggctgactgggccaacgacgataagaaactgctgacctggcgtgaagagactggcaaaaccgcattcgaaaccgcgccgcagtatgaaggcaaaatcggtgaacaggagtacttcgataaaggcgtactgatgatcgcgatggtaaaagcaggcgttgagttggcgtttgaaaccatggttgattccggcatcatcgaagaatctgcttactatgaatcactgcacgaactgccgctgattgccaacaccatcgcccgtaagcgtctgtacgaaatgaacgtggttatctccgatactgccgagtacggtaactatctgttctcttacgcttgtgtgccactgctgaaaccgtttatggcagagctgcaaccgggcgacctgggtaaagctattccggaaggtgcggtagataacgcgcagctgcgtgatgtaaatgaagcgattcgcagccatgcgattgagcaggtaggtaagaaactgcgcggctatatgacggatatgaaacgtattgctgttgcgggttaa

SEQ ID NO: 56 - DNA, 1416 bp, Proteus vulgaris; identical to SEQ ID NO: 24
  L-amino acid deaminase L-AAD

SEQ ID NO: 57 - protein, 471 aa, Artificial Sequence; identical to SEQ ID NO: 23
  Synthetic: amino acid sequence

SEQ ID NO: 58 - DNA, 1101 bp, Bacillus cereus
  Leucine dehydrogenase leuDH
atgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctga

SEQ ID NO: 59 - protein, 366 aa, Artificial Sequence
  Synthetic: amino acid sequence
MTLEIFEYLEKYDYEQVVFCQDKESGLKAIIAIHDTTLGPALGGTRMWTYDSEEAAIEDALRLAKGMTYKNAAAGLNLGGAKTVIIGDPRKDKSEAMFRALGRYIQGLNGRYITAEDVGTTVDDMDIIHEETDFVTGISPSFGSSGNPSPVTAYGVYRGMKAAAKEAFGTDNLEGKVIAVQGVGNVAYHLCKHLHAEGAKLIVTDINKEAVQRAVEEFGASAVEPNEIYGVECDIYAPCALGATVNDETIPQLKAKVIAGSANNQLKEDRHGDIIHEMGIVYAPDYVINAGGVINVADELYGYNRERALKRVESIYDTIAKVIEISKRDGIATYVAADRLAEERIASLKNSRSTYLRNGHDIISRR

SEQ ID NO: 60 - DNA, 1164 bp, Escherichia coli
  Alcohol dehydrogenase YqhD
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtactggaatttggcggtattgaaccaaacccggcttatgaaacgctgatgaacgccgtgaaactggttcgcgaacagaaagtgacgttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagcccgtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctgatcgaagatggtccgaaagccctgaaagagccagaaaactacgatgtgcgcgccaacgtcatgtgggcggcgactcaggcgctgaacggtttgatcggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttcagacgatgagcgtattgacgccgcgattgccgcaacccgcaatttctttgagcaattaggcgtgctgacccacctctccgactacggtctggacggcagctccatcccggctttgctgaaaaaactggaagagcacggcatgacccaactgggcgaaaatcatgacattacgctggatgtcagccgccgtatatacgaagccgcccgctaa

SEQ ID NO: 61 - protein, 387 aa, Artificial Sequence
  Synthetic: amino acid sequence
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVLTHLSDYGLDGSSIPALLKKLEEHGMTQLGENHDITLDVSRRIYEAAR

SEQ ID NO: 62 - DNA, 1500 bp, Escherichia coli
  Aldehyde dehydrogenase PadA
atgacagagccgcatgtagcagtattaagccaggtccaacagtttctcgatcgtcaacacggtctttatattgatggtcgtcctggccccgcacaaagtgaaaaacggttggcgatctttgatccggccaccgggcaagaaattgcgtctactgctgatgccaacgaagcggatgtagataacgcagtcatgtctgcctggcgggcctttgtctcgcgtcgctgggccgggcgattacccgcagagcgtgaacgtattctgctacgttttgctgatctggtggagcagcacagtgaggagctggcgcaactggaaaccctggagcaaggcaagtcaattgccatttcccgtgcttttgaagtgggctgtacgctgaactggatgcgttataccgccgggttaacgaccaaaatcgcgggtaaaacgctggacttgtcgattcccttaccccagggggcgcgttatcaggcctggacgcgtaaagagccggttggcgtagtggcgggaattgtgccatggaactttccgttgatgattggtatgtggaaggtgatgccagcactggcagcaggctgttcaatcgtgattaagccttcggaaaccacgccactgacgatgttgcgcgtggcggaactggccagcgaggctggtatccctgatggcgtttttaatgtcgtcaccgggtcaggtgctgtatgcggcgcggccctgacgtcacatcctcatgttgcgaaaatcagttttaccggttcaaccgcgacgggaaaaggtattgccagaactgctgctgatcacttaacgcgtgtaacgctggaactgggcggtaaaaacccggcaattgtattaaaagatgctgatccgcaatgggttattgaaggcttgatgaccggaagcttcctgaatcaagggcaagtatgcgccgccagttcgcgaatttatattgaagcgccgttgtttgacacgctggttagtggatttgagcaggcggtaaaatcgttgcaagtgggaccggggatgtcacctgttgcacagattaaccctttggtttctcgtgcgcactgcgacaaagtgtgttcattcctcgacgatgcgcaggcacagcaagcagagctgattcgcgggtcgaatggaccagccggagaggggtattatgttgcgccaacgctggtggtaaatcccgatgctaaattgcgcttaactcgtgaagaggtgtttggtccggtggtaaacctggtgcgagtagcggatggagaagaggcgttacaactggcaaacgacacggaatatggcttaactgccagtgtctggacgcaaaatctctcccaggctctggaatatagcgatcgcttacaggcagggacggtgtgggtaaacagccataccttaattgacgctaacttaccgtttggtgggatgaagcagtcaggaacgggccgtgattttggccccgactggctggacggttggtgtgaaactaagtcggtgtgtgtacggtattaa

SEQ ID NO: 63 - protein, 499 aa, Artificial Sequence
  Synthetic: amino acid sequence
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLETLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADHLTRVTLELGGKNPAIVLKDADPQWVIEGLMTGSFLNQGQVCAASSRIYIEAPLFDTLVSGFEQAVKSLQVGPGMSPVAQINPLVSRAHCDKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 64 - DNA, 1320 bp, Escherichia coli
  BCAA transporter BrnQ
atgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaa

SEQ ID NO: 65 - protein, 439 aa, Artificial Sequence
  Synthetic: amino acid sequence
MTHQLRSRDIIALGFMTFALFVGAGNIIFPPMVGLQAGEHVWTAAFGFLITAVGLPVLTVVALAKVGGGVDSLSTPIGKVAGVLLATVCYLAVGPLFATPRTATVSFEVGIAPLTGDSALPLFIYSLVYFAIVILVSLYPGKLLDTVGNFLAPLKIIALVILSVAAIIWPAGSISTATEAYQNAAFSNGFVNGYLTMDTLGAMVFGIVIVNAARSRGVTEARLLTRYTVWAGLMAGVGLTLLYLALFRLGSDSASLVDQSANGAAILHAYVQHTFGGGGSFLLAALIFIACLVTAVGLTCACAEFFAQYVPLSYRTLVFILGGFSMVVSNLGLSQLIQISVPVLTAIYPPCIALVVLSFTRSWWHNSSRVIAPPMFISLLFGILDGIKASAFSDILPSWAQRLPLAEQGLAWLMPTVVMVVLAIIWDRAAGRQVTSSAH

SEQ ID NO: 66 - protein, 541 aa, Streptomyces lividans
  Isovaleryl-CoA synthetase LbuL
MTAPAPQPSYAHGTSTTPLLGDTVGANLGRAIAAHPDREALVDVPSGRRWTYAEFGAAVDELARGLLAKGVTRGDRVGIWAVNCPEWVLVQYATARIGVIMVNVNPAYRAHELEYVLQQSGISLLVASLAHKSSDYRAIVEQVRGRCPALRETVYIGDPSWDALTAGAAAVEQDRVDALAAELSCDDPVNIQYTSGTTGFPKGATLSHHNILNNGYWVGRTVGYTEQDRVCLPVPFYHCFGMVMGNLGATSHGACIVIPAPSFEPAATLEAVQRERCTSLYGVPTMFIAELNLPDFASYDLTSLRTGIMAGSPCPVEVMKRVVAEMHMEQVSICYGMTETSPVSLQTRMDDDLEHRTGTVGRVLPHIEVKVVDPVTGVTLPRGEAGELRTRGYSVMLGYWEEPGKTAEAIDPGRWMHTGDLAVMREDGYVEIVGRIKDMIIRGGENIYPREVEEFLYAHPKIADVQVVGVPHERYGEEVLACVVVRDAADPLTLEELRAYCAGQLAHYKVPSRLQLLDSFPMTVSGKVRKVELRERYGTRP

SEQ ID NO: 67 - DNA, 1626 bp, Artificial Sequence
  Synthetic: codon-optimized Nucleotide sequence
atgactgcaccagcacctcaaccctcttatgcacatggcacttctaccactccgcttcttggtgatacggtgggggcaaacctgggtcgtgccatcgcggctcatcccgatcgtgaggcactggtcgatgtacccagcggacgccgttggacttacgcagagtttggcgcggccgtagatgaattagcacgcggcctgttagccaaaggggtaactcgcggtgaccgtgtgggtatttgggctgtgaactgtcccgaatgggttttggtgcaatacgctacagcccgtattggggtaatcatggttaatgtaaatcccgcttatcgcgcccacgagcttgaatatgtactgcaacagagtggcatttccttattagtggcttcacttgcacacaaaagttcagattaccgcgcaattgtggagcaagtgcgcggccgctgtcccgccttacgtgaaactgtgtacatcggtgatccatcatgggatgccttgactgcaggcgcagcggctgtcgaacaagatcgtgttgacgctctggcggcggagctttcatgtgacgaccctgtcaacattcagtacactagcggtacgactggttttccgaaaggagcaacattatctcaccataacatcttgaacaacggttattgggtagggcgcacagtcggctacactgagcaagaccgtgtctgcttaccagtcccgttctatcattgctttgggatggtgatgggaaatcttggagccacatcccatggggcctgtattgtgatcccggccccctccttcgagcctgccgcgactttagaagctgttcagcgcgaacgttgtacaagcctgtacggcgttcccacaatgtttattgcggagcttaacctgccggactttgcctcatacgatttgacgagcctgcgcactggcatcatggcagggtcgccctgcccagtagaagtcatgaagcgtgtcgttgctgagatgcatatggagcaggtctcgatttgttatggtatgacggagaccagtcccgtgagtcttcaaactcgcatggacgacgacttagaacaccgtacaggtacggtcggtcgtgtacttccgcacattgaagtcaaagtagtggaccccgtgacaggtgtaacccttccccgcggggaggcaggggagcttcgcactcgtggatacagcgtaatgctgggttattgggaggaacctggcaagacggctgaggctatcgatccgggtcgttggatgcacacaggcgatcttgcggtgatgcgtgaagatgggtatgttgagattgttgggcgcatcaaggacatgattattcgcggcggtgaaaacatttatcctcgcgaggttgaagaatttttatatgcacacccaaagatcgcagacgtacaggtagtcggcgtgccacatgagcgttatggagaagaggtactggcgtgcgttgtcgttcgcgacgcggccgacccactgaccctggaagaattacgcgcctactgtgcaggccagcttgctcattataaagtcccttcgcgtttacaacttttggattcgttccctatgaccgtgtcaggaaaggtacgtaaggttgagttacgtgagcgctacgggacacgcccgtga

SEQ ID NO: 68 - protein, 387 aa, Pseudomonas aeruginosa
  liuA
MTYPSLNFALGETIDMLRDQVRGFVAAELQPRAAQIDQDNQFPMDMWRKFGEMGLLGITVDEEYGGSALGYLAHAVVMEEISRASASVALSYGAHSNLCVNQIKRNGNAEQKARYLPALVSGEHIGALAMSEPNAGSDVVSMKLRADRVGDRFVLNGSKMWITNGPDAHTYVIYAKTDADKGAHGITAFIVERDWKGFSRGPKLDKLGMRGSNTCELIFQDVEVPEENVLGAVNGGVKVLMSGLDYERVVLSGGPVGIMQACMDVVVPYIHDRRQFGQSIGEFQLVQGKVADMYTALNASRAYLYAVAAACDRGETTRKDAAGVILYSAERATQMALDAIQILGGNGYINEFPTGRLLRDAKLYEIGAGTSEIRRMLIGRELFNETR

SEQ ID NO: 69 - protein, 535 aa, Pseudomonas aeruginosa
  LiuB
MAILHTQINPRSAEFAANAATMLEQVNALRTLLGRIHEGGGSAAQARHSARGKLLVRERINRLLDPGSPFLELSALAAHEVYGEEVAAAGIVAGIGRVEGVECMIVGNDATVKGGTYYPLTVKKHLRAQAIALENRLPCIYLVDSGGANLPRQDEVFPDREHFGRIFFNQANMSARGIPQIAVVMGSCTAGGAYVPAMSDETVMVREQATIFLAGPPLVKAATGEVVSAEELGGADVHCKVSGVADHYAEDDDHALAIARRCVANLNWRKQGQLQCRAPRAPLYPAEELYGVIPADSKQPYDVREVIARLVDGSEFDEFKALFGTTLVCGFAHLHGYPIAILANNGILFAEAAQKGAHFIELACQRGIPLLFLQNITGFMVGQKYEAGGIAKHGAKLVTAVACARVPKFTVLIGGSFGAGNYGMCGRAYDPRFLWMWPNARIGVMGGEQAAGVLAQVKREQAERAGQQLGVEEEAKIKAPILEQYEHQGHPYYSSARLWDDGVIDPAQTREVLALALSAALNAPIEPTAFGVFRM

SEQ ID NO: 70 - protein, 265 aa, Pseudomonas aeruginosa
  LiuC
MSEFQTIQLEIDPRGVATLWLDRAEKNNAFNAVVIDELLQAIDRVGSDPQVRLLVLRGRGRHFCGGADLAWMQQSVDLDYQGNLADAQRIAELMTHLYNLPKPTLAVVQGAVFGGGVGLVSCCDMAIGSDDATFCLSEVRIGLIPATIAPFVVKAIGQRAARRYSLTAERFDGRRASELGLLSESCPAAELESQAEAWIANLLQNSPRALVACKALYHEVEAAELSPALRRYTEAAIARIRISPEGQEGLRAFLEKRTPAWRNDA

SEQ ID NO: 71 - protein, 655 aa, Pseudomonas aeruginosa
  LiuD
MNPDYRSIQRLLVANRGEIACRVMRSARALGIGSVAVHSDIDRHARHVAEADIAVDLGGAKPADSYLRGDRIIAAALASGAQAIHPGYGFLSENADFARACEEAGLLFLGPPAAAIDAMGSKSAAKALMEEAGVPLVPGYHGEAQDLETFRREAGRIGYPVLLKAAAGGGGKGMKVVEREAELAEALSSAQREAKAAFGDARMLVEKYLLKPRHVEIQVFADRHGHCLYLNERDCSIQRRHQKVVEEAPAPGLGAELRRAMGEAAVRAAQAIGYVGAGTVEFLLDERGQFFFMEMNTRLQVEHPVTEAITGLDLVAWQIRVARGEALPLTQEQVPLNGHAIEVRLYAEDPEGDFLPASGRLMLYREAAAGPGRRVDSGVREGDEVSPFYDPMLAKLIAWGETREEARQRLLAMLAETSVGGLRTNLAFLRRILGHPAFAAAELDTGFIARHQDDLLPAPQALPEHFWQAAAEAWLQSEPGHRRDDDPHSPWSRNDGWRSALARESDLMLRCRDERRCVRLRHASPSQYRLDGDDLVSRVDGVTRRSAALRRGRQLFLEWEGELLAIEAVDPIAEAEAAHAHQGGLSAPMNGSIVRVLVEPGQTVEAGATLVVLEAMKMEHSIRAPHAGVVKALYCSEGELVEEGTPLVELDENQA

SEQ ID NO: 72 - protein, 300 aa, Pseudomonas aeruginosa
  LiuE
MNLPKKVRLVEVGPRDGLQNEKQPIEVADKIRLVDDLSAAGLDYIEVGSFVSPKWVPQMAGSAEVFAGIRQRPGVTYAALAPNLKGFEAALESGVKEVAVFAAASEAFSQRNINCSIKDSLERFVPVLEAARQHQVRVRGYISCVLGCPYDGDVDPRQVAWVARELQQMGCYEVSLGDTIGVGTAGATRRLIEAVASEVPRERLAGHFHDTYGQALANIYASLLEGIAVFDSSVAGLGGCPYAKGATGNVASEDVLYLLNGLEIHTGVDMHALVDAGQRICAVLGKSNGSRAAKALLAKA

SEQ ID NO: 73 - DNA, 6595 bp, Pseudomonas aeruginosa
  liuABCDE codon optimized sequence
atgacttacccgtccctgaattttgcgctgggcgaaaccattgacatgttgcgcgaccaagttcgtggcttcgttgcagcggaactgcaacctcgcgcggctcaaattgaccaggataatcagtttccgatggatatgtggcgtaagttcggtgagatggggctcttaggtattacggttgatgaggaatacggaggtagcgcgctcggttacttagcccatgcggtcgtaatggaagaaatttcccgtgcctctgcgagcgtagcgctgtcttatggtgcgcattcaaacctgtgcgttaaccagatcaaacgcaatggtaacgctgaacagaaagcgcgttatctgccggctttggtgtccggcgaacacattggcgccctcgctatgtcggaacctaacgcagggtcggatgtggtgtctatgaaactgcgcgcggatcgcgttggcgatcgtttcgtgctgaatggttccaaaatgtggatcaccaacgggcctgatgcacatacgtatgtgatctacgctaaaaccgacgcagataaaggggcccatggcatcaccgcatttattgttgagcgtgactggaaagggtttagccgtggcccaaaactggataaactcggtatgcgtggttcaaatacatgtgaactgattttccaagacgtcgaagtccccgaagaaaatgtgctgggtgcagtgaatgggggggtcaaagtgttaatgtctggtctcgattatgaacgtgtagtgctgagcggtggtccggttggtattatgcaagcctgtatggacgtggtagtgccgtacattcatgatcgccgccagttcggccagtcgatcggagaatttcagctggtgcagggtaaggttgcggacatgtataccgctctgaatgcttctcgtgcgtacttgtatgctgtcgctgcagcctgcgatcgtggagaaacgactcgcaaagacgctgctggtgtgattctctacagcgcagaacgtgctacccaaatggcacttgacgcgatccagatcttgggaggcaatgggtatatcaatgagttccccacgggccgcctgctgcgcgatgcgaagctgtatgagatcggcgcgggtacgagcgaaatccgccgtatgttaatcggtcgtgaattatttaacgagactcgctgaagcctcgctcttcccggcccttttccgccagggagagggcattccattgcatcgacaggcgcatcgccaggtcgggagcgggcgccaaccgcttccgcccacctcgacacggagccaccgccatggccatccttcacacgcagattaacccgcgttctgctgaattcgcggcgaatgccgcgaccatgctggagcaagttaacgcattgcgtacgctccttggtcgcatccacgaaggtggtggttcggcggctcaggctcgccattcggcacgtggcaaattgttggttcgcgaacgcatcaaccgcctgctggaccccggtagcccgtttttggagttgagcgcgttagcagctcatgaggtgtatggggaagaagtcgcagcagcaggtatcgtggccgggatcgggcgtgtagaaggagtagaatgtatgatcgttggtaatgatgccactgtgaaaggaggtacgtattacccgctgaccgtgaagaagcatctgcgcgcccaagcaatcgcattagaaaatcgtttgccgtgtatctatctggtcgattcgggtggcgccaatctgcctcgccaggacgaggtctttccggatcgcgagcatttcggccgcatctttttcaaccaagccaatatgagcgcccgcggtatcccgcagattgcggtggtaatgggctcatgtactgcgggtggcgcctatgtcccggccatgtccgatgaaactgtgatggtccgtgagcaggcgacgatcttcctggctggaccgcctctcgtgaaagcggccacgggtgaagtggtttcagcagaggaattgggtggcgccgacgtgcattgtaaagtgtcaggcgtggcggaccactatgccgaagatgatgaccatgcattggcgattgcgcgtcgctgtgttgcgaatttaaattggcgcaaacagggtcagcttcagtgccgtgcgccgcgtgctccgctgtatccggcggaagaactgtatggtgtgattccggcggatagcaaacagccgtatgatgtgcgcgaggtcattgcacgcctggttgatggatctgaatttgatgaattcaaggcgctgttcggaaccaccctggtgtgcggctttgcacacctgcatggctacccaattgccattctcgcaaataatggcattctgttcgcggaggcggcccagaaaggggcccatttcattgaactggcctgccaacgcggtattccattactgttcctgcaaaatatcaccggcttcatggttggtcagaagtatgaagctggcggtattgccaagcatggcgcgaaactggtcaccgcggtcgcctgcgcccgcgtgccgaaatttacagtgctgattggcggaagtttcggggcagggaactacggaatgtgtggtcgcgcgtacgatccgcgcttcctctggatgtggccgaatgcacgcattggcgtgatgggcggcgagcaggctgccggcgtcctggcacaggtcaaacgtgagcaagcggaacgcgctggccaacagctgggggtggaggaagaagcgaaaattaaagcgccgatccttgaacagtatgaacatcagggccatccgtactattcgtcagcacgtttgtgggacgatggcgtcattgatcctgcccagacacgcgaagtccttgcgctggcgctgagtgcggcgcttaacgctccgatcgaaccaactgcattcggtgtatttcgcatgtgacgagtagaccagcatgagcgaatttcagacgatccagctggaaattgatccacgtggagtggcaaccctgtggctggaccgtgctgaaaaaaataacgcatttaacgccgtcgtgatcgatgaactgctgcaggcgatcgaccgcgtaggcagcgacccccaggtccgtttgctggtcttgcgtgggcgtggccgtcatttctgtggcggcgccgacctggcgtggatgcagcagtctgttgacctggattatcagggtaaccttgctgacgcccagcgcatcgcagagctcatgacccacttgtataatctgcccaaacctactttagcggtagttcaaggcgcagttttcggcggcggggtcggtttggtgagctgctgcgacatggcaattggtagtgatgacgccactttttgcttgtcagaggtacgcattgggctgattccagcaaccatcgccccgttcgtggtgaaagctattggtcaacgcgcagcgcgccgttattcactgactgctgaacgttttgatgggcgccgcgcgtccgaactgggactgcttagcgagtcttgcccggccgcagaactggaatcccaagcggaagcatggatcgcgaatcttctccagaactctccacgtgcactcgtggcatgtaaagcgctgtatcacgaggtagaagcggctgaactgtcccctgcactgcgtcgctatacggaagccgcaattgcacgtatccgtatttcaccagaaggtcaagaaggcttgcgtgcctttttagaaaaacgcacaccggcgtggagaaacgacgcatgaacccggactaccgttcaattcagcgtctcttagtagctaaccgtggcgagattgcctgtcgcgtaatgcgttcggcccgcgcgttaggtattggatcagttgcagttcattcggatatcgaccgccacgcacgtcacgtggctgaagctgatattgcggttgacctgggcggcgccaaaccggcagattcgtatctgcgtggcgaccgtatcattgcagctgcactggcttcaggagcccaggccattcatccggggtatggctttctgtctgagaatgctgattttgcccgcgcgtgcgaagaagcaggtttactgtttttgggcccaccggctgcggcaattgatgctatggggtctaagtcagcggcgaaagctttgatggaagaggcgggagtccccctggttccaggttaccacggtgaagcgcaggacttggaaacctttcgtcgcgaggccggacgcatcggctatcccgtgctcttaaaggccgcggccggtggcggcggaaaagggatgaaagtcgtggaacgcgaggccgagctcgcagaagcgctgtccagcgcccaacgcgaagccaaagcggcctttggcgatgcgcgcatgctggtggagaagtatttgttaaaaccgcgtcacgtcgaaattcaggtctttgcagatcgtcatggtcactgtttatacctcaacgaacgtgactgttcgatccaacgtcgccatcaaaaagttgtagaagaagcgccggctcccggtttgggcgcggaactgcgtcgtgccatgggcgaagcggccgttcgcgcagcgcaagcgatcggctatgtgggggcgggcactgtagagtttctcctggacgagcgcggtcaattcttttttatggaaatgaacactcgcctgcaggttgaacaccctgtaactgaggccatcactggtctcgatttagtcgcgtggcagatccgtgtggcgcgtggtgaagcccttccgttgactcaagaacaagtaccgctgaacgggcacgcgatcgaagtccgcctgtacgcggaagaccctgaaggggattttcttccggcaagtggacgcctgatgctgtatcgtgaagccgctgcaggtccgggccgccgcgtggattcgggagtccgtgagggcgacgaagtcagccccttctacgatccgatgctggcaaaattgatcgcatggggggaaacccgtgaggaagctcgccaacgcctgctcgccatgttggccgagacctcggtcgggggcttgcgtacgaacctggcttttttacgtcgtatcttaggccatcccgcttttgccgccgctgaactggataccgggttcattgctcgtcatcaagatgacctgctgccagcaccccaggctctgccagaacacttctggcaagcagcagcagaagcttggctgcaaagcgaacctggtcatcgtcgcgatgacgatccgcattccccttggagccgtaacgatggttggcgctctgctttggcacgcgaatctgatctgatgctgcgctgtcgcgatgaacgccgttgtgtgcgtctgcgccatgcttccccatctcaatatcgtcttgacggtgatgatctggtatcccgtgttgatggcgttacccgccgctccgcagcgttgcgtcgcggccgccagctgttcttagaatgggaaggtgaactgttagcgatcgaagctgttgatccgattgcagaagccgaagcggcgcatgcccatcaaggcggtttgagcgcgccaatgaacgggtctattgtacgcgttctggttgagccggggcaaaccgtagaggcgggtgcgactcttgtggttttagaagcaatgaaaatggagcacagtatccgtgcgccacatgccggcgttgttaaagcgctgtactgttcagaaggagaattagttgaagagggcactcctctggttgaactggacgaaaaccaggcctgacagccaagacgaggaacagcatgaacctgccgaagaaagttcgtctggttgaagttggtccgcgcgatggacttcagaacgaaaaacagccgatcgaagtggctgacaaaattcgccttgttgatgacttgtcggcagccggcttagattatattgaagtgggcagtttcgtctcaccgaaatgggttccgcagatggccgggagcgccgaagtgtttgctggcattcgtcaacgccctggcgtgacctacgcggcactcgccccgaatttgaaaggcttcgaagcagctctggaatcgggtgtaaaagaagttgccgtgttcgcagcagcctccgaagcattctcccaacgcaacatcaactgctcgattaaagactcccttgagcgcttcgtcccggttctggaagcggctcgccaacatcaggtacgcgtccgcggatatatttcctgcgtattgggttgcccgtatgatggcgacgtagatccgcgccaggtcgcatgggtcgcacgtgaactccagcagatgggctgctatgaggtcagtctcggcgatacaatcggtgtgggtaccgcgggcgcgacccgccgtttaattgaggcggtggcatctgaggttccccgcgaacgccttgcaggccactttcatgatacatatggacaggcgctggctaacatctatgcttctttgctggagggcattgctgtcttcgacagttccgtagctggcctcggtggctgcccatatgcaaaaggcgctaccggcaacgtcgcgagtgaggatgtgctgtatcttttaaatggtcttgaaattcataccggtgtggacatgcatgccctggtagacgcgggacagcgcatctgtgcggtgctcggaaagtcgaatggctcccgtgctgcgaaggccctgctggccaaagcttaatga

SEQ ID NO: 74 - skipped

SEQ ID NO: 75 - DNA, 3443 bp, Artificial Sequence
  Synthetic: Tet-kivD-leuDH construct
gaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataagaaggagatatacatatgttcgacatgatggatgcagcccgcctggaaggcctgcacctcgcccaggatccagcgacgggcctgaaagcgatcatcgcgatccattccactcgcctcggcccggccttaggcggctgtcgttacctcccatatccgaatgatgaagcggccatcggcgatgccattcgcctggcgcagggcatgtcctacaaagctgcacttgcgggtctggaacaaggtggtggcaaggcggtgatcattcgcccaccccacttggataatcgcggtgccttgtttgaagcgtttggacgctttattgaaagcctgggtggccgttatatcaccgccgttgactcaggaacaagtagtgccgatatggattgcatcgcccaacagacgcgccatgtgacttcaacgacacaagccggcgacccatctccacatacggctctgggcgtctttgccggcatccgcgcctccgcgcaggctcgcctggggtccgatgacctggaaggcctgcgtgtcgcggttcagggccttggccacgtaggttatgcgttagcggagcagctggcggcggtcggcgcagaactgctggtgtgcgacctggaccccggccgcgtccagttagcggtggagcaactgggggcgcacccactggcccctgaagcattgctctctactccgtgcgacattttagcgccttgtggcctgggcggcgtgctcaccagccagtcggtgtcacagttgcgctgcgcggccgttgcaggcgcagcgaacaatcaactggagcgcccggaagttgcagacgaactggaggcgcgcgggattttatatgcgcccgattacgtgattaactcgggaggactgatttatgtggcgctgaagcatcgcggtgctgatccgcatagcattaccgcccacctcgctcgcatccctgcacgcctgacggaaatctatgcgcatgcgcaggcggatcatcagtcgcctgcgcgcatcgccgatcgtctggcggagcgcattctgtacggcccgcagtga

SEQ ID NO: 76 - DNA, 3467 bp, Artificial Sequence
  Synthetic: Tet-kivD-adh2 construct
gaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataataagaaggagatatacatatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataa

SEQ ID NO: 77 - skipped

SEQ ID NO: 78 - DNA, 4530 bp, Artificial Sequence
  Synthetic: Tet-leuDH-kivD-adh2 construct
gaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgttcgacatgatggatgcagcccgcctggaaggcctgcacctcgcccaggatccagcgacgggcctgaaagcgatcatcgcgatccattccactcgcctcggcccggccttaggcggctgtcgttacctcccatatccgaatgatgaagcggccatcggcgatgccattcgcctggcgcagggcatgtcctacaaagctgcacttgcgggtctggaacaaggtggtggcaaggcggtgatcattcgcccaccccacttggataatcgcggtgccttgtttgaagcgtttggacgctttattgaaagcctgggtggccgttatatcaccgccgttgactcaggaacaagtagtgccgatatggattgcatcgcccaacagacgcgccatgtgacttcaacgacacaagccggcgacccatctccacatacggctctgggcgtctttgccggcatccgcgcctccgcgcaggctcgcctggggtccgatgacctggaaggcctgcgtgtcgcggttcagggccttggccacgtaggttatgcgttagcggagcagctggcggcggtcggcgcagaactgctggtgtgcgacctggaccccggccgcgtccagttagcggtggagcaactgggggcgcacccactggcccctgaagcattgctctctactccgtgcgacattttagcgccttgtggcctgggcggcgtgctcaccagccagtcggtgtcacagttgcgctgcgcggccgttgcaggcgcagcgaacaatcaactggagcgcccggaagttgcagacgaactggaggcgcgcgggattttatatgcgcccgattacgtgattaactcgggaggactgatttatgtggcgctgaagcatcgcggtgctgatccgcatagcattaccgcccacctcgctcgcatccctgcacgcctgacggaaatctatgcgcatgcgcaggcggatcatcagtcgcctgcgcgcatcgccgatcgtctggcggagcgcattctgtacggcccgcagtgataagaaggagatatacatatgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataataagaaggagatatacatatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataatacgcatggcatggatgaa

SEQ ID NO: 79 - DNA, 4434 bp, Artificial Sequence
  Synthetic: Tet-ilvE-kivD-adh2 construct
gaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgaccacgaagaaagctgattacatttggttcaatggggagatggttcgctgggaagacgcgaaggtgcatgtgatgtcgcacgcgctgcactatggcacctcggtttttgaaggcatccgttgctacgactcgcacaaaggaccggttgtattccgccatcgtgagcatatgcagcgtctgcatgactccgccaaaatctatcgcttcccggtttcgcagagcattgatgagctgatggaagcttgtcgtgacgtgatccgcaaaaacaatctcaccagcgcctatatccgtccgctgatcttcgttggtgatgttggcatgggcgtaaacccgccagcgggatactcaaccgacgtgattatcgccgctttcccgtggggagcgtatctgggcgcagaagcgctggagcaggggatcgatgcgatggtttcctcctggaaccgcgcagcaccaaacaccatcccgacggcggcaaaagccggtggtaactacctctcttccctgctggtgggtagcgaagcgcgccgccacggttatcaggaaggtatcgcgttggatgtgaatggttacatctctgaaggcgcaggcgaaaacctgtttgaagtgaaagacggcgtgctgttcaccccaccgttcacctcatccgcgctgccgggtattacccgtgatgccatcatcaaactggcaaaagagctgggaattgaagtgcgtgagcaggtgctgtcgcgcgaatccctgtacctggcggatgaagtgtttatgtccggtacggcggcagaaatcacgccagtgcgcagcgtagacggtattcaggttggcgaaggccgttgtggcccggttaccaaacgcattcagcaagccttcttcggcctcttcactggcgaaaccgaagataaatggggctggttagatcaagttaatcaataataagaaggagatatacatatgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataataagaaggagatatacatatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataatacgcatggcatggatgaa

SEQ ID NO: 80 - DNA, 117 bp, Artificial Sequence
  Synthetic: FNR-responsive regulatory region Sequence
atccccatcactcttgatggagatcaattccccaagctgctagagcgttaccttgcccttaaacattagcaatgtcgatttatcagagggccgacaggctcccacaggagaaaaccg

SEQ ID NO: 81 - DNA, 108 bp, Artificial Sequence
  Synthetic: FNR-responsive regulatory region Sequence
ctcttgatcgttatcaattcccacgctgtttcagagcgttaccttgcccttaaacattagcaatgtcgatttatcagagggccgacaggctcccacaggagaaaaccg

SEQ ID NO: 82 - DNA, 290 bp, Artificial Sequence; identical to SEQ ID NO: 14
  Synthetic: FNR-responsive regulatory region Sequence: nirB1

SEQ ID NO: 83 - DNA, 433 bp, Artificial Sequence
  Synthetic: FNR-responsive regulatory region Sequence: nirB2
cggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaaatgtttgtttaactttaagaaggagatatacat

SEQ ID NO: 84 - DNA, 290 bp, Artificial Sequence
  Synthetic: FNR-responsive regulatory region Sequence: nirB3
gtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaa

SEQ ID NO: 85 - DNA, 173 bp, Artificial Sequence; identical to SEQ ID NO: 15
  Synthetic: FNR-responsive regulatory region Sequence: ydfZ

SEQ ID NO: 86 - DNA, 305 bp, Artificial Sequence; identical to SEQ ID NO: 16
  Synthetic: FNR-responsive regulatory region Sequence: nirB+RBS

SEQ ID NO: 87 - DNA, 180 bp, Artificial Sequence; identical to SEQ ID NO: 17
  Synthetic: FNR-responsive regulatory region Sequence: ydfZ+RBS

SEQ ID NO: 88 - DNA, 199 bp, Artificial Sequence; identical to SEQ ID NO: 18
  Synthetic: FNR-responsive regulatory region Sequence: fnrS1

SEQ ID NO: 89 - DNA, 207 bp, Artificial Sequence
  Synthetic: FNR-responsive regulatory region Sequence: fnrS2
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacat

SEQ ID NO: 90 - DNA, 390 bp, Artificial Sequence
  Synthetic: FNR-responsive regulatory region Sequence: nirB+crp
tcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtagaaatgtgatctagttcacatttgcggtaatagaaaagaaatcgaggcaaaaatgtttgtttaactttaagaaggagatatacat

SEQ ID NO: 91 - DNA, 4837 bp, Escherichia coli
  livKHMGF operon
atgaaacggaatgcgaaaactatcatcgcagggatgattgcactggcaatttcacacaccgctatggctgacgatattaaagtcgccgttgtcggcgcgatgtccggcccgattgcccagtggggcgatatggaatttaacggcgcgcgtcaggcaattaaagacattaatgccaaagggggaattaagggcgataaactggttggcgtggaatatgacgacgcatgcgacccgaaacaagccgttgcggtcgccaacaaaatcgttaatgacggcattaaatacgttattggtcatctgtgttcttcttctacccagcctgcgtcagatatctatgaagacgaaggtattctgatgatctcgccgggagcgaccaacccggagctgacccaacgcggttatcaacacattatgcgtactgccgggctggactcttcccaggggccaacggcggcaaaatacattcttgagacggtgaagccccagcgcatcgccatcattcacgacaaacaacagtatggcgaagggctggcgcgttcggtgcaggacgggctgaaagcggctaacgccaacgtcgtcttcttcgacggtattaccgccggggagaaagatttctccgcgctgatcgcccgcctgaaaaaagaaaacatcgacttcgtttactacggcggttactacccggaaatggggcagatgctgcgccaggcccgttccgttggcctgaaaacccagtttatggggccggaaggtgtgggtaatgcgtcgttgtcgaacattgccggtgatgccgccgaaggcatgttggtcactatgccaaaacgctatgaccaggatccggcaaaccagggcatcgttgatgcgctgaaagcagacaagaaagatccgtccgggccttatgtctggatcacctacgcggcggtgcaatctctggcgactgcccttgagcgtaccggcagcgatgagccgctggcgctggtgaaagatttaaaagctaacggtgcaaacaccgtgattgggccgctgaactgggatgaaaaaggcgatcttaagggatttgattttggtgtcttccagtggcacgccgacggttcatccacggcagccaagtgatcatcccaccgcccgtaaaatgcgggcgggtttagaaaggttaccttatgtctgagcagtttttgtatttcttgcagcagatgtttaacggcgtcacgctgggcagtacctacgcgctgatagccatcggctacaccatggtttacggcattatcggcatgatcaacttcgcccacggcgaggtttatatgattggcagctacgtctcatttatgatcatcgccgcgctgatgatgatgggcattgataccggctggctgctggtagctgcgggattcgtcggcgcaatcgtcattgccagcgcctacggctggagtatcgaacgggtggcttaccgcccggtgcgtaactctaagcgcctgattgcactcatctctgcaatcggtatgtccatcttcctgcaaaactacgtcagcctgaccgaaggttcgcgcgacgtggcgctgccgagcctgtttaacggtcagtgggtggtggggcatagcgaaaacttctctgcctctattaccaccatgcaggcggtgatctggattgttaccttcctcgccatgctggcgctgacgattttcattcgctattcccgcatgggtcgcgcgtgtcgtgcctgcgcggaagatctgaaaatggcgagtctgcttggcattaacaccgaccgggtgattgcgctgacctttgtgattggcgcggcgatggcggcggtggcgggtgtgctgctcggtcagttctacggcgtcattaacccctacatcggctttatggccgggatgaaagcctttaccgcggcggtgctcggtgggattggcagcattccgggagcgatgattggcggcctgattctggggattgcggaggcgctctcttctgcctatctgagtacggaatataaagatgtggtgtcattcgccctgctgattctggtgctgctggtgatgccgaccggtattctgggtcgcccggaggtagagaaagtatgaaaccgatgcatattgcaatggcgctgctctctgccgcgatgttctttgtgctggcgggcgtctttatgggcgtgcaactggagctggatggcaccaaactggtggtcgacacggcttcggatgtccgttggcagtgggtgtttatcggcacggcggtggtctttttcttccagcttttgcgaccggctttccagaaagggttgaaaagcgtttccggaccgaagtttattctgcccgccattgatggctccacggtgaagcagaaactgttcctcgtggcgctgttggtgcttgcggtggcgtggccgtttatggtttcacgcgggacggtggatattgccaccctgaccatgatctacattatcctcggtctggggctgaacgtggttgttggtctttctggtctgctggtgctggggtacggcggtttttacgccatcggcgcttacacttttgcgctgctcaatcactattacggcttgggcttctggacctgcctgccgattgctggattaatggcagcggcggcgggcttcctgctcggttttccggtgctgcgtttgcgcggtgactatctggcgatcgttaccctcggtttcggcgaaattgtgcgcatattgctgctcaataacaccgaaattaccggcggcccgaacggaatcagtcagatcccgaaaccgacactcttcggactcgagttcagccgtaccgctcgtgaaggcggctgggacacgttcagtaatttctttggcctgaaatacgatccctccgatcgtgtcatcttcctctacctggtggcgttgctgctggtggtgctaagcctgtttgtcattaaccgcctgctgcggatgccgctggggcgtgcgtgggaagcgttgcgtgaagatgaaatcgcctgccgttcgctgggcttaagcccgcgtcgtatcaagctgactgcctttaccataagtgccgcgtttgccggttttgccggaacgctgtttgcggcgcgtcagggctttgtcagcccggaatccttcacctttgccgaatcggcgtttgtgctggcgatagtggtgctcggcggtatgggctcgcaatttgcggtgattctggcggcaattttgctggtggtgtcgcgcgagttgatgcgtgatttcaacgaatacagcatgttaatgctcggtggtttgatggtgctgatgatgatctggcgtccgcagggcttgctgcccatgacgcgcccgcaactgaagctgaaaaacggcgcagcgaaaggagagcaggcatgagtcagccattattatctgttaacggcctgatgatgcgcttcggcggcctgctggcggtgaacaacgtcaatcttgaactgtacccgcaggagatcgtctcgttaatcggccctaacggtgccggaaaaaccacggtttttaactgtctgaccggattctacaaacccaccggcggcaccattttactgcgcgatcagcacctggaaggtttaccggggcagcaaattgcccgcatgggcgtggtgcgcaccttccagcatgtgcgtctgttccgtgaaatgacggtaattgaaaacctgctggtggcgcagcatcagcaactgaaaaccgggctgttctctggcctgttgaaaacgccatccttccgtcgcgcccagagcgaagcgctcgaccgcgccgcgacctggcttgagcgcattggtttgctggaacacgccaaccgtcaggcgagtaacctggcctatggtgaccagcgccgtcttgagattgcccgctgcatggtgacgcagccggagattttaatgctcgacgaacctgcggcaggtcttaacccgaaagagacgaaagagctggatgagctgattgccgaactgcgcaatcatcacaacaccactatcttgttgattgaacacgatatgaagctggtgatgggaatttcggaccgaatttacgtggtcaatcaggggacgccgctggcaaacggtacgccggagcagatccgtaataacccggacgtgatccgtgcctatttaggtgaggcataagatggaaaaagtcatgttgtcctttgacaaagtcagcgcccactacggcaaaatccaggcgctgcatgaggtgagcctgcatatcaatcagggcgagattgtcacgctgattggcgcgaacggggcggggaaaaccaccttgctcggcacgttatgcggcgatccgcgtgccaccagcgggcgaattgtgtttgatgataaagacattaccgactggcagacagcgaaaatcatgcgcgaagcggtggcgattgtcccggaagggcgtcgcgtcttctcgcggatgacggtggaagagaacctggcgatgggcggtttttttgctgaacgcgaccagttccaggagcgcataaagtgggtgtatgagctgtttccacgtctgcatgagcgccgtattcagcgggcgggcaccatgtccggcggtgaacagcagatgctggcgattggtcgtgcgctgatgagcaacccgcgtttgctactgcttgatgagccatcgctcggtcttgcgccgattatcatccagcaaattttcgacaccatcgagcagctgcgcgagcaggggatgactatctttctcgtcgagcagaacgccaaccaggcgctaaagctggcggatcgcggctacgtgctggaaaacggccatgtagtgctttccgatactggtgatgcgctgctggcgaatgaagcggtgagaagtgcgtatttaggcgggtaa

SEQ ID NO: 92 - protein, 369 aa, Escherichia coli
  LivK
MKRNAKTIIAGMIALAISHTAMADDIKVAVVGAMSGPIAQWGDMEFNGARQAIKDINAKGGIKGDKLVGVEYDDACDPKQAVAVANKIVNDGIKYVIGHLCSSSTQPASDIYEDEGILMISPGATNPELTQRGYQHIMRTAGLDSSQGPTAAKYILETVKPQRIAIIHDKQQYGEGLARSVQDGLKAANANVVFFDGITAGEKDFSALIARLKKENIDFVYYGGYYPEMGQMLRQARSVGLKTQFMGPEGVGNASLSNIAGDAAEGMLVTMPKRYDQDPANQGIVDALKADKKDPSGPYVWITYAAVQSLATALERTGSDEPLALVKDLKANGANTVIGPLNWDEKGDLKGFDFGVFQWHADGSSTAAK

SEQ ID NO: 93 - DNA, 1110 bp, Escherichia coli
  LivK
atgaaacggaatgcgaaaactatcatcgcagggatgattgcactggcaatttcacacaccgctatggctgacgatattaaagtcgccgttgtcggcgcgatgtccggcccgattgcccagtggggcgatatggaatttaacggcgcgcgtcaggcaattaaagacattaatgccaaagggggaattaagggcgataaactggttggcgtggaatatgacgacgcatgcgacccgaaacaagccgttgcggtcgccaacaaaatcgttaatgacggcattaaatacgttattggtcatctgtgttcttcttctacccagcctgcgtcagatatctatgaagacgaaggtattctgatgatctcgccgggagcgaccaacccggagctgacccaacgcggttatcaacacattatgcgtactgccgggctggactcttcccaggggccaacggcggcaaaatacattcttgagacggtgaagccccagcgcatcgccatcattcacgacaaacaacagtatggcgaagggctggcgcgttcggtgcaggacgggctgaaagcggctaacgccaacgtcgtcttcttcgacggtattaccgccggggagaaagatttctccgcgctgatcgcccgcctgaaaaaagaaaacatcgacttcgtttactacggcggttactacccggaaatggggcagatgctgcgccaggcccgttccgttggcctgaaaacccagtttatggggccggaaggtgtgggtaatgcgtcgttgtcgaacattgccggtgatgccgccgaaggcatgttggtcactatgccaaaacgctatgaccaggatccggcaaaccagggcatcgttgatgcgctgaaagcagacaagaaagatccgtccgggccttatgtctggatcacctacgcggcggtgcaatctctggcgactgcccttgagcgtaccggcagcgatgagccgctggcgctggtgaaagatttaaaagctaacggtgcaaacaccgtgattgggccgctgaactgggatgaaaaaggcgatcttaagggatttgattttggtgtcttccagtggcacgccgacggttcatccacggcagccaagtga

SEQ ID NO: 94 - protein, 308 aa, Escherichia coli
  LivH
MSEQFLYFLQQMFNGVTLGSTYALIAIGYTMVYGIIGMINFAHGEVYMIGSYVSFMIIAALMMMGIDTGWLLVAAGFVGAIVIASAYGWSIERVAYRPVRNSKRLIALISAIGMSIFLQNYVSLTEGSRDVALPSLFNGQWVVGHSENFSASITTMQAVIWIVTFLAMLALTIFIRYSRMGRACRACAEDLKMASLLGINTDRVIALTFVIGAAMAAVAGVLLGQFYGVINPYIGFMAGMKAFTAAVLGGIGSIPGAMIGGLILGIAEALSSAYLSTEYKDVVSFALLILVLLVMPTGILGRPEVEKV

SEQ ID NO: 95 - DNA, 927 bp, Escherichia coli
  LivH
atgtctgagcagtttttgtatttcttgcagcagatgtttaacggcgtcacgctgggcagtacctacgcgctgatagccatcggctacaccatggtttacggcattatcggcatgatcaacttcgcccacggcgaggtttatatgattggcagctacgtctcatttatgatcatcgccgcgctgatgatgatgggcattgataccggctggctgctggtagctgcgggattcgtcggcgcaatcgtcattgccagcgcctacggctggagtatcgaacgggtggcttaccgcccggtgcgtaactctaagcgcctgattgcactcatctctgcaatcggtatgtccatcttcctgcaaaactacgtcagcctgaccgaaggttcgcgcgacgtggcgctgccgagcctgtttaacggtcagtgggtggtggggcatagcgaaaacttctctgcctctattaccaccatgcaggcggtgatctggattgttaccttcctcgccatgctggcgctgacgattttcattcgctattcccgcatgggtcgcgcgtgtcgtgcctgcgcggaagatctgaaaatggcgagtctgcttggcattaacaccgaccgggtgattgcgctgacctttgtgattggcgcggcgatggcggcggtggcgggtgtgctgctcggtcagttctacggcgtcattaacccctacatcggctttatggccgggatgaaagcctttaccgcggcggtgctcggtgggattggcagcattccgggagcgatgattggcggcctgattctggggattgcggaggcgctctcttctgcctatctgagtacggaatataaagatgtggtgtcattcgccctgctgattctggtgctgctggtgatgccgaccggtattctgggtcgcccggaggtagagaaagtatga

SEQ ID NO: 96 - protein, 425 aa, Escherichia coli
  LivM
MKPMHIAMALLSAAMFFVLAGVFMGVQLELDGTKLVVDTASDVRWQWVFIGTAVVFFFQLLRPAFQKGLKSVSGPKFILPAIDGSTVKQKLFLVALLVLAVAWPFMVSRGTVDIATLTMIYIILGLGLNVVVGLSGLLVLGYGGFYAIGAYTFALLNHYYGLGFWTCLPIAGLMAAAAGFLLGFPVLRLRGDYLAIVTLGFGEIVRILLLNNTEITGGPNGISQIPKPTLFGLEFSRTAREGGWDTFSNFFGLKYDPSDRVIFLYLVALLLVVLSLFVINRLLRMPLGRAWEALREDEIACRSLGLSPRRIKLTAFTISAAFAGFAGTLFAARQGFVSPESFTFAESAFVLAIVVLGGMGSQFAVILAAILLVVSRELMRDFNEYSMLMLGGLMVLMMIWRPQGLLPMTRPQLKLKNGAAKGEQA

SEQ ID NO: 97 - DNA, 1278 bp, Escherichia coli
  LivM
atgaaaccgatgcatattgcaatggcgctgctctctgccgcgatgttctttgtgctggcgggcgtctttatgggcgtgcaactggagctggatggcaccaaactggtggtcgacacggcttcggatgtccgttggcagtgggtgtttatcggcacggcggtggtctttttcttccagcttttgcgaccggctttccagaaagggttgaaaagcgtttccggaccgaagtttattctgcccgccattgatggctccacggtgaagcagaaactgttcctcgtggcgctgttggtgcttgcggtggcgtggccgtttatggtttcacgcgggacggtggatattgccaccctgaccatgatctacattatcctcggtctggggctgaacgtggttgttggtctttctggtctgctggtgctggggtacggcggtttttacgccatcggcgcttacacttttgcgctgctcaatcactattacggcttgggcttctggacctgcctgccgattgctggattaatggcagcggcggcgggcttcctgctcggttttccggtgctgcgtttgcgcggtgactatctggcgatcgttaccctcggtttcggcgaaattgtgcgcatattgctgctcaataacaccgaaattaccggcggcccgaacggaatcagtcagatcccgaaaccgacactcttcggactcgagttcagccgtaccgctcgtgaaggcggctgggacacgttcagtaatttctttggcctgaaatacgatccctccgatcgtgtcatcttcctctacctggtggcgttgctgctggtggtgctaagcctgtttgtcattaaccgcctgctgcggatgccgctggggcgtgcgtgggaagcgttgcgtgaagatgaaatcgcctgccgttcgctgggcttaagcccgcgtcgtatcaagctgactgcctttaccataagtgccgcgtttgccggttttgccggaacgctgtttgcggcgcgtcagggctttgtcagcccggaatccttcacctttgccgaatcggcgtttgtgctggcgatagtggtgctcggcggtatgggctcgcaatttgcggtgattctggcggcaattttgctggtggtgtcgcgcgagttgatgcgtgatttcaacgaatacagcatgttaatgctcggtggtttgatggtgctgatgatgatctggcgtccgcagggcttgctgcccatgacgcgcccgcaactgaagctgaaaaacggcgcagcgaaaggagagcaggcatga

SEQ ID NO: 98 - protein, 255 aa, Escherichia coli
  LivG
MSQPLLSVNGLMMRFGGLLAVNNVNLELYPQEIVSLIGPNGAGKTTVFNCLTGFYKPTGGTILLRDQHLEGLPGQQIARMGVVRTFQHVRLFREMTVIENLLVAQHQQLKTGLFSGLLKTPSFRRAQSEALDRAATWLERIGLLEHANRQASNLAYGDQRRLEIARCMVTQPEILMLDEPAAGLNPKETKELDELIAELRNHHNTTILLIEHDMKLVMGISDRIYVVNQGTPLANGTPEQIRNNPDVIRAYLGEA

SEQ ID NO: 99 - skipped

SEQ ID NO: 100 - DNA, 768 bp, Escherichia coli
  LivG
atgagtcagccattattatctgttaacggcctgatgatgcgcttcggcggcctgctggcggtgaacaacgtcaatcttgaactgtacccgcaggagatcgtctcgttaatcggccctaacggtgccggaaaaaccacggtttttaactgtctgaccggattctacaaacccaccggcggcaccattttactgcgcgatcagcacctggaaggtttaccggggcagcaaattgcccgcatgggcgtggtgcgcaccttccagcatgtgcgtctgttccgtgaaatgacggtaattgaaaacctgctggtggcgcagcatcagcaactgaaaaccgggctgttctctggcctgttgaaaacgccatccttccgtcgcgcccagagcgaagcgctcgaccgcgccgcgacctggcttgagcgcattggtttgctggaacacgccaaccgtcaggcgagtaacctggcctatggtgaccagcgccgtcttgagattgcccgctgcatggtgacgcagccggagattttaatgctcgacgaacctgcggcaggtcttaacccgaaagagacgaaagagctggatgagctgattgccgaactgcgcaatcatcacaacaccactatcttgttgattgaacacgatatgaagctggtgatgggaatttcggaccgaatttacgtggtcaatcaggggacgccgctggcaaacggtacgccggagcagatccgtaataacccggacgtgatccgtgcctatttaggtgaggcataa

SEQ ID NO: 101 - protein, 237 aa, Escherichia coli [sequence omitted]

SEQ ID NO: 102 - DNA, 714 bp, Escherichia coli
  LivF
atggaaaaagtcatgttgtcctttgacaaagtcagcgcccactacggcaaaatccaggcgctgcatgaggtgagcctgcatatcaatcagggcgagattgtcacgctgattggcgcgaacggggcggggaaaaccaccttgctcggcacgttatgcggcgatccgcgtgccaccagcgggcgaattgtgtttgatgataaagacattaccgactggcagacagcgaaaatcatgcgcgaagcggtggcgattgtcccggaagggcgtcgcgtcttctcgcggatgacggtggaagagaacctggcgatgggcggtttttttgctgaacgcgaccagttccaggagcgcataaagtgggtgtatgagctgtttccacgtctgcatgagcgccgtattcagcgggcgggcaccatgtccggcggtgaacagcagatgctggcgattggtcgtgcgctgatgagcaacccgcgtttgctactgcttgatgagccatcgctcggtcttgcgccgattatcatccagcaaattttcgacaccatcgagcagctgcgcgagcaggggatgactatctttctcgtcgagcagaacgccaaccaggcgctaaagctggcggatcgcggctacgtgctggaaaacggccatgtagtgctttccgatactggtgatgcgctgctggcgaatgaagcggtgagaagtgcgtatttaggcgggtaa

SEQ ID NO: 103 - DNA, 305 bp, Artificial Sequence
  Synthetic: Arabinose Promoter region
cagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccatacctctagaaataattttgtttaactttaagaaggagatatacat

SEQ ID NO: 104 - DNA, 897 bp, Artificial Sequence
  Synthetic: AraC
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcat

SEQ ID NO: 105 - protein, 298 aa, Artificial Sequence
  Synthetic: AraC polypeptide
MQYGQLVSSLNGGSMKSMAEAQNDPLLPGYSFNAHLVAGLTPIEANGYLDFFIDRPLGMKGYILNLTIRGQGVVKNQGREFVCRPGDILLFPPGEIHHYGRHPEAHEWYHQWVYFRPRAYWHEWLNWPSIFANTGFFRPDEAHQPHFSDLFGQIINAGQGEGRYSELLAINLLEQLLLRRMEAINESLHPPMDNRVREACQYISDHLADSNFDIASVAQHVCLSPSRLSHLFRQQLGISVLSWREDQRISQAKLLLSTTRMPIATVGRNVGFDDQLYFSRVFKKCTGASPSEFRAGCE

SEQ ID NO: 106 - DNA, 280 bp, Artificial Sequence
  Synthetic: Region comprising rhamnose inducible promoter
cggtgagcatcacatcaccacaattcagcaaattgtgaacatcatcacgttcatctttccctggttgccaatggcccattttcctgtcagtaacgagaaggtcgcgaatcaggcgctttttagactggtcgtaatgaaattcagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 107 - DNA, 326 bp, Artificial Sequence
  Synthetic: Lac Promoter region
attcaccaccctgaattgactctcttccgggcgctatcatgccataccgcgaaaggttttgcgccattcgatggcgcgccgcttcgtcaggccacatagctttcttgttctgatcggaacgatcgttggctgtgttgacaattaatcatcggctcgtataatgtgtggaattgtgagcgctcacaattagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 108 - DNA, 22 bp, Artificial Sequence
  Synthetic: LacO
ggaattgtgagcgctcacaatt

SEQ ID NO: 109 - DNA, 1083 bp, Artificial Sequence
  Synthetic: LacI
tcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcat

SEQ ID NO: 110 - protein, 360 aa, Artificial Sequence
  Synthetic: LacI polypeptide sequence
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 111 - DNA, 748 bp, Artificial Sequence
  Synthetic: TetR-tet promoter construct
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacat

SEQ ID NO: 112 - DNA, 222 bp, Artificial Sequence
  Synthetic: Region comprising Temperature sensitive promoter
acgttaaatctatcaccgcaagggataaatatctaacaccgtgcgtgttgactattttacctctggcggtgataatggttgcatagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 113 - DNA, 714 bp, Artificial Sequence
  Synthetic: mutant cI857 repressor
tcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcat

SEQ ID NO: 114 - DNA, 43 bp, Artificial Sequence
  Synthetic: RBS and leader region
ctctagaaataattttgtttaactttaagaaggagatatacat

SEQ ID NO: 115 - protein, 237 aa, Artificial Sequence
  Synthetic: mutant cI857 repressor polypeptide sequence
MSTKKKPLTQEQLEDARRLKAIYEKKKNELGLSQESVADKMGMGQSGVGALFNGINALNAYNAALLTKILKVSVEEFSPSIAREIYEMYEAVSMQPSLRSEYEYPVFSHVQAGMFSPKLRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 116 - DNA, 225 bp, Artificial Sequence
  Synthetic: PssB promoter
tcacctttcccggattaaacgcttttttgcccggtggcatggtgctaccggcgatcacaaacggttaattatgacacaaattgacctgaatgaatatacagtattggaatgcattacccggagtgttgtgtaacaatgtctggccaggtttgtttcccggaaccgaggtcacaacatagtaaaagcgctattggtaatggtacaatcgcgcgtttacacttattc

SEQ ID NO: 117 - DNA, 207 bp, Artificial Sequence; identical to SEQ ID NO: 89
  Synthetic: FNR promoter with RBS and leader region

SEQ ID NO: 118 - DNA, 14 bp, Artificial Sequence
  Synthetic: FNR binding site
ttgagcgaagtcaa

SEQ ID NO: 119 - DNA, 164 bp, Artificial Sequence
  Synthetic: FNR promoter without RBS and leader region
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaa

SEQ ID NO: 120 - DNA, 43 bp, Artificial Sequence; identical to SEQ ID NO: 114
  Synthetic: RBS and leader region

SEQ ID NO: 121 - DNA, 5169 bp, Artificial Sequence
  Synthetic: LeuDH-kivD-adh2-brnQ construct
atgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgtctattccagaaacgcagaaagccatcatattttatgaatcgaacggaaaacttgagcacaaggacatccccgtcccgaagccaaaacctaatgagttgcttatcaacgttaagtattcgggcgtatgccacacagacttgcacgcatggcacggggattggcccttaccgactaagttgccgttagtgggcggacatgagggggcgggagtcgtagtgggaatgggagagaacgtgaagggttggaagattggagattatgctgggattaagtggttgaatgggagctgcatggcctgcgaatattgtgaacttggaaatgagagcaattgcccacatgctgacttgtccggttacacacatgacggttcattccaggaatatgctacggctgatgcagtccaagcagcgcatatcccgcaagggacggacttagcagaagtagcgcccattctttgcgctgggatcaccgtatataaagcgttaaagagcgcaaatttacgggccggacattgggcggcgatcagcggggccgcaggggggctgggcagcttggccgtccagtacgctaaagctatgggttatcgggttttgggcattgacggaggaccgggaaaggaggaattattcacgtccttgggaggagaggtattcattgactttaccaaggaaaaagatatcgtctctgctgtagtaaaggctaccaatggcggtgcccacggaatcataaatgtttcagtttctgaagcggcgatcgaagcgtccactagatattgccgtgcaaatgggacagtcgtacttgtaggacttccggctggcgccaaatgcagctccgatgtatttaatcatgtcgtgaagtcaatctctatcgttggttcatatgtaggaaaccgcgccgatactcgtgaggctcttgacttttttgccagaggcctggttaagtcccccataaaagttgttggcttatccagcttacccgaaatatacgagaagatggagaagggccagatcgcggggagatacgttgttgacacttctaaataataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaa

SEQ ID NO: 122 - DNA, 5532 bp, Artificial Sequence
  Synthetic: Pfnrs-LeuDH-kivD-adh2-brnQ construct (with terminator)
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacatatgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgtctattccagaaacgcagaaagccatcatattttatgaatcgaacggaaaacttgagcacaaggacatccccgtcccgaagccaaaacctaatgagttgcttatcaacgttaagtattcgggcgtatgccacacagacttgcacgcatggcacggggattggcccttaccgactaagttgccgttagtgggcggacatgagggggcgggagtcgtagtgggaatgggagagaacgtgaagggttggaagattggagattatgctgggattaagtggttgaatgggagctgcatggcctgcgaatattgtgaacttggaaatgagagcaattgcccacatgctgacttgtccggttacacacatgacggttcattccaggaatatgctacggctgatgcagtccaagcagcgcatatcccgcaagggacggacttagcagaagtagcgcccattctttgcgctgggatcaccgtatataaagcgttaaagagcgcaaatttacgggccggacattgggcggcgatcagcggggccgcaggggggctgggcagcttggccgtccagtacgctaaagctatgggttatcgggttttgggcattgacggaggaccgggaaaggaggaattattcacgtccttgggaggagaggtattcattgactttaccaaggaaaaagatatcgtctctgctgtagtaaaggctaccaatggcggtgcccacggaatcataaatgtttcagtttctgaagcggcgatcgaagcgtccactagatattgccgtgcaaatgggacagtcgtacttgtaggacttccggctggcgccaaatgcagctccgatgtatttaatcatgtcgtgaagtcaatctctatcgttggttcatatgtaggaaaccgcgccgatactcgtgaggctcttgacttttttgccagaggcctggttaagtcccccataaaagttgttggcttatccagcttacccgaaatatacgagaagatggagaagggccagatcgcggggagatacgttgttgacacttctaaataataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaatacgcatggcatggatgaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaat

SEQ ID NO: 123 - DNA, 6223 bp, Artificial Sequence
  Synthetic: Tet-LeuDH-kivD-adh2-brnQ construct
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgtctattccagaaacgcagaaagccatcatattttatgaatcgaacggaaaacttgagcacaaggacatccccgtcccgaagccaaaacctaatgagttgcttatcaacgttaagtattcgggcgtatgccacacagacttgcacgcatggcacggggattggcccttaccgactaagttgccgttagtgggcggacatgagggggcgggagtcgtagtgggaatgggagagaacgtgaagggttggaagattggagattatgctgggattaagtggttgaatgggagctgcatggcctgcgaatattgtgaacttggaaatgagagcaattgcccacatgctgacttgtccggttacacacatgacggttcattccaggaatatgctacggctgatgcagtccaagcagcgcatatcccgcaagggacggacttagcagaagtagcgcccattctttgcgctgggatcaccgtatataaagcgttaaagagcgcaaatttacgggccggacattgggcggcgatcagcggggccgcaggggggctgggcagcttggccgtccagtacgctaaagctatgggttatcgggttttgggcattgacggaggaccgggaaaggaggaattattcacgtccttgggaggagaggtattcattgactttaccaaggaaaaagatatcgtctctgctgtagtaaaggctaccaatggcggtgcccacggaatcataaatgtttcagtttctgaagcggcgatcgaagcgtccactagatattgccgtgcaaatgggacagtcgtacttgtaggacttccggctggcgccaaatgcagctccgatgtatttaatcatgtcgtgaagtcaatctctatcgttggttcatatgtaggaaaccgcgccgatactcgtgaggctcttgacttttttgccagaggcctggttaagtcccccataaaagttgttggcttatccagcttacccgaaatatacgagaagatggagaagggccagatcgcggggagatacgttgttgacacttctaaataataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaatacgcatggcatggatgaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcgtgc

SEQ ID NO: 124 - DNA, 6676 bp, Artificial Sequence
  Synthetic: Tet-LeuDH-kivD-padA-brnQ construct
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgacagagccgcatgtagcagtattaagccaggtccaacagtttctcgatcgtcaacacggtctttatattgatggtcgtcctggccccgcacaaagtgaaaaacggttggcgatctttgatccggccaccgggcaagaaattgcgtctactgctgatgccaacgaagcggatgtagataacgcagtcatgtctgcctggcgggcctttgtctcgcgtcgctgggccgggcgattacccgcagagcgtgaacgtattctgctacgttttgctgatctggtggagcagcacagtgaggagctggcgcaactggaaaccctggagcaaggcaagtcaattgccatttcccgtgcttttgaagtgggctgtacgctgaactggatgcgttataccgccgggttaacgaccaaaatcgcgggtaaaacgctggacttgtcgattcccttaccccagggggcgcgttatcaggcctggacgcgtaaagagccggttggcgtagtggcgggaattgtgccatggaactttccgttgatgattggtatgtggaaggtgatgccagcactggcagcaggctgttcaatcgtgattaagccttcggaaaccacgccactgacgatgttgcgcgtggcggaactggccagcgaggctggtatccctgatggcgtttttaatgtcgtcaccgggtcaggtgctgtatgcggcgcggccctgacgtcacatcctcatgttgcgaaaatcagttttaccggttcaaccgcgacgggaaaaggtattgccagaactgctgctgatcacttaacgcgtgtaacgctggaactgggcggtaaaaacccggcaattgtattaaaagatgctgatccgcaatgggttattgaaggcttgatgaccggaagcttcctgaatcaagggcaagtatgcgccgccagttcgcgaatttatattgaagcgccgttgtttgacacgctggttagtggatttgagcaggcggtaaaatcgttgcaagtgggaccggggatgtcacctgttgcacagattaaccctttggtttctcgtgcgcactgcgacaaagtgtgttcattcctcgacgatgcgcaggcacagcaagcagagctgattcgcgggtcgaatggaccagccggagaggggtattatgttgcgccaacgctggtggtaaatcccgatgctaaattgcgcttaactcgtgaagaggtgtttggtccggtggtaaacctggtgcgagtagcggatggagaagaggcgttacaactggcaaacgacacggaatatggcttaactgccagtgtctggacgcaaaatctctcccaggctctggaatatagcgatcgcttacaggcagggacggtgtgggtaaacagccataccttaattgacgctaacttaccgtttggtgggatgaagcagtcaggaacgggccgtgattttggccccgactggctggacggttggtgtgaaactaagtcggtgtgtgtacggtattaataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaatacgcatggcatggatgaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcgtgc

SEQ ID NO: 125 - DNA, 5622 bp, Artificial Sequence
  Synthetic: LeuDH-kivD-padA-brnQ
atgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgacagagccgcatgtagcagtattaagccaggtccaacagtttctcgatcgtcaacacggtctttatattgatggtcgtcctggccccgcacaaagtgaaaaacggttggcgatctttgatccggccaccgggcaagaaattgcgtctactgctgatgccaacgaagcggatgtagataacgcagtcatgtctgcctggcgggcctttgtctcgcgtcgctgggccgggcgattacccgcagagcgtgaacgtattctgctacgttttgctgatctggtggagcagcacagtgaggagctggcgcaactggaaaccctggagcaaggcaagtcaattgccatttcccgtgcttttgaagtgggctgtacgctgaactggatgcgttataccgccgggttaacgaccaaaatcgcgggtaaaacgctggacttgtcgattcccttaccccagggggcgcgttatcaggcctggacgcgtaaagagccggttggcgtagtggcgggaattgtgccatggaactttccgttgatgattggtatgtggaaggtgatgccagcactggcagcaggctgttcaatcgtgattaagccttcggaaaccacgccactgacgatgttgcgcgtggcggaactggccagcgaggctggtatccctgatggcgtttttaatgtcgtcaccgggtcaggtgctgtatgcggcgcggccctgacgtcacatcctcatgttgcgaaaatcagttttaccggttcaaccgcgacgggaaaaggtattgccagaactgctgctgatcacttaacgcgtgtaacgctggaactgggcggtaaaaacccggcaattgtattaaaagatgctgatccgcaatgggttattgaaggcttgatgaccggaagcttcctgaatcaagggcaagtatgcgccgccagttcgcgaatttatattgaagcgccgttgtttgacacgctggttagtggatttgagcaggcggtaaaatcgttgcaagtgggaccggggatgtcacctgttgcacagattaaccctttggtttctcgtgcgcactgcgacaaagtgtgttcattcctcgacgatgcgcaggcacagcaagcagagctgattcgcgggtcgaatggaccagccggagaggggtattatgttgcgccaacgctggtggtaaatcccgatgctaaattgcgcttaactcgtgaagaggtgtttggtccggtggtaaacctggtgcgagtagcggatggagaagaggcgttacaactggcaaacgacacggaatatggcttaactgccagtgtctggacgcaaaatctctcccaggctctggaatatagcgatcgcttacaggcagggacggtgtgggtaaacagccataccttaattgacgctaacttaccgtttggtgggatgaagcagtcaggaacgggccgtgattttggccccgactggctggacggttggtgtgaaactaagtcggtgtgtgtacggtattaataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaa

SEQ ID NO: 126 - DNA, 6135 bp, Artificial Sequence
  Synthetic: Fnrs-LeuDH-kivD-padA-brnQ
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacatatgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgacagagccgcatgtagcagtattaagccaggtccaacagtttctcgatcgtcaacacggtctttatattgatggtcgtcctggccccgcacaaagtgaaaaacggttggcgatctttgatccggccaccgggcaagaaattgcgtctactgctgatgccaacgaagcggatgtagataacgcagtcatgtctgcctggcgggcctttgtctcgcgtcgctgggccgggcgattacccgcagagcgtgaacgtattctgctacgttttgctgatctggtggagcagcacagtgaggagctggcgcaactggaaaccctggagcaaggcaagtcaattgccatttcccgtgcttttgaagtgggctgtacgctgaactggatgcgttataccgccgggttaacgaccaaaatcgcgggtaaaacgctggacttgtcgattcccttaccccagggggcgcgttatcaggcctggacgcgtaaagagccggttggcgtagtggcgggaattgtgccatggaactttccgttgatgattggtatgtggaaggtgatgccagcactggcagcaggctgttcaatcgtgattaagccttcggaaaccacgccactgacgatgttgcgcgtggcggaactggccagcgaggctggtatccctgatggcgtttttaatgtcgtcaccgggtcaggtgctgtatgcggcgcggccctgacgtcacatcctcatgttgcgaaaatcagttttaccggttcaaccgcgacgggaaaaggtattgccagaactgctgctgatcacttaacgcgtgtaacgctggaactgggcggtaaaaacccggcaattgtattaaaagatgctgatccgcaatgggttattgaaggcttgatgaccggaagcttcctgaatcaagggcaagtatgcgccgccagttcgcgaatttatattgaagcgccgttgtttgacacgctggttagtggatttgagcaggcggtaaaatcgttgcaagtgggaccggggatgtcacctgttgcacagattaaccctttggtttctcgtgcgcactgcgacaaagtgtgttcattcctcgacgatgcgcaggcacagcaagcagagctgattcgcgggtcgaatggaccagccggagaggggtattatgttgcgccaacgctggtggtaaatcccgatgctaaattgcgcttaactcgtgaagaggtgtttggtccggtggtaaacctggtgcgagtagcggatggagaagaggcgttacaactggcaaacgacacggaatatggcttaactgccagtgtctggacgcaaaatctctcccaggctctggaatatagcgatcgcttacaggcagggacggtgtgggtaaacagccataccttaattgacgctaacttaccgtttggtgggatgaagcagtcaggaacgggccgtgattttggccccgactggctggacggttggtgtgaaactaagtcggtgtgtgtacggtattaataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaatacgcatggcatggatgaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcgtgc

SEQ ID NO: 127 - DNA, 6340 bp, Artificial Sequence
  Synthetic: Ptet-LeuDH-kivD-yqhD-brnQ construct
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtactggaatttggcggtattgaaccaaacccggcttatgaaacgctgatgaacgccgtgaaactggttcgcgaacagaaagtgacgttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagcccgtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctgatcgaagatggtccgaaagccctgaaagagccagaaaactacgatgtgcgcgccaacgtcatgtgggcggcgactcaggcgctgaacggtttgatcggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttcagacgatgagcgtattgacgccgcgattgccgcaacccgcaatttctttgagcaattaggcgtgctgacccacctctccgactacggtctggacggcagctccatcccggctttgctgaaaaaactggaagagcacggcatgacccaactgggcgaaaatcatgacattacgctggatgtcagccgccgtatatacgaagccgcccgctaataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaatacgcatggcatggatgaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcgtgc

SEQ ID NO: 128 - DNA, 5286 bp, Artificial Sequence
  Synthetic: LeuDH-kivD-yqhD-brnQ construct
atgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtactggaatttggcggtattgaaccaaacccggcttatgaaacgctgatgaacgccgtgaaactggttcgcgaacagaaagtgacgttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagcccgtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctgatcgaagatggtccgaaagccctgaaagagccagaaaactacgatgtgcgcgccaacgtcatgtgggcggcgactcaggcgctgaacggtttgatcggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttcagacgatgagcgtattgacgccgcgattgccgcaacccgcaatttctttgagcaattaggcgtgctgacccacctctccgactacggtctggacggcagctccatcccggctttgctgaaaaaactggaagagcacggcatgacccaactgggcgaaaatcatgacattacgctggatgtcagccgccgtatatacgaagccgcccgctaataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaa

SEQ ID NO: 129 - DNA, 5799 bp, Artificial Sequence
  Synthetic: Pfnrs-LeuDH-kivD-yqhD-brnQ construct
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacatatgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtactggaatttggcggtattgaaccaaacccggcttatgaaacgctgatgaacgccgtgaaactggttcgcgaacagaaagtgacgttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagcccgtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctgatcgaagatggtccgaaagccctgaaagagccagaaaactacgatgtgcgcgccaacgtcatgtgggcggcgactcaggcgctgaacggtttgatcggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttcagacgatgagcgtattgacgccgcgattgccgcaacccgcaatttctttgagcaattaggcgtgctgacccacctctccgactacggtctggacggcagctccatcccggctttgctgaaaaaactggaagagcacggcatgacccaactgggcgaaaatcatgacattacgctggatgtcagccgccgtatatacgaagccgcccgctaataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaatacgcatggcatggatgaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcgtgc

SEQ ID NO: 130 - DNA, 60 bp, Artificial Sequence
  Synthetic: SR36 Primer
tagaactgatgcaaaaagtgctcgacgaaggcacacagatgtgtaggctggagctgcttc

SEQ ID NO: 131 - DNA, 60 bp, Artificial Sequence
  Synthetic: SR38 Primer
gtttcgtaattagatagccaccggcgctttaatgcccggacatatgaatatcctccttag

SEQ ID NO: 132 - DNA, 52 bp, Artificial Sequence
  Synthetic: SR33 Primer
caacacgtttcctgaggaaccatgaaacagtatttagaactgatgcaaaaag

SEQ ID NO: 133 - DNA, 46 bp, Artificial Sequence
  Synthetic: SR34 Primer
cgcacactggcgtcggctctggcaggatgtttcgtaattagatagc

SEQ ID NO: 134 - DNA, 36 bp, Artificial Sequence
  Synthetic: SR43 Primer
atatcgtcgcagcccacagcaacacgtttcctgagg

SEQ ID NO: 135 - DNA, 47 bp, Artificial Sequence
  Synthetic: SR44 Primer
aagaatttaacggagggcaaaaaaaaccgacgcacactggcgtcggc

SEQ ID NO: 136 - DNA, 3383 bp, Artificial Sequence
  Synthetic: Nucleotide sequence of Pfnr1-lacZ construct, low-copy
ggtaccgtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaaatgagcaaagtcagactcgcaattatggatcctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 137 - DNA, 3258 bp, Artificial Sequence
  Synthetic: Nucleotide sequences of Pfnr2-lacZ construct, low-copy
ggtacccatttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacctatgacgacctacgatcgggatcctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 138 - DNA, 3386 bp, Artificial Sequence
  Synthetic: Nucleotide sequences of Pfnr3-lacZ construct, low-copy
ggtaccgtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacatatgactatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 139 - DNA, 3261 bp, Artificial Sequence
  Synthetic: Nucleotide sequences of Pfnr4-lacZ construct, low-copy
ggtacccatttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacatatgactatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 140 - DNA, 3279 bp, Artificial Sequence
  Synthetic: Nucleotide sequences of Pfnrs-lacZ construct, low-copy
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacatatgctatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 141 - DNA, 967 bp, Artificial Sequence
  Synthetic: Wild-type clbA
caaatatcacataatcttaacatatcaataaacacagtaaagtttcatgtgaaaaacatcaaacataaaatacaagctcggaatacgaatcacgctatacacattgctaacaggaatgagattatctaaatgaggattgatatattaattggacatactagtttttttcatcaaaccagtagagataacttccttcactatctcaatgaggaagaaataaaacgctatgatcagtttcattttgtgagtgataaagaactctatattttaagccgtatcctgctcaaaacagcactaaaaagatatcaacctgatgtctcattacaatcatggcaatttagtacgtgcaaatatggcaaaccatttatagtttttcctcagttggcaaaaaagattttttttaacctttcccatactatagatacagtagccgttgctattagttctcactgcgagcttggtgtcgatattgaacaaataagagatttagacaactcttatctgaatatcagtcagcatttttttactccacaggaagctactaacatagtttcacttcctcgttatgaaggtcaattacttttttggaaaatgtggacgctcaaagaagcttacatcaaatatcgaggtaaaggcctatctttaggactggattgtattgaatttcatttaacaaataaaaaactaacttcaaaatatagaggttcacctgtttatttctctcaatggaaaatatgtaactcatttctcgcattagcctctccactcatcacccctaaaataactattgagctatttcctatgcagtcccaactttatcaccacgactatcagctaattcattcgtcaaatgggcagaattgaatcgccacggataatctagacacttctgagccgtcgataatattgattttcatattccgtcggtggtgtaagtatcccgcataatcgtgccattcacatttag

SEQ ID NO: 142 - DNA, 424 bp, Artificial Sequence
  Synthetic: clbA knockout
ggatggggggaaacatggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaaatgaggattgatgtgtaggctggagctgcttcgaagttcctatactttctagagaataggaacttcggaataggaacttcggaataggaactaaggaggatattcatatgtcgtcaaatgggcagaattgaatcgccacggataatctagacacttctgagccgtcgataatattgattttcatattccgtcggtgg

SEQ ID NO: 143 - DNA, 200 bp, Artificial Sequence
  Synthetic: FNR-responsive regulatory region Sequence: fnrS+crp
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcaaatgtgatctagttcacattttttgtttaactttaagaaggagatatacat

SEQ ID NO: 144 - DNA, 18 bp, Artificial Sequence
  Synthetic: consensus sequence
ttgttgayryrtcaacwa

SEQ ID NO: 145 - DNA, 1278 bp, Artificial Sequence
  Description of Artificial Sequence: Synthetic polynucleotide
atgaacacacctagtgaagtaaagaaggatttgctgggtgttgcagaacatttacgtccgtcggagcccattacgctgggtccaggtgcgagtgcttggcagctggtattgaagaagatcaaggagttgagtattagcggtcgtcaagacgctttcatggttgcagaccttgatgtccttgtgtctcgtcatcggacgttcttacaagcactgcctagagtacaacccttctatgcagtaaagtgcaatagtaacccatgggtgttacttgtgttggcagctcttggcacgggatttgattgtgcttctcagggagaattggagcaagttttgggcttgggtgtagcgccgtcacggataatcttcgcaaatccctgtaaagcagtcagccacattcagtttgcagctcggtgcggtgtgcaattgttgacattcgacaatgaagaggagttaatcaagttagcgcgttaccatccacgtgcacggttggtgcttcggattcaaaccctggactcacaatcaacgttcccacttcataccaagttcggtgctcacttagaagcatgtggacaccttctgcaggttgccagagagctgggtcttgccgtggtaggtgctagctttcatgtaggaagcgactgccacacacctgagagttaccgtcaggccatcgcagattgtcatcgtgtgttcgagatgggctgtaaggcaggtcatcacatgtcgcttcttgatttgggtggagggttcccaggtgtgaaaggttccgaggcgaagtttgaggaggttgcaagagtaatcaataccgctcttgctcagtactttccggaagagactggcatcgaggtgatcgcggaacctggtcgtttctacgctgggtcggtgtgcactgcagctgtgaacatcatcgtaaagaagtctagtttggacccaggtggtcatcgtaagcttgcttactaccttaatgaaggacattacggtgtattcagattgttcttgcgtgatccagtgcctcgtattcccatcgtggtgaaagagttcccatccgaaccaccactgtttccttgcactttgtacggtcccacatgtgacgcctatgatcggttgttttccaccgaggtacaattgccagagctggatgttggagattggttgatcttcccagatatgggtgcctattcgtcctcaatgtcctcgaccttcaacggatttccaatagccaccgtgtatgatgcaatgtcaccgcagttacgctccctgttggagactgtaccataa

SEQ ID NO: 146 - DNA, 290 bp, Artificial Sequence; identical to SEQ ID NO: 14
  Synthetic: FNR Responsive Promoter

SEQ ID NO: 147 - DNA, 173 bp, Artificial Sequence; identical to SEQ ID NO: 15
  Synthetic: FNR Responsive Promoter

SEQ ID NO: 148 - DNA, 305 bp, Artificial Sequence; identical to SEQ ID NO: 16
  Synthetic: FNR Responsive Promoter

SEQ ID NO: 149 - DNA, 180 bp, Artificial Sequence; identical to SEQ ID NO: 17
  Synthetic: FNR Responsive Promoter

SEQ ID NO: 150 - DNA, 199 bp, Artificial Sequence; identical to SEQ ID NO: 18
  Synthetic: FNR Responsive Promoter

SEQ ID NO: 151 - DNA, 117 bp, Artificial Sequence; identical to SEQ ID NO: 80
  Synthetic: FNR-responsive regulatory region Sequence

SEQ ID NO: 152 - DNA, 108 bp, Artificial Sequence; identical to SEQ ID NO: 81
  Synthetic: FNR-responsive regulatory region Sequence

SEQ ID NO: 153 - DNA, 290 bp, Artificial Sequence; identical to SEQ ID NO: 14
  Synthetic: FNR-responsive regulatory region Sequence: nirB1

SEQ ID NO: 154 - DNA, 433 bp, Artificial Sequence; identical to SEQ ID NO: 83
  Synthetic: FNR-responsive regulatory region Sequence: nirB2

SEQ ID NO: 155 - DNA, 290 bp, Artificial Sequence; identical to SEQ ID NO: 84
  Synthetic: FNR-responsive regulatory region Sequence: nirB3

SEQ ID NO: 156 - DNA, 173 bp, Artificial Sequence; identical to SEQ ID NO: 15
  Synthetic: FNR-responsive regulatory region Sequence: ydfZ

SEQ ID NO: 157 - DNA, 305 bp, Artificial Sequence; identical to SEQ ID NO: 16
  Synthetic: FNR-responsive regulatory region Sequence: nirB+RBS

SEQ ID NO: 158 - DNA, 180 bp, Artificial Sequence; identical to SEQ ID NO: 17
  Synthetic: FNR-responsive regulatory region Sequence: ydfZ+RBS

SEQ ID NO: 159 - DNA, 199 bp, Artificial Sequence; identical to SEQ ID NO: 18
  Synthetic: FNR-responsive regulatory region Sequence: fnrS1

SEQ ID NO: 160 - DNA, 207 bp, Artificial Sequence; identical to SEQ ID NO: 89
  Synthetic: FNR-responsive regulatory region Sequence: fnrS2

SEQ ID NO: 161 - DNA, 390 bp, Artificial Sequence; identical to SEQ ID NO: 90
  Synthetic: FNR-responsive regulatory region Sequence: nirB+crp

SEQ ID NO: 162 - DNA, 200 bp, Artificial Sequence; identical to SEQ ID NO: 143
  Synthetic: FNR-responsive regulatory region Sequence: fnrS+crp

SEQ ID NO: 163 - DNA, 355 bp, Artificial Sequence
  Synthetic: Regulatory sequence: katG
tgtggcttttatgaaaatcacacagtgatcacaaattttaaacagagcacaaaatgctgcctcgaaatgagggcgggaaaataaggttatcagccttgttttctccctcattacttgaaggatatgaagctaaaacccttttttataaagcatttgtccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagaggggagcacattgatgcgaattcattaaagaggagaaaggtacc

SEQ ID NO: 164 - DNA, 228 bp, Artificial Sequence
  Synthetic: Regulatory sequence: dps
ttccgaaaattcctggcgagcagataaataagaattgttcttatcaatatatctaactcattgaatctttattagttttgtttttcacgcttgttaccactattagtgtgataggaacagccagaatagcggaacacatagccggtgctatacttaatctcgttaattactgggacataacatcaagaggatatgaaattcgaattcattaaagaggagaaaggtacc

SEQ ID NO: 165 - DNA, 334 bp, Artificial Sequence
  Synthetic: Regulatory sequence: ahpC
gcttagatcaggtgattgccctttgtttatgagggtgttgtaatccatgtcgttgttgcatttgtaagggcaacacctcagcctgcaggcaggcactgaagataccaaagggtagttcagattacacggtcacctggaaagggggccattttactttttatcgccgctggcggtgcaaagttcacaaagttgtcttacgaaggttgtaaggtaaaacttatcgatttgataatggaaacgcattagccgaatcggcaaaaattggttaccttacatctcatcgaaaacacggaggaagtatagatgcgaattcattaaagaggagaaaggtacc

SEQ ID NO: 166 - DNA, 134 bp, Artificial Sequence
  Synthetic: Regulatory sequence: oxyS
ctcgagttcattatccatcctccatcgccacgatagttcatggcgataggtagaatagcaatgaacgattatccctatcaagcattctgactgataattgctcacacgaattcattaaagaggagaaaggtacc

SEQ ID NO: 167 - protein, 425 aa, Artificial Sequence
  Description of Artificial Sequence: Synthetic polypeptide
MNTPSEVKKDLLGVAEHLRPSEPITLGPGASAWQLVLKKIKELSISGRQDAFMVADLDVLVSRHRTFLQALPRVQPFYAVKCNSNPWVLLVLAALGTGFDCASQGELEQVLGLGVAPSRIIFANPCKAVSHIQFAARCGVQLLTFDNEEELIKLARYHPRARLVLRIQTLDSQSTFPLHTKFGAHLEACGHLLQVARELGLAVVGASFHVGSDCHTPESYRQAIADCHRVFEMGCKAGHHMSLLDLGGGFPGVKGSEAKFEEVARVINTALAQYFPEETGIEVIAEPGRFYAGSVCTAAVNIIVKKSSLDPGGHRKLAYYLNEGHYGVFRLFLRDPVPRIPIVVKEFPSEPPLFPCTLYGPTCDAYDRLFSTEVQLPELDVGDWLIFPDMGAYSSSMSSTFNGFPIATVYDAMSPQLRSLLETVP

SEQ ID NO: 168 - DNA, 18 bp, Artificial Sequence
  Description of Artificial Sequence: Synthetic consensus oligonucleotide
ttgttgayryrtcaacwa

SEQ ID NO: 169 - DNA, 15 bp, Artificial Sequence
  Description of Artificial Sequence: Synthetic consensus oligonucleotide
  modified_base LOCATION: (8)..(8): a, c, t or g
ttataatnattataa